(19) United States
(12) Patent Application Publication
(10) Pub. No.: US 2025/0223358 A1
(43) Pub. Date: Jul. 10, 2025

(54) NOVEL ANTI-CD3 ANTIBODIES AND USES THEREOF

(71) Applicant: ANTENGENE BIOLOGICS LIMITED, Shanghai (CN)

(72) Inventors: Hui YUWEN, Shanghai (CN); Yijing REN, Shanghai (CN); Tengteng LI, Shanghai (CN); Peng CHEN, Shanghai (CN); Bing HOU, Shanghai (CN); Bo SHAN, Shanghai (CN); Jay MEI, Shanghai (CN)

(21) Appl. No.: 18/844,977

(22) PCT Filed: Mar. 7, 2023
(86) PCT No.: PCT/CN2023/080090

§ 371 Date: Sep. 9, 2024

(30) Foreign Application Priority Data
Mar. 9, 2022 (WO) PCT/CN2022/079943
Sep. 29, 2022 (WO) PCT/CN2022/122679
Feb. 22, 2023 (WO) PCT/CN2023/077693

(51) Int. Cl. C07K 16/28; A61K 40/11; A61K 40/31; A61K 40/42; A61K 45/06; C07K 14/725; G01N 33/577

(52) U.S. Cl. CPC C07K 16/2809; A61K 40/11; A61K 40/31; A61K 40/421; A61K 45/06; C07K 14/7051; G01N 33/577; A61K 2239/13; A61K 2239/21; A61K 2239/22; C07K 2317/24; C07K 2317/31; C07K 2317/52; C07K 2317/565; C07K 2317/622; C07K 2317/92; C07K 2319/02; C07K 2319/03; G01N 2333/7051

(57) ABSTRACT

Provided are anti-CD3 antibodies or antigen-binding fragments thereof, isolated polynucleotides encoding the same, pharmaceutical composition comprising the same and the uses thereof.

### FIELD OF THE INVENTION

The present disclosure generally relates to novel anti-CD3 antibodies, antigen-binding fragments thereof, and uses of the same.

### BACKGROUND

The CD3 (cluster of differentiation 3) T-cell co-receptor is a protein complex and is composed of four distinct chains, a CD3gamma chain, a CD3delta chain, and two CD3epsilon chains. These chains associate with a molecule known as the T-cell receptor (TCR) and the zeta-chain to generate activation signal in T lymphocytes. The TCR, zeta-chain, and CD3 molecules together form the TCR-CD3 complex, in which TCR as a subunit recognizes and binds to antigen, and CD3 as a subunit transfers and conveys the antigen-stimulation to signaling pathway, and ultimately regulates T-cell activity. The CD3 protein is virtually present in all T cells.

Mouse monoclonal antibodies specific for human CD3, such as OKT3 (Kung et al., (1979) Science 206:347-9), were the first generation CD3 antibodies for treatment. Although OKT3 has strong immunosuppressive potency, its clinical use was hampered by serious side effects linked to its immunogenic and mitogenic potentials (Chatenoud (2003) Nature Reviews Immunology 3:123-132). OKT3 induced an anti-globulin response, promoting its own rapid clearance and neutralization (Chatenoud et al., (1982) Eur. J. Immunol. 137:830-8). In addition, OKT3 induced T-cell proliferation and cytokine production in vitro, and led to a large scale release of cytokine in vivo (Hirsch et al., (1989) J. Immunol 142:737-43). The cytokine release (also referred to as “cytokine storm”) in turn led to a “flu-like” syndrome, characterized by fever, chills, headaches, nausea, vomiting, diarrhea, respiratory distress, septic meningitis and hypotension (Chatenoud (2003) Nature Reviews Immunology 3:123-132). Such serious side effects limited the more widespread use of OKT3 in transplantation as well as the extension of its use to other clinical fields such as autoimmunity.

A more recent application of CD3 antibodies is in the form of bispecific antibodies, binding CD3 on the one hand and a tumor cell antigen on the other hand. The simultaneous binding of such an antibody to both of its targets will force a temporary interaction between target cell and T cell, causing activation of any cytotoxic T cell and subsequent lysis of the target cell.

Needs remain for novel anti-CD3 antibodies.

### SUMMARY OF THE INVENTION

Throughout the present disclosure, the articles “a”, “an”, and “the” are used herein to refer to one or to more than one (i.e., to at least one) of the grammatical object of the article. By way of example, “an antibody” means one antibody or more than one antibody.

In one respect, the present disclosure provides an antibody or antigen-binding fragment thereof which specifically binds to CD3, comprising:
one or two or three heavy chain complementarity determining regions (HCDR1, HCDR2 and/or HCDR3) contained within any one of the heavy chain variable (VH) region sequences selected from the group consisting of SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 and 245; and/or
one or two or three light chain complementarity determining regions (LCDR1, LCDR2 and/or LCDR3) contained within any one of the light chain variable (VL) region sequences selected from the group consisting of SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 and 232.

In some embodiments, the antibody or antigen-binding fragment thereof of the present disclosure comprises at least one heavy or light chain complementarity determining region (CDR) comprising an amino acid sequence selected from the group consisting of SEQ ID NOs: 1, 2, 3, 4, 5, 6, 9, 10, 11, 12, 13, 14, 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 81, 82, 83, 84, 85, 86, 89, 90, 91, 92, 93, 94, 102, 103, 104, 105, 106, 107, 110, 111, 112, 113, 114, 115, 118, 119, 120, 121, 122, 123, 126, 127, 128, 129, 130, 131, 134, 135, 136, 137, 138, 139, 142, 143, 144, 145, 146, 147, 150, 151, 152, 153, 154, 155, 171, 172, 173, 174, 175, 176, 177, 178, 179, 180, 181, 182, 183, 184, 185, 201, 162, 167, 210, 211, 212, 214, 215, 216, 217, 218, 219, 220, 221, 222, 223, 224, 225, 226, 227, 228 and 229.

In some embodiments, the antibody or antigen-binding fragment thereof of the present disclosure comprises a VH region comprising one or two or three of HCDR1, HCDR2 and HCDR3 comprising an amino acid sequence selected from the group consisting of SEQ ID NOs: 1, 2, 3, 9, 10, 11, 17, 18, 19, 25, 26, 27, 33, 34, 35, 41, 42, 43, 49, 50, 51, 57, 58, 59, 65, 66, 67, 73, 74, 75, 81, 82, 83, 89, 90, 91, 102, 103, 104, 110, 111, 112, 118, 119, 120, 126, 127, 128, 134, 135, 136, 142, 143, 144, 150, 151, 152, 171, 172, 173, 174, 175, 176, 177, 178, 179, 180, 181, 201, 162, 210, 211, 212, 217, 218, 219, 220, 221, 222, 223, 224, 225, 226, 227, 228 and 229.

In some embodiments, the antibody or antigen-binding fragment thereof of the present disclosure comprises a VL region comprising one or two or three of LCDR1, LCDR2 and LCDR3 comprising an amino acid sequence selected from the group consisting of SEQ ID NOs: 4, 5, 6, 12, 13, 14, 20, 21, 22, 28, 29, 30, 36, 37, 38, 44, 45, 46, 52, 53, 54, 60, 61, 62, 68, 69, 70, 76, 77, 78, 84, 85, 86, 92, 93, 94, 105, 106, 107, 113, 114, 115, 121, 122, 123, 129, 130, 131, 137, 138, 139, 145, 146, 147, 153, 154, 155, 182, 183, 184, 185, 167, 214, 215 and 216.

In some embodiments, the antibody or antigen-binding fragment thereof of the present disclosure comprises:
(a) a HCDR1 comprising an amino acid sequence selected from the group consisting of SEQ ID NOs: 1, 9, 17, 25, 33, 41, 49, 57, 65, 73, 81, 89, 102, 110, 118, 126, 134, 142, 150, 217, 218 and 219;
(b) a HCDR2 comprising an amino acid sequence selected from the group consisting of SEQ ID NOs: 2, 10, 18, 26, 34, 42, 50, 58, 66, 74, 82, 90, 103, 111, 119, 127, 135, 143, 151, 171, 172, 173, 201, 210, 211, 212, 220 and 222; and
(c) a HCDR3 comprising an amino acid sequence selected from the group consisting of SEQ ID NOs: 3, 11, 19, 27, 35, 43, 51, 59, 67, 75, 83, 91, 104, 112, 120, 128, 136, 144, 152, 174, 175, 176, 177, 178, 179, 180, 181, 221, 223, 224, 225, 226, 227, 228, 229 and 162.

In some embodiments, the antibody or antigen-binding fragment thereof of the present disclosure comprises:
(a) a LCDR1 comprising an amino acid sequence selected from the group consisting of SEQ ID NOs: 4, 12, 20, 28, 36, 44, 52, 60, 68, 76, 84, 92, 105, 113, 121, 129, 137, 145 and 153;
(b) a LCDR2 comprising an amino acid sequence selected from the group consisting of SEQ ID NOs: 5, 13, 21, 29, 37, 45, 53, 61, 69, 77, 85, 93, 106, 114, 122, 130, 138, 146 and 154; and
(c) a LCDR3 comprising an amino acid sequence selected from the group consisting of SEQ ID NOs: 6, 14, 22, 30, 38, 46, 54, 62, 70, 78, 86, 94, 107, 115, 123, 131, 139, 147, 155, 182, 183, 184, 185, 214, 215, 216 and 167.

In some embodiments, the antibody or antigen-binding fragment thereof of the present disclosure comprises:
(a) a HCDR1 comprising an amino acid sequence as set forth in SEQ ID NO: 1, a HCDR2 comprising an amino acid sequence as set forth in SEQ ID NO: 2, and a HCDR3 comprising an amino acid sequence as set forth in SEQ ID NO: 3;
(b) a HCDR1 comprising an amino acid sequence as set forth in SEQ ID NO: 9, a HCDR2 comprising an amino acid sequence as set forth in SEQ ID NO: 10, and a HCDR3 comprising an amino acid sequence as set forth in SEQ ID NO: 11;
(c) a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
(d) a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
(e) a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
(f) a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
(g) a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
(h) a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
(i) a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
(j) a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
(k) a HCDR1 comprising an amino acid sequence as set forth in SEQ ID NO: 81, a HCDR2 comprising an amino acid sequence as set forth in SEQ ID NO: 82, and a HCDR3 comprising an amino acid sequence as set forth in SEQ ID NO: 83;
(l) a HCDR1 comprising an amino acid sequence as set forth in SEQ ID NO: 89, a HCDR2 comprising an amino acid sequence as set forth in SEQ ID NO: 90, and a HCDR3 comprising an amino acid sequence as set forth in SEQ ID NO: 91;
(m) a HCDR1 comprising an amino acid sequence as set forth in SEQ ID NO: 102, a HCDR2 comprising an amino acid sequence as set forth in SEQ ID NO: 103, and a HCDR3 comprising an amino acid sequence as set forth in SEQ ID NO: 104;
(n) a HCDR1 comprising an amino acid sequence as set forth in SEQ ID NO: 110, a HCDR2 comprising an amino acid sequence as set forth in SEQ ID NO: 111, and a HCDR3 comprising an amino acid sequence as set forth in SEQ ID NO: 112;
(o) a HCDR1 comprising an amino acid sequence as set forth in SEQ ID NO: 118, a HCDR2 comprising an amino acid sequence as set forth in SEQ ID NO: 119, and a HCDR3 comprising an amino acid sequence as set forth in SEQ ID NO: 120;
(p) a HCDR1 comprising an amino acid sequence as set forth in SEQ ID NO: 126, a HCDR2 comprising an amino acid sequence as set forth in SEQ ID NO: 127, and a HCDR3 comprising an amino acid sequence as set forth in SEQ ID NO: 128;
(q) a HCDR1 comprising an amino acid sequence as set forth in SEQ ID NO: 134, a HCDR2 comprising an amino acid sequence as set forth in SEQ ID NO: 135, and a HCDR3 comprising an amino acid sequence as set forth in SEQ ID NO: 136;
(r) a HCDR1 comprising an amino acid sequence as set forth in SEQ ID NO: 142, a HCDR2 comprising an amino acid sequence as set forth in SEQ ID NO: 143, and a HCDR3 comprising an amino acid sequence as set forth in SEQ ID NO: 144;
(s) a HCDR1 comprising an amino acid sequence as set forth in SEQ ID NO: 150, a HCDR2 comprising an amino acid sequence as set forth in SEQ ID NO: 151, and a HCDR3 comprising an amino acid sequence as set forth in SEQ ID NO: 152;
(t) a HCDR1 comprising an amino acid sequence as set forth in SEQ ID NO: 219, a HCDR2 comprising an amino acid sequence as set forth in SEQ ID NO: 201, and a HCDR3 comprising an amino acid sequence as set forth in SEQ ID NO: 162;
(u) a HCDR1 comprising an amino acid sequence as set forth in SEQ ID NO: 9, 217 or 218; a HCDR2 comprising an amino acid sequence as set forth in SEQ ID NO: 10, 171, 172, 173, 220 or 222; a HCDR3 comprising an amino acid sequence as set forth in SEQ ID NO: 11, 174, 175, 176, 177, 178, 179, 180, 181, 221, 223, 224, 225, 226, 227, 228 or 229;
(v) a HCDR1 comprising an amino acid sequence as set forth in SEQ ID NO: 150, a HCDR2 comprising an amino acid sequence as set forth in SEQ ID NO: 212, and a HCDR3 comprising an amino acid sequence as set forth in SEQ ID NO: 152;
(w) a HCDR1 comprising an amino acid sequence as set forth in SEQ ID NO: 150, a HCDR2 comprising an amino acid sequence as set forth in SEQ ID NO: 210, and a HCDR3 comprising an amino acid sequence as set forth in SEQ ID NO: 152; or
(x) a HCDR1 comprising an amino acid sequence as set forth in SEQ ID NO: 150, a HCDR2 comprising an amino acid sequence as set forth in SEQ ID NO: 211, and a HCDR3 comprising an amino acid sequence as set forth in SEQ ID NO: 152.

In some embodiments, the antibody or antigen-binding fragment thereof of the present disclosure comprises:
(a) a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
(b) a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(c) a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
(d) a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
(e) a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
(f) a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
(g) a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
(h) a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
(i) a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
(j) a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
(k) a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
(l) a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
(m) a LCDR1 comprising an amino acid sequence as set forth in SEQ ID NO: 105, a LCDR2 comprising an amino acid sequence as set forth in SEQ ID NO: 106, and a LCDR3 comprising an amino acid sequence as set forth in SEQ ID NO: 107;
(n) a LCDR1 comprising an amino acid sequence as set forth in SEQ ID NO: 113, a LCDR2 comprising an amino acid sequence as set forth in SEQ ID NO: 114, and a LCDR3 comprising an amino acid sequence as set forth in SEQ ID NO: 115;
(o) a LCDR1 comprising an amino acid sequence as set forth in SEQ ID NO: 121, a LCDR2 comprising an amino acid sequence as set forth in SEQ ID NO: 122, and a LCDR3 comprising an amino acid sequence as set forth in SEQ ID NO: 123;
(p) a LCDR1 comprising an amino acid sequence as set forth in SEQ ID NO: 129, a LCDR2 comprising an amino acid sequence as set forth in SEQ ID NO: 130, and a LCDR3 comprising an amino acid sequence as set forth in SEQ ID NO: 131;
(q) a LCDR1 comprising an amino acid sequence as set forth in SEQ ID NO: 137, a LCDR2 comprising an amino acid sequence as set forth in SEQ ID NO: 138, and a LCDR3 comprising an amino acid sequence as set forth in SEQ ID NO: 139;
(r) a LCDR1 comprising an amino acid sequence as set forth in SEQ ID NO: 145, a LCDR2 comprising an amino acid sequence as set forth in SEQ ID NO: 146, and a LCDR3 comprising an amino acid sequence as set forth in SEQ ID NO: 147;
(s) a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
(t) a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 167; or
(u) a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14, 182, 183, 184, 185, 214, 215 or 216.

In some embodiments, the antibody or antigen-binding fragment thereof of the present disclosure comprises:
(a) a HCDR1 comprising an amino acid sequence as set forth in SEQ ID NO: 1, a HCDR2 comprising an amino acid sequence as set forth in SEQ ID NO: 2, a HCDR3 comprising an amino acid sequence as set forth in SEQ ID NO: 3, a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
(b) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(c) a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
(d) a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
(e) a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
(f) a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
(g) a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
(h) a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
(i) a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
(j) a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
(k) a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
(l) a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
(m) a HCDR1 comprising an amino acid sequence as set forth in SEQ ID NO: 102, a HCDR2 comprising an amino acid sequence as set forth in SEQ ID NO: 103, a HCDR3 comprising an amino acid sequence as set forth in SEQ ID NO: 104, a LCDR1 comprising an amino acid sequence as set forth in SEQ ID NO: 105, a LCDR2 comprising an amino acid sequence as set forth in SEQ ID NO: 106, and a LCDR3 comprising an amino acid sequence as set forth in SEQ ID NO: 107;
(n) a HCDR1 comprising an amino acid sequence as set forth in SEQ ID NO: 110, a HCDR2 comprising an amino acid sequence as set forth in SEQ ID NO: 111, a HCDR3 comprising an amino acid sequence as set forth in SEQ ID NO: 112, a LCDR1 comprising an amino acid sequence as set forth in SEQ ID NO: 113, a LCDR2 comprising an amino acid sequence as set forth in SEQ ID NO: 114, and a LCDR3 comprising an amino acid sequence as set forth in SEQ ID NO: 115;
(o) a HCDR1 comprising an amino acid sequence as set forth in SEQ ID NO: 118, a HCDR2 comprising an amino acid sequence as set forth in SEQ ID NO: 119, a HCDR3 comprising an amino acid sequence as set forth in SEQ ID NO: 120, a LCDR1 comprising an amino acid sequence as set forth in SEQ ID NO: 121, a LCDR2 comprising an amino acid sequence as set forth in SEQ ID NO: 122, and a LCDR3 comprising an amino acid sequence as set forth in SEQ ID NO: 123;
(p) a HCDR1 comprising an amino acid sequence as set forth in SEQ ID NO: 126, a HCDR2 comprising an amino acid sequence as set forth in SEQ ID NO: 127, a HCDR3 comprising an amino acid sequence as set forth in SEQ ID NO: 128, a LCDR1 comprising an amino acid sequence as set forth in SEQ ID NO: 129, a LCDR2 comprising an amino acid sequence as set forth in SEQ ID NO: 130, and a LCDR3 comprising an amino acid sequence as set forth in SEQ ID NO: 131;
(q) a HCDR1 comprising an amino acid sequence as set forth in SEQ ID NO: 134, a HCDR2 comprising an amino acid sequence as set forth in SEQ ID NO: 135, a HCDR3 comprising an amino acid sequence as set forth in SEQ ID NO: 136, a LCDR1 comprising an amino acid sequence as set forth in SEQ ID NO: 137, a LCDR2 comprising an amino acid sequence as set forth in SEQ ID NO: 138, and a LCDR3 comprising an amino acid sequence as set forth in SEQ ID NO: 139;
(r) a HCDR1 comprising an amino acid sequence as set forth in SEQ ID NO: 142, a HCDR2 comprising an amino acid sequence as set forth in SEQ ID NO: 143, a HCDR3 comprising an amino acid sequence as set forth in SEQ ID NO: 144, a LCDR1 comprising an amino acid sequence as set forth in SEQ ID NO: 145, a LCDR2 comprising an amino acid sequence as set forth in SEQ ID NO: 146, and a LCDR3 comprising an amino acid sequence as set forth in SEQ ID NO: 147;
(s) a HCDR1 comprising an amino acid sequence as set forth in SEQ ID NO: 150, a HCDR2 comprising an amino acid sequence as set forth in SEQ ID NO: 151, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
(t) a HCDR1 comprising an amino acid sequence as set forth in SEQ ID NO: 9, a HCDR2 comprising an amino acid sequence as set forth in SEQ ID NO: 171, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(u) a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(v) a HCDR1 comprising an amino acid sequence as set forth in SEQ ID NO: 9, a HCDR2 comprising an amino acid sequence as set forth in SEQ ID NO: 173, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(w) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 174, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(x) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 175, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(y) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 176, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(z) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 177, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(aa) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 178, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(bb) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 179, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(cc) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(dd) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 181, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(ee) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 182;
(ff) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 183;
(gg) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 184; or
(hh) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 185;
(ii) a HCDR1 comprising an amino acid sequence as set forth in SEQ ID NO: 9, a HCDR2 comprising an amino acid sequence as set forth in SEQ ID NO: 220, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(jj) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 221, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(kk) a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(ll) a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(mm) a HCDR1 comprising an amino acid sequence as set forth in SEQ ID NO: 9, a HCDR2 comprising an amino acid sequence as set forth in SEQ ID NO: 171, a HCDR3 comprising an amino acid sequence as set forth in SEQ ID NO: 224, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(nn) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
(oo) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
(pp) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 216;
(qq) a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
(rr) a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
(ss) a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
(tt) a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
(uu) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 225, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(vv) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 226, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(ww) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 227, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(xx) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 228, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(yy) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 174, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(zz) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 229, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(aaa) a HCDR1 comprising an amino acid sequence as set forth in SEQ ID NO: 217, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(bbb) a HCDR1 comprising an amino acid sequence as set forth in SEQ ID NO: 218, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(ccc) a HCDR1 comprising an amino acid sequence as set forth in SEQ ID NO: 219, a HCDR2 comprising an amino acid sequence as set forth in SEQ ID NO: 201, a HCDR3 comprising an amino acid sequence as set forth in SEQ ID NO: 162, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 167;
(ddd) a HCDR1 comprising an amino acid sequence as set forth in SEQ ID NO: 150, a HCDR2 comprising an amino acid sequence as set forth in SEQ ID NO: 212, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
(eee) a HCDR1 comprising an amino acid sequence as set forth in SEQ ID NO: 150, a HCDR2 comprising an amino acid sequence as set forth in SEQ ID NO: 210, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155; or
(fff) a HCDR1 comprising an amino acid sequence as set forth in SEQ ID NO: 150, a HCDR2 comprising an amino acid sequence as set forth in SEQ ID NO: 211, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155.

In some embodiments, the antibody or antigen-binding fragment thereof of the present disclosure comprises a VH region having an amino acid sequence as set forth in SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 or 245, or a homologous sequence thereof having at least 80% sequence identity to SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 or 245.

In some embodiments, the antibody or antigen-binding fragment thereof of the present disclosure comprises a VL region having an amino acid sequence as set forth in SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 or 232, or a homologous sequence thereof having at least 80% sequence identity to SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 or 232.

In some embodiments, the antibody or antigen-binding fragment thereof of the present disclosure comprises a VH/VL amino acid sequence pair selected from the group consisting of SEQ ID NOs: 7/8, 15/16, 23/24, 31/32, 39/40, 47/48, 55/56, 63/64, 71/72, 79/80, 87/88, 95/96, 108/109, 116/117, 124/125, 132/133, 140/141, 148/149, 156/157, 163/168, 163/169, 163/170, 164/168, 164/169, 164/170, 165/168, 165/169, 165/170, 166/168, 166/169, 166/170, 186/169, 187/169, 188/169, 189/169, 190/169, 191/169, 192/169, 193/169, 194/169, 195/169, 196/169, 165/197, 165/198, 165/199, 165/200, 202/207, 202/208, 202/209, 203/207, 203/208, 203/209, 204/207, 205/207, 206/207, 206/208, 206/209, 233/169, 234/169, 235/169, 236/169, 237/169, 238/169, 239/169, 240/169, 241/169, 242/169, 243/169, 244/169, 245/169, 165/230, 165/231, 165/232, 235/230, 235/231, 236/230 and 236/231.

In some embodiments, the antibody or antigen-binding fragment thereof of the present disclosure further comprises one or more amino acid residue substitutions or modifications yet retains specific binding affinity to CD3. In some embodiments, at least one of the substitutions or modifications is in one or more of the CDR sequences of the VH region or VL region. In some embodiments, at least one of the substitutions or modifications is in one or more of the non-CDR sequences of the VH region or VL region. In some embodiments, the antibody or antigen-binding fragment thereof of the present disclosure further comprises one or more non-natural amino acid (NNAA) substitution. In some embodiments, the NNAA is capable of being conjugated.

In some embodiments, the antibody or antigen-binding fragment thereof of the present disclosure has one or more binding properties to CD3 selected from the group consisting of:
(a) being capable of specifically binding to human CD3 as measured by FACS assay;
(b) being with a T cell activation capability as measured by Jurkat NFAT-Luciferase activation assay; and
(c) being with a PBMC activation capability as measured by ELISA assay.

In another aspect, the present disclosure provides an antibody or antigen-binding fragment thereof, which competes for binding to CD3 with the antibody or antigen-binding fragment thereof as described above.

In some embodiments, the antibody or antigen-binding fragment thereof of the present disclosure is a chimeric, a humanized or a human antibody or antigen-binding fragment thereof.

In some embodiments, the antibody or antigen-binding fragment thereof of the present disclosure is a labeled antibody, a bivalent antibody, an anti-idiotypic antibody or a fusion protein.

In some embodiments, the antibody or antigen-binding fragment thereof of the present disclosure is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

In some embodiments, the antibody or antigen-binding fragment thereof of the present disclosure further comprises an Fc region. In some embodiments, the Fc region is an Fc region of human immunoglobulin (Ig). In some embodiments, the Fc region is an Fc region of human IgG. In some embodiments, the Fc region is derived from human IgG1, IgG2, IgG3, or IgG4. In some embodiments, the Fc region is derived from human IgG1. In some embodiments, the Fc region comprises an amino acid sequence selected from the group consisting of SEQ ID NOs: 97-99.

In some embodiments, the light chain of the antibody or antigen-binding fragment thereof of the present disclosure is a λ light chain or a κ light chain.

In some embodiments, the antibody or antigen-binding fragment thereof of the present disclosure is a bispecific or multi-specific antibody or antigen-binding fragment thereof. In some embodiments, the antibody or antigen-binding fragment thereof of the present disclosure is capable of specifically binding to one or more additional antigens other than CD3, or a second epitope on CD3. In some embodiments, the one or more additional antigens other than CD3 are selected from the group consisting of CD16a, CD33, CD38, CD45, CD123, CD146, CD228, CLL-1, FLT3, FLT3L, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, LILRB (e.g., LILRB1, LILRB2, LILRB3, LILRB4, LILRB5), c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPa, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), CD160, CTLA-4 (CD152), GPRC5D, IDO (e.g., IDO1, IDO2), TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), TLR3, CLEC9A, DEC-205, STING, and TGFβ.

In some embodiments, the antibody or antigen-binding fragment thereof of the present disclosure is linked to one or more conjugate moieties. In some embodiments, the conjugate moiety comprises a clearance-modifying agent, a chemotherapeutic agent, a toxin, a radioactive isotope, a lanthanide, a detectable label (e.g., a luminescent label, a fluorescent label), an enzyme-substrate label, a DNA-alkylator, a topoisomerase inhibitor, a tubulin-binder, a purification moiety or other anticancer drugs. In some embodiments, the conjugate moiety is covalently attached either directly or via a linker.

In another aspect, the present disclosure provides a pharmaceutical composition comprising the antibody or antigen-binding fragment thereof of the present disclosure, and one or more pharmaceutically acceptable carriers.

In another aspect, the present disclosure provides a chimeric antigen receptor comprising the antibody or antigen-binding fragment thereof of the present disclosure, a transmembrane region and an intracellular signal region. In some embodiments, the transmembrane region comprises a transmembrane region of CD3, CD4, CD8 or CD28. In some embodiments, the intracellular signal region is selected from the group consisting of: an intracellular signal regions sequence of CD3, Fc γ RI, CD27, CD28, CD137, CD134, MyD88, CD40, CD278, TLRs, or a combination thereof. In some embodiments, the antigen-binding fragment of the chimeric antigen receptor is a scFv.

In another aspect, the present disclosure provides an isolated polynucleotide encoding the antibody or antigen-binding fragment thereof of the present disclosure, and/or the chimeric antigen receptor of the present disclosure.

In another aspect, the present disclosure provides a vector comprising the isolated polynucleotide of the present disclosure.

In another aspect, the present disclosure provides a host expression system comprising the vector of the present disclosure or having the polynucleotide of the present disclosure integrated into genome thereof. In some embodiments, the host expression system of the present disclosure is a microorganism, a yeast, or a mammalian cell. In some embodiments, the microorganism is selected from the group consisting of E. coli and B. subtilis. In some embodiments, the yeast is Saccharomyces. In some embodiments, the mammalian cell is selected from the group consisting of COS, CHO-S, CHO-K1, HEK-293, and 3T3 cells.

In another aspect, the present disclosure provides a virus comprising the vector of the present disclosure.

In another aspect, the present disclosure provides a kit comprising the antibody or antigen-binding fragment thereof of the present disclosure and/or the pharmaceutical composition of the present disclosure and/or the chimeric antigen receptor of the present disclosure, and a second therapeutic agent.

In another aspect, the present disclosure provides a method of expressing the antibody or antigen-binding fragment thereof, or the chimeric antigen receptor of the present disclosure, comprising culturing the host expression system of the present disclosure under the condition at which the antibody or antigen-binding fragment thereof of the present disclosure or the chimeric antigen receptor of the present disclosure is expressed.

In another aspect, the present disclosure provides a method of treating, preventing or alleviating a disease, disorder or condition in a subject, comprising administering to the subject a therapeutically effective amount of the antibody or antigen-binding fragment thereof, and/or the pharmaceutical composition, and/or the chimeric antigen receptor of the present disclosure.

In another aspect, the present disclosure provides use of the antibody or antigen-binding fragment thereof, and/or the pharmaceutical composition, and/or the chimeric antigen receptor of the present disclosure in the manufacture of a medicament for treating a CD3 related disease, disorder or condition in a subject.

In another aspect, the present disclosure provides use of the antibody or antigen-binding fragment thereof, and/or the chimeric antigen receptor and/or the pharmaceutical composition of the present disclosure in the manufacture of a diagnostic reagent for diagnosing a CD3-related disease, disorder or condition.

In some embodiments, the disease, disorder or condition is immune disease, inflammatory disease, cancer or neurological disease. In some embodiments, the cancer is a solid tumor or hematologic tumor. In some embodiments, the disease, disorder or condition is selected from the group consisting of lung cancer (e.g., non-small-cell lung cancer (NSCLC), small cell lung cancer (SCLC), adenocarcinoma of the lung, or squamous cell carcinoma of the lung), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), melanoma (e.g., cutaneous or intraocular melanoma), thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma.

In some embodiments, the subject is human.

In some embodiments, the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

In some embodiments, the method of treating, preventing or alleviating a disease, disorder or condition in a subject further includes administering to the subject in need thereof an additional therapeutic agent. In some embodiments, the additional therapeutic agent is selected from the group consisting of: an active agent, an imaging agent, a cytotoxic agent, and angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, and a microRNA. In some embodiments, the additional therapeutic agent is administered to the subject in need before, after or simultaneously with the antibody or antigen-binding fragment thereof, and/or the pharmaceutical composition, and/or the chimeric antigen receptor of the present disclosure.

In another aspect, the present disclosure provides a method of activating a CD3-expressing T cell in vivo or in vitro, comprising contacting the CD3-expressing T cell with the antibody or antigen-binding fragment thereof, and/or the pharmaceutical composition, and/or the chimeric antigen receptor of the present disclosure.

In another aspect, the present disclosure provides a method of modulating CD3 activity in a CD3-expressing cell, comprising exposing the CD3-expressing cell to the antibody or antigen-binding fragment thereof, and/or the pharmaceutical composition, and/or the chimeric antigen receptor of the present disclosure.

In another aspect, the present disclosure provides a method of promoting in vivo or in vitro processing of a second antigen by a CD3-expressing T cell, comprising contacting the CD3-expressing T cell with the bispecific antibody or antigen-binding fragment thereof of the present disclosure, wherein the bispecific antibody or antigen-binding fragment thereof is capable of specifically binding to both the CD3-expressing T cell and a second antigen thereby bringing both in close proximity.

In another aspect, the present disclosure provides a method of detecting presence or amount of CD3 in a sample, comprising contacting the sample with the antibody or antigen-binding fragment thereof, and/or the pharmaceutical composition, and/or the chimeric antigen receptor of the present disclosure, and determining the presence or the amount of CD3 in the sample.

In another aspect, the present disclosure provides a method of diagnosing a CD3 related disease or condition in a subject, comprising: a) contacting a sample obtained from the subject with the antibody or antigen-binding fragment thereof, and/or the chimeric antigen receptor and/or the pharmaceutical composition of the present disclosure; b) determining presence or amount of CD3 in the sample; and c) correlating the presence or the amount of CD3 to existence or status of the CD3 related disease or condition in the subject.

In another aspect, the present disclosure provides a kit comprising the antibody or antigen-binding fragment thereof and/or the chimeric antigen receptor and/or the pharmaceutical composition of the present disclosure, useful in detecting CD3, optionally recombinant CD3, CD3 expressed on cell surface, or CD3-expressing cells.

### BRIEF DESCRIPTION OF THE DRAWINGS

FIG. 1 shows activation capacity of several selected antibodies on Jurkat-NFAT-Luc cells.

FIG. 2 shows the FACS analysis results of the binding affinity of several selected chimeric antibodies and benchmark antibody OKT3 to Jurkat cells.

FIG. 3 shows the FACS analysis results of the binding affinity of several selected chimeric antibodies and benchmark antibodies BMK-B219 and BMK-TCB to Jurkat cells.

FIG. 4 shows binding affinity of several selected chimeric antibodies to 293T-cynoCD3mix cells.

FIG. 5 shows the activation capacity of several selected chimeric antibodies and benchmark antibody OKT3 on Jurkat-NFAT-Luc cells.

FIG. 6 shows the IL-2 release induced by several selected chimeric antibodies and benchmark antibody OKT3 in PBMC activation assay.

FIG. 7 shows the IFNγ release induced by several selected chimeric antibodies and benchmark antibody OKT3 in PBMC activation assay.

FIG. 8 shows CD25 expression of CD3+ human T cells induced by several selected chimeric antibodies and benchmark antibodies BMK-B219 and BMK-TCB in PBMC activation assay.

FIG. 9A and FIG. 9B show the binding affinity of several produced humanized or affinity maturated 40-C12-C10-E9 antibodies to Jurkat cells.

FIGS. 10A˜F show the activation capacity of several humanized or affinity maturated 40-C12-C10-E9 antibodies and benchmark antibody BMK-TCB on Jurkat-NFAT-Luc cells.

FIG. 11 shows the activation effect of several humanized or affinity maturated 40-C12-C10-E9 antibodies and benchmark antibody BMK-TCB in PBMC activation assay.

FIG. 12 shows the binding affinity of several produced humanized 147E11E2 antibodies to Jurkat cells.

FIG. 13 shows the binding affinity of several produced humanized 147E11E2 antibodies to 293T-cynoCD3mix cells.

FIGS. 14A-C show the activation capacity of several humanized 147E11E2 antibodies and benchmark antibody BMK-TCB on Jurkat-NFAT-Luc cells.

FIG. 15 shows the activation effect of several humanized 147E11E2 antibodies and benchmark antibody BMK-TCB in PBMC activation assay.

FIG. 16A and FIG. 16B show the binding affinity of several produced humanized or affinity maturated 40-C12-C10-E9 antibodies to Jurkat cells.

FIGS. 17A˜E show the activation capacity of several humanized or affinity maturated 40-C12-C10-E9 antibodies on Jurkat-NFAT-Luc cells.

FIG. 18 shows the activation effect of several humanized or affinity maturated 40-C12-C10-E9 antibodies and benchmark antibody BMK-TCB in PBMC activation assay.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to a person skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entireties.

### Definitions

The term “antibody” as used herein includes any immunoglobulin, monoclonal antibody, polyclonal antibody, multivalent antibody, bivalent antibody, monovalent antibody, multi-specific antibody, or bispecific antibody that binds to a specific antigen. A native intact antibody comprises two heavy (H) chains and two light (L) chains. Mammalian heavy chains are classified as alpha, delta, epsilon, gamma, and mu, each heavy chain comprises a variable region (VH) and a first, second, third, and optionally fourth constant region (CH1, CH2, CH3, CH4 respectively); mammalian light chains are classified as λ or κ, while each light chain comprises a variable region (VL) and a constant region. The antibody has a “Y” shape, with the stem of the Y comprising the second and third constant regions of two heavy chains bound together via disulfide bonding. Each arm of the Y includes the variable region and first constant region of a single heavy chain bound to the variable and constant regions of a single light chain. The variable regions of the light and heavy chains are responsible for antigen binding. The variable regions in both chains generally contain three highly variable loops called the complementarity determining regions (CDRs) (light chain CDRs including LCDR1, LCDR2, and LCDR3, heavy chain CDRs including HCDR1, HCDR2, HCDR3). The three CDRs are interposed between flanking stretches known as framework regions (FRs) (light chain FRs including LFR1, LFR2, LFR3, and LFR4, heavy chain FRs including HFR1, HFR2, HFR3, and HFR4), which are more highly conserved than the CDRs and form a scaffold to support the highly variable loops. The constant regions of the heavy and light chains are not involved in antigen-binding, but exhibit various effector functions. Antibodies are assigned to classes based on the amino acid sequences of the constant regions of their heavy chains. The five major classes or isotypes of antibodies are IgA, IgD, IgE, IgG, and IgM, which are characterized by the presence of alpha, delta, epsilon, gamma, and mu heavy chains, respectively. Several of the major antibody classes are divided into subclasses such as IgG1 (gamma1 heavy chain), IgG2 (gamma2 heavy chain), IgG3 (gamma3 heavy chain), IgG4 (gamma4 heavy chain), IgA1 (alpha1 heavy chain), or IgA2 (alpha2 heavy chain).

In certain embodiments, the antibody provided herein encompasses any antigen-binding fragments thereof. The term “antigen-binding fragment” as used herein refers to an antibody fragment formed from a portion of an antibody comprising one or more (e.g., 1, 2, 3, 4, 5, or 6) CDRs, or any other antibody fragment that binds to an antigen but does not comprise an intact native antibody structure. Examples of antigen-binding fragments include, without limitation, a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a bispecific antibody, a multi-specific antibody, a camelized single domain antibody, a nanobody, a domain antibody, or a bivalent domain antibody. An antigen-binding fragment is capable of binding to the same antigen or epitope to which the parent antibody binds.

“Fab” with regard to an antibody refers to that portion of the antibody consisting of a single light chain (both variable and constant regions) bound to the variable region and first constant region of a single heavy chain by a disulfide bond.

“Fab′” refers to a Fab fragment that includes a portion of the hinge region. “F(ab′)₂” refers to a dimer of Fab′.

“Fc” with regard to an antibody (e.g., of IgG, IgA, or IgD isotype) refers to that portion of the antibody consisting of the second and third constant domains of a first heavy chain bound to the second and third constant domains of a second heavy chain via disulfide bonding. Fc with regard to antibody of IgM and IgE isotype further comprises a fourth constant domain. The Fc portion of the antibody is responsible for various effector functions such as antibody-dependent cell-mediated cytotoxicity (ADCC), and complement dependent cytotoxicity (CDC), but does not function in antigen binding.

“Fv” with regard to an antibody refers to the smallest fragment of the antibody to bear the complete antigen binding site. An Fv fragment consists of the variable region of a single light chain bound to the variable region of a single heavy chain.

“Single-chain Fv antibody” or “scFv” refers to an engineered antibody consisting of a light chain variable region and a heavy chain variable region connected to one another directly or via a linker (e.g., a peptide sequence) (Huston J S et al., Proc Natl Acad Sci USA, 85: 5879 (1988)).

“Single-chain Fv-Fc antibody” or “scFv-Fc” refers to an engineered antibody consisting of a scFv connected to the Fc region of an antibody.

“Camelized single domain antibody”, “heavy chain antibody”, or “HCAb” refers to an antibody that contains two VH domains and no light chains (Riechmann L. and Muyldermans S., J Immunol Methods. December 10; 231(1-2): 25-38 (1999); Muyldermans S., J Biotechnol. June; 74 (4): 277-302 (2001); WO94/04678; WO94/25591; U.S. Pat. No. 6,005,079). Heavy chain antibodies were originally derived from Camelidae (camels, dromedaries, and llamas). Although devoid of light chains, camelized antibodies have an authentic antigen-binding repertoire (Hamers-Casterman C. et al., Nature. June 3; 363(6428): 446-8 (1993); Nguyen V K. et al., Immunogenetics. April; 54 (1): 39-47 (2002); Nguyen V K. et al., Immunology. May; 109(1): 93-101 (2003)). The variable domain of a heavy chain antibody (VHH domain) represents the smallest known antigen-binding unit generated by adaptive immune responses (Koch-Nolte F. et al., FASEB J. November; 21(13): 3490-8. Epub 2007 Jun. 15 (2007)).

A “nanobody” refers to an antibody fragment that consists of a VHH domain from a heavy chain antibody and two constant domains, CH2 and CH3.

A “diabody” or “dAb” includes small antibody fragments with two antigen-binding sites, wherein the fragments comprise a VH domain connected to a VL domain in the same polypeptide chain (VH-VL or VL-VH) (see, e.g., Holliger P. et al., Proc Natl Acad Sci USA. July 15; 90 (14): 6444-8 (1993); EP404097; WO93/11161). By using a linker that is too short to allow pairing between the two domains on the same chain, the domains are forced to pair with the complementary domains of another chain, thereby creating two antigen-binding sites. The antigen-binding sites may target the same or different antigens (or epitopes). In certain embodiments, a “bispecific ds diabody” is a diabody target two different antigens (or epitopes).

A “domain antibody” refers to an antibody fragment containing only the variable region of a heavy chain or the variable region of a light chain. In certain instances, two or more VH domains are covalently joined with a peptide linker to create a bivalent or multivalent domain antibody. The two VH domains of a bivalent domain antibody may target the same or different antigens.

The term “valent” as used herein refers to the presence of a specified number of antigen binding sites in a given molecule. The term “monovalent” refers to an antibody or an antigen-binding fragment having only one single antigen-binding site; and the term “multivalent” refers to an antibody or antigen-binding fragment having multiple antigen-binding sites. As such, the terms “bivalent”, “tetravalent”, and “hexavalent” denote the presence of two antigen-binding sites, four antigen-binding sites, and six antigen-binding sites, respectively, in an antigen-binding molecule. In some embodiments, the antibody or antigen-binding fragment thereof is bivalent.

As used herein, a “bispecific” antibody refers to an artificial antibody which has fragments derived from two different monoclonal antibodies and is capable of binding to two different epitopes. The two epitopes may present on the same antigen, or they may present on two different antigens.

As used herein, a “multi-specific” antibody refers to an antibody that specifically binds to at least two distinct antigens or at least two distinct epitopes within the same antigen. Multi-specific antibody may bind for example two, three, four, five or more distinct antigens or distinct epitopes within the same antigen.

In certain embodiments, an “scFv dimer” is a bivalent diabody or bispecific scFv (BsFv) comprising VH-VL (linked by a peptide linker) dimerized with another VH-VL moiety such that VH's of one moiety coordinate with the VL's of the other moiety and form two binding sites which can target the same antigens (or epitopes) or different antigens (or epitopes). In other embodiments, an “scFv dimer” is a bispecific diabody comprising VH1-VL2 (linked by a peptide linker) associated with VL1-VH2 (also linked by a peptide linker) such that VH1 and VL1 coordinate and VH2 and VL2 coordinate and each coordinated pair has a different antigen specificity.

A “dsFv” refers to a disulfide-stabilized Fv fragment that the linkage between the variable region of a single light chain and the variable region of a single heavy chain is a disulfide bond. In some embodiments, a “(dsFv) 2” or “(dsFv-dsFv′)” comprises three peptide chains: two VH moieties linked by a peptide linker (e.g., a long flexible linker) and bound to two VL moieties, respectively, via disulfide bridges. In some embodiments, dsFv-dsFv′ is bispecific in which each disulfide paired heavy and light chain has a different antigen specificity.

The term “chimeric” as used herein, means an antibody or antigen-binding fragment, having a portion of heavy and/or light chain derived from one species, and the rest of the heavy and/or light chain derived from a different species. In an illustrative example, a chimeric antibody may comprise a constant region derived from human and a variable region from a non-human animal, such as from mouse. In some embodiments, the non-human animal is a mammal, for example, a mouse, a rat, a rabbit, a goat, a sheep, a guinea pig, or a hamster.

The term “humanized” as used herein means that the antibody or antigen-binding fragment comprises CDRs derived from non-human animals, FR regions derived from human, and when applicable, the constant regions derived from human. The CDRs of humanized antibodies provided in the present disclosure may contain mutation(s) compared to the CDRs of their parent antibodies.

The term “affinity” as used herein refers to the strength of non-covalent interaction between an immunoglobulin molecule (i.e., antibody) or antigen-binding fragment thereof and an antigen.

An antibody or antigen-binding fragment thereof that “specifically binds” or “specific binding” to a target (e.g., an epitope) is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. For example, an antibody that specifically binds to a CD3 epitope is an antibody that binds this CD3 epitope with greater affinity, avidity, more readily, and/or with greater duration than it binds to other CD3 epitopes or non-CD3 epitopes. It is also understood by reading this definition that, for example, an antibody (or moiety or epitope) that specifically binds to a first target may or may not specifically bind to a second target. As such, “specific binding” or “specifically bind” does not necessarily require (although it can include) exclusive binding. Generally, but not necessarily, reference to binding means specific binding.

The ability to “compete for binding to CD3” as used herein refers to the ability of a first antibody or antigen-binding fragment to inhibit the binding interaction between CD3 and a second anti-CD3 antibody to any detectable degree. In certain embodiments, an antibody or antigen-binding fragment that competes for binding to CD3 inhibits the binding interaction between CD3 and a second anti-CD3 antibody by at least 85%, or at least 90%. In certain embodiments, this inhibition may be greater than 95%, or greater than 99%.

The term “epitope” as used herein refers to the specific group of atoms or amino acids on an antigen to which an antibody binds. Two antibodies may bind the same or a closely related epitope within an antigen if they exhibit competitive binding for the antigen. An epitope can be linear or conformational (i.e., including amino acid residues spaced apart). For example, if an antibody or antigen-binding fragment blocks binding of a reference antibody to the antigen by at least 85%, or at least 90%, or at least 95%, then the antibody or antigen-binding fragment may be considered to bind the same/closely related epitope as the reference antibody.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure, which are summarized as follows.

| Names | Three-letter Code | Single-letter Code |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

A “conservative substitution” with reference to an amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among amino acid residues with neutral hydrophilic side chains (e.g., Cys, Ser, Thr, Asn and Gln), among amino acid residues with acidic side chains (e.g., Asp, Glu), among amino acid residues with basic side chains (e.g., His, Lys, and Arg), or among amino acid residues with aromatic side chains (e.g., Trp, Tyr, and Phe). As known in the art, conservative substitution usually does not cause significant change in the protein conformational structure, and therefore could retain the biological activity of a protein.

The term “homologous” as used herein refers to a nucleic acid sequence (or its complementary strand) or an amino acid sequence that has sequence identity of at least 60% (e.g., at least 65%, 70%, 75%, 80%, 85%, 88%, 90%, 91%, 92%, 93%, 94%, 95%, 96%, 97%, 98%, or 99%) to another sequence when optimally aligned.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). In other words, percent (%) sequence identity of an amino acid sequence (or nucleic acid sequence) can be calculated by dividing the number of amino acid residues (or bases) that are identical relative to the reference sequence to which it is being compared by the total number of the amino acid residues (or bases) in the candidate sequence or in the reference sequence, whichever is shorter. Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215:403-410 (1990); Stephen F. et al., Nucleic Acids Res., 25:3389-3402 (1997)), ClustalW2 (available on the website of European Bioinformatics Institute, see also, Higgins D. G. et al., Methods in Enzymology, 266:383-402 (1996); Larkin M. A. et al., Bioinformatics (Oxford, England), 23(21): 2947-8 (2007)), and ALIGN or Megalign (DNASTAR) software. A person skilled in the art may use the default parameters provided by the tool, or may customize the parameters as appropriate for the alignment, such as for example, by selecting a suitable algorithm.

“Effector functions” as used herein refer to biological activities attributable to the binding of Fc region of an antibody to its effectors such as Cl complex and Fc receptor. Exemplary effector functions include: complement dependent cytotoxicity (CDC) mediated by interaction of antibodies and C1q on the C1 complex; antibody-dependent cell-mediated cytotoxicity (ADCC) mediated by binding of Fc region of an antibody to Fc receptor on an effector cell; and phagocytosis. Effector functions can be evaluated using various assays such as Fc receptor binding assay, C1q binding assay, and cell lysis assay.

“Antibody-dependent cell-mediated cytotoxicity” or “ADCC” as used herein refers to a cell-mediated reaction in which effector cells that express Fc receptors (FcRs) recognize bound antibody or antigen-binding fragment on a target cell and subsequently cause lysis of the target cell. “ADCC activity” or “ADCC effect” refers to the ability of the antibody or antigen-binding fragment which is bound on the target cell to elicit an ADCC reaction as described above.

“Complement dependent cytotoxicity” or “CDC” as used herein refers to a mechanism by which antibodies can mediate specific target cell lysis through activation of an organism's complement system. In CDC, the C1q binds the antibody and this binding triggers the complement cascade which leads to the formation of the membrane attack complex (MAC) (C5b to C9) at the surface of the target cell, as a result of the classical pathway complement activation. “CDC activity” or “CDC effect” refers to the ability of the antibody or antigen-binding fragment which is bound on the target cell to elicit a CDC reaction as described above.

“Target cells” as used herein refer to cells to which antibodies comprising an Fc region specifically bind, generally via the protein part that is C-terminal to the Fc region. “Effector cells” are leukocytes which express one or more Fc receptors and perform effector functions. Examples of human leukocytes which mediate ADCC include peripheral blood mononuclear cells (PBMCs), natural killer (NK) cells, monocytes, cytotoxic T cells and neutrophils; with PBMCs and NK cells being preferred. The effector cells may be isolated from a native source thereof, e.g., from blood or PBMCs as is known in the art.

An “isolated” substance has been altered by the hand of man from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide is “isolated” if it has been sufficiently separated from the coexisting materials of its natural state so as to exist in a substantially pure state. An “isolated nucleic acid sequence” refers to the sequence of an isolated nucleic acid molecule. In certain embodiments, an “isolated antibody or an antigen-binding fragment thereof” refers to the antibody or antigen-binding fragments thereof having a purity of at least 60%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% as determined by electrophoretic methods (such as SDS-PAGE, isoelectric focusing, capillary electrophoresis), or chromatographic methods (such as ion exchange chromatography or reverse phase HPLC).

The term “vector” as used herein refers to a vehicle into which a polynucleotide encoding a protein may be operably inserted so as to bring about the expression of that protein. A vector may be used to transform, transduce, or transfect a host cell so as to bring about expression of the genetic element it carries within the host cell. Examples of vectors include plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector may contain an origin of replication. A vector may also include materials to aid in its entry into the cell, including but not limited to a viral particle, a liposome, or a protein coating. A vector can be an expression vector or a cloning vector. The present disclosure provides vectors (e.g., expression vectors) containing the nucleic acid sequence provided herein encoding the antibody or antigen-binding fragment thereof, at least one promoter (e.g., SV40, CMV, EF-1α) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

The phrase “host cell” as used herein refers to a cell into which an exogenous polynucleotide and/or a vector can be or has been introduced.

The term “subject” includes human and non-human animals. Non-human animals include all vertebrates, e.g., mammals and non-mammals, such as non-human primates, mice, rats, cats, rabbits, sheep, dogs, cows, chickens, amphibians, and reptiles. Except when noted, the terms “patient”, “subject” or “individual” are used herein interchangeably.

The term “anti-tumor activity” means a reduction in tumor cell proliferation, viability, or metastatic activity. For example, anti-tumor activity can be shown by a decline in growth rate of abnormal cells that arises during therapy or tumor size stability or reduction, or longer survival due to therapy as compared to control without therapy. Such activity can be assessed using accepted in vitro or in vivo tumor models, including but not limited to xenograft models, allograft models, mouse mammary tumor virus (MMTV) models, and other known models known in the art to investigate anti-tumor activity.

“Treating” or “treatment” of a disease, disorder or condition as used herein includes preventing or alleviating a disease, disorder or condition, slowing the onset or rate of development of a disease, disorder or condition, reducing the risk of developing a disease, disorder or condition, preventing or delaying the development of symptoms associated with a disease, disorder or condition, reducing or ending symptoms associated with a disease, disorder or condition, generating a complete or partial regression of a disease, disorder or condition, curing a disease, disorder or condition, or some combination thereof.

The term “diagnosis”, “diagnose” or “diagnosing” refers to the identification of a pathological state, disease or condition, such as identification of a CD3 related disease, or refers to identification of a subject with a CD3 related disease who may benefit from a particular treatment regimen. In some embodiments, diagnosis contains the identification of abnormal amount or activity of CD3. In some embodiments, diagnosis refers to the identification of a cancer in a subject.

As used herein, the term “biological sample” or “sample” refers to a biological composition that is obtained or derived from a subject of interest that contains a cellular and/or other molecular entity that is to be characterized and/or identified, for example based on physical, biochemical, chemical and/or physiological characteristics. A biological sample includes, but is not limited to, cells, tissues, organs and/or biological fluids of a subject, obtained by any method known by those of skill in the art. In some embodiments, the biological sample is a fluid sample. In some embodiments, the fluid sample is whole blood, plasma, blood serum, mucus (including nasal drainage and phlegm), peritoneal fluid, pleural fluid, chest fluid, saliva, urine, synovial fluid, cerebrospinal fluid (CSF), thoracentesis fluid, abdominal fluid, ascites or pericardial fluid. In some embodiments, the biological sample is a tissue or cell obtained from stomach, heart, liver, spleen, lung, kidney, skin or blood vessels of the subject.

“CD3” as used herein, refers to the Cluster of Differentiation 3 protein and includes any variants, conformations, isoforms and species homologs of CD3 which are naturally expressed by cells or are expressed by cells transfected with the CD3 gene. For example, CD3 described herein may refer to the Cluster of Differentiation 3 protein derived from any vertebrate source, including mammals such as primates (e.g., humans, monkeys) and rodents (e.g., mice and rats). In mammals, the CD3 molecule is a multi-protein complex of six chains, including: a CD3gamma chain, a CD3delta chain, two CD3epsilon chains, and a homodimer of CD3zeta chains, wherein the CD3zeta chain is the intracellular tail of CD3 molecule, and the CD3gamma, CD3delta and CD3epsilon chains all contain extracellular domain (ECD) expressed on surface of T cells. Exemplary sequence of human CD3 includes human CD3epsilon protein (NCBI Ref Seq No. NP_000724), human CD3 delta protein (NCBI Ref Seq No. NP_000723), and human CD3gamma protein (NCBI Ref Seq No. NP_000064). Exemplary sequence of non-human CD3 includes Macaca fascicularis (monkey) CD3epsilon protein (NCBI Ref Seq No. NP_001270544), Macaca fascicularis (monkey) CD 3delta protein (NCBI Ref Seq No. NP_001274617), Macaca fascicularis (monkey) CD3gamma protein (NCBI Ref Seq No. NP_001270839); Mus musculus (mouse) CD3epsilon protein (NCBI Ref Seq No. NP_031674), Mus musculus (mouse) CD3delta protein (NCBI Ref Seq No. NP_038515), Mus musculus domesticus (mouse) CD3gamma protein (NCBI Ref Seq No. AAA37400); Rattus norvegicus (Rat) CD3epsilon protein (NCBI Ref Seq No. NP_001101610), Rattus norvegicus (Rat) CD3delta protein (NCBI Ref Seq No. NP_037301), Rattus norvegicus (Rat) CD3gamma protein (NCBI Ref Seq No. NP_001071114). In certain embodiments, CD3 used herein can also be recombinant CD3, for example, including recombinant CD3epsilon protein, recombinant CD3delta protein, and recombinant CD3gamma protein, which may optionally be expressed as a recombinant CD3 complex. The recombinant CD3 complex may be expressed on a cell surface, or alternatively may be expressed as a soluble form which is not associated on a cell surface. In certain embodiments, the CD3 is human CD3. The terms “CD3”, “CD-3”, “CD 3”, “cluster of differentiation 3” may be used interchangeably in the present disclosure.

The term “anti-CD3 antibody” refers to an antibody that specifically binds to CD3 (e.g., human CD3). The term “anti-human CD3 antibody” refers to an antibody that specifically binds to human CD3. In some embodiments, the anti-CD3 antibody provided herein specifically binds to a CD3gamma protein. In some embodiments, the anti-CD3 antibody provided herein specifically binds to a CD3delta protein. In some embodiments, the anti-CD3 antibody provided herein specifically binds to a CD3epsilon protein.

The term “CD3gamma” as used herein is intended to encompass any form of CD3gamma, for example, 1) native unprocessed CD3gamma molecule, “full-length” CD3gamma chain or naturally occurring variants of CD3gamma, including, for example, splice variants or allelic variants; 2) any form of CD3gamma that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3gamma subunit generated through recombinant method.

The term “CD3delta” as used herein is intended to encompass any form of CD3delta, for example, 1) native unprocessed CD3delta molecule, “full-length” CD3delta chain or naturally occurring variants of CD3delta, including, for example, splice variants or allelic variants; 2) any form of CD3delta that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3delta subunit generated through recombinant method.

The term “CD3epsilon” as used herein is intended to encompass any form of CD3epsilon, for example, 1) native unprocessed CD3epsilon molecule, “full-length” CD3epsilon chain or naturally occurring variants of CD3epsilon, including, for example, splice variants or allelic variants; 2) any form of CD3epsilon that results from processing in the cell; or 3) full length, a fragment (e.g., a truncated form, an extracellular/transmembrane domain) or a modified form (e.g., a mutated form, a glycosylated/PEGylated, a His-tag/immunofluorescence fused form) of CD3epsilon subunit generated through recombinant method.

In some embodiments, the anti-CD3 antibody provided herein specifically binds to CD3epsilon, but not binding to CD3gamma (or CD3delta) or binding less well to CD3gamma (or CD3delta), e.g., the binding affinity to CD3epsilon is at least 10-fold lower than that to CD3gamma (or CD3delta), or at least 50-fold lower, or at least 100-fold lower, or at least 200-fold lower than that to CD3gamma (or CD3delta). In some embodiments, the anti-CD3 antibody provided herein does not have detectable binding affinity to CD3gamma (or CD3delta). In some embodiments, the binding affinity is determined by FACS assay. In some embodiments, the binding affinity is determined by Mean Fluorescence Intensity (MFI) detected by FACS assay.

A “CD3 related” or “CD3-related” disease, disorder or condition as used herein refers to any disease, disorder or condition caused by, exacerbated by, or otherwise linked to increased or decreased expression or activities of CD3. In some embodiments, the CD3 related disease, disorder or condition is a disorder related to excessive cell proliferation, such as, for example, cancer. In certain embodiments, the CD3 related disease or condition is characterized in expressing or over-expressing of CD3 and/or CD3 related genes.

The term “pharmaceutically acceptable” indicates that the designated carrier, vehicle, diluent, excipient(s), and/or salt is generally chemically and/or physically compatible with the other ingredients comprising the formulation, and physiologically compatible with the recipient thereof.

The term “CD3-expressing cell” as used herein refer to a cell that expresses CD3 on the surface of the cell.

### Anti-CD3 Antibodies

The present disclosure provides anti-CD3 antibodies and antigen-binding fragments thereof. The anti-CD3 antibodies and antigen-binding fragments provided herein are capable of binding (e.g., specifically binding) to CD3 (e.g., human CD3).

Binding affinity of the antibody or antigen-binding fragment thereof provided herein can be represented by K_{D} value, which represents the ratio of dissociation rate to association rate (k_{off}/kₒₙ) when the binding between the antigen and antigen-binding molecule reaches equilibrium. The antigen-binding affinity (e.g., K_{D}) can be appropriately determined using suitable methods known in the art, including, for example, flow cytometry assay. In some embodiments, binding of the antibody or antigen-binding fragment thereof to the antigen at different concentrations can be determined by flow cytometry, the determined mean fluorescence intensity (MFI) can be firstly plotted against antibody concentration, K_{D} value can then be calculated by fitting the dependence of specific binding fluorescence intensity (Y) and the concentration of antibodies (X) into the one site saturation equation: Y=Bₘₐₓ*X/(K_{D}+X) using Prism version 5 (GraphPad Software, San Diego, CA), wherein Bₘₐₓ refers to the maximum specific binding of the tested antibody to the antigen.

Binding of the antibodies or the antigen-binding fragments thereof provided herein to CD3 can also be represented by “half maximal effective concentration” (EC₅₀) value, which refers to the concentration of an antibody where 50% of its maximal binding is observed. The EC₅₀ value can be measured by binding assays known in the art, for example, direct or indirect binding assay such as enzyme-linked immunosorbent assay (ELISA), FACS assay, and other binding assays. In certain embodiments, the antibodies or antigen-binding fragments thereof provided herein are capable of specifically binding to human CD3 (e.g., as measured by FACS assay). In certain embodiments, the antibodies or antigen-binding fragments thereof provided herein are capable of binding to both human and cynomolgus CD3 (e.g., as measured by FACS assay).

In certain embodiments, the antibodies or antigen-binding fragments thereof provided herein are with a T cell activation capability. In certain embodiments, the antibodies or antigen-binding fragments thereof provided herein are with a higher T cell activation capability than OKT3. OKT3 is the first monoclonal antibody drug with specificity for the human CD3 antigen approved by the U.S. FDA in 1986. OKT3 has been described in the prior art as a potent T cell mitogen (Van Wauve, J. Immunol. 124 (1980), 2708-18) as well as a potent T cell killer (Wong et al., Transplantation 50 (1990), 683-9). In certain embodiments, the antibodies or antigen-binding fragments thereof provided herein are with a higher or at least comparable T cell activation capability compared to BMK-B219 or BMK-TCB. BMK-B219 is an anti-CD3 antibody developed by Johnson & Johnson, and its information can be found in, for example, WO2019224717A2. BMK-TCB is an anti-CD3 antibody developed by Roche, and its information can be found in, for example, WO2019154890A1.

The T cell activation capability of anti-CD3 antibodies can be measured by well-known methods in the art, for example, can be measured by Jurkat NFAT-Luciferase activation assay. In certain embodiments, the T cell activation capability is measured by the method as described in Example 2.3, Example 3.2 and Example 4.4 of the present disclosure.

In certain embodiments, the antibodies or antigen-binding fragments thereof provided herein are with a PBMC activation capability. In certain embodiments, the antibodies or antigen-binding fragments thereof provided herein are with a higher PBMC activation capability than OKT3, BMK-B219 and/or BMK-TCB. The PBMC activation capability of anti-CD3 antibodies can be determined by well-known methods in the art, for example, can be determined by ELISA assay, e.g., measuring IL-2 and/or IFNγ release level, measuring CD25 expression on CD3⁺ T cells. In certain embodiments, the PBMC activation capability is measured by the method as described in Example 3.6 of the present disclosure.

### Illustrative Anti-CD3 Antibodies

In certain embodiments, the present disclosure provides antibodies or antigen-binding fragments thereof which specifically bind to CD3, comprising:
one or two or three heavy chain complementarity determining regions (HCDR1, HCDR2 and/or HCDR3) contained within any one of the heavy chain variable (VH) region sequences selected from the group consisting of SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 and 245; and/or
one or two or three light chain complementarity determining regions (LCDR1, LCDR2 and/or LCDR3) contained within any one of the light chain variable (VL) region sequences selected from the group consisting of SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 and 232.

A person skilled in the art can define or identify CDR boundaries of a VH or VL region by well-known methods in the art as long as the amino acid sequence of the VH or VL region is known. For example, CDR boundaries for an antibody or antigen-binding fragment thereof may be defined or identified by the conventions of Kabat, IMGT, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3): 651-63 (1985); Chothia, C. and Lesk, A. M., J. Mol. Biol., 196,901 (1987); Chothia, C. et al., Nature. December 21-28; 342(6252): 877-83 (1989); Kabat E. A. et al., Sequences of Proteins of immunological Interest, 5th Ed. Public Health Service, National Institutes of Health, Bethesda, Md. (1991); Marie-Paule Lefranc et al., Developmental and Comparative Immunology, 27:55-77 (2003); Marie-Paule Lefranc et al., Immunome Research, 1(3), (2005); Marie-Paule Lefranc, Molecular Biology of B cells (second edition), chapter 26, 481-514, (2015)). In some embodiments, the CDR boundaries of the antibodies or antigen-binding fragments thereof provided herein are identified by the convention of Kabat. In some embodiments, the CDR boundaries of the antibodies or antigen-binding fragments thereof provided herein are identified by the convention of IMGT. In some embodiments, the CDR boundaries of the antibodies or antigen-binding fragments thereof provided herein are identified by the convention of Chothia. In some embodiments, the CDR boundaries of the antibodies or antigen-binding fragments thereof provided herein are identified by the convention of Al-Lazikani.

In certain embodiments, the present disclosure provides antibodies or antigen-binding fragments thereof which specifically bind to CD3 comprising one or more (e.g., 1, 2, 3, 4, 5, or 6) CDR sequences of an anti-CD3 antibody 25-G12-G6-C12, 40-C12-C10-E9, 8-B12-F9-B11, 31-F8-F5-C5, 16-F2-C11-D9, 20-E11-E11-C2, 7-D9-G10-H2, 7-D8-G12-E4, 2-F12-A6-G2, 3-C6-C11-F12, 4-F12-F1-A4, 3-F3-G12-E2, 124E3D6, 126A11A4, 127E2D3, 133B4C7, 140D2B10, 147C6F3 or 147E11E2.

Antibody “25-G12-G6-C12” as used herein refers to a mouse monoclonal antibody comprising a heavy chain variable region having the sequence of SEQ ID NO: 7, and a light chain variable region having the sequence of SEQ ID NO: 8.

Antibody “40-C12-C10-E9” as used herein refers to a mouse monoclonal antibody comprising a heavy chain variable region having the sequence of SEQ ID NO: 15, and a light chain variable region having the sequence of SEQ ID NO: 16.

Antibody “8-B12-F9-B11” as used herein refers to a mouse monoclonal antibody comprising a heavy chain variable region having the sequence of SEQ ID NO: 23, and a light chain variable region having the sequence of SEQ ID NO: 24.

Antibody “31-F8-F5-C5” as used herein refers to a mouse monoclonal antibody comprising a heavy chain variable region having the sequence of SEQ ID NO: 31, and a light chain variable region having the sequence of SEQ ID NO: 32.

Antibody “16-F2-C11-D9” as used herein refers to a mouse monoclonal antibody comprising a heavy chain variable region having the sequence of SEQ ID NO: 39, and a light chain variable region having the sequence of SEQ ID NO: 40.

Antibody “20-E11-E11-C2” as used herein refers to a mouse monoclonal antibody comprising a heavy chain variable region having the sequence of SEQ ID NO: 47, and a light chain variable region having the sequence of SEQ ID NO: 48.

Antibody “7-D9-G10-H2” as used herein refers to a mouse monoclonal antibody comprising a heavy chain variable region having the sequence of SEQ ID NO: 55, and a light chain variable region having the sequence of SEQ ID NO: 56.

Antibody “7-D8-G12-E4” as used herein refers to a mouse monoclonal antibody comprising a heavy chain variable region having the sequence of SEQ ID NO: 63, and a light chain variable region having the sequence of SEQ ID NO: 64.

Antibody “2-F12-A6-G2” as used herein refers to a mouse monoclonal antibody comprising a heavy chain variable region having the sequence of SEQ ID NO: 71, and a light chain variable region having the sequence of SEQ ID NO: 72.

Antibody “3-C6-C11-F12” as used herein refers to a mouse monoclonal antibody comprising a heavy chain variable region having the sequence of SEQ ID NO: 79, and a light chain variable region having the sequence of SEQ ID NO: 80.

Antibody “4-F12-F1-A4” as used herein refers to a mouse monoclonal antibody comprising a heavy chain variable region having the sequence of SEQ ID NO: 87, and a light chain variable region having the sequence of SEQ ID NO: 88.

Antibody “3-F3-G12-E2” as used herein refers to a mouse monoclonal antibody comprising a heavy chain variable region having the sequence of SEQ ID NO: 95, and a light chain variable region having the sequence of SEQ ID NO: 96.

Antibody “124E3D6” as used herein refers to a mouse monoclonal antibody comprising a heavy chain variable region having the sequence of SEQ ID NO: 108, and a light chain variable region having the sequence of SEQ ID NO: 109.

Antibody “126A11A4” as used herein refers to a mouse monoclonal antibody comprising a heavy chain variable region having the sequence of SEQ ID NO: 116, and a light chain variable region having the sequence of SEQ ID NO: 117.

Antibody “127E2D3” as used herein refers to a mouse monoclonal antibody comprising a heavy chain variable region having the sequence of SEQ ID NO: 124, and a light chain variable region having the sequence of SEQ ID NO: 125.

Antibody “133B4C7” as used herein refers to a mouse monoclonal antibody comprising a heavy chain variable region having the sequence of SEQ ID NO: 132, and a light chain variable region having the sequence of SEQ ID NO: 133.

Antibody “140D2B10” as used herein refers to a mouse monoclonal antibody comprising a heavy chain variable region having the sequence of SEQ ID NO: 140, and a light chain variable region having the sequence of SEQ ID NO: 141.

Antibody “147C6F3” as used herein refers to a mouse monoclonal antibody comprising a heavy chain variable region having the sequence of SEQ ID NO: 148, and a light chain variable region having the sequence of SEQ ID NO: 149.

Antibody “147E11E2” as used herein refers to a mouse monoclonal antibody comprising a heavy chain variable region having the sequence of SEQ ID NO: 156, and a light chain variable region having the sequence of SEQ ID NO: 157.

The specific amino acid sequences of the heavy chain variable region and light chain variable region of each exemplary antibody as described above are shown in Table 3 below.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 7, and three light chain CDRs (LCDR1, LCDR2 and LCDR3) contained within the VL region sequence as set forth in SEQ ID NO: 8.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 15, and three light chain CDRs (LCDR1, LCDR2 and LCDR3) contained within the VL region sequence as set forth in SEQ ID NO: 16.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 23, and three light chain CDRs (LCDR1, LCDR2 and LCDR3) contained within the VL region sequence as set forth in SEQ ID NO: 24.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 31, and three light chain CDRs (LCDR1, LCDR2 and LCDR3) contained within the VL region sequence as set forth in SEQ ID NO: 32.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 39, and three light chain CDRs (LCDR1, LCDR2 and LCDR3) contained within the VL region sequence as set forth in SEQ ID NO: 40.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 47, and three light chain CDRs (LCDR1, LCDR2 and LCDR3) contained within the VL region sequence as set forth in SEQ ID NO: 48.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 55, and three light chain CDRs (LCDR1, LCDR2 and LCDR3) contained within the VL region sequence as set forth in SEQ ID NO: 56.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 63, and three light chain CDRs (LCDR1, LCDR2 and LCDR3) contained within the VL region sequence as set forth in SEQ ID NO: 64.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 71, and three light chain CDRs (LCDR1, LCDR2 and LCDR3) contained within the VL region sequence as set forth in SEQ ID NO: 72.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 79, and three light chain CDRs (LCDR1, LCDR2 and LCDR3) contained within the VL region sequence as set forth in SEQ ID NO: 80.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 87, and three light chain CDRs (LCDR1, LCDR2 and LCDR3) contained within the VL region sequence as set forth in SEQ ID NO: 88.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 95, and three light chain CDRs (LCDR1, LCDR2 and LCDR3) contained within the VL region sequence as set forth in SEQ ID NO: 96.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 108, and three light chain CDRs (LCDR1, LCDR2 and LCDR3) contained within the VL region sequence as set forth in SEQ ID NO: 109.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 116, and three light chain CDRs (LCDR1, LCDR2 and LCDR3) contained within the VL region sequence as set forth in SEQ ID NO: 117.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 124, and three light chain CDRs (LCDR1, LCDR2 and LCDR3) contained within the VL region sequence as set forth in SEQ ID NO: 125.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 132, and three light chain CDRs (LCDR1, LCDR2 and LCDR3) contained within the VL region sequence as set forth in SEQ ID NO: 133.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 140, and three light chain CDRs (LCDR1, LCDR2 and LCDR3) contained within the VL region sequence as set forth in SEQ ID NO: 141.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 148, and three light chain CDRs (LCDR1, LCDR2 and LCDR3) contained within the VL region sequence as set forth in SEQ ID NO: 149.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 156, and three light chain CDRs (LCDR1, LCDR2 and LCDR3) contained within the VL region sequence as set forth in SEQ ID NO: 157.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 163, and three light chain CDRs (LCDR1, LCDR2 and LCDR3) contained within the VL region sequence as set forth in SEQ ID NO: 168.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 163, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 163, and three light chain CDRs (LCDR1, LCDR2 and LCDR3) contained within the VL region sequence as set forth in SEQ ID NO: 170.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 164, and three light chain CDRs (LCDR1, LCDR2 and LCDR3) contained within the VL region sequence as set forth in SEQ ID NO: 168.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 164, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 164, and three light chain CDRs (LCDR1, LCDR2 and LCDR3) contained within the VL region sequence as set forth in SEQ ID NO: 170.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 168.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 170.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 166, and three light chain CDRs (LCDR1, LCDR2 and LCDR3) contained within the VL region sequence as set forth in SEQ ID NO: 168.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 166, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 166, and three light chain CDRs (LCDR1, LCDR2 and LCDR3) contained within the VL region sequence as set forth in SEQ ID NO: 170.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 186, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 187, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 188, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 189, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 190, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 191, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 192, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 193, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 194, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 195, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 196, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 197.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 198.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 199.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 200.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 202, and three light chain CDRs (LCDR1, LCDR2 and LCDR3) contained within the VL region sequence as set forth in SEQ ID NO: 207.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 202, and three light chain CDRs (LCDR1, LCDR2 and LCDR3) contained within the VL region sequence as set forth in SEQ ID NO: 208.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 202, and three light chain CDRs (LCDR1, LCDR2 and LCDR3) contained within the VL region sequence as set forth in SEQ ID NO: 209.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 203, and three light chain CDRs (LCDR1, LCDR2 and LCDR3) contained within the VL region sequence as set forth in SEQ ID NO: 207.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 203, and three light chain CDRs (LCDR1, LCDR2 and LCDR3) contained within the VL region sequence as set forth in SEQ ID NO: 208.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 203, and three light chain CDRs (LCDR1, LCDR2 and LCDR3) contained within the VL region sequence as set forth in SEQ ID NO: 209.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 204, and three light chain CDRs (LCDR1, LCDR2 and LCDR3) contained within the VL region sequence as set forth in SEQ ID NO: 207.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 205, and three light chain CDRs (LCDR1, LCDR2 and LCDR3) contained within the VL region sequence as set forth in SEQ ID NO: 207.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 206, and three light chain CDRs (LCDR1, LCDR2 and LCDR3) contained within the VL region sequence as set forth in SEQ ID NO: 207.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 206, and three light chain CDRs (LCDR1, LCDR2 and LCDR3) contained within the VL region sequence as set forth in SEQ ID NO: 208.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 206, and three light chain CDRs (LCDR1, LCDR2 and LCDR3) contained within the VL region sequence as set forth in SEQ ID NO: 209.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 233, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 234, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 235, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 236, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 237, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 238, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 239, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 240, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 241, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 242, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 243, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 244, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 245, and three light chain CDRs (LCDR1, LCDR2 and LCDR3) contained within the VL region sequence as set forth in SEQ ID NO: 169.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 230.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 231.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 165, and three light chain CDRs (LCDR1, LCDR2 and LCDR3) contained within the VL region sequence as set forth in SEQ ID NO: 232.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 235, and three light chain CDRs (LCDR1, LCDR2 and LCDR3) contained within the VL region sequence as set forth in SEQ ID NO: 230.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 235, and three light chain CDRs (LCDR1, LCDR2 and LCDR3) contained within the VL region sequence as set forth in SEQ ID NO: 231.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 236, and three light chain CDRs (LCDR1, LCDR2 and LCDR3) contained within the VL region sequence as set forth in SEQ ID NO: 230.

In certain embodiments, the antibodies or antigen-binding fragments thereof provided herein comprises three heavy chain CDRs (HCDR1, HCDR2 and HCDR3) contained within the VH region sequence as set forth in SEQ ID NO: 236, and three light chain CDRs (LCDR1, LCDR2 and LCDR3) contained within the VL region sequence as set forth in SEQ ID NO: 231.

In certain embodiments, the antibodies or antigen-binding fragments thereof provided herein comprise at least one (e.g., 1, 2, or 3) heavy or light chain CDR comprising an amino acid sequence selected from the group consisting of SEQ ID NOs: 1, 2, 3, 4, 5, 6, 9, 10, 11, 12, 13, 14, 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 81, 82, 83, 84, 85, 86, 89, 90, 91, 92, 93, 94, 102, 103, 104, 105, 106, 107, 110, 111, 112, 113, 114, 115, 118, 119, 120, 121, 122, 123, 126, 127, 128, 129, 130, 131, 134, 135, 136, 137, 138, 139, 142, 143, 144, 145, 146, 147, 150, 151, 152, 153, 154, 155, 171, 172, 173, 174, 175, 176, 177, 178, 179, 180, 181, 182, 183, 184, 185, 201, 162, 167, 210, 211, 212, 214, 215, 216, 217, 218, 219, 220, 221, 222, 223, 224, 225, 226, 227, 228 and 229.

In certain embodiments, the antibodies or antigen-binding fragments thereof provided herein comprise a VH region comprising one or two or three of HCDR1, HCDR2 and HCDR3 comprising an amino acid sequence selected from the group consisting of SEQ ID NOs: 1, 2, 3, 9, 10, 11, 17, 18, 19, 25, 26, 27, 33, 34, 35, 41, 42, 43, 49, 50, 51, 57, 58, 59, 65, 66, 67, 73, 74, 75, 81, 82, 83, 89, 90, 91, 102, 103, 104, 110, 111, 112, 118, 119, 120, 126, 127, 128, 134, 135, 136, 142, 143, 144, 150, 151, 152, 171, 172, 173, 174, 175, 176, 177, 178, 179, 180, 181, 201, 162, 210, 211, 212, 217, 218, 219, 220, 221, 222, 223, 224, 225, 226, 227, 228 and 229.

In certain embodiments, the antibodies or antigen-binding fragments thereof provided herein comprise a VL region comprising one or two or three of LCDR1, LCDR2 and LCDR3 comprising an amino acid sequence selected from the group consisting of SEQ ID NOs: 4, 5, 6, 12, 13, 14, 20, 21, 22, 28, 29, 30, 36, 37, 38, 44, 45, 46, 52, 53, 54, 60, 61, 62, 68, 69, 70, 76, 77, 78, 84, 85, 86, 92, 93, 94, 105, 106, 107, 113, 114, 115, 121, 122, 123, 129, 130, 131, 137, 138, 139, 145, 146, 147, 153, 154, 155, 182, 183, 184, 185, 167, 214, 215 and 216.

In certain embodiments, the antibodies or antigen-binding fragments thereof provided herein comprise a HCDR1 comprising an amino acid sequence selected from the group consisting of SEQ ID NOs: 1, 9, 17, 25, 33, 41, 49, 57, 65, 73, 81, 89, 102, 110, 118, 126, 134, 142, 150, 217, 218 and 219; a HCDR2 comprising an amino acid sequence selected from the group consisting of SEQ ID NOs: 2, 10, 18, 26, 34, 42, 50, 58, 66, 74, 82, 90, 103, 111, 119, 127, 135, 143, 151, 171, 172, 173, 201, 210, 211, 212, 220 and 222; and a HCDR3 comprising an amino acid sequence selected from the group consisting of SEQ ID NOs: 3, 11, 19, 27, 35, 43, 51, 59, 67, 75, 83, 91, 104, 112, 120, 128, 136, 144, 152, 174, 175, 176, 177, 178, 179, 180, 181, 221, 223, 224, 225, 226, 227, 228, 229 and 162.

In certain embodiments, the antibodies or antigen-binding fragments thereof provided herein comprise a LCDR1 comprising an amino acid sequence selected from the group consisting of SEQ ID NOs: 4, 12, 20, 28, 36, 44, 52, 60, 68, 76, 84, 92, 105, 113, 121, 129, 137, 145 and 153; a LCDR2 comprising an amino acid sequence selected from the group consisting of SEQ ID NOs: 5, 13, 21, 29, 37, 45, 53, 61, 69, 77, 85, 93, 106, 114, 122, 130, 138, 146 and 154; and a LCDR3 comprising an amino acid sequence selected from the group consisting of SEQ ID NOs: 6, 14, 22, 30, 38, 46, 54, 62, 70, 78, 86, 94, 107, 115, 123, 131, 139, 147, 155, 182, 183, 184, 185, 214, 215, 216 and 167.

In certain embodiments, the antibodies or antigen-binding fragments thereof provided herein comprise:
(a) a HCDR1 comprising an amino acid sequence as set forth in SEQ ID NO: 1, a HCDR2 comprising an amino acid sequence as set forth in SEQ ID NO: 2, and a HCDR3 comprising an amino acid sequence as set forth in SEQ ID NO: 3;
(b) a HCDR1 comprising an amino acid sequence as set forth in SEQ ID NO: 9, a HCDR2 comprising an amino acid sequence as set forth in SEQ ID NO: 10, and a HCDR3 comprising an amino acid sequence as set forth in SEQ ID NO: 11;
(c) a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
(d) a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
(e) a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
(f) a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
(g) a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
(h) a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
(i) a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
(j) a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
(k) a HCDR1 comprising an amino acid sequence as set forth in SEQ ID NO: 81, a HCDR2 comprising an amino acid sequence as set forth in SEQ ID NO: 82, and a HCDR3 comprising an amino acid sequence as set forth in SEQ ID NO: 83;
(l) a HCDR1 comprising an amino acid sequence as set forth in SEQ ID NO: 89, a HCDR2 comprising an amino acid sequence as set forth in SEQ ID NO: 90, and a HCDR3 comprising an amino acid sequence as set forth in SEQ ID NO: 91;
(m) a HCDR1 comprising an amino acid sequence as set forth in SEQ ID NO: 102, a HCDR2 comprising an amino acid sequence as set forth in SEQ ID NO: 103, and a HCDR3 comprising an amino acid sequence as set forth in SEQ ID NO: 104;
(n) a HCDR1 comprising an amino acid sequence as set forth in SEQ ID NO: 110, a HCDR2 comprising an amino acid sequence as set forth in SEQ ID NO: 111, and a HCDR3 comprising an amino acid sequence as set forth in SEQ ID NO: 112;
(o) a HCDR1 comprising an amino acid sequence as set forth in SEQ ID NO: 118, a HCDR2 comprising an amino acid sequence as set forth in SEQ ID NO: 119, and a HCDR3 comprising an amino acid sequence as set forth in SEQ ID NO: 120;
(p) a HCDR1 comprising an amino acid sequence as set forth in SEQ ID NO: 126, a HCDR2 comprising an amino acid sequence as set forth in SEQ ID NO: 127, and a HCDR3 comprising an amino acid sequence as set forth in SEQ ID NO: 128;
(q) a HCDR1 comprising an amino acid sequence as set forth in SEQ ID NO: 134, a HCDR2 comprising an amino acid sequence as set forth in SEQ ID NO: 135, and a HCDR3 comprising an amino acid sequence as set forth in SEQ ID NO: 136;
(r) a HCDR1 comprising an amino acid sequence as set forth in SEQ ID NO: 142, a HCDR2 comprising an amino acid sequence as set forth in SEQ ID NO: 143, and a HCDR3 comprising an amino acid sequence as set forth in SEQ ID NO: 144;
(s) a HCDR1 comprising an amino acid sequence as set forth in SEQ ID NO: 150, a HCDR2 comprising an amino acid sequence as set forth in SEQ ID NO: 151, and a HCDR3 comprising an amino acid sequence as set forth in SEQ ID NO: 152;
(t) a HCDR1 comprising an amino acid sequence as set forth in SEQ ID NO: 219, a HCDR2 comprising an amino acid sequence as set forth in SEQ ID NO: 201, and a HCDR3 comprising an amino acid sequence as set forth in SEQ ID NO: 162;
(u) a HCDR1 comprising an amino acid sequence as set forth in SEQ ID NO: 9, 217 or 218; a HCDR2 comprising an amino acid sequence as set forth in SEQ ID NO: 10, 171, 172, 173, 220 or 222; a HCDR3 comprising an amino acid sequence as set forth in SEQ ID NO: 11, 174, 175, 176, 177, 178, 179, 180, 181, 221, 223, 224, 225, 226, 227, 228 or 229;
(v) a HCDR1 comprising an amino acid sequence as set forth in SEQ ID NO: 150, a HCDR2 comprising an amino acid sequence as set forth in SEQ ID NO: 212, and a HCDR3 comprising an amino acid sequence as set forth in SEQ ID NO: 152;
(w) a HCDR1 comprising an amino acid sequence as set forth in SEQ ID NO: 150, a HCDR2 comprising an amino acid sequence as set forth in SEQ ID NO: 210, and a HCDR3 comprising an amino acid sequence as set forth in SEQ ID NO: 152; or
(x) a HCDR1 comprising an amino acid sequence as set forth in SEQ ID NO: 150, a HCDR2 comprising an amino acid sequence as set forth in SEQ ID NO: 211, and a HCDR3 comprising an amino acid sequence as set forth in SEQ ID NO: 152.

In certain embodiments, the antibodies or antigen-binding fragments thereof provided herein comprise:
(a) a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
(b) a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(c) a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
(d) a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
(e) a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
(f) a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
(g) a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
(h) a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
(i) a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
(j) a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
(k) a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
(l) a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
(m) a LCDR1 comprising an amino acid sequence as set forth in SEQ ID NO: 105, a LCDR2 comprising an amino acid sequence as set forth in SEQ ID NO: 106, and a LCDR3 comprising an amino acid sequence as set forth in SEQ ID NO: 107;
(n) a LCDR1 comprising an amino acid sequence as set forth in SEQ ID NO: 113, a LCDR2 comprising an amino acid sequence as set forth in SEQ ID NO: 114, and a LCDR3 comprising an amino acid sequence as set forth in SEQ ID NO: 115;
(o) a LCDR1 comprising an amino acid sequence as set forth in SEQ ID NO: 121, a LCDR2 comprising an amino acid sequence as set forth in SEQ ID NO: 122, and a LCDR3 comprising an amino acid sequence as set forth in SEQ ID NO: 123;
(p) a LCDR1 comprising an amino acid sequence as set forth in SEQ ID NO: 129, a LCDR2 comprising an amino acid sequence as set forth in SEQ ID NO: 130, and a LCDR3 comprising an amino acid sequence as set forth in SEQ ID NO: 131;
(q) a LCDR1 comprising an amino acid sequence as set forth in SEQ ID NO: 137, a LCDR2 comprising an amino acid sequence as set forth in SEQ ID NO: 138, and a LCDR3 comprising an amino acid sequence as set forth in SEQ ID NO: 139;
(r) a LCDR1 comprising an amino acid sequence as set forth in SEQ ID NO: 145, a LCDR2 comprising an amino acid sequence as set forth in SEQ ID NO: 146, and a LCDR3 comprising an amino acid sequence as set forth in SEQ ID NO: 147;
(s) a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
(t) a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 167; or
(u) a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14, 182, 183, 184, 185, 214, 215 or 216.

In certain embodiments, the antibodies or antigen-binding fragments thereof provided herein comprise:
(a) a HCDR1 comprising an amino acid sequence as set forth in SEQ ID NO: 1, a HCDR2 comprising an amino acid sequence as set forth in SEQ ID NO: 2, a HCDR3 comprising an amino acid sequence as set forth in SEQ ID NO: 3, a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
(b) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(c) a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
(d) a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
(e) a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
(f) a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
(g) a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
(h) a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
(i) a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
(j) a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
(k) a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
(l) a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
(m) a HCDR1 comprising an amino acid sequence as set forth in SEQ ID NO: 102, a HCDR2 comprising an amino acid sequence as set forth in SEQ ID NO: 103, a HCDR3 comprising an amino acid sequence as set forth in SEQ ID NO: 104, a LCDR1 comprising an amino acid sequence as set forth in SEQ ID NO: 105, a LCDR2 comprising an amino acid sequence as set forth in SEQ ID NO: 106, and a LCDR3 comprising an amino acid sequence as set forth in SEQ ID NO: 107;
(n) a HCDR1 comprising an amino acid sequence as set forth in SEQ ID NO: 110, a HCDR2 comprising an amino acid sequence as set forth in SEQ ID NO: 111, a HCDR3 comprising an amino acid sequence as set forth in SEQ ID NO: 112, a LCDR1 comprising an amino acid sequence as set forth in SEQ ID NO: 113, a LCDR2 comprising an amino acid sequence as set forth in SEQ ID NO: 114, and a LCDR3 comprising an amino acid sequence as set forth in SEQ ID NO: 115;
(o) a HCDR1 comprising an amino acid sequence as set forth in SEQ ID NO: 118, a HCDR2 comprising an amino acid sequence as set forth in SEQ ID NO: 119, a HCDR3 comprising an amino acid sequence as set forth in SEQ ID NO: 120, a LCDR1 comprising an amino acid sequence as set forth in SEQ ID NO: 121, a LCDR2 comprising an amino acid sequence as set forth in SEQ ID NO: 122, and a LCDR3 comprising an amino acid sequence as set forth in SEQ ID NO: 123;
(p) a HCDR1 comprising an amino acid sequence as set forth in SEQ ID NO: 126, a HCDR2 comprising an amino acid sequence as set forth in SEQ ID NO: 127, a HCDR3 comprising an amino acid sequence as set forth in SEQ ID NO: 128, a LCDR1 comprising an amino acid sequence as set forth in SEQ ID NO: 129, a LCDR2 comprising an amino acid sequence as set forth in SEQ ID NO: 130, and a LCDR3 comprising an amino acid sequence as set forth in SEQ ID NO: 131;
(q) a HCDR1 comprising an amino acid sequence as set forth in SEQ ID NO: 134, a HCDR2 comprising an amino acid sequence as set forth in SEQ ID NO: 135, a HCDR3 comprising an amino acid sequence as set forth in SEQ ID NO: 136, a LCDR1 comprising an amino acid sequence as set forth in SEQ ID NO: 137, a LCDR2 comprising an amino acid sequence as set forth in SEQ ID NO: 138, and a LCDR3 comprising an amino acid sequence as set forth in SEQ ID NO: 139;
(r) a HCDR1 comprising an amino acid sequence as set forth in SEQ ID NO: 142, a HCDR2 comprising an amino acid sequence as set forth in SEQ ID NO: 143, a HCDR3 comprising an amino acid sequence as set forth in SEQ ID NO: 144, a LCDR1 comprising an amino acid sequence as set forth in SEQ ID NO: 145, a LCDR2 comprising an amino acid sequence as set forth in SEQ ID NO: 146, and a LCDR3 comprising an amino acid sequence as set forth in SEQ ID NO: 147;
(s) a HCDR1 comprising an amino acid sequence as set forth in SEQ ID NO: 150, a HCDR2 comprising an amino acid sequence as set forth in SEQ ID NO: 151, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
(t) a HCDR1 comprising an amino acid sequence as set forth in SEQ ID NO: 9, a HCDR2 comprising an amino acid sequence as set forth in SEQ ID NO: 171, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(u) a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(v) a HCDR1 comprising an amino acid sequence as set forth in SEQ ID NO: 9, a HCDR2 comprising an amino acid sequence as set forth in SEQ ID NO: 173, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(w) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: NO: 12, a LCDR2 comprising an amino acid sequence as set forth in SEQ ID NO: 13, and a LCDR3 comprising an amino acid sequence as set forth in SEQ ID NO: 14;
(x) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 175, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(y) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 176, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(z) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 177, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(aa) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 178, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(bb) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 179, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(cc) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(dd) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 181, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(ee) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 182;
(ff) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 183;
(gg) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 184;
(hh) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 185;
(ii) a HCDR1 comprising an amino acid sequence as set forth in SEQ ID NO: 9, a HCDR2 comprising an amino acid sequence as set forth in SEQ ID NO: 220, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(jj) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 221, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(kk) a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(ll) a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(mm) a HCDR1 comprising an amino acid sequence as set forth in SEQ ID NO: 9, a HCDR2 comprising an amino acid sequence as set forth in SEQ ID NO: 171, a HCDR3 comprising an amino acid sequence as set forth in SEQ ID NO: 224, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(nn) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
(oo) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
(pp) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 216;
(qq) a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
(rr) a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
(ss) a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
(tt) a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
(uu) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 225, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(vv) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 226, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(ww) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 227, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(xx) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 228, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(yy) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: NO: 12, a LCDR2 comprising an amino acid sequence as set forth in SEQ ID NO: 13, and a LCDR3 comprising an amino acid sequence as set forth in SEQ ID NO: 14;
(zz) a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 229, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(aaa) a HCDR1 comprising an amino acid sequence as set forth in SEQ ID NO: 217, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(bbb) a HCDR1 comprising an amino acid sequence as set forth in SEQ ID NO: 218, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
(ccc) a HCDR1 comprising an amino acid sequence as set forth in SEQ ID NO: 219, a HCDR2 comprising an amino acid sequence as set forth in SEQ ID NO: 201, a HCDR3 comprising an amino acid sequence as set forth in SEQ ID NO: 162, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 167;
(ddd) a HCDR1 comprising an amino acid sequence as set forth in SEQ ID NO: 150, a HCDR2 comprising an amino acid sequence as set forth in SEQ ID NO: 212, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
(eee) a HCDR1 comprising an amino acid sequence as set forth in SEQ ID NO: 150, a HCDR2 comprising an amino acid sequence as set forth in SEQ ID NO: 210, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155; or
(fff) a HCDR1 comprising an amino acid sequence as set forth in SEQ ID NO: 150, a HCDR2 comprising an amino acid sequence as set forth in SEQ ID NO: 211, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155.

The SEQ ID NOs of the heavy chain (denoted as “H”) variable region, light chain (denoted as “L”) variable region, HCDRs and LCDRs of each of the 19 monoclonal antibodies described above are shown in Table 1 below. Unless otherwise indicated, the CDR boundaries as described herein were defined or identified by the convention of Kabat. The amino acid sequences of each CDR of the 19 exemplary monoclonal antibodies are shown in Table 2 below. The amino acid sequences of each VH and VL of the 19 exemplary monoclonal antibodies are shown in Table 3 below.

TABLE 1
SEQ ID NOs of VH, VL, HCDRs and LCDRs of
19 exemplary monoclonal antibodies.

| | | Variable | | | |
|---|---|---|---|---|---|
| | | Region | CDR1 | CDR2 | CDR3 |
| | | (SEQ | (SEQ | (SEQ | (SEQ |
| Antibody | Chain | ID NO) | ID NO) | ID NO) | ID NO) |
| 25-G12-G6-C12 | H | 7 | 1 | 2 | 3 |
| | L | 8 | 4 | 5 | 6 |
| 40-C12-C10-E9 | H | 15 | 9 | 10 | 11 |
| | L | 16 | 12 | 13 | 14 |
| 8-B12-F9-B11 | H | 23 | 17 | 18 | 19 |
| | L | 24 | 20 | 21 | 22 |
| 31-F8-F5-C5 | H | 31 | 25 | 26 | 27 |
| | L | 32 | 28 | 29 | 30 |
| 16-F2-C11-D9 | H | 39 | 33 | 34 | 35 |
| | L | 40 | 36 | 37 | 38 |
| 20-E11-E11-C2 | H | 47 | 41 | 42 | 43 |
| | L | 48 | 44 | 45 | 46 |
| 7-D9-G10-H2 | H | 55 | 49 | 50 | 51 |
| | L | 56 | 52 | 53 | 54 |
| 7-D8-G12-E4 | H | 63 | 57 | 58 | 59 |
| | L | 64 | 60 | 61 | 62 |
| 2-F12-A6-G2 | H | 71 | 65 | 66 | 67 |
| | L | 72 | 68 | 69 | 70 |
| 3-C6-C11-F12 | H | 79 | 73 | 74 | 75 |
| | I | 80 | 76 | 77 | 78 |
| 4-F12-F1-A4 | H | 87 | 81 | 82 | 83 |
| | L | 88 | 84 | 85 | 86 |
| 3-F3-G12-E2 | H | 95 | 89 | 90 | 91 |
| | L | 96 | 92 | 93 | 94 |
| 124E3D6 | H | 108 | 102 | 103 | 104 |
| | L | 109 | 105 | 106 | 107 |
| 126A11A4 | H | 116 | 110 | 111 | 112 |
| | L | 117 | 113 | 114 | 115 |
| 127E2D3 | H | 124 | 118 | 119 | 120 |
| | L | 125 | 121 | 122 | 123 |
| 133B4C7 | H | 132 | 126 | 127 | 128 |
| | L | 133 | 129 | 130 | 131 |
| 140D2B10 | H | 140 | 134 | 135 | 136 |
| | L | 141 | 137 | 138 | 139 |
| 147C6F3 | H | 148 | 142 | 143 | 144 |
| | L | 149 | 145 | 146 | 147 |
| 147E11E2 | H | 156 | 150 | 151 | 152 |
| | L | 157 | 153 | 154 | 155 |

TABLE 2
Amino acid sequences of each CDR of 19
exemplary monoclonal antibodies.

| Description | SEQ ID NO | Amino Acid Sequence |
|---|---|---|
| HCDR1 | 1 | SYVIY |
| | 9 | SYVIH |
| | 17 | DYGMH |
| | 25 | RYPIH |
| | 33 | DFNIH |
| | 41 | DYYMN |
| | 49 | GYFMN |
| | 57 | SYPMS |
| | 65 | SYGVS |
| | 73 | TYGMH |
| | 81 | SYTMS |
| | 89 | DYYMN |
| | 102 | SYWMH |
| | 110 | SYWMH |
| | 118 | SYWMH |
| | 126 | SYWMH |
| | 134 | SYWMH |
| | 142 | SYWMH |
| | 150 | SYWMH |
| HCDR2 | 2 | YINPYKDVTKYSEKFND |
| | 10 | YINPYNDVIKYNEKFKG |
| | 18 | YISSASGKIYYADTVKG |
| | 26 | YINPRNDYTKYTQRFKD |
| | 34 | YINPNDGGSTYNQKFKG |
| | 42 | DINPIHGGISSNQKFSD |
| | 50 | RFNPYNGDTFYNQNFKG |
| | 58 | TISSGGGNTYYPDSVKG |
| | 66 | VIWGDGNTNYNSDLKT |
| | 74 | YISNGGSTIYYADSVKG |
| | 82 | TISSGGGNTFYADSVKG |
| | 90 | EINPNNGGTTYRQTFKG |
| | 103 | NINPSNGDTNYNEKFKS |
| | 111 | NINPSNGDTNYNEKFKS |
| | 119 | NIYPGSSSINYNEKFKS |
| | 127 | NINPSNGDTNFNEKFKS |
| | 135 | NINPSNGESNYNEKFKS |
| | 143 | NLNPSNGEINYNEKFKH |
| | 151 | NINPSNGATNYNEKFKS |
| HCDR3 | 3 | AIYHDYDGFGY |
| | 11 | DSYYSYDGFAY |
| | 19 | VLRWSVNYVMDY |
| | 27 | SINDYDGFAY |
| | 35 | PFLDS |
| | 43 | RIYYGYDWYFDV |
| | 51 | GDY |
| | 59 | GLYWYFDV |
| | 67 | VSYWYFDV |
| | 75 | PTWGGFAY |
| | 83 | SLYYYDGTYVRKDYYAMDY |
| | 91 | SGY |
| | 104 | DRSSQYYFDY |
| | 112 | DRSSQYYFDY |
| | 120 | DRSSQYYFDY |
| | 128 | DRSSQYYFDY |
| | 136 | DRSSQYYFDY |
| | 144 | DRSSQYYFDY |
| | 152 | DRSSQYYFDY |
| LCDR1 | 4 | SASSSVTYIN |
| | 12 | SASSSVSYMH |
| | 20 | ESSQSVLYSSNQKNYLA |
| | 28 | SASSSVSYMN |
| | 36 | KASQDINSYLR |
| | 44 | SASSSVTSRYFF |
| | 52 | KSSQSLLDSDGKTYLN |
| | 60 | RSSQSIAHSNGDTYLE |
| | 68 | RASENIYSYLA |
| | 76 | RSSQSIAHSNGDTYLE |
| | 84 | RASENIFSNLA |
| | 92 | RSSQSIVHSNGNTYLE |
| | 105 | KSSQSLLNSRTRKNYLA |
| | 113 | KSSQSLLNSRTRKNYLA |
| | 121 | KSSQSLLNSRTRKNYLA |
| | 129 | KSSQSLLNSRTRKNYLA |
| | 137 | KSSQSLLNSRTRKNYLA |
| | 145 | KSSQSLLNSRTRKNYLA |
| | 153 | KSSQSLLNSRSRKNYLA |
| LCDR2 | 5 | DTSKLAS |
| | 13 | DTSKLAS |
| | 21 | WASTRES |
| | 29 | DTSKLAS |
| | 37 | GASSLVD |
| | 45 | STSNLAS |
| | 53 | LVSKLDS |
| | 61 | KVSNRIS |
| | 69 | NAKTLPE |
| | 77 | KVSNRIS |
| | 85 | AATNLAE |
| | 93 | KVSNRFS |
| | 106 | WASTRES |
| | 114 | WASTRES |
| | 122 | WASTRES |
| | 130 | WASTRES |
| | 138 | WASTRES |
| | 146 | WASTRES |
| | 154 | WASTRES |
| LCDR3 | 6 | QQWSRNPLT |
| | 14 | QQWSSNPPT |
| | 22 | HQYLSSWT |
| | 30 | QQWSSNPLT |
| | 38 | LQYDEVPLT |
| | 46 | QQYSTSPST |
| | 54 | WQGTHFPFT |
| | 62 | FQGSHVPYT |
| | 70 | QHHYGTPPT |
| | 78 | FQGSHVPYT |
| | 86 | QQFWGTPWT |
| | 94 | FQGSHVPRT |
| | 107 | KQSYNLRT |
| | 115 | KQSFILRT |
| | 123 | TQSYNLRT |
| | 131 | KQSYTLRT |
| | 139 | KQSYTLRT |
| | 147 | KQSYILRT |
| | 155 | TQSYTLRT |

TABLE 3
Amino acid sequences of each VH and VL of
19 exemplary monoclonal antibodies.

| Antibody | VH (SEQ ID NO) | VL (SEQ ID NO) |
|---|---|---|
| 25-G12- | EVQLQQSGPELVKPGASV | QIVLTQSPAIMSASPGEKVT |
| G6-C12 | KMSCKASGYKFTSYVIY | MTCSASSSVTYINWYQQM |
| | WVKQKPGQGLEWIGYIN | SGTSPKRWIYDTSKLASGV |
| | PYKDVTKYSEKFNDKATL | PARFSGSGSGTSYSLTISSLE |
| | TSDKSSSTAYMEISSLTSE | AEDAATYYCQQWSRNPLT |
| | DSAVYYCARAIYHDYDGF | FGAGTKLELK |
| | GYWGQGTLVIVSA | (SEQ ID NO: 8) |
| | (SEQ ID NO: 7) | |
| 40-C12- | EVQLQLSGPELVKPGASV | QIVLTQSPAIMSASPGEKVT |
| C10-E9 | KMSCKASGYTFSSYVIHW | MTCSASSSVSYMHWYQQK |
| | VKQKPGQGLEWIGYINPY | SGTSPKRWIYDTSKLASGV |
| | NDVIKYNEKFKGKATLTS | PARFSGSGSGTSYSLTISSM |
| | DKSSSTAYMELSSLTSEDS | EAEDAATYYCQQWSSNPPT |
| | AVYYCARDSYYSYDGFA | FGGGTKVEIK |
| | YWGQGTLVTVSA | (SEQ ID NO: 16) |
| | (SEQ ID NO: 15) | |
| 8-B12- | EVQLVESGGGIVKPGGSR | DIVMTQSPSSLAVSAGEKV |
| F9-B11 | KLSCAASGFTFSDYGMH | TMNCESSQSVLYSSNQKNY |
| | WVRQAPEKGLEWIAYISS | LAWYQQKPGQSPKLLIYW |
| | ASGKIYYADTVKGRFTISR | ASTRESGVPDRFTGTGSGT |
| | DNAKNILFLQMTSLRSED | DFTLTISSVQAEDLAVYYC |
| | TAMYYCARVLRWSVNYV | HQYLSSWTFGGGTKLEIK |
| | MDYWGQGTSVTVSS | (SEQ ID NO: 24) |
| | (SEQ ID NO: 23) | |
| 31-F8- | EVQLQQSGAELARPGASV | DIVLSQSPTILSASPGEKVT |
| F5-C5 | RMSCKASGYTFTRYPIHW | MTCSASSSVSYMNWYQQR |
| | VKQRPGQGLEWIGYINPR | SGTSPKRWIYDTSKLASGV |
| | NDYTKYTQRFKDKATLTA | PARFSGSGSGTSYSLTISSM |
| | DKSSSTAYMQLSSLTSEDS | EAEDAATYYCQQWSSNPLT |
| | AVYYCAKSINDYDGFAY | FGAGTKLELK |
| | WGQGTLVTVSA | (SEQ ID NO: 32) |
| | (SEQ ID NO: 31) | |
| 16-F2- | EVQLQQSGPELVKPGASV | TLMMTQSPSSMYASLGERV |
| C11-D9 | KMSCKASGYTFTDFNIH | TITCKASQDINSYLRWYQQ |
| | WVKQSLGKSLEWIGYINP | KPGKSPKTLIYGASSLVDG |
| | NDGGSTYNQKFKGKATLT | VPSRFSGRGSGQDYSLTISS |
| | INKSSSSAYMELRSLTSED | LEYEDMGIYYCLQYDEVPL |
| | SAVYYCAIPFLDSWGQGT | TFGAGTKLELK |
| | SVTVSS | (SEQ ID NO: 40) |
| | (SEQ ID NO: 39) | |
| 20-E11- | EVQLQQSGPEVVKPGASV | DIVLSQSPAIMSASPGERVT |
| E11-C2 | KISCKASGYTFTDYYMN | MTCSASSSVTSRYFFWYQQ |
| | WVKQSHGKNLEWIGDIN | KPGSSPKLWIYSTSNLASG |
| | PIHGGISSNQKFSDKATLT | VPVRFSGSGSGTSYSLTINN |
| | VDKSSNTAYLELRSLTSED | MAAEDAATYYCQQYSTSP |
| | SAIYYCARRIYYGYDWYF | STFGAGTKLELK |
| | DVWGTGTTVTVSS | (SEQ ID NO: 48) |
| | (SEQ ID NO: 47) | |
| 7-D9- | EVQLQQSGPELVKPGASV | DIQMTQSPLTLSVTIGQPASI |
| G10-H2 | KISCKASGYSFTGYFMNW | SCKSSQSLLDSDGKTYLNW |
| | VKQSHGKSLEWIGRFNPY | LLQRPGQSPKRLIYLVSKLD |
| | NGDTFYNQNFKGKATLTV | SGVPDRFTGSGSGTDFTLKI |
| | DKSSSTAHMELRSLTSEDS | SRVEAEDLGVYYCWQGTH |
| | AVYYCARGDYWGQGTTL | FPFTFGSGTKLEIK |
| | TVSS | (SEQ ID NO: 56) |
| | (SEQ ID NO: 55) | |
| 7-D8- | EVKLVESGGGLVKPGGSL | DIVLTQSPLSLPVSLGDQAS |
| G12-E4 | KLSCAASGFTFSSYPMSW | ISCRSSQSIAHSNGDTYLEW |
| | VRQTPAKRLEWVATISSG | YLQNPGQSPKLLIYKVSNRI |
| | GGNTYYPDSVKGRFTISR | SGVPDRFSGSGSGTDFTLKI |
| | DNARNTLYLQMSSLRSED | SRVEAEDLGVYYCFQGSH |
| | TAMYYCARGLYWYFDV | VPYTFGGGTKLEIK |
| | WGAGTTVTVSS | (SEQ ID NO: 64) |
| | (SEQ ID NO: 63) | |
| 2-F12- | EVQLVESRPGLVAPSQSLS | DIQLTQPPASLSASVGETVT |
| A6-G2 | ITCTVSGFSLTSYGVSWV | ITCRASENIYSYLAWYQQK |
| | RQPPGKGLEWLGVIWGD | QGKSPQLLVYNAKTLPEGV |
| | GNTNYNSDLKTSLSISKD | PSRFSGSGSGTQFSLKINSL |
| | NSKSQVFLKLDSLQTDDT | QPEDFGTYYCQHHYGTPPT |
| | ATYYCAKVSYWYFDVW | FGTGTKLELK |
| | GTGTTVTVSS | (SEQ ID NO: 72) |
| | (SEQ ID NO: 71) | |
| 3-C6- | EVHLVESGGGLVQPGGSR | DIVLTQSPLSLPVSLGDQAS |
| C11-F12 | KLSCAGSGFTFSTYGMH | ISCRSSQSIAHSNGDTYLEW |
| | WVRQAPEKGLEWVAYIS | YLQNPGQSPKLLIYKVSNRI |
| | NGGSTIYYADSVKGRFTIS | SGVPDRFSGSGSGTDFTLKI |
| | RDNPNDTLFLHMTSLGSE | SRVEAEDLGVYYCFQGSH |
| | DTAMYYCARPTWGGFAY | VPYTFGGGTKLEIK |
| | WGQGTLVTVSA | (SEQ ID NO: 80) |
| | (SEQ ID NO: 79) | |
| 4-F12- | EVKLVESGGGLVKPGGSL | DIQMTQSPASLSVSVGETV |
| F1-A4 | KLSCAASGFTFSSYTMSW | TISCRASENIFSNLAWYQQ |
| | IRQTPAKRLEWVATISSGG | KQGKSPQLLVYAATNLAEG |
| | GNTFYADSVKGRFTISRD | VPSRFSGSRSGTQFSLKINS |
| | NARNTLNLQMSSLRSEDT | LQSEDFGSYYCQQFWGTP |
| | AMYYCARSLYYYDGTYV | WTFGGGTKLEVK |
| | RKDYYAMDYWGQGSSV | (SEQ ID NO: 88) |
| | TVSS | |
| | (SEQ ID NO: 87) | |
| 3-F3- | EVQLQQSGPELVKPGASV | DIVMTQSPLSLPVSLGDQV |
| G12-E2 | KISCKASGNTFTDYYMN | AISCRSSQSIVHSNGNTYLE |
| | WVKQSHGKSLEWIGEINP | WYLQKPGQSPKLLIYKVSN |
| | NNGGTTYRQTFKGKATLT | RFSGVPDRFSGSGSGTDFTL |
| | VDKSSTTAYMELRSLTSE | KISRVEAEDLGVYYCFQGS |
| | DSAVYYCVLSGYWGQGT | HVPRTFGGGTKLELK |
| | GLTVSS | (SEQ ID NO: 96) |
| | (SEQ ID NO: 95) | |
| 124E3D6 | QVQLQQPGTELVKPGASV | DIVMSQSPSSLAVSAGEKV |
| | KLSCKASGYTFTSYWMH | TMSCKSSQSLLNSRTRKNY |
| | WVKQRPGQGLEWIGNIN | LAWYQQKPGQSPKLLIYW |
| | PSNGDTNYNEKFKSKATL | ASTRESGVPDRFTGSGSGT |
| | TVDKSSSTAYMQLSSLTSE | DFTLTISSVQAEDLAVYYC |
| | DSAVYYCARDRSSQYYFD | KQSYNLRTFGGGTKLEIK |
| | YWGQGTTLTVSS | (SEQ ID NO: 109) |
| | (SEQ ID NO: 108) | |
| 126A11A4 | QVQLQQPGTELVKPGASV | DIVMSQSPSSLAVSAGEKV |
| | KLSCKASGYTFTSYWMH | TMSCKSSQSLLNSRTRKNY |
| | WVKQRPEQGLEWIGNINP | LAWYQQKPGQSPKLLIYW |
| | SNGDTNYNEKFKSKATLT | ASTRESGVPDRFTGSGSGT |
| | VDKASNTAYMQLSSLTSE | DFTLTISSVQAEDLAVYFCK |
| | DSAVYYCGRDRSSQYYFD | QSFILRTFGGGTKLEIK |
| | YWGQGTTLTVSS | (SEQ ID NO: 117) |
| | (SEQ ID NO: 116) | |
| 127E2D3 | QVQLQQPGTELVKPGASV | DIVMSQSPSSLAVSAGEKV |
| | KLSCKASGYTFTSYWMH | TMSCKSSQSLLNSRTRKNY |
| | WVKQRPGEGLEWIGNIYP | LAWYQQKPGQSPKLLIYW |
| | GSSSINYNEKFKSKATLTV | ASTRESGVPDRFTGSGSGT |
| | DKSSSTAYMQLSSLTSEDS | DFTLTISSVQAEDLAVYYCT |
| | AVYYCARDRSSQYYFDY | QSYNLRTFGGGTKLEIK |
| | WGQGTTLTVSS | (SEQ ID NO: 125) |
| | (SEQ ID NO: 124) | |
| 133B4C7 | QVQLQQPGTELVKPGASV | DIVMSQSPSSLAVSAGEKV |
| | KLSCKASGYTFTSYWMH | TMSCNSSQSLLNSRTRKNY |
| | WVKQRPGQGLEWIGNIN | LAWYQQKPGQSPKLLIYW |
| | PSNGDTNFNEKFKSKATL | ASTRESGVPDRFTGSGSGT |
| | TVDKSSSTAYMQLSSLTSE | DFTLTISSVQAEDLAVYYC |
| | DSAVYYCARDRSSQYYFD | KQSYTLRTFGGGTKLEIK |
| | YWGQGTTLTVSS | (SEQ ID NO: 133) |
| | (SEQ ID NO: 132) | |
| 140D2B10 | QVQLQQPGTELVKPGASV | DIVMSQSPSSLAVSAGEKV |
| | KLSCKASGYTFTSYWMH | TMSCKSSQSLLNSRTRKNY |
| | WVKQRPGQGLEWIGNIN | LAWYQQKPGQSPKLLIYW |
| | PSNGESNYNEKFKSKATL | ASTRESGVPDRFTGSGSGT |
| | TVDKSSSTAYMQLSSLTSE | DFTLTISSVQAEDLAVYYC |
| | DYAVYYCARDRSSQYYF | KQSYTLRTFGGGTKLEIK |
| | DYWGQGTTLTVSS | (SEQ ID NO: 141) |
| | (SEQ ID NO: 140) | |
| 147C6F3 | QVQLQQPGTELVKPGASV | DIVMSQSPSSLAVSAGEKV |
| | KLSCKASGYTFDSYWMH | TLSCKSSQSLLNSRTRKNY |
| | WVKQRPGQGLEWIGNLN | LAWYQQKSGQSPKLLMY |
| | PSNGEINYNEKFKHKATL | WASTRESGVPDRFTGSGSG |
| | TVDKSSSTAYMQLSSLTSE | TDFTLTITNVQAEDLAVYY |
| | DSAVYYCSRDRSSQYYFD | CKQSYILRTFGGGTKLEIK |
| | YWGQGTTLTVSS | (SEQ ID NO: 149) |
| | (SEQ ID NO: 148) | |
| 147E11E2 | QVQLQQPGTELVKPGTSV | DIVMSQSPSSLAVSAGEKV |
| | KLSCKASGYTFTSYWMH | TMSCKSSQSLLNSRSRKNY |
| | WVKQRPGQGLEWIGNIN | LAWYQQKPGQSPKLLIYW |
| | PSNGATNYNEKFKSKATL | ASTRESGVPDRFTGSGSGT |
| | TVDKSSSTAYMQLSSLTSE | DFTLTISSVQAEDLAVYYCT |
| | DSAVYYCARDRSSQYYFD | QSYTLRTFGGGTKLEIK |
| | YWGQGTTLTVSS | (SEQ ID NO: 157) |
| | (SEQ ID NO: 156) | |

Given that each of the 19 exemplary monoclonal antibodies can bind to CD3 and that antigen-binding specificity is provided primarily by the CDR1, CDR2 and CDR3 regions, the HCDR1, HCDR2 and HCDR3 sequences and LCDR1, LCDR2 and LCDR3 sequences of each of the 19 exemplary monoclonal antibodies can be “mixed and matched” (i.e., CDRs from different antibodies can be mixed and matched, but each antibody must contain a HCDR1, HCDR2 and HCDR3 and a LCDR1, LCDR2 and LCDR3) to create anti-CD3 antibodies or antigen-binding fragments thereof of the present disclosure. CD3 binding of such “mixed and matched” antibodies can be tested using the binding assays described above and in the Examples. Preferably, when VH CDR sequences are mixed and matched, the HCDR1, HCDR2 and/or HCDR3 sequence from a particular VH sequence is replaced with a structurally similar CDR sequence(s). Likewise, when VL CDR sequences are mixed and matched, the LCDR1, LCDR2 and/or LCDR3 sequence from a particular VL sequence preferably is replaced with a structurally similar CDR sequence(s). For example, the HCDR1s of antibodies 25-G12-G6-C12 and 40-C12-C10-E9 share some structural similarity and therefore are amenable to mixing and matching. It will be readily apparent to a person skilled in the art that novel VH and VL sequences can be created by substituting one or more VH and/or VL CDR sequences with structurally similar sequences from the CDR sequences disclosed herein for the 19 exemplary monoclonal antibodies.

CDRs are known to be responsible for antigen binding. However, it has been found that not all of the 6 CDRs are indispensable or unchangeable. In other words, it is possible to replace or change or modify one or more CDRs in each of the 19 exemplary monoclonal antibodies, yet substantially retain the specific binding affinity to CD3.

In certain embodiments, the anti-CD3 antibodies and the antigen-binding fragments provided herein comprise a heavy chain CDR3 sequence of one of the anti-CD3 antibodies 25-G12-G6-C12, 40-C12-C10-E9, 8-B12-F9-B11, 31-F8-F5-C5, 16-F2-C11-D9, 20-E11-E11-C2, 7-D9-G10-H2, 7-D8-G12-E4, 2-F12-A6-G2, 3-C6-C11-F12, 4-F12-F1-A4, 3-F3-G12-E2, 124E3D6, 126A11A4, 127E2D3, 133B4C7, 140D2B10, 147C6F3 and 147E11E2. In certain embodiments, the anti-CD3 antibodies and the antigen-binding fragments provided herein comprise a heavy chain CDR3 sequence selected from the group consisting of SEQ ID NOs: 3, 11, 19, 27, 35, 43, 51, 59, 67, 75, 83, 91, 104, 112, 120, 128, 136, 144 and 152. Heavy chain CDR3 regions are located at the center of the antigen-binding site, and therefore are believed to make the most contact with antigen and provide the most free energy to the affinity of antibody to antigen. It is also believed that the heavy chain CDR3 is by far the most diverse CDR of the antigen-binding site in terms of length, amino acid composition and conformation by multiple diversification mechanisms (Tonegawa S. Nature. 302:575-81). The diversity in the heavy chain CDR3 is sufficient to produce most antibody specificities (Xu J L, Davis M M. Immunity. 13:37-45) as well as desirable antigen-binding affinity (Schier R, et al., J Mol Biol. 263:551-67).

In certain embodiments, the antibodies or antigen-binding fragments thereof provided herein comprise a VH region having an amino acid sequence as set forth in SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 or 245, or a homologous sequence thereof having at least 80% (e.g., at least 85%, at least 90%, at least 91%, at least 92%, at least 93%, at least 94%, at least 95%, at least 96%, at least 97%, at least 98%, or at least 99%) sequence identity to SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 or 245.

In certain embodiments, the antibodies or antigen-binding fragments thereof provided herein comprise a VL region having an amino acid sequence as set forth in SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 or 232, or a homologous sequence thereof having at least 80% (e.g., at least 85%, at least 90%, at least 91%, at least 92%, at least 93%, at least 94%, at least 95%, at least 96%, at least 97%, at least 98%, or at least 99%) sequence identity to SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 or 232.

In certain embodiments, the antibodies or antigen-binding fragments thereof provided herein comprise a VH/VL amino acid sequence pair selected from the group consisting of SEQ ID NOs: 7/8, 15/16, 23/24, 31/32, 39/40, 47/48, 55/56, 63/64, 71/72, 79/80, 87/88, 95/96, 108/109, 116/117, 124/125, 132/133, 140/141, 148/149, 156/157, 163/168, 163/169, 163/170, 164/168, 164/169, 164/170, 165/168, 165/169, 165/170, 166/168, 166/169, 166/170, 186/169, 187/169, 188/169, 189/169, 190/169, 191/169, 192/169, 193/169, 194/169, 195/169, 196/169, 165/197, 165/198, 165/199, 165/200, 202/207, 202/208, 202/209, 203/207, 203/208, 203/209, 204/207, 205/207, 206/207, 206/208, 206/209, 233/169, 234/169, 235/169, 236/169, 237/169, 238/169, 239/169, 240/169, 241/169, 242/169, 243/169, 244/169, 245/169, 165/230, 165/231, 165/232, 235/230, 235/231, 236/230 and 236/231.

In certain embodiments, the antibodies and antigen-binding fragments thereof provided herein comprise suitable framework region (FR) sequences, as long as the antibodies and antigen-binding fragments thereof can specifically bind to CD3. The CDR sequences provided in Table 2 above are obtained from mouse antibodies, but they can be grafted to any suitable FR sequences of any suitable species such as mouse, human, rat, rabbit, among others, using suitable methods known in the art such as recombinant techniques.

In certain embodiments, the antibodies and antigen-binding fragments thereof provided herein are humanized. A humanized antibody or antigen-binding fragment thereof is desirable in its reduced immunogenicity in human. A humanized antibody is chimeric in its variable regions, as non-human CDR sequences are grafted to human or substantially human FR sequences. Humanization of an antibody or antigen-binding fragment can be essentially performed by substituting the non-human (such as murine) CDR genes for the corresponding human CDR genes in a human immunoglobulin gene (see, for example, Jones et al., (1986) Nature 321:522-525; Riechmann et al., (1988) Nature 332:323-327; Verhoeyen et al., (1988) Science 239:1534-1536).

Suitable human heavy chain and light chain variable domains can be selected to achieve this purpose using methods known in the art. In an illustrative example, “best-fit” approach can be used, where a non-human (e.g., rodent) antibody variable domain sequence is screened or BLASTed against a database of known human variable domain sequences, and the human sequence closest to the non-human query sequence is identified and used as the human scaffold for grafting the non-human CDR sequences (see, for example, Sims et al., (1993) J. Immunol. 151:2296; Chothia et al., (1987) J. Mot. Biol. 196:901). Alternatively, a framework derived from the consensus sequence of all human antibodies may be used for the grafting of the non-human CDRs (see, for example, Carter et al., (1992) Proc. Natl. Acad. Sci. USA, 89:4285; Presta et al., (1993) J. Immunol., 151:2623).

In some embodiments, the anti-CD3 antibodies or antigen-binding fragments thereof provided herein are humanized. In certain embodiments, the humanized antibodies or antigen-binding fragments thereof provided herein are composed of substantially all human sequences except for the CDR sequences which are non-human. In some embodiments, the variable region FRs, and constant regions if present, are entirely or substantially from human immunoglobulin sequences. The human FR sequences and human constant region sequences may be derived from different human immunoglobulin genes, for example, FR sequences derived from one human antibody and constant region from another human antibody. In some embodiments, the humanized antibody or antigen-binding fragment thereof comprises human heavy chain HFR1, HFR2, HFR3 and HFR4, and/or light chain LFR1, LFR2, LFR3 and LFR4.

In some embodiments, the FR regions derived from human may comprise the same amino acid sequence as the human immunoglobulin from which it is derived. In some embodiments, one or more amino acid residues of the human FR are substituted with the corresponding residues from the parent non-human antibody. This may be desirable in certain embodiments to make the humanized antibody or its fragment closely approximate the non-human parent antibody structure, so as to optimize binding characteristics (for example, increase binding affinity). In certain embodiments, the humanized antibody or antigen-binding fragment thereof provided herein comprises no more than 10, 9, 8, 7, 6, 5, 4, 3, 2, or 1 amino acid residue substitutions in each of the human FR sequences, or no more than 10, 9, 8, 7, 6, 5, 4, 3, 2, or 1 amino acid residue substitutions in all the FR sequences of a heavy or a light chain variable domain. In some embodiments, such change in amino acid residue could be present in heavy chain FR regions only, in light chain FR regions only, or in both chains. In certain embodiments, one or more amino acids of the human FR sequences are randomly mutated to increase binding affinity. In certain embodiments, one or more amino acids of the human FR sequences are back mutated to the corresponding amino acid(s) of the parent non-human antibody so as to increase binding affinity.

In some embodiments, one, two, three or four amino acids at position(s) selected from the group consisting of positions 45, 46, 48 and 70 relative to SEQ ID NO: 168 (corresponding to positions 46, 47, 49 and 71 respectively according to Kabat numbering, or corresponding to positions 52, 53, 55 and 87 respectively according to IMGT numbering) of the human light chain FR sequences of the humanized anti-CD3 antibodies or antigen-binding fragments thereof provided herein is (are) back mutated to the corresponding amino acid(s) of the parent mouse antibody.

For example, one amino acid at position 45 relative to SEQ ID NO: 168 (corresponding to position 46 according to Kabat numbering, or position 52 according to IMGT numbering) of the human light chain FR sequences of the humanized anti-CD3 antibodies or antigen-binding fragments thereof provided herein is back mutated to the corresponding amino acid of the parent mouse antibody. In one embodiment, leucine at position 45 relative to SEQ ID NO: 168 (corresponding to position 46 according to Kabat numbering, or position 52 according to IMGT numbering) of the human light chain FR sequences of the humanized anti-CD3 antibodies or antigen-binding fragments thereof provided herein is back mutated to the corresponding amino acid of the parent mouse antibody. In another embodiment, leucine at position 45 relative to SEQ ID NO: 168 (corresponding to position 46 according to Kabat numbering, or position 52 according to IMGT numbering) of the human light chain FR sequences of the humanized anti-CD3 antibodies or antigen-binding fragments thereof provided herein is mutated to arginine (i.e., L45R).

For another example, one amino acid at position 46 relative to SEQ ID NO: 168 (corresponding to position 47 according to Kabat numbering, or position 53 according to IMGT numbering) of the human light chain FR sequences of the humanized anti-CD3 antibodies or antigen-binding fragments thereof provided herein is back mutated to the corresponding amino acid of the parent mouse antibody. In one embodiment, leucine at position 46 relative to SEQ ID NO: 168 (corresponding to position 47 according to Kabat numbering, or position 53 according to IMGT numbering) of the human light chain FR sequences of the humanized anti-CD3 antibodies or antigen-binding fragments thereof provided herein is back mutated to the corresponding amino acid of the parent mouse antibody. In another embodiment, leucine at position 46 relative to SEQ ID NO: 168 (corresponding to position 47 according to Kabat numbering, or position 53 according to IMGT numbering) of the human light chain FR sequences of the humanized anti-CD3 antibodies or antigen-binding fragments thereof provided herein is mutated to tryptophan (i.e., L46W).

For yet another example, one amino acid at position 48 relative to SEQ ID NO: 168 (corresponding to position 49 according to Kabat numbering, or position 55 according to IMGT numbering) of the human light chain FR sequences of the humanized anti-CD3 antibodies or antigen-binding fragments thereof provided herein is back mutated to the corresponding amino acid of the parent mouse antibody. In one embodiment, lysine at position 48 relative to SEQ ID NO: 168 (corresponding to position 49 according to Kabat numbering, or position 55 according to IMGT numbering) of the human light chain FR sequences of the humanized anti-CD3 antibodies or antigen-binding fragments thereof provided herein is back mutated to the corresponding amino acid of the parent mouse antibody. In another embodiment, lysine at position 48 relative to SEQ ID NO: 168 (corresponding to position 49 according to Kabat numbering, or position 55 according to IMGT numbering) of the human light chain FR sequences of the humanized anti-CD3 antibodies or antigen-binding fragments thereof provided herein is mutated to tyrosine (i.e., K48Y).

For yet another example, one amino acid at position 70 relative to SEQ ID NO: 168 (corresponding to position 71 according to Kabat numbering, or position 87 according to IMGT numbering) of the human light chain FR sequences of the humanized anti-CD3 antibodies or antigen-binding fragments thereof provided herein is back mutated to the corresponding amino acid of the parent mouse antibody. In one embodiment, phenylalanine at position 70 relative to SEQ ID NO: 168 (corresponding to position 71 according to Kabat numbering, or position 87 according to IMGT numbering) of the human light chain FR sequences of the humanized anti-CD3 antibodies or antigen-binding fragments thereof provided herein is back mutated to the corresponding amino acid of the parent mouse antibody. In another embodiment, phenylalanine at position 70 relative to SEQ ID NO: 168 (corresponding to position 71 according to Kabat numbering, or position 87 according to IMGT numbering) of the human light chain FR sequences of the humanized anti-CD3 antibodies or antigen-binding fragments thereof provided herein is mutated to tyrosine (i.e., F70Y).

For yet another example, one amino acid at position 45 relative to SEQ ID NO: 168 (corresponding to position 46 according to Kabat numbering, or position 52 according to IMGT numbering) and one amino acid at position 48 relative to SEQ ID NO: 168 (corresponding to position 49 according to Kabat numbering, or position 55 according to IMGT numbering) of the human light chain FR sequences of the humanized anti-CD3 antibodies or antigen-binding fragments thereof provided herein are back mutated to the corresponding amino acids of the parent mouse antibody. In one embodiment, leucine at position 45 relative to SEQ ID NO: 168 (corresponding to position 46 according to Kabat numbering, or position 52 according to IMGT numbering) and lysine at position 48 relative to SEQ ID NO: 168 (corresponding to position 49 according to Kabat numbering, or position 55 according to IMGT numbering) of the human light chain FR sequences of the humanized anti-CD3 antibodies or antigen-binding fragments thereof provided herein are back mutated to the corresponding amino acids of the parent mouse antibody. In another embodiment, leucine at position 45 relative to SEQ ID NO: 168 (corresponding to position 46 according to Kabat numbering, or position 52 according to IMGT numbering) and lysine at position 48 relative to SEQ ID NO: 168 (corresponding to position 49 according to Kabat numbering, or position 55 according to IMGT numbering) of the human light chain FR sequences of the humanized anti-CD3 antibodies or antigen-binding fragments thereof provided herein are back mutated to arginine and tyrosine, respectively (i.e., L45R+K48Y).

For yet another example, each amino acid at positions 45, 46, 48 and 70 relative to SEQ ID NO: 168 (corresponding to positions 46, 47, 49 and 71 respectively according to Kabat numbering, or corresponding to positions 52, 53, 55 and 87 respectively according to IMGT numbering) of the human light chain FR sequences of the humanized anti-CD3 antibodies or antigen-binding fragments thereof provided herein is back mutated to the corresponding amino acid of the parent mouse antibody. In one embodiment, leucine, leucine, lysine and phenylalanine at positions 45, 46, 48 and 70 respectively relative to SEQ ID NO: 168 (corresponding to positions 46, 47, 49 and 71 respectively according to Kabat numbering, or corresponding to positions 52, 53, 55 and 87 respectively according to IMGT numbering) of the human light chain FR sequences of the humanized anti-CD3 antibodies or antigen-binding fragments thereof provided herein is back mutated to the corresponding amino acid of the parent mouse antibody. In another embodiments, leucine, leucine, lysine and phenylalanine at positions 45, 46, 48 and 70 respectively relative to SEQ ID NO: 168 (corresponding to positions 46, 47, 49 and 71 respectively according to Kabat numbering, or corresponding to positions 52, 53, 55 and 87 respectively according to IMGT numbering) of the human light chain FR sequences of the humanized anti-CD3 antibodies or antigen-binding fragments thereof provided herein is mutated to arginine, tryptophan, tyrosine and tyrosine, respectively (i.e., L45R+L46W+K48Y+F70Y).

In some embodiments, one, two, three, four or five amino acids at position(s) selected from the group consisting of positions 27, 48, 68, 70 and 72 relative to SEQ ID NO: 163 (corresponding to positions 27, 48, 67, 69 and 71 respectively according to Kabat numbering, or corresponding to positions 28, 53, 76, 78 and 80 respectively according to IMGT numbering) of the human heavy chain FR sequences of the humanized anti-CD3 antibodies or antigen-binding fragments thereof provided herein is (are) back mutated to the corresponding amino acid(s) of the parent mouse antibody.

For example, one amino acid at position 27 relative to SEQ ID NO: 163 (corresponding to position 27 according to Kabat numbering, or corresponding to position 28 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, glycine at position 27 relative to SEQ ID NO: 163 (corresponding to position 27 according to Kabat numbering, or corresponding to position 28 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, glycine at position 27 relative to SEQ ID NO: 163 (corresponding to position 27 according to Kabat numbering, or corresponding to position 28 according to IMGT numbering) of the human heavy chain FR sequences of the humanized anti-CD3 antibodies or antigen-binding fragments thereof provided herein is back mutated to tyrosine (i.e., G27Y).

For another example, one amino acid at position 48 relative to SEQ ID NO: 163 (corresponding to position 48 according to Kabat numbering, or corresponding to position 53 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, methionine at position 48 relative to SEQ ID NO: 163 (corresponding to position 48 according to Kabat numbering, or corresponding to position 53 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, methionine at position 48 relative to SEQ ID NO: 163 (corresponding to position 48 according to Kabat numbering, or corresponding to position 53 according to IMGT numbering) of the human heavy chain FR sequences of the humanized anti-CD3 antibodies or antigen-binding fragments thereof provided herein is back mutated to isoleucine (i.e., M48I).

For another example, one amino acid at position 68 relative to SEQ ID NO: 163 (corresponding to position 67 according to Kabat numbering, or corresponding to position 76 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, valine at position 68 relative to SEQ ID NO: 163 (corresponding to position 67 according to Kabat numbering, or corresponding to position 76 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, valine at position 68 relative to SEQ ID NO: 163 (corresponding to position 67 according to Kabat numbering, or corresponding to position 76 according to IMGT numbering) of the human heavy chain FR sequences of the humanized anti-CD3 antibodies or antigen-binding fragments thereof provided herein is back mutated to alanine (i.e., V68A).

For another example, one amino acid at position 70 relative to SEQ ID NO: 163 (corresponding to position 69 according to Kabat numbering, or corresponding to position 78 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, isoleucine at position 70 relative to SEQ ID NO: 163 (corresponding to position 69 according to Kabat numbering, or corresponding to position 78 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, isoleucine at position 70 relative to SEQ ID NO: 163 (corresponding to position 69 according to Kabat numbering, or corresponding to position 78 according to IMGT numbering) of the human heavy chain FR sequences of the humanized anti-CD3 antibodies or antigen-binding fragments thereof provided herein is back mutated to leucine (i.e., I70L).

For another example, one amino acid at position 72 relative to SEQ ID NO: 163 (corresponding to position 71 according to Kabat numbering, or corresponding to position 80 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, alanine at position 72 relative to SEQ ID NO: 163 (corresponding to position 71 according to Kabat numbering, or corresponding to position 80 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, alanine at position 72 relative to SEQ ID NO: 163 (corresponding to position 71 according to Kabat numbering, or corresponding to position 80 according to IMGT numbering) of the human heavy chain FR sequences of the humanized anti-CD3 antibodies or antigen-binding fragments thereof provided herein is back mutated to serine (i.e., A72S).

For yet another example, each amino acid at positions 27, 70 and 72 relative to SEQ ID NO: 163 (corresponding to position 27, 69 and 71 respectively according to Kabat numbering, or corresponding to position 28, 78 and 80 respectively according to IMGT numbering) of the human heavy chain FR sequences of the humanized anti-CD3 antibodies or antigen-binding fragments thereof provided herein is back mutated to the corresponding amino acid of the parent mouse antibody. In one embodiment, glycine, isoleucine and alanine at positions 27, 70 and 72 respectively relative to SEQ ID NO: 163 (corresponding to position 27, 69 and 71 respectively according to Kabat numbering, or corresponding to position 28, 78 and 80 respectively according to IMGT numbering) of the human heavy chain FR sequences of the humanized anti-CD3 antibodies or antigen-binding fragments thereof provided herein is mutated to tyrosine, leucine and serine, respectively (i.e., G27Y+I70L+A72S).

For yet another example, each amino acid at positions 27, 48, 68, 70 and 72 relative to SEQ ID NO: 163 (corresponding to position 27, 48, 67, 69 and 71 respectively according to Kabat numbering, or corresponding to position 28, 53, 76, 78 and 80 respectively according to IMGT numbering) of the human heavy chain FR sequences of the humanized anti-CD3 antibodies or antigen-binding fragments thereof provided herein is back mutated to the corresponding amino acid of the parent mouse antibody. In one embodiment, glycine, methionine, valine, isoleucine and alanine at positions 27, 48, 68, 70 and 72 respectively relative to SEQ ID NO: 163 (corresponding to position 27, 48, 67, 69 and 71 respectively according to Kabat numbering, or corresponding to position 28, 53, 76, 78 and 80 respectively according to IMGT numbering) of the human heavy chain FR sequences of the humanized anti-CD3 antibodies or antigen-binding fragments thereof provided herein is mutated to tyrosine, isoleucine, alanine, leucine and serine, respectively (i.e., G27Y+M48I+V68A+I70L+A72S).

In some embodiments, one, two, three, four, five or six amino acids at position(s) selected from the group consisting of position 27, 30, 48, 68, 70 and 72 relative to SEQ ID NO: 213 (corresponding to position 27, 30, 48, 67, 69 and 71 respectively according to Kabat numbering, or corresponding to position 28, 35, 53, 76, 78 and 80 respectively according to IMGT numbering) of the human heavy chain FR sequences of the humanized anti-CD3 antibodies or antigen-binding fragments thereof provided herein is (are) back mutated to the corresponding amino acid(s) of the parent mouse antibody.

For example, one amino acid at position 27 relative to SEQ ID NO: 213 (corresponding to position 27 according to Kabat numbering, or corresponding to position 28 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, glycine at position 27 relative to SEQ ID NO: 213 (corresponding to position 27 according to Kabat numbering, or corresponding to position 28 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, glycine at position 27 relative to SEQ ID NO: 213 (corresponding to position 27 according to Kabat numbering, or corresponding to position 28 according to IMGT numbering) of the human heavy chain FR sequences of the humanized anti-CD3 antibodies or antigen-binding fragments thereof provided herein is back mutated to tyrosine (i.e., G27Y).

For another example, one amino acid at position 30 relative to SEQ ID NO: 213 (corresponding to position 30 according to Kabat numbering, or corresponding to position 35 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, serine at position 30 relative to SEQ ID NO: 213 (corresponding to position 30 according to Kabat numbering, or corresponding to position 35 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, serine at position 30 relative to SEQ ID NO: 213 (corresponding to position 30 according to Kabat numbering, or corresponding to position 35 according to IMGT numbering) of the human heavy chain FR sequences of the humanized anti-CD3 antibodies or antigen-binding fragments thereof provided herein is back mutated to threonine (i.e., S30T).

For another example, one amino acid at position 48 relative to SEQ ID NO: 213 (corresponding to position 48 according to Kabat numbering, or corresponding to position 53 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, methionine at position 48 relative to SEQ ID NO: 213 (corresponding to position 48 according to Kabat numbering, or corresponding to position 53 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, methionine at position 48 relative to SEQ ID NO: 213 (corresponding to position 48 according to Kabat numbering, or corresponding to position 53 according to IMGT numbering) of the human heavy chain FR sequences of the humanized anti-CD3 antibodies or antigen-binding fragments thereof provided herein is back mutated to isoleucine (i.e., M48I).

For another example, one amino acid at position 68 relative to SEQ ID NO: 213 (corresponding to position 67 according to Kabat numbering, or corresponding to position 76 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, valine at position 68 relative to SEQ ID NO: 213 (corresponding to position 67 according to Kabat numbering, or corresponding to position 76 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, valine at position 68 relative to SEQ ID NO: 213 (corresponding to position 67 according to Kabat numbering, or corresponding to position 76 according to IMGT numbering) of the human heavy chain FR sequences of the humanized anti-CD3 antibodies or antigen-binding fragments thereof provided herein is back mutated to alanine (i.e., V68A).

For another example, one amino acid at position 70 relative to SEQ ID NO: 213 (corresponding to position 69 according to Kabat numbering, or corresponding to position 78 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, isoleucine at position 70 relative to SEQ ID NO: 213 (corresponding to position 69 according to Kabat numbering, or corresponding to position 78 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, isoleucine at position 70 relative to SEQ ID NO: 213 (corresponding to position 69 according to Kabat numbering, or corresponding to position 78 according to IMGT numbering) of the human heavy chain FR sequences of the humanized anti-CD3 antibodies or antigen-binding fragments thereof provided herein is back mutated to leucine (i.e., I70L).

For another example, one amino acid at position 72 relative to SEQ ID NO: 213 (corresponding to position 71 according to Kabat numbering, or corresponding to position 80 according to IMGT numbering) of the human heavy chain FR sequences of the humanized anti-CD3 antibodies or antigen-binding fragments thereof provided herein is back mutated to the corresponding amino acid of the parent mouse antibody. In one embodiment, alanine at position 72 relative to SEQ ID NO: 213 (corresponding to position 71 according to Kabat numbering, or corresponding to position 80 according to IMGT numbering) of the human heavy chain FR sequences of the humanized anti-CD3 antibodies or antigen-binding fragments thereof provided herein is back mutated to the corresponding amino acid of the parent mouse antibody. In another embodiment, alanine at position 72 relative to SEQ ID NO: 213 (corresponding to position 71 according to Kabat numbering, or corresponding to position 80 according to IMGT numbering) of the human heavy chain FR sequences of the humanized anti-CD3 antibodies or antigen-binding fragments thereof provided herein is back mutated to valine (i.e., A72V).

For yet another example, each amino acid at positions 27 and 30 relative to SEQ ID NO: 213 (corresponding to position 27 and 30 respectively according to Kabat numbering, or corresponding to position 28 and 35 respectively according to IMGT numbering) of the human heavy chain FR sequences of the humanized anti-CD3 antibodies or antigen-binding fragments thereof provided herein is back mutated to the corresponding amino acid of the parent mouse antibody. In one embodiment, glycine and serine at positions 27 and 30 respectively relative to SEQ ID NO: 213 (corresponding to position 27 and 30 respectively according to Kabat numbering, or corresponding to position 28 and 35 respectively according to IMGT numbering) of the human heavy chain FR sequences of the humanized anti-CD3 antibodies or antigen-binding fragments thereof provided herein are back mutated to the corresponding amino acids of the parent mouse antibody. In another embodiment, glycine and serine at positions 27 and 30 respectively relative to SEQ ID NO: 213 (corresponding to position 27 and 30 respectively according to Kabat numbering, or corresponding to position 28 and 35 respectively according to IMGT numbering) of the human heavy chain FR sequences of the humanized anti-CD3 antibodies or antigen-binding fragments thereof provided herein are mutated to tyrosine and threonine, respectively (i.e., G27Y+S30T).

For yet another example, each amino acid at positions 27, 30, 70 and 72 relative to SEQ ID NO: 213 (corresponding to position 27, 30, 69 and 71 respectively according to Kabat numbering, or corresponding to position 28, 35, 78 and 80 respectively according to IMGT numbering) of the human heavy chain FR sequences of the humanized anti-CD3 antibodies or antigen-binding fragments thereof provided herein is back mutated to the corresponding amino acid of the parent mouse antibody. In one embodiment, glycine, serine, isoleucine and alanine at positions 27, 30, 70 and 72 respectively relative to SEQ ID NO: 213 (corresponding to position 27, 30, 69 and 71 respectively according to Kabat numbering, or corresponding to position 28, 35, 78 and 80 respectively according to IMGT numbering) of the human heavy chain FR sequences of the humanized anti-CD3 antibodies or antigen-binding fragments thereof provided herein are back mutated to the corresponding amino acids of the parent mouse antibody. In another embodiment, glycine, serine, isoleucine and alanine at positions 27, 30, 70 and 72 respectively relative to SEQ ID NO: 213 (corresponding to position 27, 30, 69 and 71 respectively according to Kabat numbering, or corresponding to position 28, 35, 78 and 80 respectively according to IMGT numbering) of the human heavy chain FR sequences of the humanized anti-CD3 antibodies or antigen-binding fragments thereof provided herein are mutated to tyrosine, threonine, leucine and valine, respectively (i.e., G27Y+S30T+I70L+A72V).

For yet another example, each amino acid at positions 27, 30, 48, 68, 70 and 72 relative to SEQ ID NO: 213 (corresponding to position 27, 30, 48, 67, 69 and 71 respectively according to Kabat numbering, or corresponding to position 28, 35, 53, 76, 78 and 80 respectively according to IMGT numbering) of the human heavy chain FR sequences of the humanized anti-CD3 antibodies or antigen-binding fragments thereof provided herein is back mutated to the corresponding amino acid of the parent mouse antibody. In one embodiment, glycine, serine, methionine, valine, isoleucine and alanine at positions 27, 30, 48, 68, 70 and 72 respectively relative to SEQ ID NO: 213 (corresponding to position 27, 30, 48, 67, 69 and 71 respectively according to Kabat numbering, or corresponding to position 28, 35, 53, 76, 78 and 80 respectively according to IMGT numbering) of the human heavy chain FR sequences of the humanized anti-CD3 antibodies or antigen-binding fragments thereof provided herein are back mutated to the corresponding amino acids of the parent mouse antibody. In another embodiment, glycine, serine, methionine, valine, isoleucine and alanine at positions 27, 30, 48, 68, 70 and 72 respectively relative to SEQ ID NO: 213 (corresponding to position 27, 30, 48, 67, 69 and 71 respectively according to Kabat numbering, or corresponding to position 28, 35, 53, 76, 78 and 80 respectively according to IMGT numbering) of the human heavy chain FR sequences of the humanized anti-CD3 antibodies or antigen-binding fragments thereof provided herein are mutated to tyrosine, threonine, isoleucine, alanine, leucine and valine, respectively (i.e., G27Y+S30T+M48I+V68A+I70L+A72V).

In some embodiments, one amino acid at position 49 relative to SEQ ID NO: 207 (corresponding to position 43 according to Kabat numbering, or corresponding to position 49 according to IMGT numbering) of the human light chain FR sequences of the humanized anti-CD3 antibodies or antigen-binding fragments thereof provided herein is back mutated to the corresponding amino acid of the parent mouse antibody. In one embodiment, proline at position 49 relative to SEQ ID NO: 207 (corresponding to position 43 according to Kabat numbering, or corresponding to position 49 according to IMGT numbering) of the human light chain FR sequences of the humanized anti-CD3 antibodies or antigen-binding fragments thereof provided herein is back mutated to the corresponding amino acid of the parent mouse antibody. In another embodiment, proline at position 49 relative to SEQ ID NO: 207 (corresponding to position 43 according to Kabat numbering, or corresponding to position 49 according to IMGT numbering) of the human light chain FR sequences of the humanized anti-CD3 antibodies or antigen-binding fragments thereof provided herein is back mutated to serine (i.e., P49S).

In some embodiments, the present disclosure provides a humanized antibody or antigen-binding fragment thereof of clone 40-C12-C10-E9 (also referred to as “humanized 40-C12-C10-E9” in the present disclosure). In some embodiments, the present disclosure provides 12 humanized 40-C12-C10-E9, which are designated as hu40E9-L1H1, hu40E9-L1H2, hu40E9-L1H3, hu40E9-L1H4, hu40E9-L2H1, hu40E9-L2H2, hu40E9-L2H3, hu40E9-L2H4, hu40E9-L3H1, hu40E9-L3H2, hu40E9-L3H3 and hu40E9-L3H4, respectively. The SEQ ID NOs of the heavy and light chain variable regions of each humanized 40-C12-C10-E9 are shown in Table 20 below (the CDR sequences identified by the convention of Kabat are underlined). Each of the 12 humanized 40-C12-C10-E9 clones comprises a HCDR1, a HCDR2, a HCDR3, a LCDR1, a LCDR2 and a LCDR3 comprising an amino acid sequence as set forth in SEQ ID NOs: 9, 10, 11, 12, 13 and 14, respectively.

In some embodiments, the present disclosure provides a humanized antibody or antigen-binding fragment thereof of clone 147E11E2 (also referred to as “humanized 147E11E2” in the present disclosure). In some embodiments, the present disclosure provides 11 humanized 147E11E2, which are designated as hu147E2-L1H2, hu147E2-L1H3, hu147E2-L1H3a, hu147E2-L1H3b, hu147E2-L1H4, hu147E2-L2H2, hu147E2-L2H3, hu147E2-L2H4, hu147E2-L3H2, hu147E2-L3H3 and hu147E2-L3H4, respectively. The SEQ ID NOs of the heavy and light chain variable regions of each humanized 147E11E2 are shown in Table 20 below (the CDR sequences identified by the convention of Kabat are underlined). Each of the 11 humanized 147E11E2 clones comprises a HCDR1, a HCDR2, a HCDR3, a LCDR1, a LCDR2 and a LCDR3 comprising an amino acid sequence as set forth in SEQ ID NOs: 150, 212, 152, 153, 154 and 155, respectively.

In some embodiments, some motifs (e.g., N55G56 motif) which are liable to deamination may be identified within one or more CDRs (e.g., HCDR2) of humanized antibodies or antigen-binding fragments thereof. To remove such deamidation sites, different mutations can be introduced to the motifs, yet still retain specific binding to CD3. In some embodiments, a deamination site is identified in the amino acid sequence (i.e., SEQ ID NO: 203) of hu147E2-H3, which have the same CDRs as the heavy chain of clone 147E11E2. In some embodiments, one or more amino acids within one or more CDRs of a candidate antibody is mutated to remove the deamination site. In some embodiments, the mutation occurs within HCDR2 (e.g., position 55 relative to SEQ ID NO: 213) of the candidate antibody. Accordingly, the present disclosure provides anti-CD3 antibodies or antigen-binding fragments thereof which have the same CDRs as clone 147E11E2 except that the amino acid sequence of HCDR2 is different. For example, the anti-CD3 antibodies or antigen-binding fragments thereof provided herein comprise a HCDR2 comprising an amino acid sequence as set forth in SEQ ID NO: 212. In some embodiments, the present disclosure provides a humanized antibody hu147E2-L1H3a, which comprises a HCDR2 comprising an amino acid sequence as set forth in SEQ ID NO: 210, which carries a single mutation (i.e., N55Q) compared to the parental HCDR2 (i.e., SEQ ID NO: 151), to remove the deamination site NG motif. In some embodiments, the present disclosure provides a humanized antibody hu147E2-L1H3b, which comprises a HCDR2 comprising an amino acid sequence as set forth in SEQ ID NO: 211, which carries a single mutation (i.e., N55S) compared to the parental HCDR2 (i.e., SEQ ID NO: 151), to remove the deamination site NG motif.

In some embodiments, the anti-CD3 antibodies or antigen-binding fragments thereof provided herein comprise a HCDR1 comprising an amino acid sequence as set forth in SEQ ID NO: 150, a HCDR2 comprising an amino acid sequence as set forth in SEQ ID NO: 212, and a HCDR3 comprising an amino acid sequence as set forth in SEQ ID NO: 152. In some embodiments, the anti-CD3 antibodies or antigen-binding fragments thereof provided herein comprises a HCDR1, HCDR2, HCDR3, LCDR1, LCDR2 and LCDR3 comprising an amino acid sequence as set forth in SEQ ID NO: 150, 212, 152, 153, 154 and 155, respectively. In some embodiments, the anti-CD3 antibodies or antigen-binding fragments thereof provided herein comprises a HCDR1, HCDR2, HCDR3, LCDR1, LCDR2 and LCDR3 comprising an amino acid sequence as set forth in SEQ ID NO: 150, 210, 152, 153, 154 and 155, respectively. In some embodiments, the anti-CD3 antibodies or antigen-binding fragments thereof provided herein comprises a HCDR1, HCDR2, HCDR3, LCDR1, LCDR2 and LCDR3 comprising an amino acid sequence as set forth in SEQ ID NO: 150, 211, 152, 153, 154 and 155, respectively. The amino acid sequences of SEQ ID NOs: 210, 211 and 212 are shown in Table 22 below.

In some embodiments, the humanized antibody or antigen-binding fragment thereof provided herein comprises a VH region having an amino acid sequence as set forth in SEQ ID NOs: 163, 164, 165, 166, 213, 202, 203, 204, 205, 206, or a homologous sequence thereof having at least 80% (e.g., at least 85%, at least 90%, at least 91%, at least 92%, at least 93%, at least 94%, at least 95%, at least 96%, at least 97%, at least 98%, or at least 99%) identity to SEQ ID NOs: 163, 164, 165, 166, 213, 202, 203, 204, 205 or 206. In some embodiments, the humanized antibody or antigen-binding fragment thereof provided herein comprises a VL region having an amino acid sequence as set forth in SEQ ID NOs: 168, 169, 170, 207, 208, 209, or a homologous sequence thereof having at least 80% (e.g., at least 85%, at least 90%, at least 91%, at least 92%, at least 93%, at least 94%, at least 95%, at least 96%, at least 97%, at least 98%, or at least 99%) identity to SEQ ID NOs: 168, 169, 170, 207, 208 or 209.

The present disclosure also provides exemplary humanized antibodies of clone 40-C12-C10-E9, including:
1) “hu40E9-L1H1” comprising the heavy chain variable region of hu40E9-H1 (SEQ ID NO: 163) and the light chain variable region of hu40E9-L1 (SEQ ID NO: 168);
2) “hu40E9-L1H2” comprising the heavy chain variable region of hu40E9-H2 (SEQ ID NO: 164) and the light chain variable region of hu40E9-L1 (SEQ ID NO: 168);
3) “hu40E9-L1H3” comprising the heavy chain variable region of hu40E9-H3 (SEQ ID NO: 165) and the light chain variable region of hu40E9-L1 (SEQ ID NO: 168);
4) “hu40E9-L1H4” comprising the heavy chain variable region of hu40E9-H4 (SEQ ID NO: 166) and the light chain variable region of hu40E9-L1 (SEQ ID NO: 168);
5) “hu40E9-L2H1” comprising the heavy chain variable region of hu40E9-H1 (SEQ ID NO: 163) and the light chain variable region of hu40E9-L2 (SEQ ID NO: 169);
6) “hu40E9-L2H2” comprising the heavy chain variable region of hu40E9-H2 (SEQ ID NO: 164) and the light chain variable region of hu40E9-L2 (SEQ ID NO: 169);
7) “hu40E9-L2H3” comprising the heavy chain variable region of hu40E9-H3 (SEQ ID NO: 165) and the light chain variable region of hu40E9-L2 (SEQ ID NO: 169);
8) “hu40E9-L2H4” comprising the heavy chain variable region of hu40E9-H4 (SEQ ID NO: 166) and the light chain variable region of hu40E9-L2 (SEQ ID NO: 169);
9) “hu40E9-L3H1” comprising the heavy chain variable region of hu40E9-H1 (SEQ ID NO: 163) and the light chain variable region of hu40E9-L3 (SEQ ID NO: 170);
10) “hu40E9-L3H2” comprising the heavy chain variable region of hu40E9-H2 (SEQ ID NO: 164) and the light chain variable region of hu40E9-L3 (SEQ ID NO: 170);
11) “hu40E9-L3H3” comprising the heavy chain variable region of hu40E9-H3 (SEQ ID NO: 165) and the light chain variable region of hu40E9-L3 (SEQ ID NO: 170); or
12) “hu40E9-L3H4” comprising the heavy chain variable region of hu40E9-H4 (SEQ ID NO: 166) and the light chain variable region of hu40E9-L3 (SEQ ID NO: 170).

The present disclosure also provides exemplary humanized antibodies of clone 147E211E2, including:
1) “hu14E2-L1H2” comprising the heavy chain variable region of hu14E2-H2 (SEQ ID NO: 202) and the light chain variable region of hu14E2-L1 (SEQ ID NO: 207);
2) “hu14E2-L1H3” comprising the heavy chain variable region of hu14E2-H3 (SEQ ID NO: 203) and the light chain variable region of hu14E2-L1 (SEQ ID NO: 207);
3) “hu14E2-L1H3a” comprising the heavy chain variable region of hu14E2-H3a (SEQ ID NO: 204) and the light chain variable region of hu14E2-L1 (SEQ ID NO: 207);
4) “hu14E2-L1H3b” comprising the heavy chain variable region of hu14E2-H3b (SEQ ID NO: 205) and the light chain variable region of hu14E2-L1 (SEQ ID NO: 207);
5) “hu14E2-L1H4” comprising the heavy chain variable region of hu14E2-H4 (SEQ ID NO: 206) and the light chain variable region of hu14E2-L1 (SEQ ID NO: 207);
6) “hu14E2-L2H2” comprising the heavy chain variable region of hu14E2-H2 (SEQ ID NO: 202) and the light chain variable region of hu14E2-L2 (SEQ ID NO: 208);
7) “hu14E2-L2H3” comprising the heavy chain variable region of hu14E2-H3 (SEQ ID NO: 203) and the light chain variable region of hu14E2-L2 (SEQ ID NO: 208);
8) “hu14E2-L2H4” comprising the heavy chain variable region of hu14E2-H4 (SEQ ID NO: 206) and the light chain variable region of hu14E2-L2 (SEQ ID NO: 208);
9) “hu14E2-L3H2” comprising the heavy chain variable region of hu14E2-H2 (SEQ ID NO: 202) and the light chain variable region of hu14E2-L3 (SEQ ID NO: 209);
10) “hu14E2-L3H3” comprising the heavy chain variable region of hu14E2-H3 (SEQ ID NO: 203) and the light chain variable region of hu14E2-L3 (SEQ ID NO: 209); or
11) “hu14E2-L3H4” comprising the heavy chain variable region of hu14E2-H4 (SEQ ID NO: 206) and the light chain variable region of hu14E2-L3 (SEQ ID NO: 209).

TABLE 20
Amino acid sequences of each VH and VL of
humanized 40-C12-C10-E9 and 147E11E2

| | SEQ | |
|---|---|---|
| | ID | |
| Description | NO | Amino Acid Sequence |
| hu40E9-H1 | 163 | EVQLVQSGAEVKKPGSSVKVSCKASGGTFSSY |
| | | VIHWVRQAPGQGLEWMGYINPYNDVIKYNEK |
| | | FKGRVTITADKSTSTAYMELSSLRSEDTAVYYC |
| | | ARDSYYSYDGFAYWGQGTLVTVSS |
| hu40E9-H2 | 164 | EVQLVQSGAEVKKPGSSVKVSCKASGYTFSSY |
| | | VIHWVRQAPGQGLEWMGYINPYNDVIKYNEK |
| | | FKGRVTITADKSTSTAYMELSSLRSEDTAVYYC |
| | | ARDSYYSYDGFAYWGQGTLVTVSS |
| hu40E9-H3 | 165 | EVQLVQSGAEVKKPGSSVKVSCKASGYTFSSY |
| | | VIHWVRQAPGQGLEWMGYINPYNDVIKYNEK |
| | | FKGRVTLTSDKSTSTAYMELSSLRSEDTAVYY |
| | | CARDSYYSYDGFAYWGQGTLVTVSS |
| hu40E9-H4 | 166 | EVQLVQSGAEVKKPGSSVKVSCKASGYTFSSY |
| | | VIHWVRQAPGQGLEWIGYINPYNDVIKYNEKF |
| | | KGRATLTSDKSTSTAYMELSSLRSEDTAVYYC |
| | | ARDSYYSYDGFAYWGQGTLVTVSS |
| hu40E9-L1 | 168 | EIVLTQSPDFQSVTPKEKVTITCSASSSVSYMH |
| | | WYQQKPDQSPKLLIKDTSKLASGVPSRFSGSGS |
| | | GTDFTLTINSLEAEDAATYYCQQWSSNPPTFGG |
| | | GTKVEIK |
| hu40E9-L2 | 169 | EIVLTQSPDFQSVTPKEKVTITCSASSSVSYMH |
| | | WYQQKPDQSPKRLIYDTSKLASGVPSRFSGSGS |
| | | GTDFTLTINSLEAEDAATYYCQQWSSNPPTFGG |
| | | GTKVEIK |
| hu40E9-L3 | 170 | EIVLTQSPDFQSVTPKEKVTITCSASSSVSYMH |
| | | WYQQKPDQSPKRWIYDTSKLASGVPSRFSGSG |
| | | SGTDYTLTINSLEAEDAATYYCQQWSSNPPTFG |
| | | GGTKVEIK |
| hu147E2-H1 | 213 | EVQLVQSGAEVKKPGSSVKVSCKASGGTFSSY |
| | | WMHWVRQAPGQGLEWMGNINPSNGATNYNE |
| | | KFKSRVTITADKSTSTAYMELSSLRSEDTAVYY |
| | | CARDRSSQYYFDYWGQGTTVTVSS |
| hu147E2-H2 | 202 | EVQLVQSGAEVKKPGSSVKVSCKASGYTFTSY |
| | | WMHWVRQAPGQGLEWMGNINPSNGATNYNE |
| | | KFKSRVTITADKSTSTAYMELSSLRSEDTAVYY |
| | | CARDRSSQYYFDYWGQGTTVTVSS |
| hu147E2-H3 | 203 | EVQLVQSGAEVKKPGSSVKVSCKASGYTFTSY |
| | | WMHWVRQAPGQGLEWMGNINPSNGATNYNE |
| | | KFKSRVTLTVDKSTSTAYMELSSLRSEDTAVY |
| | | YCARDRSSQYYFDYWGQGTTVTVSS |
| hu147E2-H3a | 204 | EVQLVQSGAEVKKPGSSVKVSCKASGYTFTSY |
| | | WMHWVRQAPGQGLEWMGNINPSQGATNYNE |
| | | KFKSRVTLTVDKSTSTAYMELSSLRSEDTAVY |
| | | YCARDRSSQYYFDYWGQGTTVTVSS |
| hu147E2-H3b | 205 | EVQLVQSGAEVKKPGSSVKVSCKASGYTFTSY |
| | | WMHWVRQAPGQGLEWMGNINPSSGATNYNE |
| | | KFKSRVTLTVDKSTSTAYMELSSLRSEDTAVY |
| | | YCARDRSSQYYFDYWGQGTTVTVSS |
| hu147E2-H4 | 206 | EVQLVQSGAEVKKPGSSVKVSCKASGYTFTSY |
| | | WMHWVRQAPGQGLEWIGNINPSNGATNYNEK |
| | | FKSRATLTVDKSTSTAYMELSSLRSEDTAVYY |
| | | CARDRSSQYYFDYWGQGTTVTVSS |
| hu147E2-L1 | 207 | DIVMTQSPDSLAVSLGERATINCKSSQSLLNSRS |
| | | RKNYLAWYQQKPGQPPKLLIYWASTRESGVPD |
| | | RFSGSGSGTDFTLTISSLQAEDVAVYYCTQSYT |
| | | LRTFGGGTKVEIK |
| hu147E2-L2 | 208 | DIVMTQSPDSLAVSLGERATINCKSSQSLLNSRS |
| | | RKNYLAWYQQKPGQSPKLLIYWASTRESGVPD |
| | | RFSGSGSGTDFTLTISSLQAEDVAVYYCTQSYT |
| | | LRTFGGGTKVEIK |
| hu147E2-L3 | 209 | DIQMTQSPSSLSASVGDRVTITCKSSQSLLNSRS |
| | | RKNYLAWYQQKPGKAPKLLIYWASTRESGVPS |
| | | RFSGSGSGTDFTLTISSLQPEDFATYYCTQSYTL |
| | | RTFGGGTKVEIK |

The present disclosure also provides humanized and affinity improved antibodies or antigen-binding fragments thereof which specifically bind to CD3. In some embodiments, the affinity maturation is performed based on the sequences of hu40E9-H3 and hu40E9-L2, which have the same CDRs as clone 40-C12-C10-E9. In some embodiments, one or more amino acids within one or more CDRs of a candidate antibody is mutated to improve affinity, for example, the mutation occurs within HCDR1 (e.g., positions 31˜33 relative to SEQ ID NO: 165), HCDR2 (e.g., positions 54˜56 relative to SEQ ID NO: 165), HCDR3 (e.g., positions 99˜106 relative to SEQ ID NO: 165) or LCDR3 (e.g., positions 91˜93 relative to SEQ ID NO: 169) of the candidate antibody. Accordingly, the present disclosure provides anti-CD3 antibodies or antigen-binding fragments thereof which have the same CDRs as clone 40-C12-C10-E9 except that the amino acid sequences of HCDR1, HCDR2, HCDR3 and/or LCDR3 are different.

In some embodiments, the anti-CD3 antibodies or antigen-binding fragments thereof provided herein comprises a HCDR1 comprising an amino acid sequence as set forth in SEQ ID NO: 219. In some embodiments, the anti-CD3 antibodies or antigen-binding fragments thereof provided herein comprises a HCDR1 comprising an amino acid sequence as set forth in SEQ ID NO: 9, 217 or 218.

In some embodiments, the anti-CD3 antibodies or antigen-binding fragments thereof provided herein comprise a HCDR2 comprising an amino acid sequence as set forth in SEQ ID NO: 201. In some embodiments, the anti-CD3 antibodies or antigen-binding fragments thereof provided herein comprise a HCDR2 comprising an amino acid sequence as set forth in SEQ ID NO: 10, 171, 172, 173, 220 or 222.

In some embodiments, the anti-CD3 antibodies or antigen-binding fragments thereof provided herein comprise a HCDR3 comprising an amino acid sequence as set forth in SEQ ID NO: 162. In some embodiments, the anti-CD3 antibodies or antigen-binding fragments thereof provided herein comprise a HCDR3 comprising an amino acid sequence as set forth in SEQ ID NO: 11, 174, 175, 176, 177, 178, 179, 180, 181, 221, 223, 224, 225, 226, 227, 228 or 229.

In some embodiments, the anti-CD3 antibodies or antigen-binding fragments thereof provided herein comprise a LCDR3 comprising an amino acid sequence as set forth in SEQ ID NO: 167. In some embodiments, the anti-CD3 antibodies or antigen-binding fragments thereof provided herein comprise a LCDR3 comprising an amino acid sequence as set forth in SEQ ID NO: 14, 182, 183, 184, 185, 214, 215 or 216.

In some embodiments, the anti-CD3 antibodies or antigen-binding fragments thereof provided herein comprise a HCDR1 comprising an amino acid sequence as set forth in SEQ ID NO: 219, a HCDR2 comprising an amino acid sequence as set forth in SEQ ID NO: 201, and a HCDR3 comprising an amino acid sequence as set forth in SEQ ID NO: 162. In some embodiments, the anti-CD3 antibodies or antigen-binding fragments thereof provided herein comprise a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 167. In some embodiments, the anti-CD3 antibodies or antigen-binding fragments thereof provided herein comprise a HCDR1, HCDR2, HCDR3, LCDR1, LCDR2 and LCDR3 comprising an amino acid sequence as set forth in SEQ ID NO: 219, 201, 162, 12, 13 and 167, respectively.

In some embodiments, the anti-CD3 antibodies or antigen-binding fragments thereof provided herein comprise a HCDR1 comprising an amino acid sequence as set forth in SEQ ID NO: 9, 217 or 218; a HCDR2 comprising an amino acid sequence as set forth in SEQ ID NO: 10, 171, 172, 173, 220 or 222; a HCDR3 comprising an amino acid sequence as set forth in SEQ ID NO: 11, 174, 175, 176, 177, 178, 179, 180, 181, 221, 223, 224, 225, 226, 227, 228 or 229. In some embodiments, the anti-CD3 antibodies or antigen-binding fragments thereof provided herein comprise a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14, 182, 183, 184, 185, 214, 215 or 216. In some embodiments, the anti-CD3 antibodies or antigen-binding fragments thereof provided herein comprise a HCDR1 comprising an amino acid sequence as set forth in SEQ ID NO: 9, 217 or 218; a HCDR2 comprising an amino acid sequence as set forth in SEQ ID NO: 10, 171, 172, 173, 220 or 222; a HCDR3 comprising an amino acid sequence as set forth in SEQ ID NO: 11, 174, 175, 176, 177, 178, 179, 180, 181, 221, 223, 224, 225, 226, 227, 228 or 229; a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14, 182, 183, 184, 185, 214, 215 or 216.

The amino acid sequences of SEQ ID NO: 171˜185, 201, 162, 167 and 214˜229 are shown in Table 22 below. As shown in Table 22 below, each of the hu40E9 HCDRs contains one or more mutations compared to their corresponding HCDRs contained within hu40E9-H3 (i.e., SEQ ID NO: 165), and the amino acid(s) after mutation is (are) underlined; similarly, each of the hu40E9 LCDR3 contains one or more mutations compared to the LCDR3 contained within hu40E9-L2 (i.e., SEQ ID NO: 169), and the amino acid(s) after mutation is (are) also underlined; similarly, each of the hu147E2 HCDR2 contains one mutation compared to the HCDR2 contained within hu40E9-H3 (i.e., SEQ ID NO: 165), and the amino acid after mutation is also underlined.

In some embodiments, the present disclosure provides antibodies or antigen-binding fragments thereof which specifically bind to CD3, comprising one or two or three HCDRs contained within any one of the VH region sequences selected from the group consisting of SEQ ID NOs: 186, 187, 188, 189, 190, 191, 192, 193, 194, 195, 196, 233, 234, 235, 236, 237, 238, 239, 240, 241, 242, 243, 244 and 245; and/or one or two or three LCDRs contained within any one of the VL region sequences selected from the group consisting of SEQ ID NOs: 197, 198, 199, 200, 230, 231 and 232. In some embodiments, the present disclosure also provides anti-CD3 antibodies or antigen-binding fragments thereof comprising HCDR1, HCDR2 and HCDR3 contained within any one of the VH region sequences selected from the group consisting of SEQ ID NOs: 186, 187, 188, 189, 190, 191, 192, 193, 194, 195, 196, 233, 234, 235, 236, 237, 238, 239, 240, 241, 242, 243, 244 and 245; and LCDR1, LCDR2 and LCDR3 contained within any one of the VL region sequences selected from the group consisting of SEQ ID NOs: 197, 198, 199, 200, 230, 231 and 232.

In some embodiments, the present disclosure provides antibodies or antigen-binding fragments thereof which specifically bind to CD3, comprising one or two or three HCDRs contained within any one of the VH region sequences selected from the group consisting of SEQ ID NOs: 213, 202, 203, 204, 205 and 206; and/or one or two or three LCDRs contained within any one of the VL region sequences selected from the group consisting of SEQ ID NOs: 207, 208 and 209. In some embodiments, the present disclosure also provides anti-CD3 antibodies or antigen-binding fragments thereof comprising HCDR1, HCDR2 and HCDR3 contained within any one of the VH region sequences selected from the group consisting of SEQ ID NOs: 213, 202, 203, 204, 205 and 206; and LCDR1, LCDR2 and LCDR3 contained within any one of the VL region sequences selected from the group consisting of SEQ ID NOs: 207, 208 and 209.

The amino acid sequences of SEQ ID NO: 186˜200 and 230˜245 are shown in Table 21 below. As shown in Table 21 below, each of the VH regions contains one, two or three mutations compared to the amino acid sequence of hu40E9-H3 (i.e., SEQ ID NO: 165), and the amino acid(s) after mutation is (are) underlined; similarly, each of the VL regions contains one or two mutations compared to the amino acid sequence of hu40E9-L2 (i.e., SEQ ID NO: 169), and the amino acid(s) after mutation is (are) also underlined.

TABLE 21
Amino acid sequences of exemplary
VH and VL regions

| | SEQ | |
|---|---|---|
| Description | ID NO | Amino Acid Sequence |
| hu40E9-H3- | 186 | EVQLVQSGAEVKKPGSSVKVSCKASGYTFSSY |
| N55S.H | | VIHWVRQAPGQGLEWMGYINPYSDVIKYNEKF |
| | | KGRVTLTSDKSTSTAYMELSSLRSEDTAVYYC |
| | | ARDSYYSYDGFAYWGQGTLVTVSS |
| hu40E9-H3- | 187 | EVQLVQSGAEVKKPGSSVKVSCKASGYTFSSY |
| D99E.H | | VIHWVRQAPGQGLEWMGYINPYNDVIKYNEK |
| | | FKGRVTLTSDKSTSTAYMELSSLRSEDTAVYY |
| | | CARESYYSYDGFAYWGQGTLVTVSS |
| hu40E9-H3- | 188 | EVQLVQSGAEVKKPGSSVKVSCKASGYTFSSY |
| Y101F.H | | VIHWVRQAPGQGLEWMGYINPYNDVIKYNEK |
| | | FKGRVTLTSDKSTSTAYMELSSLRSEDTAVYY |
| | | CARDSFYSYDGFAYWGQGTLVTVSS |
| hu40E9-H3- | 189 | EVQLVQSGAEVKKPGSSVKVSCKASGYTFSSY |
| D105E.H | | VIHWVRQAPGQGLEWMGYINPYNDVIKYNEK |
| | | FKGRVTLTSDKSTSTAYMELSSLRSEDTAVYY |
| | | CARDSYYSYEGFAYWGQGTLVTVSS |
| hu40E9-H3- | 190 | EVQLVQSGAEVKKPGSSVKVSCKASGYTFSSY |
| G106A.H | | VIHWVRQAPGQGLEWMGYINPYNDVIKYNEK |
| | | FKGRVTLTSDKSTSTAYMELSSLRSEDTAVYY |
| | | CARDSYYSYDAFAYWGQGTLVTVSS |
| hu40E9-H3- | 191 | EVQLVQSGAEVKKPGSSVKVSCKASGYTFSSY |
| Y54G.H | | VIHWVRQAPGQGLEWMGYINPGNDVIKYNEK |
| | | FKGRVTLTSDKSTSTAYMELSSLRSEDTAVYY |
| | | CARDSYYSYDGFAYWGQGTLVTVSS |
| hu40E9-H3- | 192 | EVQLVQSGAEVKKPGSSVKVSCKASGYTFSSY |
| D56G.H | | VIHWVRQAPGQGLEWMGYINPYNGVIKYNEK |
| | | FKGRVTLTSDKSTSTAYMELSSLRSEDTAVYY |
| | | CARDSYYSYDGFAYWGQGTLVTVSS |
| hu40E9-H3- | 193 | EVQLVQSGAEVKKPGSSVKVSCKASGYTFSSY |
| D99R.H | | VIHWVRQAPGQGLEWMGYINPYNDVIKYNEK |
| | | FKGRVTLTSDKSTSTAYMELSSLRSEDTAVYY |
| | | CARRSYYSYDGFAYWGQGTLVTVSS |
| hu40E9-H3- | 194 | EVQLVQSGAEVKKPGSSVKVSCKASGYTFSSY |
| S100R.H | | VIHWVRQAPGQGLEWMGYINPYNDVIKYNEK |
| | | FKGRVTLTSDKSTSTAYMELSSLRSEDTAVYY |
| | | CARDRYYSYDGFAYWGQGTLVTVSS |
| hu40E9-H3- | 195 | EVQLVQSGAEVKKPGSSVKVSCKASGYTFSSY |
| Y102S.H | | VIHWVRQAPGQGLEWMGYINPYNDVIKYNEK |
| | | FKGRVTLTSDKSTSTAYMELSSLRSEDTAVYY |
| | | CARDSYSSYDGFAYWGQGTLVTVSS |
| hu40E9-H3- | 196 | EVQLVQSGAEVKKPGSSVKVSCKASGYTFSSY |
| D105R.H | | VIHWVRQAPGQGLEWMGYINPYNDVIKYNEK |
| | | FKGRVTLTSDKSTSTAYMELSSLRSEDTAVYY |
| | | CARDSYYSYRGFAYWGQGTLVTVSS |
| hu40E9-H5 | 233 | EVQLVQSGAEVKKPGSSVKVSCKASGYTFSSY |
| | | VIHWVRQAPGQGLEWMGYINPGSGVIKYNEKF |
| | | KGRVTLTSDKSTSTAYMELSSLRSEDTAVYYC |
| | | ARDSYYSYDGFAYWGQGTLVTVSS |
| hu40E9-H6 | 234 | EVQLVQSGAEVKKPGSSVKVSCKASGYTFSSY |
| | | VIHWVRQAPGQGLEWMGYINPYNDVIKYNEK |
| | | FKGRVTLTSDKSTSTAYMELSSLRSEDTAVYY |
| | | CARESFSSYDGFAYWGQGTLVTVSS |
| hu40E9-H7 | 235 | EVQLVQSGAEVKKPGSSVKVSCKASGYTFSSY |
| | | VIHWVRQAPGQGLEWMGYINPGNGVIKYNEK |
| | | FKGRVTLTSDKSTSTAYMELSSLRSEDTAVYY |
| | | CARDSYSSYDGFAYWGQGTLVTVSS |
| hu40E9-H8 | 236 | EVQLVQSGAEVKKPGSSVKVSCKASGYTFSSY |
| | | VIHWVRQAPGQGLEWMGYINPGNDVIKYNEK |
| | | FKGRVTLTSDKSTSTAYMELSSLRSEDTAVYY |
| | | CARESYSSYDGFAYWGQGTLVTVSS |
| hu40E9-H9 | 237 | EVQLVQSGAEVKKPGSSVKVSCKASGYTFSSY |
| | | VIHWVRQAPGQGLEWMGYINPYSDVIKYNEKF |
| | | KGRVTLTSDKSTSTAYMELSSLRSEDTAVYYC |
| | | ARESFYSYDGFAYWGQGTLVTVSS |
| hu40E9-H10 | 238 | EVQLVQSGAEVKKPGSSVKVSCKASGYTFSSY |
| | | VIHWVRQAPGQGLEWMGYINPYNDVIKYNEK |
| | | FKGRVTLTSDKSTSTAYMELSSLRSEDTAVYY |
| | | CARDSYYSYEAFAYWGQGTLVTVSS |
| hu40E9-H11 | 239 | EVQLVQSGAEVKKPGSSVKVSCKASGYTFSSY |
| | | VIHWVRQAPGQGLEWMGYINPYNDVIKYNEK |
| | | FKGRVTLTSDKSTSTAYMELSSLRSEDTAVYY |
| | | CARDSYYSYRAFAYWGQGTLVTVSS |
| hu40E9-H12 | 240 | EVQLVQSGAEVKKPGSSVKVSCKASGYTFSSY |
| | | VIHWVRQAPGQGLEWMGYINPYNDVIKYNEK |
| | | FKGRVTLTSDKSTSTAYMELSSLRSEDTAVYY |
| | | CARDDYYSYDGFAYWGQGTLVTVSS |
| hu40E9-H13 | 241 | EVQLVQSGAEVKKPGSSVKVSCKASGYTFSSY |
| | | VIHWVRQAPGQGLEWMGYINPYNDVIKYNEK |
| | | FKGRVTLTSDKSTSTAYMELSSLRSEDTAVYY |
| | | CARDGYYSYDGFAYWGQGTLVTVSS |
| hu40E9-H14 | 242 | EVQLVQSGAEVKKPGSSVKVSCKASGYTFSSY |
| | | VIHWVRQAPGQGLEWMGYINPYNDVIKYNEK |
| | | FKGRVTLTSDKSTSTAYMELSSLRSEDTAVYY |
| | | CARESYYSYDGFAYWGQGTLVTVSS |
| hu40E9-H15 | 243 | EVQLVQSGAEVKKPGSSVKVSCKASGYTFSSY |
| | | VIHWVRQAPGQGLEWMGYINPYNDVIKYNEK |
| | | FKGRVTLTSDKSTSTAYMELSSLRSEDTAVYY |
| | | CARGSYYSYDGFAYWGQGTLVTVSS |
| hu40E9-H16 | 244 | EVQLVQSGAEVKKPGSSVKVSCKASGYTFSDY |
| | | VIHWVRQAPGQGLEWMGYINPYNDVIKYNEK |
| | | FKGRVTLTSDKSTSTAYMELSSLRSEDTAVYY |
| | | CARDSYYSYDGFAYWGQGTLVTVSS |
| hu40E9-H17 | 245 | EVQLVQSGAEVKKPGSSVKVSCKASGYTFSSY |
| | | YIHWVRQAPGQGLEWMGYINPYNDVIKYNEK |
| | | FKGRVTLTSDKSTSTAYMELSSLRSEDTAVYY |
| | | CARDSYYSYDGFAYWGQGTLVTVSS |
| hu40E9-L2- | 197 | EIVLTQSPDFQSVTPKEKVTITCSASSSVSYMH |
| N93S.L | | WYQQKPDQSPKRLIYDTSKLASGVPSRFSGSGS |
| | | GTDFTLTINSLEAEDAATYYCQQWSSSPPTFGG |
| | | GTKVEIK |
| hu40E9-L2- | 198 | EIVLTQSPDFQSVTPKEKVTITCSASSSVSYMH |
| S91R.L | | WYQQKPDQSPKRLIYDTSKLASGVPSRFSGSGS |
| | | GTDFTLTINSLEAEDAATYYCQQWRSNPPTFG |
| | | GGTKVEIK |
| hu40E9-L2- | 199 | EIVLTQSPDFQSVTPKEKVTITCSASSSVSYMH |
| N93R.L | | WYQQKPDQSPKRLIYDTSKLASGVPSRFSGSGS |
| | | GTDFTLTINSLEAEDAATYYCQQWSSRPPTFGG |
| | | GTKVEIK |
| hu40E9-L2- | 200 | EIVLTQSPDFQSVTPKEKVTITCSASSSVSYMH |
| N93W.L | | WYQQKPDQSPKRLIYDTSKLASGVPSRFSGSGS |
| | | GTDFTLTINSLEAEDAATYYCQQWSSWPPTFG |
| | | GGTKVEIK |
| hu40E9-L4 | 230 | EIVLTQSPDFQSVTPKEKVTITCSASSSVSYMH |
| | | WYQQKPDQSPKRLIYDTSKLASGVPSRFSGSGS |
| | | GTDFTLTINSLEAEDAATYYCQQWRSRPPTFGG |
| | | GTKVEIK |
| hu40E9-L5 | 231 | EIVLTQSPDFQSVTPKEKVTITCSASSSVSYMH |
| | | WYQQKPDQSPKRLIYDTSKLASGVPSRFSGSGS |
| | | GTDFTLTINSLEAEDAATYYCQQWRSSPPTFGG |
| | | GTKVEIK |
| hu40E9-L6 | 232 | EIVLTQSPDFQSVTPKEKVTITCSASSSVSYMH |
| | | WYQQKPDQSPKRLIYDTSKLASGVPSRFSGSGS |
| | | GTDFTLTINSLEAEDAATYYCQQWRSWPPTFG |
| | | GGTKVEIK |

TABLE 22
Amino acid sequences of exemplary CDRs

| Description | SEQ ID NO | Amino Acid Sequence |
|---|---|---|
| hu40E9-H16 (HCDR1) | 217 | DYVIH |
| hu40E9-H17 (HCDR1) | 218 | SYYIH |
| Formula of hu40E9 HCDR1 | 219 | X₁₃YX₁₄IH |
| | | (wherein X₁₃ = S or D; X₁₄ = V or Y) |
| hu40E9-H3-N55S.H | 171 | YINPYSDVIKYNEKFKG |
| (HCDR2) | | |
| hu40E9-H3-Y54G.H | 172 | YINPGNDVIKYNEKFKG |
| (HCDR2) | | |
| hu40E9-H3-D56G.H | 173 | YINPYNGVIKYNEKFKG |
| (HCDR2) | | |
| hu40E9-H5 (HCDR2) | 220 | YINPGSGVIKYNEKFKG |
| hu40E9-H7 (HCDR2) | 222 | YINPGNGVIKYNEKFKG |
| Formula of hu40E9 HCDR2 | 201 | YINPX₁X₂X₃VIKYNEKFKG |
| | | (wherein X₁ = Y or G; X₂ = N or S; and X₃ = |
| | | D or G) |
| hu40E9-H3-D99E.H | 174 | ESYYSYDGFAY |
| (HCDR3) | | |
| hu40E9-H3-Y101F.H | 175 | DSFYSYDGFAY |
| (HCDR3) | | |
| hu40E9-H3-D105E.H | 176 | DSYYSYEGFAY |
| (HCDR3) | | |
| hu40E9-H3-G106A.H | 177 | DSYYSYDAFAY |
| (HCDR3) | | |
| hu40E9-H3-D99R.H | 178 | RSYYSYDGFAY |
| (HCDR3) | | |
| hu40E9-H3-S100R.H | 179 | DRYYSYDGFAY |
| (HCDR3) | | |
| hu40E9-H3-Y102S.H | 180 | DSYSSYDGFAY |
| (HCDR3) | | |
| hu40E9-H3-D105R.H | 181 | DSYYSYRGFAY |
| (HCDR3) | | |
| hu40E9-H6 (HCDR3) | 221 | ESFSSYDGFAY |
| hu40E9-H8 (HCDR3) | 223 | ESYSSYDGFAY |
| hu40E9-H9 (HCDR3) | 224 | ESFYSYDGFAY |
| hu40E9-H10 (HCDR3) | 225 | DSYYSYEAFAY |
| hu40E9-H11 (HCDR3) | 226 | DSYYSYRAFAY |
| hu40E9-H12 (HCDR3) | 227 | DDYYSYDGFAY |
| hu40E9-H13 (HCDR3) | 228 | DGYYSYDGFAY |
| hu40E9-H15 (HCDR3) | 229 | GSYYSYDGFAY |
| Formula of hu40E9 HCDR3 | 162 | X₄X₅X₆X₇SYX₈X₉FAY |
| | | (wherein X₄ = D or E or R or G; X₅ = S or R |
| | | or D or G; X₆ = Y or F; X₇ = Y or S; X₈ = D |
| | | or E or R; X₉ = G or A) |
| hu40E9-L2-N93S.L | 182 | QQWSSSPPT |
| (LCDR3) | | |
| hu40E9-L2-S91R.L | 183 | QQWRSNPPT |
| (LCDR3) | | |
| hu40E9-L2-N93R.L | 184 | QQWSSRPPT |
| (LCDR3) | | |
| hu40E9-L2-N93W.L | 185 | QQWSSWPPT |
| (LCDR3) | | |
| hu40E9-L4 (LCDR3) | 214 | QQWRSRPPT |
| hu40E9-L5 (LCDR3) | 215 | QQWRSSPPT |
| hu40E9-L6 (LCDR3) | 216 | QQWRSWPPT |
| Formula of hu40E9 LCDR3 | 167 | QQWX₁₀SX₁₁PPT |
| | | (wherein X₁₀ = S or R; X₁₁ = N or S or R or W) |
| hu147E2-H3a-N55Q | 210 | NINPSQGATNYNEKFKS |
| (HCDR2) | | |
| hu147E2-H3a-N55S | 211 | NINPSSGATNYNEKFKS |
| (HCDR2) | | |
| Formula of hu147E2 | 212 | NINPSX₁₂GATNYNEKFKS |
| HCDR2 | | (X₁₂ = Q, S or N) |

The present disclosure further provides exemplary humanized and affinity improved antibodies of clone 40-C12-C10-E9, including:
1) “hu40E9-L2H3-N55S.H” comprising the heavy chain variable region of hu40E9-H3-N55S.H (SEQ ID NO: 186) and the light chain variable region of hu40E9-L2 (SEQ ID NO: 169);
2) “hu40E9-L2H3-D99E.H” comprising the heavy chain variable region of hu40E9-H3-D99E.H (SEQ ID NO: 187) and the light chain variable region of hu40E9-L2 (SEQ ID NO: 169);
3) “hu40E9-L2H3-Y101F.H” comprising the heavy chain variable region of hu40E9-H3-Y101F.H (SEQ ID NO: 188) and the light chain variable region of hu40E9-L2 (SEQ ID NO: 169);
4) “hu40E9-L2H3-D105E.H” comprising the heavy chain variable region of hu40E9-H3-D105E.H (SEQ ID NO: 189) and the light chain variable region of hu40E9-L2 (SEQ ID NO: 169);
5) “hu40E9-L2H3-G106A.H” comprising the heavy chain variable region of hu40E9-H3-G106A.H (SEQ ID NO: 190) and the light chain variable region of hu40E9-L2 (SEQ ID NO: 169);
6) “hu40E9-L2H3-Y54G.H” comprising the heavy chain variable region of hu40E9-H3-Y54G.H (SEQ ID NO: 191) and the light chain variable region of hu40E9-L2 (SEQ ID NO: 169);
7) “hu40E9-L2H3-D56G.H” comprising the heavy chain variable region of hu40E9-H3-D56G.H (SEQ ID NO: 192) and the light chain variable region of hu40E9-L2 (SEQ ID NO: 169);
8) “hu40E9-L2H3-D99R.H” comprising the heavy chain variable region of hu40E9-H3-D99R.H (SEQ ID NO: 193) and the light chain variable region of hu40E9-L2 (SEQ ID NO: 169);
9) “hu40E9-L2H3-S100R.H” comprising the heavy chain variable region of hu40E9-H3-S100R.H (SEQ ID NO: 194) and the light chain variable region of hu40E9-L2 (SEQ ID NO: 169);
10) “hu40E9-L2H3-Y102S.H” comprising the heavy chain variable region of hu40E9-H3-Y102S.H (SEQ ID NO: 195) and the light chain variable region of hu40E9-L2 (SEQ ID NO: 169);
11) “hu40E9-L2H3-D105R.H” comprising the heavy chain variable region of hu40E9-H3-D105R.H (SEQ ID NO: 196) and the light chain variable region of hu40E9-L2 (SEQ ID NO: 169);
12) “hu40E9-L2H3-N93S.L” comprising the heavy chain variable region of hu40E9-H3 (SEQ ID NO: 165) and the light chain variable region of hu40E9-L2-N93S.L (SEQ ID NO: 197);
13) “hu40E9-L2H3-S91R.L” comprising the heavy chain variable region of hu40E9-H3 (SEQ ID NO: 165) and the light chain variable region of hu40E9-L2-S91R.L (SEQ ID NO: 198);
14) “hu40E9-L2H3-N93R.L” comprising the heavy chain variable region of hu40E9-H3 (SEQ ID NO: 165) and the light chain variable region of hu40E9-L2-N93R.L (SEQ ID NO: 199);
15) “hu40E9-L2H3-N93W.L” comprising the heavy chain variable region of hu40E9-H3 (SEQ ID NO: 165) and the light chain variable region of hu40E9-L2-N93W.L (SEQ ID NO: 200);
16) “hu40E9-L2H5” comprising the heavy chain variable region of hu40E9-H5 (SEQ ID NO: 233) and the light chain variable region of hu40E9-L2 (SEQ ID NO: 169);
17) “hu40E9-L2H6” comprising the heavy chain variable region of hu40E9-H6 (SEQ ID NO: 234) and the light chain variable region of hu40E9-L2 (SEQ ID NO: 169);
18) “hu40E9-L2H7” comprising the heavy chain variable region of hu40E9-H7 (SEQ ID NO: 235) and the light chain variable region of hu40E9-L2 (SEQ ID NO: 169);
19) “hu40E9-L2H8” comprising the heavy chain variable region of hu40E9-H8 (SEQ ID NO: 236) and the light chain variable region of hu40E9-L2 (SEQ ID NO: 169);
20) “hu40E9-L2H9” comprising the heavy chain variable region of hu40E9-H9 (SEQ ID NO: 237) and the light chain variable region of hu40E9-L2 (SEQ ID NO: 169);
21) “hu40E9-L2H10” comprising the heavy chain variable region of hu40E9-H10 (SEQ ID NO: 238) and the light chain variable region of hu40E9-L2 (SEQ ID NO: 169);
22) “hu40E9-L2H11” comprising the heavy chain variable region of hu40E9-H11 (SEQ ID NO: 239) and the light chain variable region of hu40E9-L2 (SEQ ID NO: 169);
23) “hu40E9-L2H12” comprising the heavy chain variable region of hu40E9-H12 (SEQ ID NO: 240) and the light chain variable region of hu40E9-L2 (SEQ ID NO: 169);
24) “hu40E9-L2H13” comprising the heavy chain variable region of hu40E9-H13 (SEQ ID NO: 241) and the light chain variable region of hu40E9-L2 (SEQ ID NO: 169);
25) “hu40E9-L2H14” comprising the heavy chain variable region of hu40E9-H14 (SEQ ID NO: 242) and the light chain variable region of hu40E9-L2 (SEQ ID NO: 169);
26) “hu40E9-L2H15” comprising the heavy chain variable region of hu40E9-H15 (SEQ ID NO: 243) and the light chain variable region of hu40E9-L2 (SEQ ID NO: 169);
27) “hu40E9-L2H16” comprising the heavy chain variable region of hu40E9-H16 (SEQ ID NO: 244) and the light chain variable region of hu40E9-L2 (SEQ ID NO: 169);
28) “hu40E9-L2H17” comprising the heavy chain variable region of hu40E9-H17 (SEQ ID NO: 245) and the light chain variable region of hu40E9-L2 (SEQ ID NO: 169);
29) “hu40E9-L4H3” comprising the heavy chain variable region of hu40E9-H3 (SEQ ID NO: 165) and the light chain variable region of hu40E9-L4 (SEQ ID NO: 230);
30) “hu40E9-L5H3” comprising the heavy chain variable region of hu40E9-H3 (SEQ ID NO: 165) and the light chain variable region of hu40E9-L5 (SEQ ID NO: 231);
31) “hu40E9-L6H3” comprising the heavy chain variable region of hu40E9-H3 (SEQ ID NO: 165) and the light chain variable region of hu40E9-L6 (SEQ ID NO: 232);
32) “hu40E9-L4H7” comprising the heavy chain variable region of hu40E9-H7 (SEQ ID NO: 235) and the light chain variable region of hu40E9-L4 (SEQ ID NO: 230);
33) “hu40E9-L4H8” comprising the heavy chain variable region of hu40E9-H8 (SEQ ID NO: 236) and the light chain variable region of hu40E9-L4 (SEQ ID NO: 230);
34) “hu40E9-L5H7” comprising the heavy chain variable region of hu40E9-H7 (SEQ ID NO: 235) and the light chain variable region of hu40E9-L5 (SEQ ID NO: 231); or
35) “hu40E9-L5H8” comprising the heavy chain variable region of hu40E9-H8 (SEQ ID NO: 236) and the light chain variable region of hu40E9-L5 (SEQ ID NO: 231).

In some embodiments, the anti-CD3 antibodies and antigen-binding fragments provided herein comprise all or a portion of the heavy chain variable domain and/or all or a portion of the light chain variable domain. In one embodiment, the anti-CD3 antibody or an antigen-binding fragment thereof provided herein is a single domain antibody which consists of all or a portion of the heavy chain variable domain provided herein. More information of such a single domain antibody is available in the art (see, e.g., U.S. Pat. No. 6,248,516).

In certain embodiments, the anti-CD3 antibodies or antigen-binding fragments thereof provided herein further comprise an Fc region. In certain embodiments, the anti-CD3 antibodies or antigen-binding fragments thereof provided herein further comprise an Fc region of human immunoglobulin (Ig). In certain embodiments, the anti-CD3 antibodies or antigen-binding fragments thereof provided herein further comprise a constant region, which optionally further comprises a heavy chain and/or a light chain constant region. In certain embodiments, the heavy chain constant region comprises CH1, hinge, and/or CH2-CH3 regions (or optionally CH2-CH3-CH4 regions). In certain embodiments, the anti-CD3 antibodies or the antigen-binding fragments thereof provided herein comprise heavy chain constant regions of human IgG1, IgG2, IgG3, IgG4, IgA1, IgA2 or IgM. In certain embodiments, the anti-CD3 antibodies or antigen-binding fragments thereof provided herein comprise a lambda (λ) light chain or a kappa (κ) light chain. The constant region of the anti-CD3 antibodies or the antigen-binding fragments thereof provided herein may be identical to the wild-type constant region sequence or be different in one or more mutations.

In certain embodiments, the heavy chain constant region comprises an Fc region. Fc region is known to mediate effector functions such as ADCC and complement-dependent cytotoxicity (CDC) of the antibody. Fc regions of different Ig isotypes have different abilities to induce effector functions. For example, Fc regions of IgG1 and IgG3 have been recognized to induce both ADCC and CDC more effectively than those of IgG2 and IgG4. In certain embodiments, the anti-CD3 antibodies and antigen-binding fragments thereof provided herein comprises an Fc region of IgG1, or IgG3 isotype, which could induce ADCC or CDC; or alternatively, a constant region of IgG4 or IgG2 isotype, which has reduced or depleted effector function. In some embodiments, the Fc region derived from human IgG1. In some embodiments, the Fc region derived from human IgG1 with enhanced effector functions. In some embodiments, the Fc region comprises an amino acid sequence as set forth in SEQ ID NO: 97.

(SEQ ID NO: 97)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSREEMTKNQVSLTC
LVKGFYPSDIAVEWESNGQPENNYKTTPPVLDSDGSFFLYSKLTVDKSRW
QQGNVFSCSVMHEALHNHYTQKSLSLSPGK

In certain embodiments, the antibodies or antigen-binding fragments thereof provided herein have a specific binding affinity to human CD3 which is sufficient to provide for diagnostic and/or therapeutic use.

The antibodies or antigen-binding fragments thereof provided herein can be a monoclonal antibody, a polyclonal antibody, a humanized antibody, a human antibody, a chimeric antibody, a recombinant antibody, a bispecific antibody, a multi-specific antibody, a labeled antibody, a bivalent antibody, an anti-idiotypic antibody, or a fusion protein. A recombinant antibody is an antibody prepared in vitro using recombinant methods rather than in animals.

In certain embodiments, the present disclosure provides an anti-CD3 antibody or antigen-binding fragment thereof, which competes for binding to CD3 with the antibody or antigen-binding fragment thereof provided herein. In certain embodiments, the present disclosure provides an anti-CD3 antibody or antigen-binding fragment thereof, which competes for binding to human CD3 with any one of antibodies 25-G12-G6-C12, 40-C12-C10-E9, 8-B12-F9-B11, 31-F8-F5-C5, 16-F2-C11-D9, 20-E11-E11-C2, 7-D9-G10-H2, 7-D8-G12-E4, 2-F12-A6-G2, 3-C6-C11-F12, 4-F12-F1-A4, 3-F3-G12-E2, 124E3D6, 126A11A4, 127E2D3, 133B4C7, 140D2B10, 147C6F3, 147E11E2, hu40E9-L1H1, hu40E9-L1H2, hu40E9-L1H3, hu40E9-L1H4, hu40E9-L2H1, hu40E9-L2H2, hu40E9-L2H3, hu40E9-L2H4, hu40E9-L3H1, hu40E9-L3H2, hu40E9-L3H3, hu40E9-L3H4, hu40E9-L2H3-N55S.H, hu40E9-L2H3-D99E.H, hu40E9-L2H3-Y101F.H, hu40E9-L2H3-D105E.H, hu40E9-L2H3-G106A.H, hu40E9-L2H3-Y54G.H, hu40E9-L2H3-D56G.H, hu40E9-L2H3-D99R.H, hu40E9-L2H3-S100R.H, hu40E9-L2H3-Y102S.H, hu40E9-L2H3-D105R.H, hu40E9-L2H3-N93S.L, hu40E9-L2H3-S91R.L, hu40E9-L2H3-N93R.L, hu40E9-L2H3-N93W.L, hu40E9-L2H5, hu40E9-L2H6, hu40E9-L2H7, hu40E9-L2H8, hu40E9-L2H9, hu40E9-L2H10, hu40E9-L2H11, hu40E9-L2H12, hu40E9-L2H13, hu40E9-L2H14, hu40E9-L2H15, hu40E9-L2H16, hu40E9-L2H17, hu40E9-L4H3, hu40E9-L5H3, hu40E9-L6H3, hu40E9-L4H7, hu40E9-L4H8, hu40E9-L5H7, hu40E9-L5H8, hu147E2-L1H2, hu147E2-L1H3, hu147E2-L1H3a, hu147E2-L1H3b, hu147E2-L1H4, hu147E2-L2H2, hu147E2-L2H3, hu147E2-L2H4, hu147E2-L3H2, hu147E2-L3H3 and hu147E2-L3H4. In some embodiments, the present disclosure provides an anti-CD3 antibody or antigen-binding fragment thereof, which competes for the same epitope with the antibody or antigen-binding fragment thereof provided herein.

The ability to “block binding” or “compete for binding” as used herein refers to the ability of an antibody or antigen-binding fragment to inhibit the binding interaction between two molecules (e.g., human CD3 and an anti-CD3 antibody) to any detectable degree. In certain embodiments, an antibody or antigen-binding fragment thereof blocks binding between two molecules and inhibits the binding interaction between the two molecules by at least 85%, or at least 90%. In certain embodiments, this inhibition may be greater than 85%, or greater than 90%.

Those skilled in the art will recognize that it is possible to determine, without undue experimentation, if a human monoclonal antibody binds to the same epitope as the antibody of present disclosure (e.g., mouse monoclonal antibodies 25-G12-G6-C12, 40-C12-C10-E9, 8-B12-F9-B11, 31-F8-F5-C5, 16-F2-C11-D9, 20-E11-E11-C2, 7-D9-G10-H2, 7-D8-G12-E4, 2-F12-A6-G2, 3-C6-C11-F12, 4-F12-F1-A4, 3-F3-G12-E2, 124E3D6, 126A11A4, 127E2D3, 133B4C7, 140D2B10, 147C6F3, 147E11E2, hu40E9-L1H1, hu40E9-L1H2, hu40E9-L1H3, hu40E9-L1H4, hu40E9-L2H1, hu40E9-L2H2, hu40E9-L2H3, hu40E9-L2H4, hu40E9-L3H1, hu40E9-L3H2, hu40E9-L3H3, hu40E9-L3H4, hu40E9-L2H3-N55S.H, hu40E9-L2H3-D99E.H, hu40E9-L2H3-Y101F.H, hu40E9-L2H3-D105E.H, hu40E9-L2H3-G106A.H, hu40E9-L2H3-Y54G.H, hu40E9-L2H3-D56G.H, hu40E9-L2H3-D99R.H, hu40E9-L2H3-S100R.H, hu40E9-L2H3-Y102S.H, hu40E9-L2H3-D105R.H, hu40E9-L2H3-N93S.L, hu40E9-L2H3-S91R.L, hu40E9-L2H3-N93R.L, hu40E9-L2H3-N93W.L, hu40E9-L2H5, hu40E9-L2H6, hu40E9-L2H7, hu40E9-L2H8, hu40E9-L2H9, hu40E9-L2H10, hu40E9-L2H11, hu40E9-L2H12, hu40E9-L2H13, hu40E9-L2H14, hu40E9-L2H15, hu40E9-L2H16, hu40E9-L2H17, hu40E9-L4H3, hu40E9-L5H3, hu40E9-L6H3, hu40E9-L4H7, hu40E9-L4H8, hu40E9-L5H7, hu40E9-L5H8, hu147E2-L1H2, hu147E2-L1H3, hu147E2-L1H3a, hu147E2-L1H3b, hu147E2-L1H4, hu147E2-L2H2, hu147E2-L2H3, hu147E2-L2H4, hu147E2-L3H2, hu147E2-L3H3 and hu147E2-L3H4) by ascertaining whether the former prevents the latter from binding to a CD3 antigen polypeptide. If the test antibody competes with the antibody of present disclosure, as shown by a decrease in binding by the antibody of present disclosure to the CD3 antigen polypeptide, then the two antibodies bind to the same, or a closely related, epitope. Or if the binding of a test antibody to the CD3 antigen polypeptide was inhibited by the antibody of the present disclosure, then the two antibodies bind to the same, or a closely related, epitope.

In certain embodiments, the present disclosure provides anti-CD3 antibodies or antigen-binding fragments thereof which have higher or comparable binding affinity to CD3 (e.g., human CD3 or cynomolgus CD3) compared with OKT3, BMK-B219 and/or BMK-TCB.

In certain embodiments, the anti-CD3 antibody or antigen-binding fragment which competes for binding to CD3 with the antibody or antigen-binding fragment thereof provided herein is not OKT3, BMK-B219 or BMK-TCB.

“OKT3” as used herein refers to an antibody or antigen-binding fragment thereof comprising a heavy chain variable region having an amino acid sequence of SEQ ID NO: 100, and a light chain variable region having an amino acid sequence of SEQ ID NO: 101. The CDR sequences were underlined in SEQ ID NO: 100 and SEQ ID NO: 101, respectively.

(SEQ ID NO: 100)
QVQLQQSGAELARPGASVKMSCKASGYTFTRYTMHWVKQRPGQGLEWIGY
INPSRGYTNYNQKFKDKATLTTDKSSSTAYMQLSSLTSEDSAVYYCARYY
DDHYCLDYWGQGTTLTVSS
(SEQ ID NO: 101)
QIVLTQSPAIMSASPGEKVTMTCSASSSVSYMNWYQQKSGTSPKRWIYDT
SKLASGVPAHFRGSGSGTSYSLTISGMEAEDAATYYCQQWSSNPFTFGSG
TKLEINRAD

“BMK-B219” as used herein refers to an antibody or antigen-binding fragment thereof comprising a heavy chain variable region having an amino acid sequence of SEQ ID NO: 158, and a light chain variable region having an amino acid sequence of SEQ ID NO: 159. The CDR sequences were underlined in SEQ ID NO: 158 and SEQ ID NO: 159, respectively.

(SEQ ID NO: 158)
EVQLVESGGGLVQPGGSLRLSCAASGFTFNTYAMNWVRQAPGKGLEWVAR
IRSKYNNYATYYAASVKGRFTISRDDSKNSLYLQMNSLKTEDTAVYYCAR
HGNFGNSYVSWFAYWGQGTLVTVSS
(SEQ ID NO: 159)
QTVVTQEPSLTVSPGGTVTLTCRSSTGAVTTSNYANWVQQKPGQAPRGLI
GGTNKRAPGTPARFSGSLLGGKAALTLSGVQPEDEAEYYCALWYSNLWVF
GGGTKLTVL

“BMK-TCB” as used herein refers to an antibody or antigen-binding fragment thereof comprising a heavy chain variable region having an amino acid sequence of SEQ ID NO: 160, and a light chain variable region having an amino acid sequence of SEQ ID NO: 161. The CDR sequences were underlined in SEQ ID NO: 160 and SEQ ID NO: 161, respectively.

(SEQ ID NO: 160)
EVQLLESGGGLVQPGGSLRLSCAASGFTFSTYAMNWVRQAPGKGLEWVSR
IRSKYNNYATYYADSVKGRFTISRDDSKNTLYLQMNSLRAEDTAVYYCVR
HGNFGNSYVSWFAYWGQGTLVTVSS
(SEQ ID NO: 161)
QAVVTQEPSLTVSPGGTVTLTCGSSTGAVTTSNYANWVQEKPGQAFRGLI
GGTNKRAPGTPARFSGSLLGGKAALTLSGAQPEDEAEYYCALWYSNLWVF
GGGTKLTVL

### Antibody Variants

The antibodies and antigen-binding fragments thereof provided herein also encompass various variants of the antibody sequences provided herein.

In certain embodiments, the antibody variants comprise one or more amino acid residue substitutions or modifications yet retains specific binding affinity to CD3. In certain embodiments, at least one of the substitutions or modifications is in one or more of the CDR sequences of the VH region or VL region. In certain embodiments, at least one of the substitutions or modifications is in one or more of the non-CDR sequences of the VH region or VL region. In certain embodiments, the antibodies or antigen-binding fragments thereof provided herein further comprise one or more non-natural amino acid (NNAA) substitution. In certain embodiments, the NNAA is capable of being conjugated.

For example, the antibody variants comprise one or more amino acid residue substitutions or modifications in one or more of the CDR sequences provided in Table 2 above, one or more of the non-CDR sequences of the heavy chain variable region or light chain variable region provided in Table 3 above, and/or the constant region (e.g., Fc region). Such variants retain binding specificity to CD3 of their parent antibodies, but have one or more desirable properties conferred by the modification(s) or substitution(s). For example, the antibody variants may have improved antigen-binding affinity, improved glycosylation pattern, reduced risk of glycosylation, reduced deamination, enhanced effector function(s), improved FcRn receptor binding, increased pharmacokinetic half-life, pH sensitivity, and/or compatibility to conjugation (e.g., one or more introduced cysteine residues), etc.

The parent antibody sequence may be screened to identify suitable or preferred residues to be modified or substituted, using methods known in the art, for example, “alanine scanning mutagenesis” (see, for example, Cunningham and Wells (1989) Science, 244:1081-1085). Briefly, target residues (e.g., charged residues such as Arg, Asp, His, Lys, and Glu) can be identified and replaced by a neutral or negatively charged amino acid (e.g., alanine or polyalanine), and the modified antibodies are produced and screened for the interested property. If substitution at a particular amino acid location demonstrates an interested functional change, then the position can be identified as a potential residue for modification or substitution. The potential residues may be further assessed by substituting with a different type of residue (e.g., cysteine residue, positively charged residue, etc.).

### Affinity Variants

Affinity variants of antibodies may contain modifications or substitutions in one or more CDR sequences provided in Table 2 above, one or more FR sequences, or the heavy or light chain variable region sequences provided in Table 3 above. FR sequences can be readily identified by a person skilled in the art based on the CDR sequences in Table 2 above and variable region sequences in Table 3 above, as it is well-known in the art that a CDR region is flanked by two FR regions in the variable region. The affinity variants retain specific binding affinity to CD3 of the parent antibody, or even have improved CD3 specific binding affinity over the parent antibody. In certain embodiments, at least one (or all) of the substitution(s) in the CDR sequences, FR sequences, or variable region sequences comprises a conservative substitution.

A person skilled in the art will understand that in the CDR sequences provided in Table 2 above, and variable region sequences provided in Table 3 above, one or more amino acid residues may be substituted yet the resulting antibody or antigen-binding fragment still retain the binding affinity or binding capacity to CD3, or even have an improved binding affinity or capacity. Various methods known in the art can be used to achieve this purpose. For example, a library of antibody variants (such as Fab or scFv variants) can be generated and expressed with phage display technology, and then screened for the binding affinity to human CD3. For another example, computer software can be used to virtually simulate the binding of the antibodies to human CD3, and identify the amino acid residues on the antibodies which form the binding interface. Such residues may be either avoided in the substitution so as to prevent reduction in binding affinity, or targeted for substitution to provide for a stronger binding.

In certain embodiments, the humanized antibody or antigen-binding fragment thereof provided herein comprises one or more amino acid residue substitutions in one or more of the CDR sequences, and/or one or more of the FR sequences. In certain embodiments, an affinity variant comprises no more than 20, 15, 10, 9, 8, 7, 6, 5, 4, 3, 2, or 1 substitutions in the CDR sequences and/or FR sequences in total.

In certain embodiments, the anti-CD3 antibodies or antigen-binding fragments thereof provided herein comprise 1, 2, or 3 CDR sequences having at least 80% (e.g., at least 85%, 86%, 87%, 88%, 89%, 90%, 91%, 92%, 93%, 94%, 95%, 96%, 97%, 98%, or 99%) sequence identity to that (or those) listed in Table 2 above yet retaining the specific binding affinity to CD3 at a level similar to or even higher than its parent antibody.

In certain embodiments, the anti-CD3 antibodies or antigen-binding fragments thereof comprise one or more variable region sequences having at least 80% (e.g., at least 85%, 86%, 87%, 88%, 89%, 90%, 91%, 92%, 93%, 94%, 95%, 96%, 97%, 98%, or 99%) sequence identity to that (or those) listed in Table 3 above yet retaining the specific binding affinity to CD3 at a level similar to or even higher than its parent antibody. In some embodiments, a total of 1 to 10 amino acids have been substituted, inserted, or deleted in a variable region sequence listed in Table 3 above. In some embodiments, the substitutions, insertions, or deletions occur in regions outside the CDRs (e.g., in the FRs).

### Glycosylation Variants

The anti-CD3 antibodies or antigen-binding fragments thereof provided herein also encompass glycosylation variants, which can be obtained to either increase or decrease the extent of glycosylation of the antibodies or antigen-binding fragments thereof.

The antibodies or antigen-binding fragments thereof provided herein may comprise one or more modifications that introduce or remove a glycosylation site. A glycosylation site is an amino acid residue with a side chain to which a carbohydrate moiety (e.g., an oligosaccharide structure) can be attached. Glycosylation of antibodies is typically either N-linked or O-linked. N-linked refers to the attachment of the carbohydrate moiety to the side chain of an asparagine residue, for example, an asparagine residue in a tripeptide sequence such as asparagine-X-serine and asparagine-X-threonine, where X is any amino acid except proline. O-linked glycosylation refers to the attachment of one of the sugars N-aceylgalactosamine, galactose, or xylose to a hydroxyamino acid, most commonly to serine or threonine. Removal of a native glycosylation site can be conveniently accomplished, for example, by altering the amino acid sequence such that one of the above-described tripeptide sequences (for N-linked glycosylation sites) or serine or threonine residues (for O-linked glycosylation sites) present in the sequence is substituted. A new glycosylation site can be created in a similar way by introducing such a tripeptide sequence or serine or threonine residue.

### Cysteine-Engineered Variants

The anti-CD3 antibodies or antigen-binding fragments thereof provided herein also encompass cysteine-engineered variants, which comprise one or more introduced free cysteine amino acid residues.

A free cysteine residue is one which is not part of a disulfide bridge. A cysteine-engineered variant is useful for conjugation with for example, a cytotoxic and/or imaging compound, a label, or a radioisoptype among others, at the site of the engineered cysteine, through for example a maleimide or haloacetyl. Methods for engineering antibodies or antigen-binding fragments thereof to introduce free cysteine residues are known in the art, see, for example, WO2006/034488.

### Fc Variants

The anti-CD3 antibodies and antigen-binding fragments provided herein also encompass an Fc variant, which comprises one or more amino acid residue modifications or substitutions at its Fc region and/or hinge region.

In certain embodiments, the anti-CD3 antibodies or antigen-binding fragments thereof provided herein comprise one or more amino acid substitution(s) that improves pH-dependent binding to neonatal Fc receptor (FcRn). Such a variant can have an extended pharmacokinetic half-life, as it binds to FcRn at acidic pH which allows it to escape from degradation in the lysosome and then be translocated and released out of the cell. Methods of engineering an antibody or antigen-binding fragment thereof to improve binding affinity with FcRn are well-known in the art, see, for example, Vaughn, D. et al., Structure, 6(1): 63-73, 1998; Kontermann, R. et al., Antibody Engineering, Volume 1, Chapter 27: Engineering of the Fc region for improved PK, published by Springer, 2010; Yeung, Y. et al., Cancer Research, 70:3269-3277 (2010); and Hinton, P. et al., J. Immunology, 176:346-356 (2006).

In certain embodiments, the anti-CD3 antibodies or antigen-binding fragments thereof provided herein comprise one or more amino acid substitution(s) that alters ADCC. Certain amino acid residues at CH2 domain of the Fc region can be substituted to provide for enhanced ADCC activity. Alternatively or additionally, carbohydrate structures on the antibody can be changed to enhance ADCC activity. Methods of altering ADCC activity by antibody engineering have been described in the art, see for example, Shields R L. et al., J Biol Chem. 2001. 276(9): 6591-604; Idusogie E E. et al., J Immunol. 2000.164(8): 4178-84; Steurer W. et al., J Immunol. 1995, 155(3): 1165-74; Idusogie E E. et al., J Immunol. 2001, 166(4): 2571-5; Lazar G A. et al., PNAS, 2006, 103(11): 4005-4010; Ryan M C. et al., Mol. Cancer Ther., 2007, 6:3009-3018; Richards J O, et al., Mol Cancer Ther. 2008, 7(8): 2517-27; Shields R. L. et al., J. Biol. Chem, 2002, 277:26733-26740; Shinkawa T. et al., J. Biol. Chem, 2003, 278:3466-3473.

In certain embodiments, the anti-CD3 antibodies or antigen-binding fragments thereof comprise one or more amino acid substitution(s) that alters CDC, for example, by improving or diminishing C1q binding and/or CDC (see, for example, WO99/51642; Duncan & Winter Nature 322:738-40 (1988); U.S. Pat. Nos. 5,648,260; 5,624,821); and WO94/29351 concerning other examples of Fc region variants. One or more amino acids selected from amino acid residues 329, 331 and 322 of the Fc region can be replaced with a different amino acid residue to alter C1q binding and/or enhance CDC (see, U.S. Pat. No. 6,194,551 by Idusogie et al.). One or more amino acid substitution(s) can also be introduced to alter the ability of the antibody to fix complement (see PCT Publication WO 94/29351 by Bodmer et al.).

In certain embodiments, the anti-CD3 antibodies or antigen-binding fragments thereof provided herein comprise one or more amino acid substitution(s) in human immunoglobulin (e.g., IgG1) at position 234 and/or 235 (according to EU numbering). In certain embodiments, the anti-CD3 antibodies or antigen-binding fragments thereof provided herein comprise two amino acid substitutions in human immunoglobulin (e.g., IgG1) at positions 234 and 235 (according to EU numbering). In certain embodiments, the anti-CD3 antibodies or antigen-binding fragments thereof provided herein comprise L234A and L235A (according to EU numbering) amino acid substitutions.

In certain embodiments, the anti-CD3 antibodies or antigen-binding fragments thereof provided herein comprise one or more amino acid substitution(s) in human immunoglobulin (e.g., IgG4) at position 228 (according to EU numbering). In certain embodiments, the anti-CD3 antibodies or antigen-binding fragments thereof provided herein comprise S228P (according to EU numbering) amino acid substitution.

In certain embodiments, the anti-CD3 antibodies or antigen-binding fragments thereof provided herein comprise one or more amino acid substitution(s) in the interface of the Fc region to facilitate and/or promote heterodimerization. These modifications comprise introduction of a protuberance into a first Fc polypeptide and a cavity into a second Fc polypeptide, wherein the protuberance can be positioned in the cavity so as to promote interaction of the first and second Fc polypeptides to form a heterodimer or a complex. Methods of generating antibodies with these modifications are known in the art, e.g., as described in U.S. Pat. No. 5,731,168.

In certain embodiments, the anti-CD3 antibodies or antigen-binding fragments thereof provided herein comprise an amino acid substitution at position 366 (according to EU numbering) of a first Fc polypeptide, and comprises one, two or three amino acid substitutions at one, two or three positions of 366, 368, and 407 (according to EU numbering) of a second Fc polypeptide. In certain embodiments, the anti-CD3 antibodies or antigen-binding fragments thereof provided herein comprise a T366W substitution (according to EU numbering) of a first Fc polypeptide, and comprise T366S+L368A+Y407V substitutions (according to EU numbering) of a second Fc polypeptide.

In certain embodiments, the anti-CD3 antibodies or antigen-binding fragments thereof provided herein further comprise one or more amino acid substitution(s) of a first Fc polypeptide, and comprise one or more amino acid substitution(s) of a second Fc polypeptide, so as to introduce a non-natural disulfide bond between the two Fc polypeptides. For example, the anti-CD3 antibodies or antigen-binding fragments thereof provided herein comprise an amino acid substitution at position 354 (according to EU numbering) of a first Fc polypeptide, and comprise an amino acid substitution at position 349 (according to EU numbering) of a second Fc polypeptide. In certain embodiments, the anti-CD3 antibodies or antigen-binding fragments thereof provided herein comprise S354C substitution (according to EU numbering) of a first Fc polypeptide, and comprise Y349C substitution (according to EU numbering) of a second Fc polypeptide.

In certain embodiments, anti-CD3 antibodies or antigen-binding fragments thereof provided herein comprise a first Fc polypeptide comprising the amino acid sequence as set forth in SEQ ID NO: 98, and a second Fc polypeptide comprising the amino acid sequence as set forth in SEQ ID NO: 99.

(SEQ ID NO: 98)
KTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVYTLPPCREEMTKNQVSLWCLVKG
FYPSDIAVEWESNGQPENNYKTTPPVLDSDGSFFLYSKLTVDKSRWQQGN
VFSCSVMHEALHNHYTQKSLSLSPGK
(SEQ ID NO: 99)
KTHTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQVCTLPPSREEMTKNQVSLSCAVKG
FYPSDIAVEWESNGQPENNYKTTPPVLDSDGSFFLVSKLTVDKSRWQQGN
VFSCSVMHEALHNHYTQKSLSLSPGK

### Antigen-Binding Fragments

Provided herein are also anti-CD3 antigen-binding fragments. Various types of antigen-binding fragments are known in the art and can be developed based on the anti-CD3 antibodies provided herein, including for example, the exemplary antibodies whose CDRs are shown in Table 2 above, and variable sequences are shown in Table 3 above, and their different variants (such as affinity variants, glycosylation variants, Fc variants, cysteine-engineered variants and so on).

In certain embodiments, an anti-CD3 antigen-binding fragment provided herein is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a multi-specific antibody, a camelized single domain antibody, a nanobody, a domain antibody, or a bivalent domain antibody.

Various techniques can be used for the production of such antigen-binding fragments. Illustrative methods include, enzymatic digestion of intact antibodies (see, e.g., Morimoto et al., Journal of Biochemical and Biophysical Methods 24:107-117 (1992); and Brennan et al., Science, 229:81 (1985)), recombinant expression by host cells such as E. coli (e.g., for Fab, Fv and ScFv antibody fragments), screening from a phage display library as discussed above (e.g., for ScFv), and chemical coupling of two Fab′-SH fragments to form F (ab′) 2 fragments (Carter et al., Bio/Technology 10:163-167 (1992)). Other techniques for the production of antibody fragments will be apparent to a person skilled in the art.

In certain embodiments, the antigen-binding fragment is a scFv. Generation of scFv is described in, for example, WO 93/16185; U.S. Pat. Nos. 5,571,894; and 5,587,458. ScFv may be fused to an effector protein at either the amino or the carboxyl terminus to provide for a fusion protein (see, for example, Antibody Engineering, ed. Borrebaeck).

In certain embodiments, the anti-CD3 antibodies or antigen-binding fragments thereof provided herein are bivalent, tetravalent, hexavalent, or multivalent. Any molecule being more than bivalent is considered multivalent, encompassing for example, trivalent, tetravalent, hexavalent, and so on.

A bivalent molecule can be monospecific if the two binding sites are both specific for binding to the same antigen or the same epitope. This, in certain embodiments, provides for stronger binding to the antigen or the epitope than a monovalent counterpart. Similar, a multivalent molecule may also be monospecific. In certain embodiments, in a bivalent or multivalent antigen-binding moiety, the first valent of binding site and the second valent of binding site are structurally identical (i.e., having the same sequences), or structurally different (i.e., having different sequences albeit with the same specificity).

A bivalent can also be bispecific, if the two binding sites are specific for different antigens or epitopes. This also applies to a multivalent molecule. For example, a trivalent molecule can be bispecific when two binding sites are monospecific for a first antigen (or epitope) and the third binding site is specific for a second antigen (or epitope).

### Bispecific or Multi-Specific Antibodies

In certain embodiments, the anti-CD3 antibody or an antigen-binding fragment thereof provided herein is bispecific or multi-specific. In certain embodiments, the anti-CD3 antibody or antigen-binding fragment thereof provided herein is further linked to a second functional moiety having a different binding specificity from said anti-CD3 antibody or antigen-binding fragment thereof. In some embodiments, the bispecific or multi-specific antibodies and antigen-binding fragments thereof provided herein has a first specificity for CD3, and a second specificity. In some embodiments, the second specificity is for CD3 but to different epitopes. In some embodiments, the second specificity is for a second antigen different from CD3 and whose presence in proximity to CD3-expressing T cells is desirable for the second antigen to be recognized by immune system. For example, bringing CD3-expressing T cells in close proximity to a tumor antigen or a pathogen antigen and hence promoting recognition or elimination of such an antigen by the immune system.

In certain embodiments, the second specificity is for a tumor associated antigen or an epitope thereof. The term “tumor associated antigen” refers to an antigen that is or can be presented on a tumor cell surface and that is located on or within tumor cells. In some embodiments, the tumor associated antigens can be presented only by tumor cells and not by normal cells, i.e., non-tumor cells. In some other embodiments, the tumor associated antigens can be exclusively expressed on tumor cells or may represent a tumor specific mutation compared to non-tumor cells. In some other embodiments, the tumor associated antigens can be found in both tumor cells and non-tumor cells, but are overexpressed on tumor cells when compared to non-tumor cells or are accessible for antibody binding in tumor cells due to the less compact structure of the tumor tissue compared to non-tumor tissue. In some embodiments, the tumor associated antigen is located on the vasculature of a tumor.

In certain embodiments, the bispecific or multi-specific antibodies or antigen-binding fragments thereof provided herein are capable of specifically binding to one or more (e.g., 1, 2, 3, 4, 5 or more) additional antigens other than CD3, or a second epitope on CD3. In certain embodiments, the one or more additional antigens other than CD3 are selected from the group consisting of CD16a, CD33, CD38, CD45, CD123, CD146, CD228, CLL-1, FLT3, FLT3L, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, LILRB (e.g., LILRB1, LILRB2, LILRB3, LILRB4, LILRB5), c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPa, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), CD160, CTLA-4 (CD152), GPRC5D, IDO (e.g., IDO1, IDO2), TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), TLR3, CLEC9A, DEC-205, STING, and TGFβ.

In certain embodiments, the bispecific antibodies or antigen-binding fragments thereof provided herein are capable of specifically binding to CD3 and CD19. In certain embodiments, the bispecific antibodies or antigen-binding fragments thereof provided herein are capable of specifically binding to CD3 and CLDN18 (e.g., CLDN18.2). In certain embodiments, the bispecific antibodies or antigen-binding fragments thereof provided herein are capable of specifically binding to CD3 and PD-L1. In certain embodiments, the bispecific antibodies or antigen-binding fragments thereof provided herein are capable of specifically binding to CD3 and BCMA. In certain embodiments, the bispecific antibodies or antigen-binding fragments thereof provided herein are capable of specifically binding to CD3 and GPRC5D.

### Conjugates

In some embodiments, the anti-CD3 antibodies or antigen-binding fragments thereof provided herein further comprise one or more conjugate moieties. The conjugate moiety can be linked to the antibodies or antigen-binding fragments thereof. A conjugate moiety is a moiety that can be attached to the antibody or antigen-binding fragment thereof. It is contemplated that a variety of conjugate moieties may be linked to the antibodies or antigen-binding fragments thereof provided herein (see, for example, “Conjugate Vaccines”, Contributions to Microbiology and Immunology, J. M. Cruse and R. E. Lewis, Jr. (eds.), Carger Press, New York, (1989)). These conjugate moieties may be linked to the antibodies or antigen-binding fragments thereof by covalent binding (e.g., disulfide bond), affinity binding, intercalation, coordinate binding, complexation, association, blending, or addition, among other methods. In some embodiments, the antibodies or antigen-binding fragments thereof can be linked to one or more conjugates via a linker or a crosslinking agent. The linker or crosslinking agent comprises a reactive chemical group that can react with the anti-CD3 antibodies or antigen-binding fragments thereof. The reactive chemical groups can be N-succinimidyl esters and N-sulfosuccinimidyl esters. Additionally, the linker comprises a reactive chemical group, which can be a dithiopyridyl group that can react with the drug to form a disulfide bond. Linker molecules include, for example, N-succinimidyl 4-(maleimidomethyl) cyclohexanecarboxylate (SMCC), N-succinimidyl 3-(2-pyridyldithio) propionate (SPDP) (see, e.g., Carlsson et al., Biochem. J., 173:723-737 (1978)), N-succinimidyl 4-(2-pyridyldithio)butanoate (SPDB) (see, e.g., U.S. Pat. No. 4,563,304), N-succinimidyl 4-(2-pyridyldithio)2-sulfobutanoate (sulfo-SPDB) (see US Publication No. 20090274713), N-succinimidyl 4-(2-pyridyldithio) pentanoate (SPP) (see, e.g., CAS Registry number 341498-08-6), 2-iminothiolane, or acetylsuccinic anhydride. For example, the antibody or cell binding agent can be modified with crosslinking reagents and the antibody or cell binding agent containing free or protected thiol groups thus derived is then reacted with a disulfide- or thiol-containing maytansinoid to produce conjugates. The conjugates can be purified by chromatography, including but not limited to HPLC, size-exclusion, adsorption, ion exchange and affinity capture, dialysis or tangential flow filtration.

In certain embodiments, the antibodies or antigen-binding fragments thereof provided herein may be engineered to contain specific sites outside the epitope binding portion that may be utilized for binding to one or more conjugate moieties. For example, such a site may include one or more reactive amino acid residues, such as for example cysteine or histidine residues, to facilitate covalent linkage to a conjugate moiety.

In certain embodiments, the antibodies or antigen-binding fragments thereof provided herein may be linked to a conjugate moiety indirectly, or through another conjugate moiety. For example, the antibodies or antigen-binding fragments thereof provided herein may be conjugated to biotin, then indirectly conjugated to a second conjugate that is conjugated to avidin. In some embodiments, the conjugate moiety comprises a clearance-modifying agent (e.g., a polymer such as PEG which extends half-life), a chemotherapeutic agent, a toxin, a radioactive isotope, a lanthanide, a detectable label (e.g., a luminescent label, a fluorescent label, an enzyme-substrate label), a DNA-alkylator, a topoisomerase inhibitor, a tubulin-binder, a purification moiety or other anticancer drugs (e.g., agonist of toll-like receptor 7 (TLR-7), TLR-8 and/or TLR-9, siRNA, antibody or antigen-binding fragments thereof, a peptide (such as a short peptide), etc.).

A “toxin” can be any agent that is detrimental to cells or that can damage or kill cells. Examples of toxin include, without limitation, taxol, taxoids, CC-1065 and CC-1065 analogs, duocarmycins and duocarmycin analogs, enediynes such as calicheamicins, dolastatin and dolastatin analogs including auristatins, tomaymycin derivatives, leptomycin derivatives, cisplatin, carboplatin, daunorubicin, doxorubicin, vincristine, vinblastine, melphalan, mitomycin C, chlorambucil and morpholino doxorubicin, cytochalasin B, gramicidin D, ethidium bromide, emetine, mitomycin, etoposide, tenoposide, vincristine, MMAE, MMAF, DM1, DM4, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin and analogs thereof, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), anti-mitotic agents (e.g., vincristine and vinblastine), a topoisomerase inhibitor, and a tubulin-binders.

Examples of detectable label may include a fluorescent label (e.g., fluorescein, rhodamine, dansyl, phycoerythrin, or Texas Red), an enzyme-substrate label (e.g., horseradish peroxidase, alkaline phosphatase, luceriferases, glucoamylase, lysozyme, saccharide oxidases or β-D-galactosidase), a radioisotope (e.g., ¹²³I, ¹²⁴I, ¹²⁵I, ¹³¹I, ³⁵S, ³H, ¹¹¹In, ¹¹²In, ¹⁴C, ⁶⁴Cu, ⁶⁷Cu, ⁸⁶Y, ⁸⁸Y, ⁹⁰Y, ¹⁷⁷Lu, ²¹¹At, ¹⁸⁶Re, ¹⁸⁸Re, ¹⁵³Sm, ²¹²Bi, and ³²P, other lanthanides), a luminescent label, a chromophoric moiety, digoxigenin, biotin/avidin, a DNA molecule or gold for detection.

In certain embodiments, the conjugate moiety can be a clearance-modifying agent which helps increase half-life of the antibody. Illustrative examples include water-soluble polymers, such as PEG, carboxymethylcellulose, dextran, polyvinyl alcohol, polyvinyl pyrrolidone, copolymers of ethylene glycol/propylene glycol, and the like. The polymer may be of any molecular weight, and may be branched or unbranched. The number of polymers attached to the antibody may vary, and if more than one polymer are attached, they can be the same or different molecules.

In certain embodiments, the conjugate moiety can be a purification moiety such as a magnetic bead.

In certain embodiments, the antibody or an antigen-binding fragment thereof provided herein is used as a base for a conjugate.

In certain embodiments, the antibody or an antigen-binding fragment thereof provided herein is conjugated to a signal peptide. A signal peptide (sometimes referred to as signal sequence, leader sequence or leader peptide) can be used to facilitate secretion and isolation of the antibodies or antigen-binding fragments thereof provided herein. Signal peptides are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a nucleic acid sequence encoding a signal sequence can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

### Chimeric Antigen Receptor

In certain embodiments, the present disclosure provides a chimeric antigen receptor comprising the antibody or an antigen-binding fragment thereof provided herein, a transmembrane region and an intracellular signal region.

The term “chimeric antigen receptor” or “CAR” or “CARs” as used herein refers to engineered receptors, which graft an antigen specificity onto cells (for example, T cells such as naive T cells, central memory T cells, effector memory T cells, regulatory T cells, natural killer cells, or combination thereof). CARs are also known as artificial T-cell receptors, chimeric T-cell receptors or chimeric immunoreceptors. In some embodiments, CARs comprise an antigen-specific targeting region (for example, the antigen-binding fragments of the anti-CD3 antibody as provided herein), an extracellular region, a transmembrane region, one or more co-stimulatory regions, and an intracellular signal region.

In some embodiments, the antigen-specific targeting region is an scFv. In some embodiments, the transmembrane region comprises a transmembrane region of CD3, CD4, CD8 or CD28. In some embodiments, the co-stimulatory region comprises a co-stimulatory domain of CD28, ICOS, CD27, 4-1BB, OX40 and CD40L. In some embodiments, the intracellular signal region is selected from the group consisting of: an intracellular signal region sequence of CD3, Fc γ RI, CD27, CD28, CD137, CD134, MyD88, CD40, CD278, TLRs, or a combination thereof.

The CARs may be grafted onto various cells, for example, allogeneic cells, autologous cells or xenogeneic cells.

The term “allogeneic cell” as used herein refers to any cells derived from a different subject of the same species.

The term “autologous cell” as used herein refers to any cells derived from the same subject into which they are later to be re-introduced.

The term “xenogeneic cell” as used herein refers to any cells derived from a different subject of a different species.

In some embodiments, the CARs are grafted on immune effector cells, for example, T cells, natural killer cells, macrophage cells, tumor-infiltrating lymphocytes, etc.

### Polynucleotides and Recombinant Methods

The present disclosure provides isolated polynucleotides that encode the anti-CD3 antibodies or antigen-binding fragments thereof and/or the chimeric antigen receptors provided herein. The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

DNA encoding the antibody or antigen-binding fragment thereof, or the chimeric antigen receptor provided herein is readily isolated and sequenced using conventional procedures (e.g., by using oligonucleotide probes that are capable of binding specifically to genes encoding the heavy and light chains of the antibody). The encoding DNA may also be obtained by synthetic methods.

The isolated polynucleotide that encodes the anti-CD3 antibodies or antigen-binding fragments thereof provided herein and/or the chimeric antigen receptors provided herein can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1a), and a transcription termination sequence.

The present disclosure provides vectors comprising the isolated polynucleotides provided herein. In certain embodiments, the polynucleotides provided herein encodes the antibodies or antigen-binding fragments thereof and/or the chimeric antigen receptors provided herein, at least one promoter (e.g., SV40, CMV, EF-la) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g., SV40), lambda phage, and M13 phage, plasmid pcDNA3.3, pMD18-T, pOptivec, pCMV, PEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT.RTM., pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof and/or the chimeric antigen receptor provided herein can be introduced to a host expression system (e.g., a host cell) for cloning or gene expression. In certain embodiments, the host expression system provided herein is a microorganism, a yeast, or a mammalian cell. In certain embodiments, the microorganism is selected from the group consisting of E. coli and B. subtilis. In certain embodiments, the yeast is Saccharomyces. In certain embodiments, the mammalian cell is selected from the group consisting of COS, CHO-S, CHO-K1, HEK-293, and 3T3 cells.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-CD3 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Suitable host cells for the expression of glycosylated antibodies or antigen-fragment thereof provided herein are derived from multicellular organisms. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruiffly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells. Plant cell cultures of cotton, corn, potato, soybean, petunia, tomato, and tobacco can also be utilized as hosts.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; mouse forestomach carcinoma cells (MFC), SNU620 cells, and a human hepatoma line (Hep G2). In some embodiments, the host cell is a mammalian cultured cell line, such as CHO, BHK, NS0, 293, MFC, SNU620 and their derivatives.

Host cells are transformed with the above-described expression or cloning vectors for anti-CD3 antibody production and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable of producing the antibody or antigen-binding fragment thereof provided herein.

The present disclosure also provides a method of expressing the antibody or an antigen-binding fragment thereof and/or the chimeric antigen receptor provided herein, comprising culturing the host expression system provided herein under the condition at which the antibody or antigen-binding fragment thereof and/or the chimeric antigen receptor is expressed. The host expression systems used to produce the antibodies or antigen-binding fragments thereof and/or chimeric antigen receptor provided herein may be cultured in a variety of media. Commercially available media such as Ham's F10 (Sigma), Minimal Essential Medium (MEM) (Sigma), RPMI-1640 (Sigma), and Dulbecco's Modified Eagle's Medium (DMEM) (Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

The anti-CD3 antibodies or antigen-binding fragments thereof and/or the chimeric antigen receptors prepared from the host expression systems can be purified using, for example, hydroxylapatite chromatography, gel electrophoresis, dialysis, DEAE-cellulose ion exchange chromatography, ammonium sulfate precipitation, salting out, and affinity chromatography, with affinity chromatography being the preferred purification technique.

In certain embodiments, Protein A immobilized on a solid phase is used for immunoaffinity purification of the antibody and antigen-binding fragment thereof and/or the chimeric antigen receptor provided herein. The suitability of protein A as an affinity ligand depends on the species and isotype of any immunoglobulin Fc domain that is present in the antibody. Protein A can be used to purify antibodies that are based on human gamma1, gamma2, or gamma4 heavy chains (Lindmark et al., J. Immunol. Meth. 62:1-13 (1983)). Protein G is recommended for all mouse isotypes and for human gamma3 (Guss et al., EMBO J. 5:1567 1575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a CH3 domain, the Bakerbond ABX™ resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25M salt).

### Pharmaceutical Composition

The present disclosure further provides pharmaceutical compositions comprising the anti-CD3 antibodies or antigen-binding fragments thereof and/or the chimeric antigen receptors provided herein, and one or more pharmaceutically acceptable carriers.

Pharmaceutical acceptable carriers for use in the pharmaceutical compositions disclosed herein may include, for example, pharmaceutically acceptable liquid, gels, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxanisol, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine. Further provided are methods for preventing oxidation of, extending the shelf-life of, and/or improving the efficacy of an antibody or antigen-binding fragment provided herein by mixing the antibody or antigen-binding fragment with one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcelluose, hydroxypropyl methylcellulose, or polyvinylpyrrolidone, emulsifying agents such as Polysorbate 80 (TWEEN-80), sequestering or chelating agents such as EDTA (ethylenediaminetetraacetic acid) or EGTA (ethylene glycol tetraacetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, mercurials, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

The pharmaceutical compositions can be a liquid solution, suspension, emulsion, pill, capsule, tablet, sustained release formulation, or powder. Oral formulations can include standard carriers such as pharmaceutical grades of mannitol, lactose, starch, magnesium stearate, polyvinyl pyrollidone, sodium saccharine, cellulose, magnesium carbonate, etc.

In certain embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In certain embodiments, unit-dose parenteral preparations are packaged in an ampoule, a vial or a syringe with a needle. All preparations for parenteral administration should be sterile and not pyretic, as is known and practiced in the art.

In certain embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbital, fructose, corn syrup, xylitol, glycerin, glucose, sucrose or other suitable agent. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the anti-CD3 antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given, and can be empirically determined.

### Kits

In certain embodiments, the present disclosure provides a kit comprising the antibody or an antigen-binding fragment thereof provided herein and/or the pharmaceutical composition provided herein and/or the chimeric antigen receptor provided herein. In certain embodiments, the present disclosure provides a kit comprising the antibody or an antigen-binding fragment thereof provided herein and/or the pharmaceutical composition provided herein and/or the chimeric antigen receptor provided herein, and a second therapeutic agent. In certain embodiments, the second therapeutic agent is selected from the group consisting of a chemotherapeutic agent, an anti-cancer drug, radiation therapy agent, an immunotherapy agent, an anti-angiogenesis agent, a targeted therapy agent, a cellular therapy agent, a gene therapy agent, a hormonal therapy agent, an antiviral agent, an antibiotic, an analgesics, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

### Methods of Use

The present disclosure also provides methods of treating, preventing or alleviating a disease, disorder or condition in a subject, comprising administering to the subject a therapeutically effective amount of the antibody or antigen-binding fragment thereof provided herein, and/or the pharmaceutical composition provided herein, and/or the chimeric antigen receptor provided herein. In certain embodiments, the disease, disorder or condition is a CD3 related disease, disorder or condition. In certain embodiments, the subject is human.

In some embodiments, the CD3 related disease, disorder or condition is characterized in expressing or over-expressing of CD3.

In certain embodiments, the disease, disorder or condition is immune disease, inflammatory disease, cancer or neurological disease. In certain embodiments, the cancer is a CD3-expressing cancer. “CD3-expressing” cancer as used herein refers to a cancer characterized in expressing CD3 protein in a cancer cell, a tumor infiltrating immune cell, or expressing CD3 in a cancer cell, a tumor infiltrating immune cell at a level significantly higher than that would have been expected of a normal cell. Various methods can be used to determine the presence and/or amount of CD3 in a test biological sample from the subject. For example, the test biological sample can be exposed to an anti-CD3 antibody or antigen-binding fragment thereof, which binds to and detects the expressed CD3 protein. Alternatively, CD3 can also be detected at nucleic acid expression level, using methods such as qPCR, reverse transcriptase PCR, microarray, SAGE, FISH, and the like. In some embodiments, the test sample is derived from a cancer cell or tissue, or tumor infiltrating immune cells. The reference sample can be a control sample obtained from a healthy or non-diseased individual, or a healthy or non-diseased sample obtained from the same individual from whom the test sample is obtained. For example, the reference sample can be a non-diseased sample adjacent to or in the neighborhood of the test sample (e.g., tumor). In certain embodiments, the cancer is a solid tumor or hematologic tumor.

In certain embodiments, the disease, disorder or condition is selected from the group consisting of lung cancer (e.g., non-small-cell lung cancer (NSCLC), small cell lung cancer (SCLC), adenocarcinoma of the lung, or squamous cell carcinoma of the lung), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), melanoma (e.g., cutaneous or intraocular melanoma), thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma.

In some embodiments, the subject has been identified as having a cancer cell or tumor infiltrating immune cells expressing CD3, optionally at a level significantly higher from the level normally found on non-cancer cells.

In another aspect, methods are provided to treat, prevent or alleviate a disease, disorder or condition in a subject that would benefit from modulation of CD3 activity, comprising administering to the subject a therapeutically effective amount of the antibody or antigen-binding fragment thereof provided herein, and/or the pharmaceutical composition provided herein, and/or the chimeric antigen receptor provided herein. In certain embodiments, the disease, disorder or condition is a CD3 related disease, disorder or condition, which is defined above.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g., physician or veterinarian) as indicated by these and other circumstances or requirements.

In certain embodiments, the antibody or antigen-binding fragment thereof and/or the chimeric antigen receptor provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies or antigen-binding fragments thereof provided herein may be administered by any route known in the art, for example the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

In some embodiments, the antibodies or antigen-binding fragments thereof and/or the chimeric antigen receptors provided herein may be administered alone or in combination with a therapeutically effective amount of a second therapeutic agent. For example, the antibodies or antigen-binding fragments thereof and/or the chimeric antigen receptors disclosed herein may be administered in combination with a second therapeutic agent, for example, a chemotherapeutic agent, an anti-cancer drug, a radiation therapy agent, an immunotherapy agent, a targeted therapy agent, a cellular therapy agent, a gene therapy agent, a hormonal therapy agent, an antiviral agent, an antibiotic, an analgesics, an antioxidant, a metal chelator, cytokines, an active agent, an imaging agent, a cytotoxic agent, an angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, or a microRNA.

The term “immunotherapy” as used herein, refers to a type of therapy that stimulates immune system to fight against disease such as cancer or that boosts immune system in a general way. Examples of immunotherapy include, without limitation, checkpoint modulators, adoptive cell transfer, cytokines, oncolytic virus and therapeutic vaccines.

“Targeted therapy” is a type of therapy that acts on specific molecules associated with cancer, such as specific proteins that are present in cancer cells but not normal cells or that are more abundant in cancer cells, or the target molecules in the cancer microenvironment that contributes to cancer growth and survival. Targeted therapy targets a therapeutic agent to a tumor, thereby sparing of normal tissue from the effects of the therapeutic agent.

In certain of these embodiments, an antibody or antigen-binding fragment thereof provided herein, and/or the pharmaceutical composition provided herein, and/or the chimeric antigen receptor provided herein that is administered in combination with one or more additional therapeutic agents may be administered simultaneously with the one or more additional therapeutic agents, and in certain of these embodiments the antibody or antigen-binding fragment thereof, and/or the pharmaceutical composition provided herein, and/or the chimeric antigen receptor provided herein, and the additional therapeutic agent(s) may be administered as part of the same pharmaceutical composition. However, an antibody or antigen-binding fragment thereof, and/or a pharmaceutical composition provided herein, and/or a chimeric antigen receptor provided herein administered “in combination” with another therapeutic agent does not have to be administered simultaneously with or in the same composition as the agent. An antibody or antigen-binding fragment thereof, pharmaceutical composition, or chimeric antigen receptor administered prior to or after another agent is considered to be administered “in combination” with that agent as the phrase is used herein, even if the antibody or antigen-binding fragment, pharmaceutical composition or chimeric antigen receptor, and the second agent are administered via different routes. Where possible, additional therapeutic agents administered in combination with the antibodies or antigen-binding fragments thereof, pharmaceutical compositions, or chimeric antigen receptors disclosed herein are administered according to the schedule listed in the product information sheet of the additional therapeutic agent, or according to the Physicians' Desk Reference 2003 (Physicians' Desk Reference, 57th Ed; Medical Economics Company; ISBN: 1563634457; 57th edition (November 2002)) or protocols well known in the art.

The present disclosure further provides method of activating CD3-expressing T cells in vivo or in vitro, comprising contacting the CD3-expressing T cells with the antibody or antigen-binding fragment thereof provided herein, and/or the pharmaceutical composition provided herein, and/or the chimeric antigen receptor provided herein.

The present disclosure further provides methods of modulating CD3 activity in CD3-expressing cells, comprising exposing the CD3-expressing cells to the antibodies or antigen-binding fragments thereof provided herein, and/or the pharmaceutical composition provided herein, and/or the chimeric antigen receptor provided herein.

The present disclosure further provides methods of promoting in vivo or in vitro processing of a second antigen by CD3-expressing T cells, comprising contacting the CD3-expressing T cells with the bispecific antibodies or antigen-binding fragments thereof provided herein, wherein the bispecific antibodies or antigen-binding fragments thereof are capable of specifically binding to both the CD3-expressing T cells and a second antigen thereby bringing both in close proximity.

In another aspect, the present disclosure provides methods of detecting the presence or amount of CD3 in a sample, comprising contacting the sample with the antibody or antigen-binding fragment thereof provided herein, and/or the pharmaceutical composition provided herein, and/or the chimeric antigen receptor provided herein, and determining the presence or the amount of CD3 in the sample.

In another aspect, the present disclosure provides a method of diagnosing a CD3 related disease, disorder or condition in a subject, comprising: a) obtaining a sample from the subject, b) contacting the sample obtained from the subject with the antibody or an antigen-binding fragment thereof provided herein and/or the pharmaceutical composition provided herein and/or the chimeric antigen receptor provided herein; c) determining the presence or amount of CD3 in the sample; and d) correlating the presence or the amount of CD3 to existence or status of the CD3 related disease, disorder or condition in the subject.

In another aspect, the present disclosure provides kits comprising the antibody or antigen-binding fragment thereof provided herein and/or the pharmaceutical composition provided herein and/or the chimeric antigen receptor provided herein, optionally conjugated with a detectable moiety, which is useful in detecting CD3, optionally recombinant CD3, CD3 expressed on cell surface, or CD3-expressing cells. The kits may further comprise instructions for use.

In another aspect, the present disclosure also provides use of the antibody or antigen-binding fragment thereof provided herein and/or the pharmaceutical composition provided herein and/or the chimeric antigen receptor provided herein in the manufacture of a medicament for treating, preventing or alleviating a CD3 related disease, disorder or condition in a subject, in the manufacture of a diagnostic reagent for diagnosing a CD3 related disease, disorder or condition.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. A person skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Examples

### Example 1. Antibody Generation

### 1.1. Immunization

To generate antibodies to CD3, SJL, Balb/c and CD1 mice were immunized with cells (i.e., human CD3 overexpressing cells (Jurkat-hCD3mix or 293T-hCD3mix), human CD3⁺ T cells, activated human CD3⁺ T cells, cynomolgus CD3⁺ T cells), CD3 proteins (i.e., human CD3 δε complex and human CD3 γε complex, cynomolgus CD3 δε complex and cynomolgus CD3 γε complex protein), or genes (pTT5-Human CD3 δε plasmid, pTT5-Cynomolgus CD3 de plasmid) in each group. Balb/c and CD1 mice were immunized as shown in Table 4. SJL mice were immunized as shown in Table 5. The primary immunization was followed by several boosts until animals developed satisfactory antiserum titers suitable for hybridoma development. CPG was used as adjuvant. The immunization protocols for each group of animals were shown in Tables 6, 7, 8, 9, 10 and 11 below.

TABLE 4
Grouping of Balb/c &CD1 Mice

| | | | Animal/ | Group | |
|---|---|---|---|---|---|
| Group | Immunogen | Route | Strain | size | Dosage |
| 1 | Jurkat-hCD3mix | I.P. | Balb/c& | 5/5 | 5 × 10⁶ per mouse |
| | | | CD1 | | |
| 2 | Human CD3⁺ T | I.P. | Balb/c& | 5/5 | 5 × 10⁶ per mouse |
| | cells | | CD1 | | |
| 3 | Activated human | I.P. | Balb/c& | 5/5 | 5 × 10⁶ per mouse |
| | CD3⁺ T cells | | CD1 | | |
| 4 | Human CD3 δε | I.H. | Balb/c& | 5/5 | 25 μg |
| | complex | | CD1 | | 25 μg |
| | Human CD3 γε | | | | |
| | complex | | | | |

TABLE 5
Grouping of SJL Mice

| | | | Animal/ | Group | |
|---|---|---|---|---|---|
| Group | Immunogen | Route | Strain | size | Dosage |
| 5 | pTT5-Cynomolgus | Gene | SLJ | 3/3 | 4 μg |
| | CD3 δε plasmid | gun | | | |
| | pTT5-Human CD3 | Gene | | | 4 μg |
| | δε plasmid | gun | | | |
| | pTT5-Human CD3 | Gene | | | 2 μg + 2 μg |
| | δε plasmid + pTT5- | gun | | | 12.5 μg + 12.5 μg + |
| | Cynomolgus CD3 | S.C. | | | 12.5 μg + 12.5 μg |
| | δε plasmid | | | | |
| | & | | | | |
| | Human CD3 δε | | | | |
| | complex + | | | | |
| | Human CD3 | | | | |
| | γε complex + | | | | |
| | Cynomolgus CD3 | | | | |
| | δε complex + | | | | |
| | Cynomolgus | | | | |
| | CD3 γε complex | | | | |
| | pTT5-Human CD3 | Gene | | | 2 μg + 2 μg |
| | δε plasmid + pTT5- | gun | | | |
| | Cynomolgus CD3 | | | | |
| | δε plasmid | | | | |
| | pTT5-Human CD3 | Gene | | | 2 μg + 2 μg |
| | δε plasmid + pTT5- | gun | | | 12.5 μg + 12.5 μg + |
| | Cynomolgus CD3 | S.C. | | | 12.5 μg + 12.5 μg |
| | δε plasmid | I.P. | | | 5 × 10⁶ per mouse + |
| | & | | | | 5 × 10⁶ per mouse |
| | Human CD3 δε | | | | |
| | complex + | | | | |
| | Human CD3 | | | | |
| | γε complex + | | | | |
| | Cynomolgus CD3 | | | | |
| | δε complex + | | | | |
| | Cynomolgus | | | | |
| | CD3 γε complex | | | | |
| | & | | | | |
| | Jurkat cells + | | | | |
| | Cynomolgus | | | | |
| | CD3⁺ T cells | | | | |
| 6 | pTT5-Cynomolgus | Gene | SLJ | 2/2 | 4 μg |
| | CD3 δε plasmid | gun | | | |
| | pTT5-Human CD3 | Gene | | | 4 μg |
| | δε plasmid | gun | | | |
| | pTT5-Human CD3 | Gene | | | 2 μg + 2 μg |
| | δε plasmid + pTT5- | gun | | | 12.5 μg + 12.5 μg + |
| | Cynomolgus CD3 | S.C. | | | 12.5 μg + 12.5 μg |
| | δε plasmid | | | | |
| | & | | | | |
| | Human CD3 δε | | | | |
| | complex + | | | | |
| | Human CD3 | | | | |
| | γε complex + | | | | |
| | Cynomolgus CD3 | | | | |
| | δε complex + | | | | |
| | Cynomolgus | | | | |
| | CD3 γε complex | | | | |
| | pTT5-Human CD3 | Gene | | | 2 μg + 2 μg |
| | δε plasmid + pTT5- | gun | | | |
| | Cynomolgus CD3 | | | | |
| | δε plasmid | | | | |
| | pTT5-Human CD3 | Gene | | | 2 μg + 2 μg |
| | δε plasmid + pTT5- | gun | | | 5 × 10⁶ per mouse + |
| | Cynomolgus CD3 | I.P. | | | 5 × 10⁶ per mouse |
| | δε plasmid | | | | |
| | & | | | | |
| | 293T-hCD3mix | | | | |
| | cells + | | | | |
| | Cynomolgus | | | | |
| | CD3⁺ T cells | | | | |

TABLE 6
Immunization protocol for Group 1

| | |
|---|---|
| Day 0 | Pre-bleed (15-30 μL serum/mouse) |
| | Primary: 5 × 10⁶ cells per mouse I.P. |
| Day 21 | Boost 1: 5 × 10⁶ cells per mouse I.P. |
| Day 35 | Test Bleed (15-30 μL serum/mouse) (TB1) |
| Day 42 | Boost 2: 5 × 10⁶ cells per mouse I.P. |
| Day 56 | Test Bleed (15-30 μL serum/mouse) (TB2) |
| Day 77 | Boost 3: 5 × 10⁶ cells per mouse I.P. |
| Day 91 | Test Bleed (15-30 μL serum/mouse) (TB3) |
| | Data analysis and phase conclusion |
| Day 118 | Pre-fusion (final) Boost, for each mouse: 25 μg CD3 δε |
| | complex I.H., 25 μg CD3 γε complex, I.H., 5 × 10⁶ |
| | Jurkat-hCD3mix I.P., 5 × 10⁶ Activated CD3⁺ T cells, I.P. |

Animals not selected for cell fusion were maintained in cage and may be given additional boost immunizations.

TABLE 7
Immunization protocol for Group 2

| | |
|---|---|
| Day 0 | Pre-bleed (15-30 μL serum/mouse) |
| | Primary: 5 × 10⁶ cells per mouse I.P. |
| Day 21 | Boost 1: 5 × 10⁶ cells per mouse I.P. |
| Day 35 | Test Bleed (15-30 μL serum/mouse) (TB1) |
| Day 42 | Boost 2: 5 × 10⁶ cells per mouse I.P. |
| Day 56 | Test Bleed (15-30 μL serum/mouse) (TB2) |
| Day 77 | Boost 3: 5 × 10⁶ cells per mouse I.P. |
| Day 91 | Test Bleed (15-30 μL serum/mouse) (TB3) |
| | Data analysis and phase conclusion |
| Day 118 | Pre-fusion (final) Boost, for each mouse: 25 μg CD3 δε |
| | complex I.H., 25 μg CD3 γε complex, I.H., 5 × 10⁶ |
| | Jurkat-hCD3mix I.P., 5 × 10⁶ Activated CD3⁺ T cells, I.P. |

Animals not selected for cell fusion were maintained in cage and may be given additional boost immunizations.

TABLE 8
Immunization protocol for Group 3

| | |
|---|---|
| Day 0 | Pre-bleed (15-30 μL serum/mouse) |
| | Primary: 5 × 10⁶ cells per mouse I.P. |
| Day 21 | Boost 1: 5 × 10⁶ cells per mouse I.P. |
| Day 35 | Test Bleed (15-30 μL serum/mouse) (TB1) |
| Day 42 | Boost 2: 5 × 10⁶ cells per mouse I.P. |
| Day 56 | Test Bleed (15-30 μL serum/mouse) (TB2) |
| Day 77 | Boost 3: 5 × 10⁶ cells per mouse I.P. |
| Day 91 | Test Bleed (15-30 μL serum/mouse) (TB3) |
| | Data analysis and phase conclusion |
| Day 118 | Pre-fusion (final) Boost, for each mouse: 25 μg CD3 δε |
| | complex I.H., 25 μg CD3 γε complex, I.H., 5 × 10⁶ |
| | Jurkat-hCD3mix I.P., 5 × 10⁶ Activated CD3⁺ T cells, I.P. |

Animals not selected for cell fusion were maintained in cage and may be given additional boost immunizations.

TABLE 9
Immunization protocol for Group 4

| | |
|---|---|
| Day 0 | Pre-bleed (15-30 μL serum/mouse) |
| | Primary: 25 μg CD3 δε complex I.H., 25 μg CD3 y& |
| | complex, I.H. |
| Day 21 | Boost 1: 25 μg CD3 δε complex I.H., 25 μg CD3 γε |
| | complex, I.H. per mouse |
| Day 35 | Test Bleed (15-30 μL serum/mouse) (TB1) |
| Day 42 | Boost 2: 25 μg CD3 δε complex I.H., 25 μg CD3 γε |
| | complex, I.H. per mouse |
| Day 56 | Test Bleed (15-30 μL serum/mouse) (TB2) |
| Day 77 | Boost 3: 25 μg CD3 δε complex I.H., 25 μg CD3 γε |
| | complex, I.H. per mouse |
| Day 91 | Test Bleed (15-30 μL serum/mouse) (TB3) |
| | Data analysis and phase conclusion |
| Day 118 | Pre-fusion (final) Boost, for each mouse: 25 μg CD3 δε |
| | complex I.H., 25 μg CD3 γε complex, I.H., 5 × 10⁶ |
| | Jurkat-hCD3mix I.P., 5 × 10⁶ Activated CD3⁺ T cells, I.P. |

Animals not selected for cell fusion were maintained in cage and may be given additional boost immunizations.

TABLE 10
Immunization protocol for Group 5

| | |
|---|---|
| Day 0 | Pre-bleed (15-30 μL serum/mouse) |
| | Primary: pTT5-Cynomolgus CD3 δε plasmid 4 μg, gene |
| | gun. |
| Day 14 | Boost 1: pTT5-Human CD3 δε plasmid, 4 μg, gene gun. |
| Day 21 | Test Bleed (15-30 μL serum/mouse) (TB1) |
| Day 28 | Boost 2: pTT5-Cynomolgus CD3 δε plasmid 4 μg, gene |
| | gun. |
| Day 35 | Test Bleed (15-30 μL serum/mouse) (TB2) |
| Day 42 | Boost 3: pTT5-Human CD3 δε plasmid 2 μg + pTT5- |
| | Cynomolgus CD3 8 plasmid 2 μg, gene gun; Human CD3 |
| | δε complex 12.5 μg + Human CD3 γε complex 12.5 μg + |
| | Cynomolgus CD3 δε complex 12.5 μg + Cynomolgus CD3 |
| | γε complex 12.5 μg, S.C.. |
| Day 49 | Test Bleed (15-30 μL serum/mouse) (TB3) |
| Day 56 | Boost 4: pTT5-Human CD3 δε plasmid 2 μg + pTT5- |
| | Cynomolgus CD3 δε plasmid 2 μg, gene gun. |
| Day 78 | Boost 5: pTT5-Human CD3 δε plasmid 2 μg + pTT5- |
| | Cynomolgus CD3 δε plasmid 2 μg, gene gun; Human CD3 |
| | δε complex 12.5 μg + Human CD3 γε complex 12.5 μg + |
| | Cynomolgus CD3 δε complex12.5 μg + Cynomolgus CD3 |
| | γε complex 12.5 μg, S.C.. |
| Day 85 | Test Bleed (15-30 μL serum/mouse) (TB5) |
| | Data analysis and phase conclusion |
| Day 92 | Pre-fusion (final) Boost: pTT5-Human CD3 δε plasmid |
| | 2 μg + pTT5-Cynomolgus CD3 δε plasmid 2 μg, gene gun; |
| | Human CD3 δε complex 12.5 μg + Human CD3 γε |
| | complex 12.5 μg + Cynomolgus CD3 δε complex 12.5 μg + |
| | Cynomolgus CD3 γε complex 12.5 μg, S.C.; 5 × 10⁶ Jurkat |
| | cells + 5 × 10⁶ Cynomolgus CD3⁺ T cells, I.P.. |

Animals not selected for cell fusion were maintained in cage and may be given additional boost immunizations.

TABLE 11
Immunization protocol for Group 6

| | |
|---|---|
| Day 0 | Pre-bleed (15-30 μL serum/mouse) |
| | Primary: pTT5-Cynomolgus CD3 de plasmid 4 μg, gene gun. |
| Day 14 | Boost 1: pTT5-Human CD3 δε plasmid, 4 μg, gene gun. |
| Day 21 | Test Bleed (15-30 μL serum/mouse) (TB1) |
| Day 28 | Boost 2: pTT5-Cynomolgus CD3 δε plasmid 4 μg, gene gun. |
| Day 35 | Test Bleed (15-30 μL serum/mouse) (TB2) |
| Day 42 | Boost 3: pTT5-Human CD3 δε plasmid 2 μg + pTT5- |
| | Cynomolgus CD3 δε plasmid 2 μg, gene gun; Human CD3 & |
| | complex 12.5 μg + Human CD3 γε complex 12.5 μg + |
| | Cynomolgus CD3 δε complex 12.5 μg + Cynomolgus CD3 |
| | γε complex 12.5 μg, S.C. |
| Day 49 | Test Bleed (15-30 μL serum/mouse) (TB3) |
| Day 56 | Boost 4: pTT5-Human CD3 δε plasmid 2 μg + pTT5- |
| | Cynomolgus CD3 δε plasmid 2 μg, gene gun. |
| Day 78 | Boost 5: pTT5-Human CD3 δε plasmid 2 μg + pTT5- |
| | Cynomolgus CD3 δε plasmid 2 μg, gene gun; 5 × 10⁶ 293T- |
| | hCD3mix cells + 5 × 10⁶ Cynomolgus CD3⁺ T cells, I.P.. |
| Day 85 | Test Bleed (15-30 μL serum/mouse) (TB5) |
| | Data analysis and phase conclusion |
| Day 92 | Pre-fusion (final) Boost: pTT5-Human CD3 δε plasmid 2 μg + |
| | pTT5-Cynomolgus CD3 δε plasmid 2 μg, gene gun; |
| | Human CD3 δε complex 12.5 μg + Human CD3 γε complex |
| | 12.5 μg + Cynomolgus CD3 δε complex 12.5 μg + |
| | Cynomolgus CD3 γε complex 12.5 μg, S.C.; 5 × 10⁶ Jurkat |
| | cells + 5 × 10⁶ Cynomolgus CD3⁺ T cells, I.P.. |

Animals not selected for cell fusion were maintained in cage and may be given additional boost immunizations.

### 1.2. Hybridoma Generation and Screening

### 1.2.1. Cell Fusion and Screening

Splenocyte fusions were performed on the mice which responded the best to the immunizations as determined by test bleed FACS and/or ELISA. The FACS assay were performed on 293F, HEK 293T or Jurkat cell line stably over-expressing human CD3. The lymphocytes from spleens and lymph nodes were fused to a mouse myeloma cell line (SP2/0) using an optimized electrofusion protocol. Multiple fusions were performed to ensure success of the project.

The fusions were plated (2×10⁴ to 10⁵ cells per well) into a stack of 96-well plates. Plates were monitored for growth and fed weekly. Wells with cell growth were screened by primary screening assays in 10-14 days with FACS and/or other feasible assays such as ELISA (with biotin labeled human CD3 δε complex). Multiple fusions for each targeting antigen were performed and screened. The positive parental clones which showed positive binding with HEK293T-hCD3mix, Jurkat-hCD3mix or CHO-K1 stably over-expressing human CD3 (CHO-K1-hCD3mix) cells and positive ELISA signal (such as ELISA signal with human CD3 & complex and human CD3 γε complex) from primary screening were expanded into 24-well plates for secondary screening. Hybridomas of interest (such as hybridomas which showed positive cell-based binding with Jurkat and 293F-cynoCD3mix cells) were chosen to proceed to subcloning.

### 1.2.2. Hybridoma Subcloning, Screening and Cryopreservation

The parental hybridomas with desired reactivity and isotypes from the screening funnel above were then subcloned by multiple rounds of limiting dilution or single cell sorting until monoclonals were obtained.

The subcloning plates were screened by protein, cell-based ELISA, or cell-based binding assay (such as cell-based binding assay on CHO-K1-hCD3mix cells, ELISA assay on human CD3 δε complex and human CD3 ye complex proteins), and the subclones with good binding ability were expanded to 24-wells for confirmation tests by cell-based biding assay on HEK293T and HEK293T-hCD3mix cells, or Jurkat and 293F-cynoCD3mix cells.

The desired subclonal cell lines were sequenced and further expanded into culture flasks for cryopreservation. 4-6 vials per cell line at 0.5-13.0×10⁶ cells/vial were initially cryopreserved. Master cell bank and working cell bank were established for the selected most valuable cell lines.

As a result, 19 antibodies were discovered with unique sequences. Among the 19 antibodies, 12 antibodies showed positive binding with HEK293T cell stably over-expressing human CD3 protein (293T-hCD3mix) but not binding to parental HEK293T cells (as shown in Table 12 below), suggesting these antibodies are human CD3 recognizing antibody; the other 7 antibodies showed positive binding with Jurkat and 293F-cynoCD3mix cells but not binding to Jurkat-KO or parental 293F cells (as shown in Table 13 below), suggesting these antibodies are human and cynomolgus CD3 recognizing antibody.

TABLE 12
FACS MFI of antibodies binding
with HEK293T and 293T-hCD3mix

| | | FACS MFI |
|---|---|---|
| | FACS MFI | (HEK293T- |
| Antibody | (HEK293T) | hCD3mix) |
| 25-G12-G6-C12 | 200 | 38920 |
| 16-F2-C11-D9 | 2070 | 80290 |
| 20-E11-E11-C2 | 2140 | 55240 |
| 7-D9-G10-H2 | 4330 | 50980 |
| 31-F8-F5-C5 | 500 | 77328 |
| 7-D8-G12-E4 | 1616 | 65240 |
| 40-C12-C10-E9 | 290 | 13120 |
| 2-F12-A6-G2 | 880 | 53840 |
| 3-C6-C11-F12 | 1760 | 21750 |
| 8-B12-F9-B11 | 160 | 67080 |
| 4-F12-F1-A4 | 500 | 16760 |
| 3-F3-G12-E2 | 14080 | 92740 |

TABLE 13
MFI of antibodies binding with Jurkat, Jurkat-KO,
293F-cynoCD3mix and 293F cells

| | FACS | FACS | FACS MFI | FACS |
|---|---|---|---|---|
| | MFI | MFI | (293F- | MFI |
| Antibody | (Jurkat) | (Jurkat-KO) | cynoCD3mix) | (293F) |
| 124E3D6 | 126 | 97 | 400 | 98 |
| 126A11A4 | 1621 | 101 | 2215 | 101 |
| 127E2D3 | 930 | 109 | 1925 | 102 |
| 133B4C7 | 681 | 105 | 1785 | 100 |
| 140D2B10 | 872 | 100 | 2038 | 98 |
| 147C6F3 | 1857 | 101 | 2231 | 98 |
| 147E11E2 | 2453 | 104 | 2526 | 102 |

### Example 2. Antibody Characterization

### 2.1. Antibodies

The hybridoma antibody clones 25-G12-G6-C12, 40-C12-C10-E9, 8-B12-F9-B11, 31-F8-F5-C5, 16-F2-C11-D9, 20-E11-E11-C2, 7-D9-G10-H2, 7-D8-G12-E4, 2-F12-A6-G2, 3-C6-C11-F12, 4-F12-F1-A4, 3-F3-G12-E2, 124E3D6, 126A11A4, 127E2D3, 133B4C7, 140D2B10, 147C6F3 and 147E11E2 were characterized.

### 2.2. Hybridoma Sequencing

Total RNA was isolated from the hybridoma cells following the technical manual of RNAiso Plus (TAKARA Cat #9109). Total RNA was then reverse transcribed into cDNA using isotype-specific anti-sense primers or universal primers following the technical manual of PrimeScript II 1st Strand cDNA synthesis Kit (TAKARA Cat #6210A). The antibody fragments of VH and VL were amplified according to TakaRa Taq™ (Cat #R001A). Amplified antibody fragments were cloned into a standard cloning vector separately. Colony PCR was performed to screen for clones with inserts of correct sizes. No less than five colonies with inserts of correct sizes were sequenced for each fragment. The sequences of different clones were aligned and the consensus sequence of these clones was provided.

The variable region sequences of the hybridoma antibodies are provided herein in Table 3 above.

### 2.3. Characterization of Antibodies 25-G12-G6-C12, 40-C12-C10-E9, 8-B12-F9-B11, 31-F8-F5-C5, 16-F2-C11-D9, 20-E11-E11-C2, 7-D9-G10-H2, 7-D8-G12-E4, 2-F12-A6-G2, 3-C6-C11-F12, 4-F12-F1-A4, and 3-F3-G12-E2

The T cell activation capability of these antibodies was determined by Jurkat NFAT-Luciferase activation assay. OKT3 was used as a positive control. The protocol for activation analysis is described as follows:
Mouse antibodies were coated on 96-well plates at 37° C. for 1 hour, 50 μg/mL and 100 μl per well;
Jurkat-NFAT-Luciferase cells were seeded in each well for 24 hours, 2×10⁴ cells per well;
20 μL Bright-Glo were added to each well, the luciferase value was read with microplate reader.

The results are shown in Table 14 below. As shown in Table 14, the activation capacity of these mouse antibodies was higher than the positive control OKT3.

TABLE 14
Activation of Jurkat-NFAT-luciferase
with mouse antibodies

| | | |
|---|---|---|
| Number | Name | RLU |

Plate 1

| | | | |
|---|---|---|---|
| 1 | OKT3 | 5626 | 5711 |
| 2 | Isotype control | 564 | 582 |
| 3 | 16-F2-C11-D9 | 941 | 789 |
| 4 | 20-E11-E11-C2 | 748 | 710 |
| 5 | 7-D9-G10-H2 | 772 | 712 |
| 6 | 40-C12-C10-E9 | 1101 | 1057 |
| 7 | 2-F12-A6-G2 | 786 | 688 |
| 8 | 4-F12-F1-A4 | 1504 | 1557 |

Plate 2

| | | | |
|---|---|---|---|
| 1 | OKT3 | 2594 | 2520 |
| 2 | Isotype control | 386 | 380 |
| 3 | 25-G12-G6-C12 | 1503 | 1211 |
| 4 | 31-F8-F5-C5 | 606 | 722 |
| 5 | 7-D8-G12-E4 | 395 | 378 |
| 6 | 3-C6-C11-F12 | 509 | 513 |

Plate 3

| | | | |
|---|---|---|---|
| 1 | OKT3 | 1131 | 1045 |
| 2 | Isotype control | 237 | 231 |
| 3 | 8-B12-F9-B11 | 1039 | 1144 |

### Example 3. Chimeric Antibody Generation and Characterization

### 3.1. Chimeric Antibody Generation

Sequences of 16 hybridoma antibodies (25-G12-G6-C12, 4-F12-F1-A4, 7-D8-G12-E4, 20-E11-E11-C2, 7-D9-G10-H2, 40-C12-C10-E9, 31-F8-F5-C5, 8-B12-F9-B11, 3-F3-G12-E2, 133B4C7, 124E3D6, 147E11E2, 126A11A4, 147C6F3, 127E2D3, and 140D2B10) were selected to generate and produce human IgG1 chimeric antibodies. DNAs encoding variable regions of the 16 hybridoma antibodies were synthesized and subcloned into an expression vector where human IgG constant gene was included in advance. The vectors were transfected into mammalian cells for recombinant protein expression and the expressed antibody was purified using protein A affinity chromatography column. The resulting chimeric antibodies are referred to herein as ch25-G12-G6-C12, ch4-F12-F1-A4, ch7-D8-G12-E4, ch20-E11-E11-C2, ch7-D9-G10-H2, ch40-C12-C10-E9, ch31-F8-F5-C5, ch8-B12-F9-B11, ch3-F3-G12-E2, ch133B4C7, ch124E3D6, ch147E11E2, ch126A11A4, ch147C6F3, ch127E2D3, ch140D2B10, where the prefix “ch” indicates “chimeric”, and the following indicates the hybridoma antibody clone. For example, ch25-G12-G6-C12 indicates that it is a chimeric antibody from the hybridoma antibody 25-G12-G6-C12.

3.2. T Cell Activation Capability Assays of ch124E3D6, ch126A11A4, ch127E2D3, ch133B4C7, ch140D2B10, ch147C6F3 and ch147E11E2

The T cell activation capabilities of the chimeric antibodies ch124E3D6, ch126A11A4, ch127E2D3, ch133B4C7, ch140D2B10, ch147C6F3 and ch147E11E2 were determined by Jurkat NFAT-Luciferase activation assay. BMK-B219 (an anti-CD3 antibody developed by JNJ) and BMK-TCB (an anti-CD3 antibody developed by Roche) were used as positive controls and hIgG1 isotype was used as negative control. The protocol for activation analysis is described as follows:
Serial diluted test antibodies were coated at 96-well plate at 37° C. for 2 hours, 100 μl per well;
Jurkat-NFAT-Luciferase cells were seeded in each well and incubated for 24 hours, 2×10⁴ cells per well;
The supernatants were removed, and the cells were lysed with lysis reagent (Promega, RLB-E3971) at 25 μL per well;
The samples were cryopreserved for 30 minutes at −80° C. and then heated for 15 minutes at 37° C. in a biochemical incubator;
The 96-well plate was then centrifuged for 10 minutes at 500×g and 20 μL supernatant of each well was transferred to a new white opaque 96-well plate;
100 μL per well of Luciferase Assay Substrate (Promega, E4530) was added to the 96-well plate;
The microplate reader was used to determine the relative luminescence units (RLU) reflecting the level of luciferase activity.

The results are shown in FIG. 1 and Table 15 below. As shown in FIG. 1 and Table 15, the activation capacity of selected antibodies was higher or comparable with two benchmark antibodies.

TABLE 15
Activation of Jurkat-NFAT-luciferase
with chimeric antibodies

| | | EC₅₀ | Top |
|---|---|---|---|
| Ranking | Antibody | (nM) | signal |
| 1 | ch133B4C7 | 27.97 | 290 |
| 2 | ch124E3D6 | 32.38 | 482.3 |
| 3 | ch147E11E2 | 32.99 | 420.8 |
| 4 | ch126A11A4 | 33.56 | 681.7 |
| 5 | ch147C6F3 | 57.36 | 427.8 |
| 6 | BMK-TCB | 59.35 | 752.6 |
| 7 | ch127E2D3 | 61.68 | 336.6 |
| 8 | BMK-B219 | 68.37 | 811.3 |

### 3.3. Cell-Based Binding Affinity to Jurkat Cells

The binding affinity of the produced chimeric antibodies and benchmark antibodies (OKT3, BMK-B219 or BMK-TCB) with human patient derived lymphocyte cancer cell line Jurkat, was determined by FACS analysis, and hIgG1 isotype was used as negative control. The protocol for FACS analysis is described as follows:
(a) Jurkat cells were harvested and resuspended with FACS buffer;
(b) Cells were counted and the concentration was adjusted to 4×10\hat{ }6 cells/ml. 50 μl/well cells suspension were added to 96-well plates;
(c) 50 μl/well 2× serial diluted test antibodies were added to the plates. The plates were incubated at 4° C. for 1 hour;
(d) The plates were centrifuged at 300 g for 3 mins and the supernatant was discarded;
(e) Cells were washed in 200 μl FASC buffer for twice and step (d) was repeated;
(f) 100 μl/well secondary antibody Goat pAb to human IgG-PE or Goat pAb to mouse IgG-PE were added to plates. Plates were incubated at 4° C. for 30 mins;
(g) The plates were centrifuged at 300 g for 3 mins and the supernatant was discarded;
(h) Cells were washed in 200 μl FASC buffer for twice and step (g) was repeated;
(i) Cells were resuspended with 150 μl FASC buffer and tested on FACS.

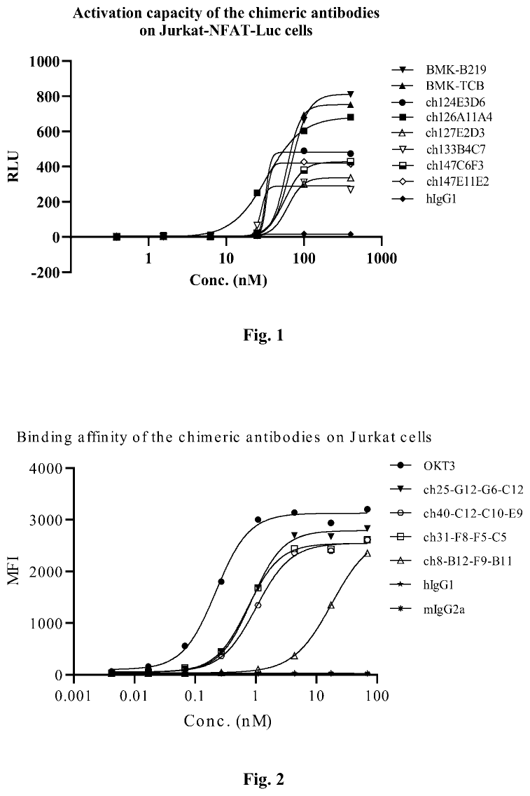

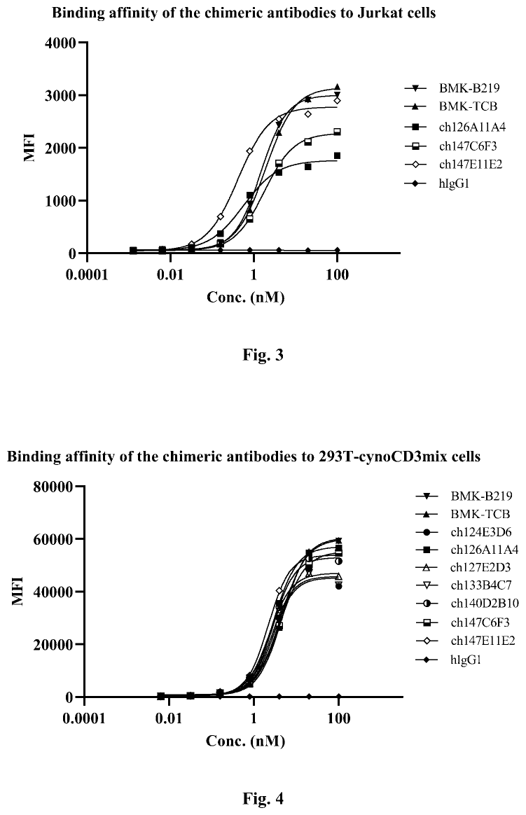

As shown in FIG. 2 and Table 16 below, the chimeric antibodies ch31-F8-F5-C5, ch25-G12-G6-C12, ch40-C12-C10-E9 and ch8-B12-F9-B11, as well as the benchmark antibody OKT3 had positive binding affinity to Jurkat cells. As shown in FIG. 3 and Table 17 below, the binding affinity of chimeric antibodies ch147E11E2, ch126A11A4 and ch147C6F3 on Jurkat cells were higher or comparable with two benchmark antibodies BMK-B219 and BMK-TCB.

TABLE 16
Binding affinity of selected chimeric
antibodies to Jurkat cells.

| | | EC₅₀ | Top FACS |
|---|---|---|---|
| Ranking | Antibody | (nM) | signal |
| 1 | OKT3 | 0.22 | 3124 |
| 2 | ch31-F8-F5-C5 | 0.74 | 2540 |
| 3 | ch25-G12-G6-C12 | 0.84 | 2790 |
| 4 | ch40-C12-C10-E9 | 1.01 | 2545 |
| 5 | ch8-B12-F9-B11 | 17.99 | 2721 |

TABLE 17
Binding affinity of selected chimeric
antibodies to Jurkat cells.

| | | EC₅₀ | Top |
|---|---|---|---|
| Ranking | Antibody | (nM) | signal |
| 1 | ch147E11E2 | 0.4192 | 2777 |
| 2 | ch126A11A4 | 0.5668 | 1764 |
| 3 | BMK-B219 | 1.47 | 3001 |
| 4 | ch147C6F3 | 1.774 | 2282 |
| 5 | BMK-TCB | 1.912 | 3145 |

### 3.4. Cell-Based Binding Affinity to 293T-cynoCD3mix Cells

The binding affinity of selected chimeric antibodies and benchmark antibodies on HEK 293T stably over-expressing cynomolgus CD3 (293T-cynoCD3mix) cells was determined by FACS analysis. BMK-B219 and BMK-TCB were used as positive controls and hIgG1 isotype was used as negative control. The protocol for FACS analysis is described as follows:
(a) 293T-cynoCD3mix cells were harvested and resuspended with FACS buffer;
(b) Cells were counted and the concentration was adjusted to 4×10\hat{ } 6 cells/ml. 50 μl/well cells suspension were added to a 96-well plate;
(c) 50 μl/well 2× serial diluted test antibodies were added to the plate and incubated at 4° C. for 1 hour;
(d) The plate was centrifuged at 300 g for 3 mins and the supernatant was discarded;
(e) Cells were washed in 200 μl FASC buffer for twice and step (d) was repeated;
(f) 100 μl/well secondary antibody Goat pAb to human IgG-PE (Abcam, ab98596) were added to plate and incubated at 4° C. for 30 mins;
(g) The plate was centrifuged at 300 g for 3 mins and the supernatant was discarded;
(h) Cells were washed in 200 μl FASC buffer for twice and step (g) was repeated;
(i) Cells were resuspended with 150 μl FASC buffer and tested on FACS.

As shown in FIG. 4 and Table 18 below, the binding affinity of the selected chimeric antibodies on 293T-cynoCD3mix cells were higher than that of two benchmark antibodies.

TABLE 18
Binding affinity to 293T-cynoCD3mix cells.

| | | EC₅₀ | Top |
|---|---|---|---|
| Ranking | Antibody | (nM) | signal |
| 1 | ch147E11E2 | 2.19 | 53876 |
| 2 | ch127E2D3 | 2.33 | 46947 |
| 3 | ch124E3D6 | 2.64 | 45795 |
| 4 | ch140D2B10 | 2.81 | 52929 |
| 5 | ch133B4C7 | 2.81 | 45246 |
| 6 | ch126A11A4 | 2.91 | 57049 |
| 7 | ch147C6F3 | 4.15 | 55201 |
| 8 | BMK-B219 | 4.34 | 60013 |
| 9 | BMK-TCB | 4.72 | 60397 |

### 3.5. Jurkat-NFAT-Luciferase Activation Assay

The Jurkat-NFAT-Luciferase activation assay of several selected chimeric antibodies and benchmark antibody (OKT3) with human patient derived lymphocyte cancer cell line Jurkat, was performed as follows.
(a) Plates were coated with 50 μl per well serial diluted test antibodies at 37° C. for 2 hours;
(b) Jurkat-NFAT-Luc cells were harvested and resuspended with 1640 medium (10% FBS, 1% P/S);
(c) Cells were counted and the concentration was adjusted to 2×10\hat{ } 5 cells/ml. 100 μl/well cells suspension were seed to 96-well cell culture plate. 100 μl medium was added as negative control;
(d) The cell plate was returned into a 37° C., 5% CO₂ incubator for 24 hours;
(e) Centrifuged at 500 g for 10 min and the supernatant was discarded;
(f) Cells were washed in PBS for once and repeat step (e);
(g) 30 μl/well 1×Lysis buffer was added, the plate was put in −80° C. freezer for 30 min, and then moved to a 37° C. incubator for 15 min;
(h) Centrifuge at 500 g for 10 min and 20 μl supernatant was transferred to another 96-well plate (Coning 3903). 100 μl/well Luciferase Assay Substrate was added and gently mixed well;
(i) Read plate at microplate reader for luminescence signal (RLU).

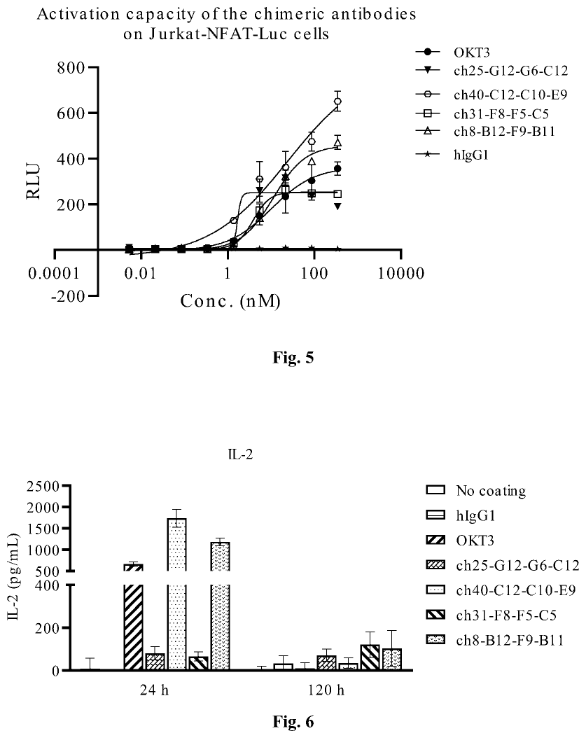

As shown in FIG. 5 and Table 19 below, the activation capacity of selected chimeric antibodies was higher or comparable with benchmark antibody OKT3.

TABLE 19
Activation capacity of the chimeric antibodies
on Jurkat-NFAT-Luc cells.

| Ranking | Antibody | EC₅₀ (nM) | Top FACS signal |
|---|---|---|---|
| 1 | ch25-G12-G6-C12 | 1.674 | 249.8 |
| 2 | ch31-F8-F5-C5 | 3.83 | 253.2 |
| 3 | OKT3 | 10.12 | 363.7 |
| 4 | ch8-B12-F9-B11 | 11.27 | 457.2 |
| 5 | ch40-C12-C10-E9 | 24.17 | 810.2 |

### 3.6. PBMC Activation Assay

### 3.6.1 Experiment I

A primary human peripheral blood mononuclear cell (PBMC) based assay was used to determine the activation capacity of several selected chimeric antibodies to T cells. The PBMC activation assay of the selected chimeric antibodies and benchmark antibody (OKT3) with PBMC were performed as follows.
(a) Plates were coated with 50 μl/well serial diluted test antibodies at 37° C. for 2 hours;
(b) Antibody solutions were discarded and plate wells were washed with 200 μl PBS for once;
(c) PBMC were isolated and resuspended with 1640 medium (10% FBS, 1% P/S);
(d) Cells were counted and the concentration was adjusted to 1×10\hat{ } 6 cells/ml. 200 μl/well cells suspension were seeded to 96-well cell culture plate. 200 μl medium was added as negative control;
(e) The cell plates were returned into a 37° C., 5% CO₂ incubator for 120 hours;
(f) Centrifuge at 400 g for 5 min and supernatant was collected to detect IL-2 and IFNγ cytokines release by ELISA assay.

The ELISA assay were performed as follows.
(a) Add 100 μl diluted Capture Ab to each well. Incubate overnight at 4° C.;
(b) Aspirate and wash 3 times;
(c) Block plates: 200 μl Assay Diluent to each well. Incubate 1 hour RT;
(d) Aspirate and wash 3 times;
(e) Add 100 μl standard or sample to each well. Incubate 2 hours RT;
(f) Aspirate and wash 5 times;
(g) Add 100 μl Working Detector (Detection Ab+SAv-HRP) to each well. Incubate 1 hour RT;
(h) Aspirate and wash 7 times (with 30 sec to 1 min soaks);
(i) Add 100 μl Substrate Solution to each well. Incubate 30 min RT in dark;
(j) Add 50 μl Stop Solution to each well;
(k) Read at 450 nm within 30 min.

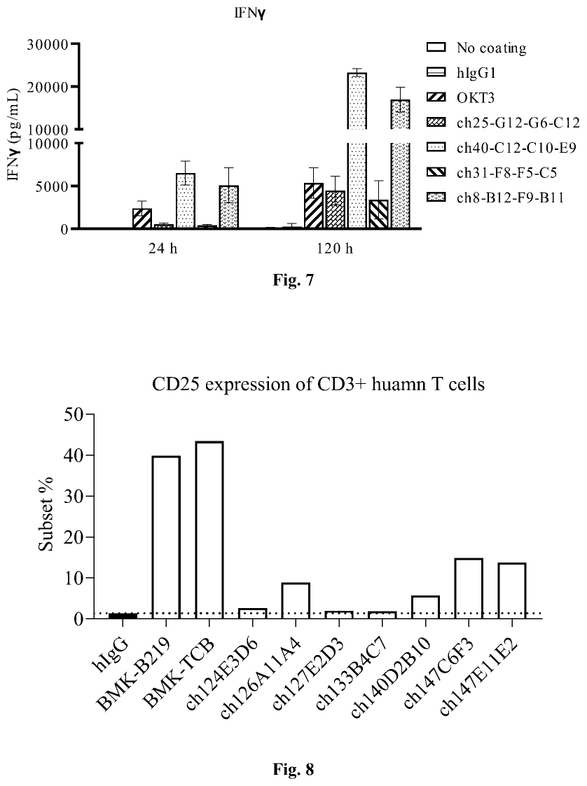

The results of IL-2 and IFNγ release are shown in FIG. 6 and FIG. 7, respectively. As shown in FIG. 6, the selected chimeric antibodies ch25-G12-G6-C12, ch40-C12-C10-E9, ch31-F8-F5-C5 and ch8-B12-F9-B11, as well as the benchmark antibody OKT3 significantly induced IL-2 release. As shown in FIG. 7, the selected chimeric antibodies ch25-G12-G6-C12, ch40-C12-C10-E9, ch31-F8-F5-C5, ch8-B12-F9-B11, as well the benchmark antibody OKT3 significantly increased IFNγ release, and the PBMC activation capacity of the selected chimeric antibodies was higher or at least comparable with the benchmark antibody OKT3.

### 3.6.2. Experiment 2

A primary human peripheral blood mononuclear cell (PBMC) based assay was used to determine the activation capacity of several other selected chimeric antibodies to T cells. It is well known that when T cells are activated, the expression of the surface protein CD25 is upregulated. BMK-B219 and BMK-TCB were used as positive controls and hIgG1 isotype was used as negative control. The protocol for activation analysis is described as follows:
(a) 100 μl per well 10 μg/ml test antibodies were coated at 96-well plate at 37° C. for 2 hours;
(b) 2×10⁵ cells per well Fresh human PBMC were seeded in each well and incubated for 3 days;
(j) Cells were collected and transferred to a new 96-well plate. The plate was centrifuged at 400 g for 3 mins and the supernatant was discarded;
(c) Cells were resuspended in 100 μl FASC buffer containing anti-human CD25 Alexa Fluor 488-conjugated antibody (R&D, FAB9926G) and anti-human CD3 Brilliant Violet 421-conjugated antibody (Biolegend, 317344);
(d) Plate was incubated at 4° C. for 30 mins;
(e) Plate was centrifuged at 400 g for 3 mins and the supernatant was discarded;
(f) Cells were washed in 200 μl FASC buffer for twice and step (d) was repeated;
(g) Cells were resuspended with 150 μl FASC buffer and tested on FACS.

As shown in FIG. 8, the positive controls BMK-B219 and BMK-TCB induced the upregulation of CD25 on human T cells as indicated by the subset rate of CD25+ cells in the CD3⁺ T cells. Among the chimeric antibodies, several (namely ch124E3D6, ch126A11A4, ch140D2B10, ch147C6F3 and ch147E11E2) induced CD25 expression to a weaker degree than the positive controls. The chimeric antibodies ch127E2D3 and ch133B4C7 did not significantly induce CD25 upregulation.

### Example 4. Humanized Antibody of 40-C12-C10-E9: Generation, Affinity Maturation and Antibody Characterization

### 4.1 Humanized Antibody Generation

Chimeric antibody ch40-C12-C10-E9 was selected as the clone for humanization. Antibody sequences were aligned with human germline sequences to identify best fit model. Best matched human germline sequences were selected as the templates for humanization based on homology to the original mouse antibody sequences. Generally, humanization of an antibody was performed by comparing IMGT (https://www.imgt.org) human antibody heavy and light chain variable strain gene database, heavy chain and light chain variable strain genes with high homology with murine-derived antibody were selected as templates, and CDRs of murine-derived antibody were transplanted into corresponding human templates. A variable region sequence with the order FR1-CDR1-FR2-CDR2-FR3-CDR3-FR4 was formed. As required, the key amino acids in the skeleton sequence were reverted and mutated to those corresponding to murine antibodies to ensure the original affinity.

A total of 12 humanized antibodies were obtained for ch40-C12-C10-E9 by mixing and matching 3 variants of humanized light chain variable regions (i.e., hu40E9-L1, hu40E9-L2 and hu40E9-L3) and 4 variants of humanized heavy chain variable regions (i.e., hu40E9-H1, hu40E9-H2, hu40E9-H3 and hu40E9-H4). The 12 humanized antibodies were designated as hu40E9-L1H1, hu40E9-L2H1, and so on, as shown in Table 25 below, by the same token. Immunogenicity risk and hotspot were evaluated on these produced humanized antibodies, and no immunogenicity risk or hotspot was identified.

TABLE 25
Heavy and light chain variable regions of exemplary humanized
antibodies for ch40-C12-C10-E9

| | VL Region | | |
|---|---|---|---|
| VH | hu40E9-L1 | hu40E9-L2 | hu40E9-L3 |
| Region | (SEQ ID NO: 168) | (SEQ ID NO: 169) | (SEQ ID NO: 170) |
| hu40E9-H1 | hu40E9-L1H1 | hu40E9-L2H1 | hu40E9-L3H1 |
| (SEQ ID | (SEQ ID NOs: | (SEQ ID NOs: | (SEQ ID NOs: |
| NO: 163) | 168/163) | 169/163) | 170/163) |
| hu40E9-H2 | hu40E9-L1H2 | hu40E9-L2H2 | hu40E9-L3H2 |
| (SEQ ID | (SEQ ID NOs: | (SEQ ID NOs: | (SEQ ID NOs: |
| NO: 164) | 168/164) | 169/164) | 170/164) |
| hu40E9-H3 | hu40E9-L1H3 | hu40E9-L2H3 | hu40E9-L3H3 |
| (SEQ ID | (SEQ ID NOs: | (SEQ ID NOs: | (SEQ ID NOs: |
| NO: 165) | 168/165) | 169/165) | 170/165) |
| hu40E9-H4 | hu40E9-L1H4 | hu40E9-L2H4 | hu40E9-L3H4 |
| (SEQ ID | (SEQ ID NOs: | (SEQ ID NOs: | (SEQ ID NOs: |
| NO: 166) | 168/166) | 169/166) | 170/166) |

### 4.2 Affinity Maturation

The hu40E9-L2 variant (i.e., SEQ ID NO: 169) and hu40E9-H3 variant (i.e., SEQ ID NO: 165) obtained in Example 4.1 were then selected to conduct affinity maturation. In particular, positions 31˜33 (relative to SEQ ID NO: 165) in CDR1 of SEQ ID NO: 165, positions 54, 55 and 56 (relative to SEQ ID NO: 165) in CDR2 of SEQ ID NO: 165, positions 99˜106 (relative to SEQ ID NO: 165) in CDR3 of SEQ ID NO: 165, and positions 91, 92 and 93 (relative to SEQ ID NO: 169) in CDR3 of SEQ ID NO: 169 were selected for affinity engineering. A total of 20 mutations were obtained after considering several factors, namely, S31D, V33Y, N55S, D99E, Y101F, D105E, G106A, Y54G, D56G, D99R, D99G, S100R, S100D, S100G, Y102S or D105R in the heavy chain variable region as set forth in SEQ ID NO: 165; or N93S, S91R, N93R, or N93W in the light chain variable region as set forth in SEQ ID NO: 169.

Accordingly, a total of 34 humanized and affinity maturated antibody clones were obtained for ch40-C12-C10-E9, mixing and matching 24 variants of humanized ch40-C12-C10-E9 heavy chain variable regions (i.e., hu40E9-H3-N55S.H, hu40E9-H3-D99E.H, hu40E9-H3-Y101F.H, hu40E9-H3-D105E.H, hu40E9-H3-G106A.H, hu40E9-H3-Y54G.H, hu40E9-H3-D56G.H, hu40E9-H3-D99R.H, hu40E9-H3-S100R.H, hu40E9-H3-Y102S.H, hu40E9-H3-D105R.H, hu40E9-H5, hu40E9-H6, hu40E9-H7, hu40E9-H8, hu40E9-H9, hu40E9-H10, hu40E9-H11, hu40E9-H12, hu40E9-H13, hu40E9-H14, hu40E9-H15, hu40E9-H16 and hu40E9-H17) and 7 variants of humanized ch40-C12-C10-E9 light chain variable regions (i.e., hu40E9-L2-N93S.L, hu40E9-L2-S91R.L, hu40E9-L2-N93R.L and hu40E9-L2-N93W.L, hu40E9-L4, hu40E9-L5 and hu40E9-L6). The 34 humanized antibody clones were designated as hu40E9-L2H3-N55S.H, hu40E9-L2H3-N93S.L, hu40E9-L2H5 and so on, as shown in Table 26, Table 27, Table 32˜34 below, where the prefix “hu” indicates “humanized”, and the suffix “L2H3-N55S.H”, for example, denotes the serial number of the humanized antibody clone of ch40-C12-C10-E9, having the hu40E9-L2 variant variable region and the hu40E9-H3-N55S.H variant variable region. For another example, the humanized antibody hu40E9-L2H3-N93S.L denotes the serial number of the humanized antibody clone of ch40-C12-C10-E9, having the hu40E9-L2-N93S.L variant variable region and the hu40E9-H3 variant variable region. For yet another example, the humanized antibody hu40E9-L2H5 denotes the serial number of the humanized antibody clone of ch40-C12-C10-E9, having the hu40E9-L2 variant variable region and the hu40E9-H5 variant variable region.

TABLE 26
Heavy and light chain variable regions of exemplary
humanized antibodies for ch40-C12-C10-E9

| | VL Region |
|---|---|
| | hu40E9-L2 |
| VH Region | (SEQ ID NO: 169) |
| hu40E9-H3-N55S.H | hu40E9-L2H3-N55S.H |
| (SEQ ID NO: 186) | (SEQ ID NOs: 169/186) |
| hu40E9-H3-D99E.H | hu40E9-L2H3-D99E.H |
| (SEQ ID NO: 187) | (SEQ ID NOs: 169/187) |
| hu40E9-H3-Y101F.H | hu40E9-L2H3-Y101F.H |
| (SEQ ID NO: 188) | (SEQ ID NOs: 169/188) |
| hu40E9-H3-D105E.H | hu40E9-L2H3-D105E.H |
| (SEQ ID NO: 189) | (SEQ ID NOs: 169/189) |
| hu40E9-H3-G106A.H | hu40E9-L2H3-G106A.H |
| (SEQ ID NO: 190) | (SEQ ID NOs: 169/190) |
| hu40E9-H3-Y54G.H | hu40E9-L2H3-Y54G.H |
| (SEQ ID NO: 191) | (SEQ ID NOs: 169/191) |
| hu40E9-H3-D56G.H | hu40E9-L2H3-D56G.H |
| (SEQ ID NO: 192) | (SEQ ID NOs: 169/192) |
| hu40E9-H3-D99R.H | hu40E9-L2H3-D99R.H |
| (SEQ ID NO: 193) | (SEQ ID NOs: 169/193) |
| hu40E9-H3-S100R.H | hu40E9-L2H3-S100R.H |
| (SEQ ID NO: 194) | (SEQ ID NOs: 169/194) |
| hu40E9-H3-Y102S.H | hu40E9-L2H3-Y102S.H |
| (SEQ ID NO: 195) | (SEQ ID NOs: 169/195) |
| hu40E9-H3-D105R.H | hu40E9-L2H3-D105R.H |
| (SEQ ID NO: 196) | (SEQ ID NOs: 169/196) |

TABLE 27
Heavy and light chain variable regions of exemplary
humanized antibodies for ch40-C12-C10-E9

| | VH Region |
|---|---|
| | hu40E9-H3 |
| VL Region | (SEQ ID NO: 165) |
| hu40E9-L2-N93S.L | hu40E9-L2H3-N93S.L |
| (SEQ ID NO: 197) | (SEQ ID NO: 197/165) |
| hu40E9-L2-S91R.L | hu40E9-L2H3-S91R.L |
| (SEQ ID NO: 198) | (SEQ ID NO: 198/165) |
| hu40E9-L2-N93R.L | hu40E9-L2H3-N93R.L |
| (SEQ ID NO: 199) | (SEQ ID NO: 199/165) |
| hu40E9-L2-N93W.L | hu40E9-L2H3-N93W.L |
| (SEQ ID NO: 200) | (SEQ ID NO: 200/165) |

TABLE 32
Heavy and light chain variable regions of exemplary humanized
antibodies for ch40-C12-C10-E9

| | VL Region |
|---|---|
| VH Region | hu40E9-L2 (SEQ ID NO: 169) |
| hu40E9-H5 (SEQ ID NO: 233) | hu40E9-L2H5 (SEQ ID NO: 169/233) |
| hu40E9-H6 (SEQ ID NO: 234) | hu40E9-L2H6 (SEQ ID NO: 169/234) |
| hu40E9-H7 (SEQ ID NO: 235) | hu40E9-L2H7 (SEQ ID NO: 169/235) |
| hu40E9-H8 (SEQ ID NO: 236) | hu40E9-L2H8 (SEQ ID NO: 169/236) |
| hu40E9-H9 (SEQ ID NO: 237) | hu40E9-L2H9 (SEQ ID NO: 169/237) |
| hu40E9-H10 (SEQ ID NO: 238) | hu40E9-L2H10 (SEQ ID NO: 169/238) |
| hu40E9-H11 (SEQ ID NO: 239) | hu40E9-L2H11 (SEQ ID NO: 169/239) |
| hu40E9-H12 (SEQ ID NO: 240) | hu40E9-L2H12 (SEQ ID NO: 169/240) |
| hu40E9-H13 (SEQ ID NO: 241) | hu40E9-L2H13 (SEQ ID NO: 169/241) |
| hu40E9-H14 (SEQ ID NO: 242) | hu40E9-L2H14 (SEQ ID NO: 169/242) |
| hu40E9-H15 (SEQ ID NO: 243) | hu40E9-L2H15 (SEQ ID NO: 169/243) |
| hu40E9-H16 (SEQ ID NO: 244) | hu40E9-L2H16 (SEQ ID NO: 169/244) |

TABLE 33
Heavy and light chain variable regions of exemplary humanized
antibodies for ch40-C12-C10-E9

| | VH Region |
|---|---|
| VL Region | hu40E9-H3 (SEQ ID NO: 165) |
| hu40E9-L4 (SEQ ID NO: 230) | hu40E9-L4H3 (SEQ ID NO: 230/165) |
| hu40E9-L5 (SEQ ID NO: 231) | hu40E9-L5H3 (SEQ ID NO: 231/165) |
| hu40E9-L6 (SEQ ID NO: 232) | hu40E9-L6H3 (SEQ ID NO: 232/165) |

TABLE 34
Heavy and light chain variable regions of exemplary
humanized antibodies for ch40-C12-C10-E9

| | VH Region | |
|---|---|---|
| | hu40E9-H7 | hu40E9-H8 |
| VL Region | (SEQ ID NO: 235) | (SEQ ID NO: 236) |
| hu40E9-L4 (SEQ | hu40E9-L4H7 | hu40E9-L4H8 |
| ID NO: 230) | (SEQ ID NO: 230/235) | (SEQ ID NO: 230/236) |
| hu40E9-L5 (SEQ | hu40E9-L5H7 | hu40E9-L5H8 |
| ID NO: 231) | (SEQ ID NO: 231/235) | (SEQ ID NO: 231/236) |

### 4.3 Cell-Based Binding Affinity to Jurkat Cells

The binding affinity of the generated humanized 40-C12-C10-E9 antibodies and affinity maturated antibodies based on 40E9-L2H3 on human patient derived lymphocyte cancer cell line Jurkat was determined by FACS analysis. The benchmark antibody BMK-TCB was used as a positive control and hIgG1 isotype was used as a negative control. The protocol for FACS analysis is described in Example 3.3 except for the test antibodies used in step (c) were different.

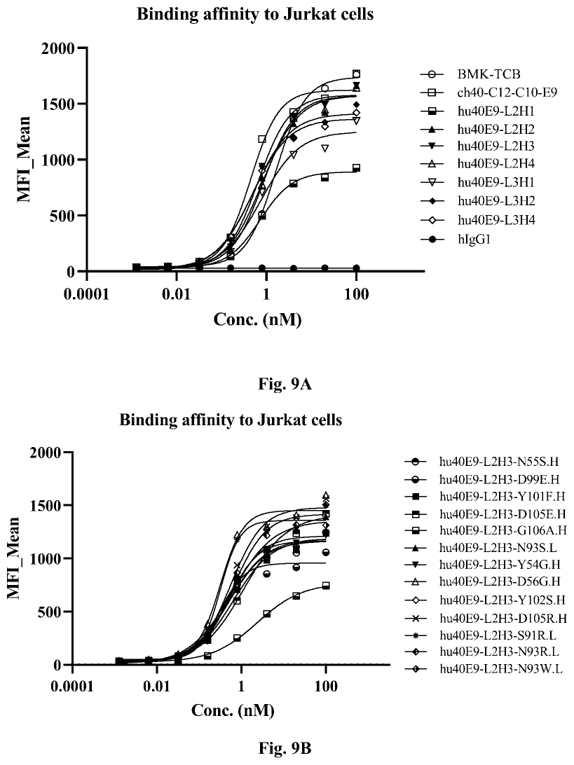

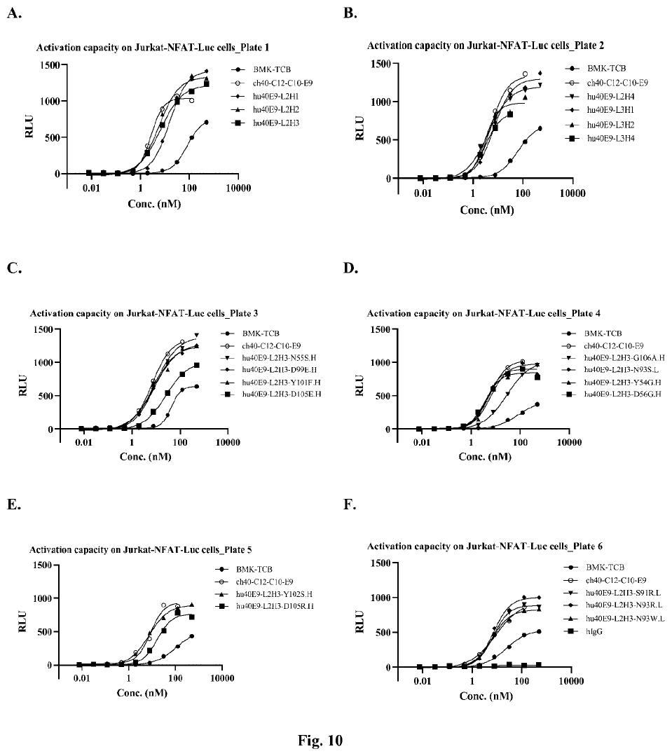

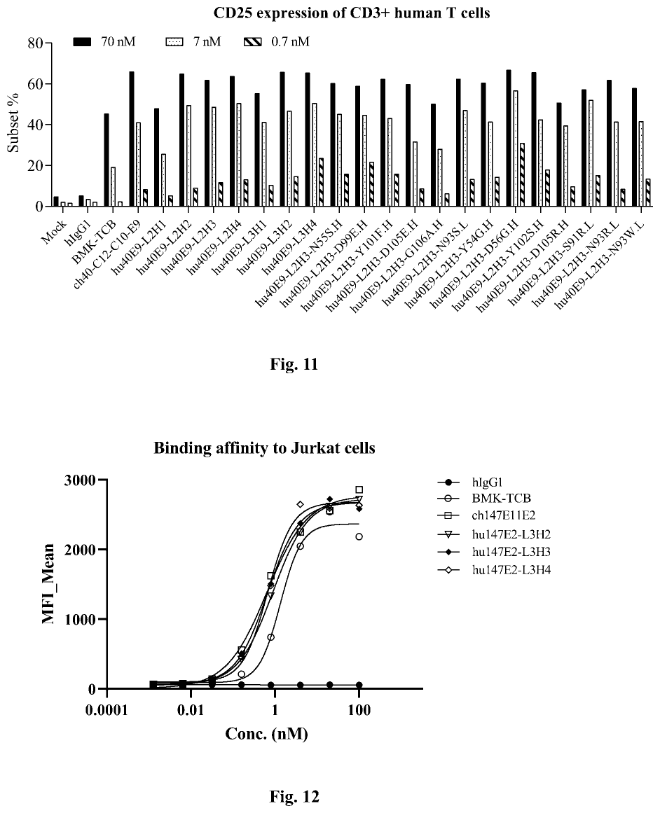

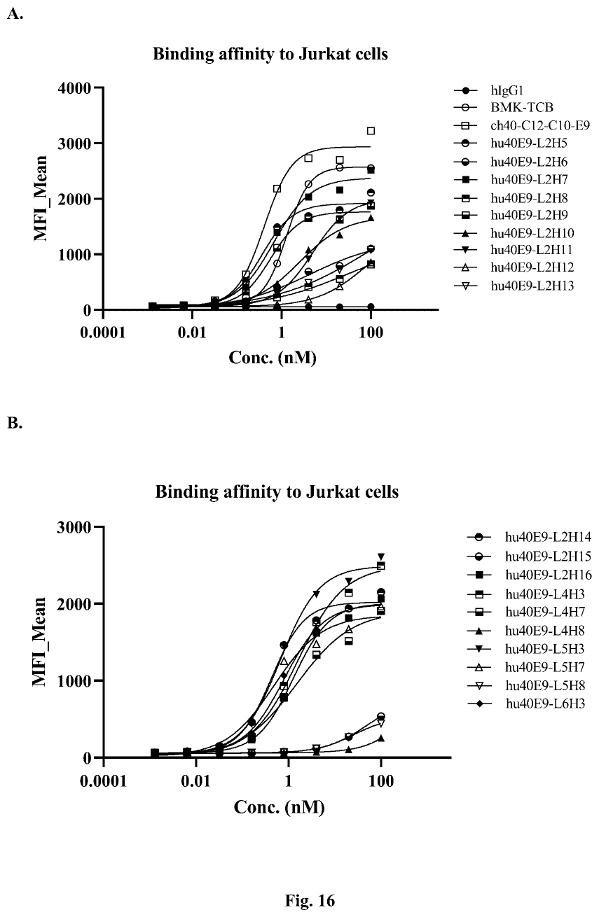

As shown in FIG. 9A, FIG. 9B and Table 23 below, the binding affinity of the produced humanized and/or affinity maturated 40-C12-C10-E9 antibodies to Jurkat cells were higher or comparable with the benchmark antibody BMK-TCB. As shown in FIG. 16A, FIG. 16B and Table 35 below, the selected humanized and/or affinity maturated 40-C12-C10-E9 antibodies were capable of binding to Jurkat cells.

TABLE 23
Binding affinity of selected
humanized antibodies to Jurkat cells.

| | | EC₅₀ | Top |
|---|---|---|---|
| Ranking | Antibody | (nM) | FACS signal |
| 1 | hu40E9-L2H3-D99E.H | 0.31 | 957 |
| 2 | hu40E9-L2H3-S91R.L | 0.32 | 1358 |
| 3 | hu40E9-L2H3-D56G.H | 0.32 | 1452 |
| 4 | ch40-C12-C10-E9 | 0.45 | 1621 |
| 5 | hu40E9-L2H3-Y102S.H | 0.47 | 1165 |
| 6 | hu40E9-L3H4 | 0.49 | 1365 |
| 7 | hu40E9-L2H3-N93W.L | 0.51 | 1210 |
| 8 | hu40E9-L2H3-Y54G.H | 0.51 | 1186 |
| 9 | hu40E9-L2H3-N55S.H | 0.51 | 1165 |
| 10 | hu40E9-L3H2 | 0.52 | 1412 |
| 11 | hu40E9-L2H3-D105R.H | 0.56 | 1479 |
| 12 | hu40E9-L2H3-N93S.L | 0.60 | 1355 |
| 13 | hu40E9-L2H3-N93R.L | 0.63 | 1417 |
| 14 | hu40E9-L2H3-Y101F.H | 0.64 | 1187 |
| 15 | hu40E9-L2H3 | 0.65 | 1578 |
| 16 | hu40E9-L2H1 | 0.73 | 892 |
| 17 | hu40E9-L3H1 | 0.75 | 1250 |
| 18 | hu40E9-L2H2 | 0.81 | 1573 |
| 19 | hu40E9-L2H4 | 0.86 | 1571 |
| 20 | hu40E9-L2H3-D105E.H | 1.16 | 1409 |
| 21 | BMK-TCB | 1.60 | 1739 |
| 22 | hu40E9-L2H3-G106A.H | 2.39 | 771 |

TABLE 35
Binding affinity of selected
humanized antibodies to Jurkat cells.

| | | EC₅₀ | Top |
|---|---|---|---|
| Ranking | Antibody | (nM) | FACS signal |
| 1 | hu40E9-L2H5 | 0.36 | 1915 |
| 2 | ch40-C12-C10-E9 | 0.41 | 2937 |
| 3 | hu40E9-L2H14 | 0.43 | 2019 |
| 4 | hu40E9-L5H7 | 0.48 | 1851 |
| 5 | hu40E9-L2H9 | 0.56 | 1764 |
| 6 | hu40E9-L2H7 | 0.66 | 2377 |
| 7 | hu40E9-L5H3 | 0.66 | 2489 |
| 8 | hu40E9-L6H3 | 0.80 | 1993 |
| 9 | hu40E9-L2H16 | 1.27 | 2006 |
| 10 | BMK-TCB | 1.30 | 2574 |
| 11 | hu40E9-L4H7 | 1.52 | 1903 |
| 12 | hu40E9-L4H3 | 1.59 | 2491 |
| 13 | hu40E9-L2H10 | 2.55 | 1682 |
| 14 | hu40E9-L2H6 | 4.17 | 1255 |
| 15 | hu40E9-L2H11 | 5.00 | 2001 |
| 16 | hu40E9-L5H8 | 25.44 | 531 |
| 17 | hu40E9-L4H8 | 26.92 | 255 |
| 18 | hu40E9-L2H8 | 27.99 | 1237 |
| 19 | hu40E9-L2H15 | 51.69 | 796 |
| 20 | hu40E9-L2H12 | 72.92 | 850 |
| 21 | hu40E9-L2H13 | 83.69 | 2051 |

### 4.4 Jurkat-NFAT-Luciferase Activation Assay

The T cell activation capability of these antibodies was determined by Jurkat NFAT-Luciferase activation assay. The benchmark antibody BMK-TCB was used as a positive control and hIgG1 isotype was used as a negative control. The protocol for activation analysis is described as follows.
a) Serial diluted test antibodies were coated at 96-well plate at 37° C. for 2 hours, 100 μl per well.
b) Jurkat-NFAT-Luciferase cells were seeded in each well and incubated for 24 hours, 2×10⁴ cells per well.
c) The supernatants were removed, and the cells were lysed with lysis reagent (Promega, RLB-E3971) at 25 L per well.
d) The samples were cryopreserved for 30 minutes at −80° C. and then heated for 15 minutes at 37° in a biochemical incubator.
e) The 96-well plate was then centrifuged for 10 minutes at 500×g and 20 μL supernatant of each well was transferred to a new white opaque 96-well plate.
f) 100 μL per well of Luciferase Assay Substrate (Promega, E4530) was added to the 96-well plate.
g) The microplate reader was used to determine the relative luminescence units (RLU) reflecting the level of luciferase activity.

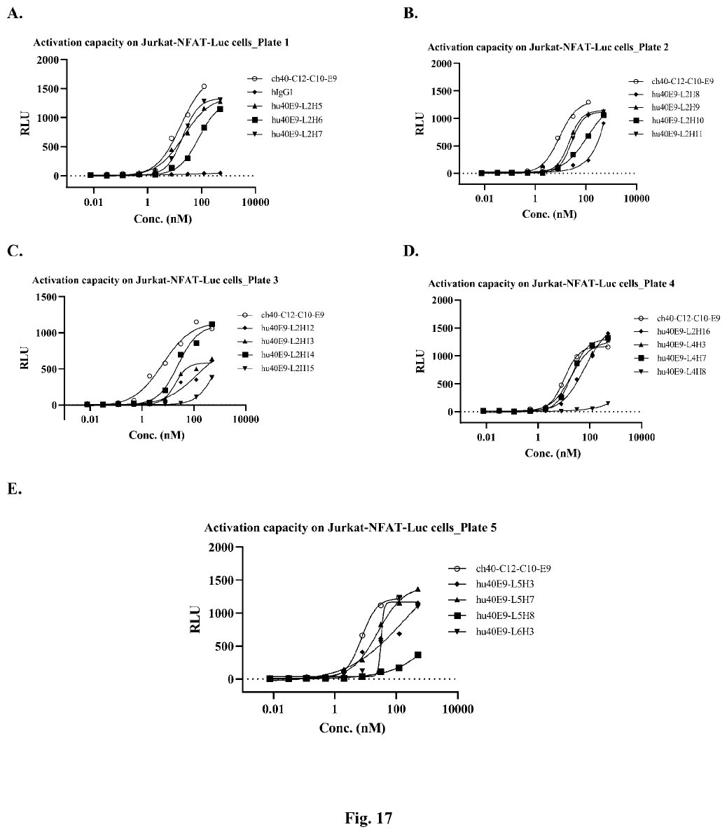

The values of the activated EC₅₀ and the top signal were normalized according to ch40-C12-C10-E9 on the same plate and the values of benchmark antibody BMK-TCB were shown as mean. As shown in FIGS. 10A˜F and Table 24 below, the activation capacity of selected humanized and/or affinity maturated antibodies was higher or comparable with benchmark antibody BMK-TCB. As shown in FIGS. 17A˜E and Table 36 below, the selected humanized and/or affinity maturated antibodies had activation capacity on Jurkat-NFAT-Luciferase cells, which means they had T cell activation capability.

TABLE 24
Activation capacity of the humanized antibodies
on Jurkat-NFAT-luciferase cells.

| | | EC₅₀_ | Top FACS |
|---|---|---|---|
| | | Fold | signal_ |
| Ranking | Antibody | change | Fold change |
| 1 | hu40E9-L3H2 | 0.57 | 0.73 |
| 2 | hu40E9-L3H4 | 0.61 | 0.64 |
| 3 | hu40E9-L2H3-D56G.H | 0.65 | 0.82 |
| 4 | hu40E9-L2H3-N93W.L | 0.72 | 0.88 |
| 5 | hu40E9-L2H3-N93S.L | 0.74 | 0.87 |
| 6 | hu40E9-L2H3-S91R.L | 0.79 | 0.95 |
| 7 | hu40E9-L2H4 | 0.80 | 0.89 |
| 8 | hu40E9-L2H3-N93R.L | 0.81 | 1.07 |
| 9 | hu40E9-L2H3-Y102S.H | 0.87 | 0.96 |
| 10 | ch40-C12-C10-E9 | 1.00 | 1.00 |
| 11 | hu40E9-L2H3-Y54G.H | 1.06 | 0.94 |
| 12 | hu40E9-L2H3-D99E.H | 1.19 | 0.91 |
| 13 | hu40E9-L2H3-Y101F.H | 1.23 | 0.94 |
| 14 | hu40E9-L3H1 | 1.33 | 0.97 |
| 15 | hu40E9-L2H3-N55S.H | 1.35 | 1.03 |
| 16 | hu40E9-L2H3-D105R.H | 1.95 | 0.81 |
| 17 | hu40E9-L2H2 | 2.14 | 1.28 |
| 18 | hu40E9-L2H3 | 2.43 | 1.18 |
| 19 | hu40E9-L2H3-D105E.H | 3.77 | 0.74 |
| 20 | hu40E9-L2H3-G106A.H | 4.95 | 0.99 |
| 21 | hu40E9-L2H1 | 5.73 | 1.37 |
| 22 | BMK-TCB | 8.17 | 0.48 |

TABLE 36
Activation capacity of the humanized antibodies
on Jurkat-NFAT-luciferase cells.

| | EC₅₀_Fold | Top signal_ |
|---|---|---|
| Antibody | change | Fold change |
| ch40-C12-C10-E9 | 1.00 | 1.00 |
| hu40E9-L2H7 | 1.18 | 0.77 |
| hu40E9-L2H5 | 1.29 | 0.79 |
| hu40E9-L4H3 | 1.82 | 1.08 |
| hu40E9-L4H7 | 1.95 | 1.12 |
| hu40E9-L2H9 | 2.49 | 0.87 |
| hu40E9-L2H11 | 2.97 | 0.85 |
| hu40E9-L2H13 | 3.08 | 0.51 |
| hu40E9-L5H7 | 3.41 | 1.15 |
| hu40E9-L2H14 | 3.86 | 0.96 |
| hu40E9-L6H3 | 4.34 | 0.95 |
| hu40E9-L2H6 | 4.57 | 0.74 |
| hu40E9-L2H16 | 6.18 | 1.35 |
| hu40E9-L2H10 | 12.57 | 1.01 |
| hu40E9-L2H8 | 18.35 | 0.69 |
| hu40E9-L2H12 | 21.28 | 0.73 |
| hu40E9-L5H3 | 23.19 | 1.47 |
| hu40E9-L2H15 | 98.56 | 0.75 |
| hu40E9-L4H8 | — | 0.32 |
| hu40E9-L5H8 | — | 0.30 |

### 4.5 Human PBMC Activation Assay

A primary human peripheral blood mononuclear cell (PBMC) based assay was used to determine the activation capacity of the humanized antibodies to T cells. It is well known that when T cells are activated, the expression of the surface protein CD25 is upregulated. The benchmark antibody BMK-TCB was used as a positive control and hIgG1 isotype was used as a negative control. The protocol for activation analysis is described in Example 3.6.2 except for the concentrations and test antibodies used in step (a) were different.

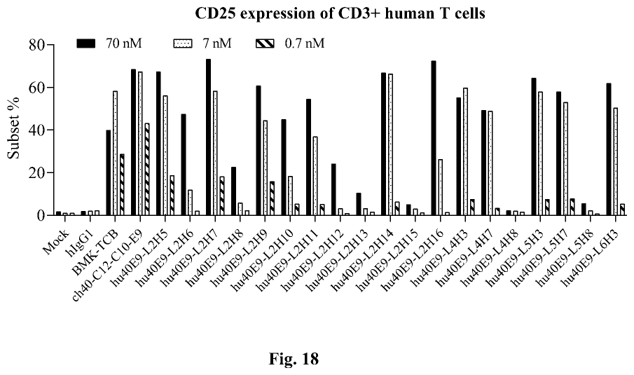

As shown in FIG. 11 and FIG. 18 below, the positive control BMK-TCB induced the upregulation of CD25 on human T cells as indicated by the subset rate of CD25⁺ cells in the CD3⁺ T cells. The activation effect of the humanized and/or affinity maturated antibodies was higher or comparable with the benchmark antibody BMK-TCB.

### Example 5. Humanized Antibody of 147E11E2: Generation, Affinity Maturation and Antibody Characterization

### 5.1 Humanized Antibody Generation

Chimeric antibody ch147E11E2 was selected as the clone for humanization. Antibody sequences were aligned with human germline sequences to identify best fit model. Best matched human germline sequences were selected as the templates for humanization based on homology to the original mouse antibody sequences. Generally, humanization of an antibody was performed by comparing IMGT (https://www.imgt.org) human antibody heavy and light chain variable strain gene database, heavy chain and light chain variable strain genes with high homology with murine-derived antibody were selected as templates, and CDRs of murine-derived antibody were transplanted into corresponding human templates. A variable region sequence with the order FR1-CDR1-FR2-CDR2-FR3-CDR3-FR4 was formed. As required, the key amino acids in the skeleton sequence were reverted and mutated to those corresponding to murine antibodies to ensure the original affinity.

Immunogenicity risk and hotspot were evaluated on these produced humanized antibodies, and no immunogenicity risk was identified. One hotspot (N55G56 motif) which liable to deamidation was identified in HCDR2 of the humanized antibody (e.g., hu147E2-L1H3) for 147E11E2 clone. To remove the deamidation site, different mutations were introduced to N55, and it was found that N55 can be mutated to a variety of residues, yet still retained the specific binding to CD3. For example, it was found that when N55 (the position is relative to SEQ ID NO: 213) was replaced by Q or S, the antibody binding affinity retained and there was no negative impact on its binding to CD3. The other mutations, such as mutations to G56, are also expected to work as well, as long as the deamidation site is removed and yet still retained the specific binding to CD3.

A total of 11 humanized antibodies were obtained from ch147E11E2 by mixing and matching 3 variants of humanized light chain variable regions (i.e., hu147E2-L1, hu147E2-L2 and hu147E2-L3) and 5 variants of humanized heavy chain variable regions (i.e., hu147E2-H2, hu147E2-H3, hu147E2-H3a, hu147E2-H3b and hu147E2-H4). The 11 humanized antibodies were designated as hu147E2-L1H2, hu147E2-L2H2, and so on, as shown in Table 28 below, by the same token.

TABLE 28
Heavy and light chain variable regions of exemplary
humanized antibodies for ch147E11E2

| | VL Region | | |
|---|---|---|---|
| | hu147E2-L1 | hu147E2-L2 | hu147E2-L3 |
| | (SEQ ID | (SEQ ID | (SEQ ID |
| VH Region | NO: 207) | NO: 208) | NO: 209) |
| hu147E2-H2 | hu147E2-L1H2 | hu147E2-L2H2 | hu147E2-L3H2 |
| (SEQ ID | (SEQ ID NOs: | (SEQ ID NOs: | (SEQ ID NOs: |
| NO: 202) | 207/202) | 208/202) | 209/202) |
| hu147E2-H3 | hu147E2-L1H3 | hu147E2-L2H3 | hu147E2-L3H3 |
| (SEQ ID | (SEQ ID NOS: | (SEQ ID NOs: | (SEQ ID NOs: |
| NO: 203) | 207/203) | 208/203) | 209/203) |
| hu147E2-H3a | hu147E2-L1H3a | N/A | N/A |
| (SEQ ID | (SEQ ID NOS: | | |
| NO: 204) | 207/204) | | |
| hu147E2-H3b | hu147E2-L1H3b | N/A | N/A |
| (SEQ ID | (SEQ ID NOS: | | |
| NO: 205) | 207/205) | | |
| hu147E2-H4 | hu147E2-L1H4 | hu147E2-L2H4 | hu147E2-L3H4 |
| (SEQ ID | (SEQ ID NOs: | (SEQ ID NOs: | (SEQ ID NOs: |
| NO: 206) | 207/206) | 208/206) | 209/206) |

### 5.2 Cell-Based Binding Affinity to Jurkat Cells

The binding affinity of several selected humanized 147E11E2 antibodies on human patient derived lymphocyte cancer cell line Jurkat was determined by FACS analysis. The benchmark antibody BMK-TCB was used as a positive control and hIgG1 isotype was used as a negative control. The protocol for FACS analysis is described in Example 3.3 except for the test antibodies used in step (c) were different.

As shown in FIG. 12 and Table 29 below, the binding affinity of several selected humanized 147E11E2 antibodies to Jurkat cells were higher than the benchmark antibody BMK-TCB.

TABLE 29
Binding affinity of selected
humanized antibodies to Jurkat cells.

| Ranking | Antibody | EC₅₀ (nM) | Top FACS signal |
|---|---|---|---|
| 1 | ch147E11E2 | 0.6494 | 2775 |
| 2 | hu147E2-L3H3 | 0.6589 | 2675 |
| 3 | hu147E2-L3H4 | 0.6715 | 2675 |
| 4 | hu147E2-L3H2 | 0.8911 | 2726 |
| 5 | BMK-TCB | 1.347 | 2366 |

### 5.3 Cell-Based Binding Affinity to 293T-cynoCD3mix Cells

The binding affinity of selected humanized antibodies and benchmark antibodies on HEK 293T stably over-expressing cynomolgus CD3 (293T-cynoCD3mix) cells was determined by FACS analysis. BMK-TCB was used as a positive control and hIgG1 isotype was used as a negative control. The protocol for FACS analysis is described in Example 3.4 except for the test antibodies used in step (c) were different.

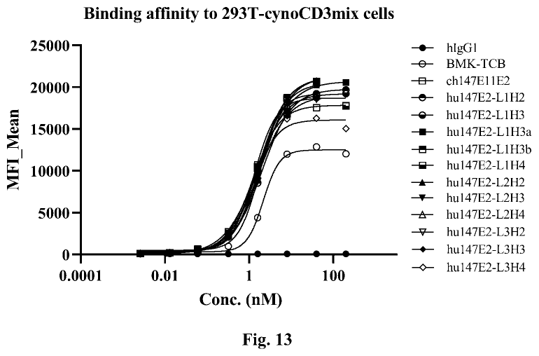

As shown in FIG. 13 and Table 30 below, the binding affinity of the produced humanized 147E11E2 antibodies to 293T-cynoCD3mix cells were higher than the benchmark antibody BMK-TCB.

TABLE 30
Binding affinity of selected humanized
antibodies to 293T-cynoCD3mix.

| Ranking | Antibody | EC50 (nM) | Top signal |
|---|---|---|---|
| 1 | ch147E11E2 | 1.23 | 17837 |
| 2 | hu147E2-L3H4 | 1.29 | 16062 |
| 3 | hu147E2-L1H4 | 1.31 | 18693 |
| 4 | hu147E2-L1H3 | 1.51 | 19199 |
| 5 | hu147E2-L3H3 | 1.63 | 18962 |
| 6 | hu147E2-L2H2 | 1.64 | 20532 |
| 7 | hu147E2-L1H3a | 1.66 | 20682 |
| 8 | hu147E2-L1H3b | 1.68 | 21141 |
| 9 | hu147E2-L3H2 | 1.77 | 19191 |
| 10 | hu147E2-L2H3 | 1.86 | 21012 |
| 11 | hu147E2-L1H2 | 2.01 | 19743 |
| 12 | hu147E2-L2H4 | 2.02 | 21234 |
| 13 | BMK-TCB | 2.16 | 12509 |

### 5.4 Jurkat-NFAT-Luciferase Activation Assay

The T cell activation capability of these antibodies was determined by Jurkat NFAT-Luciferase activation assay. The benchmark antibody BMK-TCB was used as a positive control and hIgG1 isotype was used as a negative control. The protocol for activation analysis is described in Example 4.4 except for the test antibodies used in step (a) were different.

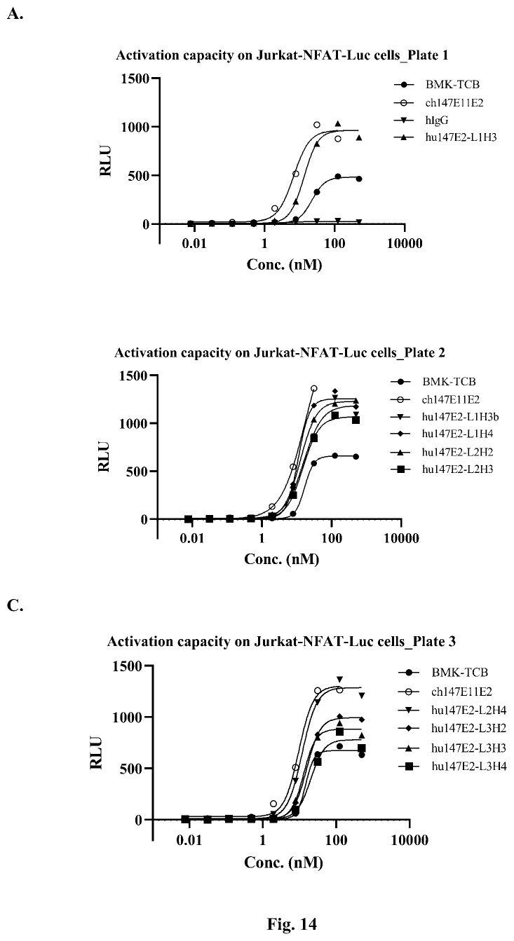

The values of the activated EC₅₀ and the top signal were normalized according to ch147E11E2 on the same plate and the values of benchmark antibody BMK-TCB were shown as mean. As shown in FIGS. 14A˜C and Table 31 below, the activation capacity of selected humanized antibodies was higher than benchmark antibody BMK-TCB.

TABLE 31
Activation capacity of the humanized antibodies
on Jurkat-NFAT-luciferase cells.

| | | EC₅₀_Fold | Top FACS |
|---|---|---|---|
| Ranking | Antibody | change | signal_Fold change |
| 1 | ch147E11E2 | 1.00 | 1.00 |
| 2 | hu147E2-L1H4 | 1.21 | 0.92 |
| 3 | hu147E2-L2H4 | 1.24 | 0.99 |
| 4 | hu147E2-L3H3 | 1.36 | 0.68 |
| 5 | hu147E2-L2H2 | 1.40 | 0.90 |
| 6 | hu147E2-L2H3 | 1.65 | 0.78 |
| 7 | hu147E2-L3H2 | 1.66 | 0.76 |
| 8 | hu147E2-L1H3b | 1.73 | 0.87 |
| 9 | hu147E2-L1H3 | 1.98 | 1.00 |
| 10 | hu147E2-L3H4 | 2.12 | 0.60 |
| 11 | BMK-TCB | 2.24 | 0.50 |

### 5.5 Human PBMC Activation Assay

A primary human peripheral blood mononuclear cell (PBMC) based assay was used to determine the activation capacity of the humanized antibodies to T cells. It is well known that when T cells are activated, the expression of the surface protein CD25 is upregulated. The hIgG1 isotype was used as a negative control. The protocol for activation analysis is described in Example 3.6.2 except for the concentrations and test antibodies used in step (a) were different.

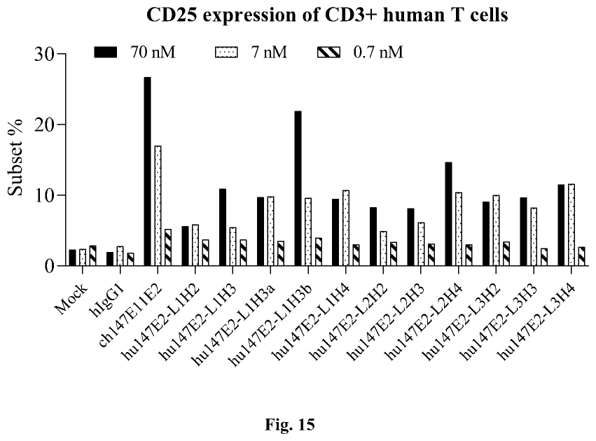

As shown in FIG. 15, the humanized antibodies induced the upregulation of CD25 on human T cells as indicated by the subset rate of CD25⁺ cells in the CD3⁺ T cells.

What claimed is:

1. An antibody or antigen-binding fragment thereof which specifically binds to CD3, comprising:
one or two or three heavy chain complementarity determining regions (HCDR1, HCDR2 and/or HCDR3) contained within any one of the heavy chain variable (VH) region sequences selected from the group consisting of SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 and 245; and/or
one or two or three light chain complementarity determining regions (LCDR1, LCDR2 and/or LCDR3) contained within any one of the light chain variable (VL) region sequences selected from the group consisting of SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 and 232.

2. The antibody or antigen-binding fragment thereof of claim 1, wherein the antibody or antigen-binding fragment comprises at least one heavy or light chain complementarity determining region (CDR) comprising an amino acid sequence selected from the group consisting of SEQ ID NOs: 1, 2, 3, 4, 5, 6, 9, 10, 11, 12, 13, 14, 17, 18, 19, 20, 21, 22, 25, 26, 27, 28, 29, 30, 33, 34, 35, 36, 37, 38, 41, 42, 43, 44, 45, 46, 49, 50, 51, 52, 53, 54, 57, 58, 59, 60, 61, 62, 65, 66, 67, 68, 69, 70, 73, 74, 75, 76, 77, 78, 81, 82, 83, 84, 85, 86, 89, 90, 91, 92, 93, 94, 102, 103, 104, 105, 106, 107, 110, 111, 112, 113, 114, 115, 118, 119, 120, 121, 122, 123, 126, 127, 128, 129, 130, 131, 134, 135, 136, 137, 138, 139, 142, 143, 144, 145, 146, 147, 150, 151, 152, 153, 154, 155, 171, 172, 173, 174, 175, 176, 177, 178, 179, 180, 181, 182, 183, 184, 185, 201, 162, 167, 210, 211, 212, 214, 215, 216, 217, 218, 219, 220, 221, 222, 223, 224, 225, 226, 227, 228 and 229.

3. The antibody or antigen-binding fragment thereof of claim 1 or 2, comprising a VH region comprising one or two or three of HCDR1, HCDR2 and HCDR3 comprising an amino acid sequence selected from the group consisting of SEQ ID NOs: 1, 2, 3, 9, 10, 11, 17, 18, 19, 25, 26, 27, 33, 34, 35, 41, 42, 43, 49, 50, 51, 57, 58, 59, 65, 66, 67, 73, 74, 75, 81, 82, 83, 89, 90, 91, 102, 103, 104, 110, 111, 112, 118, 119, 120, 126, 127, 128, 134, 135, 136, 142, 143, 144, 150, 151, 152, 171, 172, 173, 174, 175, 176, 177, 178, 179, 180, 181, 201, 162, 210, 211, 212, 217, 218, 219, 220, 221, 222, 223, 224, 225, 226, 227, 228 and 229.

4. The antibody or antigen-binding fragment thereof of any one of the preceding claims, comprising a VL region comprising one or two or three of LCDR1, LCDR2 and LCDR3 comprising an amino acid sequence selected from the group consisting of SEQ ID NOs: 4, 5, 6, 12, 13, 14, 20, 21, 22, 28, 29, 30, 36, 37, 38, 44, 45, 46, 52, 53, 54, 60, 61, 62, 68, 69, 70, 76, 77, 78, 84, 85, 86, 92, 93, 94, 105, 106, 107, 113, 114, 115, 121, 122, 123, 129, 130, 131, 137, 138, 139, 145, 146, 147, 153, 154, 155, 182, 183, 184, 185, 167, 214, 215 and 216.

5. The antibody or antigen-binding fragment thereof of any one of the preceding claims, comprising:
i. a HCDR1 comprising an amino acid sequence selected from the group consisting of SEQ ID NOs: 1, 9, 17, 25, 33, 41, 49, 57, 65, 73, 81, 89, 102, 110, 118, 126, 134, 142, 150, 217, 218 and 219;
ii. a HCDR2 comprising an amino acid sequence selected from the group consisting of SEQ ID NOs: 2, 10, 18, 26, 34, 42, 50, 58, 66, 74, 82, 90, 103, 111, 119, 127, 135, 143, 151, 171, 172, 173, 201, 210, 211, 212, 220 and 222; and
iii. a HCDR3 comprising an amino acid sequence selected from the group consisting of SEQ ID NOs: 3, 11, 19, 27, 35, 43, 51, 59, 67, 75, 83, 91, 104, 112, 120, 128, 136, 144, 152, 174, 175, 176, 177, 178, 179, 180, 181, 221, 223, 224, 225, 226, 227, 228, 229 and 162.

6. The antibody or antigen-binding fragment thereof of any one of the preceding claims, comprising:
i. a LCDR1 comprising an amino acid sequence selected from the group consisting of SEQ ID NOs: 4, 12, 20, 28, 36, 44, 52, 60, 68, 76, 84, 92, 105, 113, 121, 129, 137, 145 and 153;
ii. a LCDR2 comprising an amino acid sequence selected from the group consisting of SEQ ID NOs: 5, 13, 21, 29, 37, 45, 53, 61, 69, 77, 85, 93, 106, 114, 122, 130, 138, 146 and 154; and
iii. a LCDR3 comprising an amino acid sequence selected from the group consisting of SEQ ID NOs: 6, 14, 22, 30, 38, 46, 54, 62, 70, 78, 86, 94, 107, 115, 123, 131, 139, 147, 155, 182, 183, 184, 185, 214, 215, 216 and 167.

7. The antibody or antigen-binding fragment thereof of any one of the preceding claims, comprising:
i. a HCDR1 comprising an amino acid sequence as set forth in SEQ ID NO: 1, a HCDR2 comprising an amino acid sequence as set forth in SEQ ID NO: 2, and a HCDR3 comprising an amino acid sequence as set forth in SEQ ID NO: 3;
ii. a HCDR1 comprising an amino acid sequence as set forth in SEQ ID NO: 9, a HCDR2 comprising an amino acid sequence as set forth in SEQ ID NO: 10, and a HCDR3 comprising an amino acid sequence as set forth in SEQ ID NO: 11;
iii. a HCDR1 comprising an amino acid sequence as set forth in SEQ ID NO: 17, a HCDR2 comprising an amino acid sequence as set forth in SEQ ID NO: 18, and a HCDR3 comprising an amino acid sequence as set forth in SEQ ID NO: 19;
iv. a HCDR1 comprising an amino acid sequence as set forth in SEQ ID NO: 25, a HCDR2 comprising an amino acid sequence as set forth in SEQ ID NO: 26, and a HCDR3 comprising an amino acid sequence as set forth in SEQ ID NO: 27;
v. a HCDR1 comprising an amino acid sequence as set forth in SEQ ID NO: 33, a HCDR2 comprising an amino acid sequence as set forth in SEQ ID NO: 34, and a HCDR3 comprising an amino acid sequence as set forth in SEQ ID NO: 35;
vi. a HCDR1 comprising an amino acid sequence as set forth in SEQ ID NO: 41, a HCDR2 comprising an amino acid sequence as set forth in SEQ ID NO: 42, and a HCDR3 comprising an amino acid sequence as set forth in SEQ ID NO: 43;
vii. a HCDR1 comprising an amino acid sequence as set forth in SEQ ID NO: 49, a HCDR2 comprising an amino acid sequence as set forth in SEQ ID NO: 50, and a HCDR3 comprising an amino acid sequence as set forth in SEQ ID NO: 51;
viii. a HCDR1 comprising an amino acid sequence as set forth in SEQ ID NO: 57, a HCDR2 comprising an amino acid sequence as set forth in SEQ ID NO: 58, and a HCDR3 comprising an amino acid sequence as set forth in SEQ ID NO: 59;
ix. a HCDR1 comprising an amino acid sequence as set forth in SEQ ID NO: 65, a HCDR2 comprising an amino acid sequence as set forth in SEQ ID NO: 66, and a HCDR3 comprising an amino acid sequence as set forth in SEQ ID NO: 67;
x. a HCDR1 comprising an amino acid sequence as set forth in SEQ ID NO: 73, a HCDR2 comprising an amino acid sequence as set forth in SEQ ID NO: 74, and a HCDR3 comprising an amino acid sequence as set forth in SEQ ID NO: 75;
xi. a HCDR1 comprising an amino acid sequence as set forth in SEQ ID NO: 81, a HCDR2 comprising an amino acid sequence as set forth in SEQ ID NO: 82, and a HCDR3 comprising an amino acid sequence as set forth in SEQ ID NO: 83;
xii. a HCDR1 comprising an amino acid sequence as set forth in SEQ ID NO: 89, a HCDR2 comprising an amino acid sequence as set forth in SEQ ID NO: 90, and a HCDR3 comprising an amino acid sequence as set forth in SEQ ID NO: 91;
xiii. a HCDR1 comprising an amino acid sequence as set forth in SEQ ID NO: 102, a HCDR2 comprising an amino acid sequence as set forth in SEQ ID NO: 103, and a HCDR3 comprising an amino acid sequence as set forth in SEQ ID NO: 104;
xiv. a HCDR1 comprising an amino acid sequence as set forth in SEQ ID NO: 110, a HCDR2 comprising an amino acid sequence as set forth in SEQ ID NO: 111, and a HCDR3 comprising an amino acid sequence as set forth in SEQ ID NO: 112;
xv. a HCDR1 comprising an amino acid sequence as set forth in SEQ ID NO: 118, a HCDR2 comprising an amino acid sequence as set forth in SEQ ID NO: 119, and a HCDR3 comprising an amino acid sequence as set forth in SEQ ID NO: 120;
xvi. a HCDR1 comprising an amino acid sequence as set forth in SEQ ID NO: 126, a HCDR2 comprising an amino acid sequence as set forth in SEQ ID NO: 127, and a HCDR3 comprising an amino acid sequence as set forth in SEQ ID NO: 128;
xvii. a HCDR1 comprising an amino acid sequence as set forth in SEQ ID NO: 134, a HCDR2 comprising an amino acid sequence as set forth in SEQ ID NO: 135, and a HCDR3 comprising an amino acid sequence as set forth in SEQ ID NO: 136;
xviii. a HCDR1 comprising an amino acid sequence as set forth in SEQ ID NO: 142, a HCDR2 comprising an amino acid sequence as set forth in SEQ ID NO: 143, and a HCDR3 comprising an amino acid sequence as set forth in SEQ ID NO: 144;
xix. a HCDR1 comprising an amino acid sequence as set forth in SEQ ID NO: 150, a HCDR2 comprising an amino acid sequence as set forth in SEQ ID NO: 151, and a HCDR3 comprising an amino acid sequence as set forth in SEQ ID NO: 152;
XX. a HCDR1 comprising an amino acid sequence as set forth in SEQ ID NO: 219, a HCDR2 comprising an amino acid sequence as set forth in SEQ ID NO: 201, and a HCDR3 comprising an amino acid sequence as set forth in SEQ ID NO: 162;
xxi. a HCDR1 comprising an amino acid sequence as set forth in SEQ ID NO: 9, 217 or 218; a HCDR2 comprising an amino acid sequence as set forth in SEQ ID NO: 10, 171, 172, 173, 220 or 222; a HCDR3 comprising an amino acid sequence as set forth in SEQ ID NO: 11, 174, 175, 176, 177, 178, 179, 180, 181, 221, 223, 224, 225, 226, 227, 228 or 229;
xxii. a HCDR1 comprising an amino acid sequence as set forth in SEQ ID NO: 150, a HCDR2 comprising an amino acid sequence as set forth in SEQ ID NO: 212, and a HCDR3 comprising an amino acid sequence as set forth in SEQ ID NO: 152;
xxiii. a HCDR1 comprising an amino acid sequence as set forth in SEQ ID NO: 150, a HCDR2 comprising an amino acid sequence as set forth in SEQ ID NO: 210, and a HCDR3 comprising an amino acid sequence as set forth in SEQ ID NO: 152; or
xxiv. a HCDR1 comprising an amino acid sequence as set forth in SEQ ID NO: 150, a HCDR2 comprising an amino acid sequence as set forth in SEQ ID NO: 211, and a HCDR3 comprising an amino acid sequence as set forth in SEQ ID NO: 152.

8. The antibody or antigen-binding fragment thereof of any one of the preceding claims, comprising:
i. a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
ii. a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
iii. a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
iv. a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
v. a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
vi. a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vii. a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
viii. a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
ix. a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
x. a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
xi. a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
xii. a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
xiii. a LCDR1 comprising an amino acid sequence as set forth in SEQ ID NO: 105, a LCDR2 comprising an amino acid sequence as set forth in SEQ ID NO: 106, and a LCDR3 comprising an amino acid sequence as set forth in SEQ ID NO: 107;
xiv. a LCDR1 comprising an amino acid sequence as set forth in SEQ ID NO: 113, a LCDR2 comprising an amino acid sequence as set forth in SEQ ID NO: 114, and a LCDR3 comprising an amino acid sequence as set forth in SEQ ID NO: 115;
XV. a LCDR1 comprising an amino acid sequence as set forth in SEQ ID NO: 121, a LCDR2 comprising an amino acid sequence as set forth in SEQ ID NO: 122, and a LCDR3 comprising an amino acid sequence as set forth in SEQ ID NO: 123;
xvi. a LCDR1 comprising an amino acid sequence as set forth in SEQ ID NO: 129, a LCDR2 comprising an amino acid sequence as set forth in SEQ ID NO: 130, and a LCDR3 comprising an amino acid sequence as set forth in SEQ ID NO: 131;
xvii. a LCDR1 comprising an amino acid sequence as set forth in SEQ ID NO: 137, a LCDR2 comprising an amino acid sequence as set forth in SEQ ID NO: 138, and a LCDR3 comprising an amino acid sequence as set forth in SEQ ID NO: 139;
xviii. a LCDR1 comprising an amino acid sequence as set forth in SEQ ID NO: 145, a LCDR2 comprising an amino acid sequence as set forth in SEQ ID NO: 146, and a LCDR3 comprising an amino acid sequence as set forth in SEQ ID NO: 147;
xix. a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
xx. a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 167; or
xxi. a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14, 182, 183, 184, 185, 214, 215 or 216.

9. The antibody or antigen-binding fragment thereof of any one of the preceding claims, comprising:
i. a HCDR1 comprising an amino acid sequence as set forth in SEQ ID NO: 1, a HCDR2 comprising an amino acid sequence as set forth in SEQ ID NO: 2, a HCDR3 comprising an amino acid sequence as set forth in SEQ ID NO: 3, a LCDR1 comprising an amino acid sequence as set forth in SEQ ID NO: 4, a LCDR2 comprising an amino acid sequence as set forth in SEQ ID NO: 5, and a LCDR3 comprising an amino acid sequence as set forth in SEQ ID NO: 6;
ii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
iii. a HCDR1 comprising an amino acid sequence as set forth in SEQ ID NO: 17, a HCDR2 comprising an amino acid sequence as set forth in SEQ ID NO: 18, a HCDR3 comprising an amino acid sequence as set forth in SEQ ID NO: 19, a LCDR1 comprising an amino acid sequence as set forth in SEQ ID NO: 20, a LCDR2 comprising an amino acid sequence as set forth in SEQ ID NO: 21, and a LCDR3 comprising an amino acid sequence as set forth in SEQ ID NO: 22;
iv. a HCDR1 comprising an amino acid sequence as set forth in SEQ ID NO: 25, a HCDR2 comprising an amino acid sequence as set forth in SEQ ID NO: 26, a HCDR3 comprising an amino acid sequence as set forth in SEQ ID NO: 27, a LCDR1 comprising an amino acid sequence as set forth in SEQ ID NO: 28, a LCDR2 comprising an amino acid sequence as set forth in SEQ ID NO: 29, and a LCDR3 comprising an amino acid sequence as set forth in SEQ ID NO: 30;
v. a HCDR1 comprising an amino acid sequence as set forth in SEQ ID NO: 33, a HCDR2 comprising an amino acid sequence as set forth in SEQ ID NO: 34, a HCDR3 comprising an amino acid sequence as set forth in SEQ ID NO: 35, a LCDR1 comprising an amino acid sequence as set forth in SEQ ID NO: 36, a LCDR2 comprising an amino acid sequence as set forth in SEQ ID NO: 37, and a LCDR3 comprising an amino acid sequence as set forth in SEQ ID NO: 38;
vi. a HCDR1 comprising an amino acid sequence as set forth in SEQ ID NO: 41, a HCDR2 comprising an amino acid sequence as set forth in SEQ ID NO: 42, a HCDR3 comprising an amino acid sequence as set forth in SEQ ID NO: 43, a LCDR1 comprising an amino acid sequence as set forth in SEQ ID NO: 44, a LCDR2 comprising an amino acid sequence as set forth in SEQ ID NO: 45, and a LCDR3 comprising an amino acid sequence as set forth in SEQ ID NO: 46;
vii. a HCDR1 comprising an amino acid sequence as set forth in SEQ ID NO: 49, a HCDR2 comprising an amino acid sequence as set forth in SEQ ID NO: 50, a HCDR3 comprising an amino acid sequence as set forth in SEQ ID NO: 51, a LCDR1 comprising an amino acid sequence as set forth in SEQ ID NO: 52, a LCDR2 comprising an amino acid sequence as set forth in SEQ ID NO: 53, and a LCDR3 comprising an amino acid sequence as set forth in SEQ ID NO: 54;
viii. a HCDR1 comprising an amino acid sequence as set forth in SEQ ID NO: 57, a HCDR2 comprising an amino acid sequence as set forth in SEQ ID NO: 58, a HCDR3 comprising an amino acid sequence as set forth in SEQ ID NO: 59, a LCDR1 comprising an amino acid sequence as set forth in SEQ ID NO: 60, a LCDR2 comprising an amino acid sequence as set forth in SEQ ID NO: 61, and a LCDR3 comprising an amino acid sequence as set forth in SEQ ID NO: 62;
ix. a HCDR1 comprising an amino acid sequence as set forth in SEQ ID NO: 65, a HCDR2 comprising an amino acid sequence as set forth in SEQ ID NO: 66, a HCDR3 comprising an amino acid sequence as set forth in SEQ ID NO: 67, a LCDR1 comprising an amino acid sequence as set forth in SEQ ID NO: 68, a LCDR2 comprising an amino acid sequence as set forth in SEQ ID NO: 69, and a LCDR3 comprising an amino acid sequence as set forth in SEQ ID NO: 70;
x. a HCDR1 comprising an amino acid sequence as set forth in SEQ ID NO: 73, a HCDR2 comprising an amino acid sequence as set forth in SEQ ID NO: 74, a HCDR3 comprising an amino acid sequence as set forth in SEQ ID NO: 75, a LCDR1 comprising an amino acid sequence as set forth in SEQ ID NO: 76, a LCDR2 comprising an amino acid sequence as set forth in SEQ ID NO: 77, and a LCDR3 comprising an amino acid sequence as set forth in SEQ ID NO: 78;
xi. a HCDR1 comprising an amino acid sequence as set forth in SEQ ID NO: 81, a HCDR2 comprising an amino acid sequence as set forth in SEQ ID NO: 82, a HCDR3 comprising an amino acid sequence as set forth in SEQ ID NO: 83, a LCDR1 comprising an amino acid sequence as set forth in SEQ ID NO: 84, a LCDR2 comprising an amino acid sequence as set forth in SEQ ID NO: 85, and a LCDR3 comprising an amino acid sequence as set forth in SEQ ID NO: 86;
xii. a HCDR1 comprising an amino acid sequence as set forth in SEQ ID NO: 89, a HCDR2 comprising an amino acid sequence as set forth in SEQ ID NO: 90, a HCDR3 comprising an amino acid sequence as set forth in SEQ ID NO: 91, a LCDR1 comprising an amino acid sequence as set forth in SEQ ID NO: 92, a LCDR2 comprising an amino acid sequence as set forth in SEQ ID NO: 93, and a LCDR3 comprising an amino acid sequence as set forth in SEQ ID NO: 94;
xiii. a HCDR1 comprising an amino acid sequence as set forth in SEQ ID NO: 102, a HCDR2 comprising an amino acid sequence as set forth in SEQ ID NO: 103, a HCDR3 comprising an amino acid sequence as set forth in SEQ ID NO: 104, a LCDR1 comprising an amino acid sequence as set forth in SEQ ID NO: 105, a LCDR2 comprising an amino acid sequence as set forth in SEQ ID NO: 106, and a LCDR3 comprising an amino acid sequence as set forth in SEQ ID NO: 107;
xiv. a HCDR1 comprising an amino acid sequence as set forth in SEQ ID NO: 110, a HCDR2 comprising an amino acid sequence as set forth in SEQ ID NO: 111, a HCDR3 comprising an amino acid sequence as set forth in SEQ ID NO: 112, a LCDR1 comprising an amino acid sequence as set forth in SEQ ID NO: 113, a LCDR2 comprising an amino acid sequence as set forth in SEQ ID NO: 114, and a LCDR3 comprising an amino acid sequence as set forth in SEQ ID NO: 115;
xv. a HCDR1 comprising an amino acid sequence as set forth in SEQ ID NO: 118, a HCDR2 comprising an amino acid sequence as set forth in SEQ ID NO: 119, a HCDR3 comprising an amino acid sequence as set forth in SEQ ID NO: 120, a LCDR1 comprising an amino acid sequence as set forth in SEQ ID NO: 121, a LCDR2 comprising an amino acid sequence as set forth in SEQ ID NO: 122, and a LCDR3 comprising an amino acid sequence as set forth in SEQ ID NO: 123;
xvi. a HCDR1 comprising an amino acid sequence as set forth in SEQ ID NO: 126, a HCDR2 comprising an amino acid sequence as set forth in SEQ ID NO: 127, a HCDR3 comprising an amino acid sequence as set forth in SEQ ID NO: 128, a LCDR1 comprising an amino acid sequence as set forth in SEQ ID NO: 129, a LCDR2 comprising an amino acid sequence as set forth in SEQ ID NO: 130, and a LCDR3 comprising an amino acid sequence as set forth in SEQ ID NO: 131;
xvii. a HCDR1 comprising an amino acid sequence as set forth in SEQ ID NO: 134, a HCDR2 comprising an amino acid sequence as set forth in SEQ ID NO: 135, a HCDR3 comprising an amino acid sequence as set forth in SEQ ID NO: 136, a LCDR1 comprising an amino acid sequence as set forth in SEQ ID NO: 137, a LCDR2 comprising an amino acid sequence as set forth in SEQ ID NO: 138, and a LCDR3 comprising an amino acid sequence as set forth in SEQ ID NO: 139;
xviii. a HCDR1 comprising an amino acid sequence as set forth in SEQ ID NO: 142, a HCDR2 comprising an amino acid sequence as set forth in SEQ ID NO: 143, a HCDR3 comprising an amino acid sequence as set forth in SEQ ID NO: 144, a LCDR1 comprising an amino acid sequence as set forth in SEQ ID NO: 145, a LCDR2 comprising an amino acid sequence as set forth in SEQ ID NO: 146, and a LCDR3 comprising an amino acid sequence as set forth in SEQ ID NO: 147;
xix. a HCDR1 comprising an amino acid sequence as set forth in SEQ ID NO: 150, a HCDR2 comprising an amino acid sequence as set forth in SEQ ID NO: 151, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
xx. a HCDR1 comprising an amino acid sequence as set forth in SEQ ID NO: 9, a HCDR2 comprising an amino acid sequence as set forth in SEQ ID NO: 171, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxi. a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxii. a HCDR1 comprising an amino acid sequence as set forth in SEQ ID NO: 9, a HCDR2 comprising an amino acid sequence as set forth in SEQ ID NO: 173, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxiii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 174, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxiv. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 175, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
XXV. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 176, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxvi. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 177, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxvii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 178, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxviii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 179, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxix. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxx. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 181, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxxi. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 182;
xxxii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 183;
xxxiii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 184;
xxxiv. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 185;
xxxv. a HCDR1 comprising an amino acid sequence as set forth in SEQ ID NO: 9, a HCDR2 comprising an amino acid sequence as set forth in SEQ ID NO: 220, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxxvi. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 221, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxxvii. a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxxviii. a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xxxix. a HCDR1 comprising an amino acid sequence as set forth in SEQ ID NO: 9, a HCDR2 comprising an amino acid sequence as set forth in SEQ ID NO: 171, a HCDR3 comprising an amino acid sequence as set forth in SEQ ID NO: 224, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xl. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
xli. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
xlii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 216;
xliii. a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
xliv. a HCDR1 comprising an amino acid sequence as set forth in SEQ ID NO: 9, a HCDR2 comprising an amino acid sequence as set forth in SEQ ID NO: 222, a HCDR3 comprising an amino acid sequence as set forth in SEQ ID NO: 180, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
xlv. a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 214;
xlvi. a HCDR1 comprising an amino acid sequence as set forth in SEQ ID NO: 9, a HCDR2 comprising an amino acid sequence as set forth in SEQ ID NO: 172, a HCDR3 comprising an amino acid sequence as set forth in SEQ ID NO: 223, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 215;
xlvii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 225, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xlviii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 226, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
xlix. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 227, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
1. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 228, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
li. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 174, a LCDR1 comprising an amino acid sequence as set for th in SEQ ID NO: 12, a LCDR2 comprising an amino acid sequence as set forth in SEQ ID NO: 13, and a LCDR3 comprising an amino acid sequence as set forth in SEQ ID NO: 14;
lii. a HCDR1 comprising an amino acid sequence as set forth in SEQ ID NO: 9, a HCDR2 comprising an amino acid sequence as set forth in SEQ ID NO: 10, a HCDR3 comprising an amino acid sequence as set forth in SEQ ID NO: 229, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
liii a HCDR1 comprising an amino acid sequence as set forth in SEQ ID NO: 217, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
liv. a HCDR1 comprising an amino acid sequence as set forth in SEQ ID NO: 218, a HCDR2 comprising an amino acid sequence as set forth in SEQ ID NO: 10, a HCDR3 comprising an amino acid sequence as set forth in SEQ ID NO: 11, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 14;
lv. a HCDR1 comprising an amino acid sequence as set forth in SEQ ID NO: 219, a HCDR2 comprising an amino acid sequence as set forth in SEQ ID NO: 201, a HCDR3 comprising an amino acid sequence as set forth in SEQ ID NO: 162, a LCDR1 comprising an amino acid sequence as set forth in SEQ ID NO: 12, a LCDR2 comprising an amino acid sequence as set forth in SEQ ID NO: 13, and a LCDR3 comprising an amino acid sequence as set forth in SEQ ID NO: 167;
lvi. a HCDR1 comprising an amino acid sequence as set forth in SEQ ID NO: 150, a HCDR2 comprising an amino acid sequence as set forth in SEQ ID NO: 212, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155;
lvii. a HCDR1 comprising an amino acid sequence as set forth in SEQ ID NO: 150, a HCDR2 comprising an amino acid sequence as set forth in SEQ ID NO: 210, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155; or
lviii. a HCDR1 comprising an amino acid sequence as set forth in SEQ ID NO: 150, a HCDR2 comprising an amino acid sequence as set forth in SEQ ID NO: 211, a HCDR3 comprising an amino acid sequence as set forth in SEQ ID NO: 152, a LCDR1 comprising an amino acid sequence as set forth in SEQ ID NO: 153, a LCDR2 comprising an amino acid sequence as set forth in SEQ ID NO: 154, and a LCDR3 comprising an amino acid sequence as set forth in SEQ ID NO: 155.

10. The antibody or antigen-binding fragment thereof of any one of the preceding claims, comprising a VH region having an amino acid sequence as set forth in SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 or 245, or a homologous sequence thereof having at least 80% sequence identity to SEQ ID NOs: 7, 15, 23, 31, 39, 47, 55, 63, 71, 79, 87, 95, 108, 116, 124, 132, 140, 148, 156, 163, 164, 165, 166, 186, 187, 188, 189, 190, 191, 192, 193, 194, 195, 196, 213, 202, 203, 204, 205, 206, 233, 234, 235, 236, 237, 238, 239, 240, 241, 242, 243, 244 or 245.

11. The antibody or antigen-binding fragment thereof of any one of the preceding claims, comprising a VL region having an amino acid sequence as set forth in SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 or 232, or a homologous sequence thereof having at least 80% sequence identity to SEQ ID NOs: 8, 16, 24, 32, 40, 48, 56, 64, 72, 80, 88, 96, 109, 117, 125, 133, 141, 149, 157, 168, 169, 170, 197, 198, 199, 200, 207, 208, 209, 230, 231 or 232.

12. The antibody or antigen-binding fragment thereof of any one of the preceding claims, comprising a VH/VL amino acid sequence pair selected from the group consisting of SEQ ID NOs: 7/8, 15/16, 23/24, 31/32, 39/40, 47/48, 55/56, 63/64, 71/72, 79/80, 87/88, 95/96, 108/109, 116/117, 124/125, 132/133, 140/141, 148/149, 156/157, 163/168, 163/169, 163/170, 164/168, 164/169, 164/170, 165/168, 165/169, 165/170, 166/168, 166/169, 166/170, 186/169, 187/169, 188/169, 189/169, 190/169, 191/169, 192/169, 193/169, 194/169, 195/169, 196/169, 165/197, 165/198, 165/199, 165/200, 202/207, 202/208, 202/209, 203/207, 203/208, 203/209, 204/207, 205/207, 206/207, 206/208, 206/209, 233/169, 234/169, 235/169, 236/169, 237/169, 238/169, 239/169, 240/169, 241/169, 242/169, 243/169, 244/169, 245/169, 165/230, 165/231, 165/232, 235/230, 235/231, 236/230 and 236/231.

13. The antibody or antigen-binding fragment thereof of any one of the preceding claims, further comprising one or more amino acid residue substitutions or modifications yet retains specific binding affinity to CD3.

14. The antibody or antigen-binding fragment thereof of claim 13, wherein at least one of the substitutions or modifications is in one or more of the CDR sequences of the VH region or VL region.

15. The antibody or antigen-binding fragment thereof of claim 13, wherein at least one of the substitutions or modifications is in one or more of the non-CDR sequences of the VH region or VL region.

16. The antibody or antigen-binding fragment of the proceeding claims, further comprising one or more non-natural amino acid (NNAA) substitution.

17. The antibody or antigen-binding fragment of claim 16, wherein the NNAA is capable of being conjugated.

18. The antibody or antigen-binding fragment thereof of any one of the preceding claims, having one or more properties to CD3 selected from the group consisting of:
i. being capable of specifically binding to human CD3 (e.g., as measured by FACS assay);
ii. being capable of binding to both human and cynomolgus CD3 (e.g., as measured by FACS assay);
iii. being with a T cell activation capability (e.g., as measured by Jurkat NFAT-Luciferase activation assay); and
iv. being with a PBMC activation capability (e.g., as measured by ELISA assay).

19. An antibody or antigen-binding fragment thereof, which competes for binding to CD3 with the antibody or antigen-binding fragment thereof of any one of the preceding claims.

20. The antibody or antigen-binding fragment thereof of any one of the preceding claims, which is a chimeric, a humanized or a human antibody or an antigen-binding fragment thereof.

21. The antibody or antigen-binding fragment thereof of any one of the preceding claims, which is a labeled antibody, a bivalent antibody, an anti-idiotypic antibody or a fusion protein.

22. The antibody or antigen-binding fragment thereof of any one of the preceding claims, which is a diabody, a Fab, a Fab′, a F(ab′)₂, a Fd, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), an scFv dimer (bivalent diabody), a camelized single domain antibody, a nanobody, a domain antibody, or a bivalent domain antibody.

23. The antibody or antigen-binding fragment thereof of any one of the preceding claims, further comprising an Fc region, optionally an Fc region of human immunoglobulin (Ig), or optionally an Fc region of human IgG.

24. The antibody or antigen-binding fragment thereof of claim 23, wherein the Fc region is derived from human IgG1, IgG2, IgG3, or IgG4.

25. The antibody or antigen-binding fragment thereof of claim 24, wherein the Fc region comprises an amino acid sequence selected from the group consisting of SEQ ID NOs: 97-99.

26. The antibody or antigen-binding fragment thereof of any one of the preceding claims, wherein the light chain is a λ light chain or a κ light chain.

27. The antibody or antigen-binding fragment thereof of any one of the preceding claims, which is a bispecific or multi-specific antibody or an antigen-binding fragment thereof.

28. The antibody or antigen-binding fragment thereof of claim 27, which is capable of specifically binding to one or more additional antigens other than CD3, or a second epitope on CD3.

29. The antibody or antigen-binding fragment thereof of claim 28, wherein the one or more additional antigens other than CD3 are selected from the group consisting of CD16a, CD33, CD38, CD45, CD123, CD146, CD228, CLL-1, FLT3, FLT3L, TAF1, TgPRF, HVCN1, IL-6R, IL-11R, IL17A, IL-23R, IL-33, ILDR2, LAP, TSLP, TREM-1, ANGPT2, APOE, IFNAR, CypA, DOG-1, NKp30, CSF-1R, CCR2, LRRC15, mesothelin, Dickkopf2, DLL3, HER-2, C10orf54, TrkA, MEKK1, KRAS, ERK, XPO1, mTORC1/2, PAK4, NAMPT, ATR, EGFR, FGFR, VEGF, LILRB (e.g., LILRB1, LILRB2, LILRB3, LILRB4, LILRB5), c-MET, Her2, Her3, CTLA4, GITA, CD112R, CD2, CD7, CD16, CD19, CD20, CD24, CD27, CD30, CD34, CD37, CD39, CD70, CD73, CD83, CD28, CD80 (B7-1), CD86 (B7-2), CD40, CD40L (CD154), CD47, SIRPa, CD122, CD137, CD137L, OX40 (CD134), OX40L (CD252), BCMA (e.g., BCMA02), PSMA, CLDN18 (e.g., CLDN18.2), NKG2C, 4-1BB, LIGHT, PVRIG, SLAMF7, HVEM, BAFFR, ICAM-1, 2B4, LFA-1, GITR, ICOS (CD278), ICOSLG (CD275), LAG3 (CD223), A2AR, B7-H3 (CD276), B7-H4 (VTCN1), B7-H5, BTLA (CD272), CD160, CTLA-4 (CD152), GPRC5D, IDO (e.g., IDO1, IDO2), ILT3, TDO, KIR, LAIR-1, NOX2, PD-1, PD-L1, PD-L2, TIM-3, VISTA, SIGLEC-7 (CD328), SIGLEC-9 (CD329), SIGLEC-15, TIGIT, PVR (CD155), TLR3, CLEC9A, DEC-205, STING, and TGFβ.

30. The antibody or antigen-binding fragment thereof of any one of the preceding claims, which is linked to one or more conjugate moieties.

31. The antibody or antigen-binding fragment thereof of claim 30, wherein the conjugate moiety comprises a clearance-modifying agent, a chemotherapeutic agent, a toxin, a radioactive isotope, a lanthanide, a detectable label, a DNA-alkylator, a topoisomerase inhibitor, a tubulin-binder, a purification moiety or other anticancer drugs.

32. The antibody or antigen-binding fragment thereof of claim 30 or 31, wherein the conjugate moiety is covalently attached either directly or via a linker.

33. A chimeric antigen receptor, comprising the antibody or antigen-binding fragment of any one of claims 1-29, a transmembrane region and an intracellular signal region.

34. The chimeric antigen receptor of claim 33, wherein the transmembrane region comprises a transmembrane region of CD3, CD4, CD8 or CD28.

35. The chimeric antigen receptor of claim 33, wherein the intracellular signal region is selected from the group consisting of: an intracellular signal regions sequence of CD3, Fc γ RI, CD27, CD28, CD137, CD134, MyD88, CD40, CD278, TLRs, or a combination thereof.

36. The chimeric antigen receptor of any one of claims 33-35, wherein the antigen-binding fragment is a scFv.

37. The chimeric antigen receptor of any one of claims 33-36, wherein the chimeric antigen receptor is grafted onto an allogeneic cell, an autologous cell or a xenogeneic cell.

38. The chimeric antigen receptor of any one of claims 33-37, wherein the chimeric antigen receptor is grafted onto an immune effector cell.

39. The chimeric antigen receptor of any one of claims 33-38, wherein the chimeric antigen receptor is grafted onto a T cell, a natural killer cell, a macrophage cell, or a tumor-infiltrating lymphocyte.

40. A pharmaceutical composition comprising the antibody or an antigen-binding fragment thereof of any one of claims 1-32, or the chimeric antigen receptor of any one of claims 33-39, and one or more pharmaceutically acceptable carriers.

41. An isolated polynucleotide encoding the antibody or antigen-binding fragment thereof of any one of claims 1-29, and/or the chimeric antigen receptor of any one of claims 33-39.

42. A vector comprising the isolated polynucleotide of claim 41.

43. A host expression system comprising the vector of claim 42 or having the polynucleotide of claim 41 integrated into genome thereof.

44. The host expression system of claim 43, which is a microorganism, a yeast, or a mammalian cell, optionally, wherein the microorganism is selected from the group consisting of E. coli and B. subtilis, optionally wherein the yeast is Saccharomyces, and optionally wherein the mammalian cell is selected from the group consisting of COS, CHO-S, CHO-K1, HEK-293, and 3T3 cells.

45. A virus comprising the vector of claim 42.

46. A kit comprising the antibody or antigen-binding fragment thereof of any one of claims 1-29 and/or the chimeric antigen receptor of any one of claims 33-39 and/or the pharmaceutical composition of claim 40, and a second therapeutic agent.

47. A method of expressing the antibody or antigen-binding fragment thereof of any one of claims 1-29 or the chimeric antigen receptor of any one of claims 33-39, comprising culturing the host expression system of claim 43 under the condition at which the antibody or antigen-binding fragment of any one of claims 1-29 or the chimeric antigen receptor of any one of claims 33-39 is expressed.

48. A method of treating, preventing or alleviating a disease, disorder or condition in a subject, comprising administering to the subject a therapeutically effective amount of the antibody or antigen-binding fragment thereof of any one of claims 1-32 and/or the chimeric antigen receptor of any one of claims 33-39 and/or the pharmaceutical composition of claim 40.

49. The method of claim 48, wherein the disease, disorder or condition is immune disease, inflammatory disease, cancer or neurological disease.

50. The method of claim 49, wherein the cancer is a solid tumor or hematologic tumor.

51. The method of any one of claims 48-50, wherein the disease, disorder or condition is selected from the group consisting of lung cancer (e.g., non-small-cell lung cancer (NSCLC), small cell lung cancer (SCLC), adenocarcinoma of the lung, or squamous cell carcinoma of the lung), peritoneal cancer, carcinoid cancer, bone cancer, pancreatic cancer, primitive neuroectodermal tumor, skin cancer, gallbladder cancer, cancer of the head or neck, squamous cell cancer, uterine cancer, ovarian cancer, rectal cancer, prostate cancer, bladder cancer (e.g., urothelial cancer), cancer of the anal region (e.g., anal squamous cell carcinoma), gastric or stomach cancer (e.g., gastrointestinal cancer), esophageal cancer, colon cancer, breast cancer, uterine cancer, liver cancer (e.g., hepatoblastoma, hepatocellular carcinoma/hepatoma, or hepatic carcinoma), cholangiocarcinoma, sarcoma, colorectal cancer, carcinoma of the fallopian tubes, salivary gland carcinoma, carcinoma of the cervix, endometrial or uterine carcinoma, osteosarcoma, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, cancer of the nasopharynx, sarcoma of soft tissue, polycythemia vera, cancer of the urethra, cancer of the penis, cancer of the kidney or ureter (e.g., rhabdoid tumor of the kidney), cutaneous T-cell lymphoma, medulloblastoma, nephroblastoma, myelodysplastic syndrome, chronic and non-chronic myeloproliferative disorder, choroid plexus papilloma, renal cell carcinoma, carcinoma of the renal pelvis, neoplasms of the central nervous system (CNS), soft tissue sarcoma (e.g., rhabdomyosarcoma, fibrosarcoma, Kaposi's sarcoma), spinal axis tumors, glioma (e.g., ependymoma, astrocytoma, anaplastic astrocytoma, oligodendroglioma, eye cancer (e.g., retinoblastoma), brain stem glioma, or mixed glioma such as oligoastrocytoma), brain tumor (e.g., glioblastoma/glioblastoma multiforme (GBM), non-glioblastoma brain tumor, or meningioma), melanoma (e.g., cutaneous or intraocular melanoma), thrombocythemia, mesothelioma, mycosis fungoides, Sezary syndrome, idiopathic myelofibrosis, solitary plasmacytoma, vestibular schwannoma, Ewing's sarcoma, chondrosarcoma, MYH associated polyposis, pituitary adenoma, pediatric cancers such as pediatric sarcomas (e.g., neuroblastoma, rhabdomyosarcoma, and osteosarcoma), hematological cancer, Hodgkin's lymphoma, non-Hodgkin's lymphoma, leukemia (e.g., lymphocytic/lymphoblastic leukemia), chronic or acute leukemia, mast cell leukemia, lymphocytic lymphomas, primary CNS lymphoma, chronic lymphocytic leukemia (CLL), acute lymphocytic leukemia (ALL), chronic myeloid leukemia (CML), acute myeloid leukemia (AML), chronic myelomonocytic leukemia (CMML), chronic lymphoblastic leukemia, acute lymphoblastic leukemia, hairy cell leukemia (HCL), Burkitt's lymphoma (BL), multiple myeloma (e.g., relapsed or refractory multiple myeloma), T or B cell lymphoma, mantle cell lymphoma (MCL) (e.g., relapsed or refractory mantle cell lymphoma), malignant melanoma, diffuse large B cell lymphoma (DLBCL), DLBCL that results from follicular lymphoma, high-grade B-cell lymphoma, primary mediastinal large B-cell lymphoma, follicular lymphoma (FL), and primary mediastinal B-cell lymphoma.

52. The method of any one of claims 48-51, wherein the subject is human.

53. The method of any one of claims 48-52, wherein the administration is through a parenteral route comprising subcutaneous, intraperitoneal, intravenous, intramuscular, or intradermal injection; or a non-parenteral route comprising transdermal, oral, intranasal, intraocular, sublingual, rectal, or topical.

54. The method of any one of claims 48-53, wherein the method further includes administering to the subject in need thereof an additional therapeutic agent.

55. The method of claim 54, wherein the additional therapeutic agent is selected from the group consisting of: an active agent, an imaging agent, a cytotoxic agent, an angiogenesis inhibitor, a kinase inhibitor, a co-stimulation molecule agonist, a co-inhibition molecule blocker, an adhesion molecule blocker, an anti-cytokine antibody or functional fragment thereof, a detectable label or reporter, an antimicrobial, a gene editing agent, a beta agonist, an viral RNA inhibitor, a polymerase inhibitor, an interferon, and a microRNA.

56. The method of claim 54, wherein the additional therapeutic agent is administered to the subject in need before, after or simultaneously with the antibody or antigen-binding fragment thereof of any one of claims 1-32 and/or the chimeric antigen receptor of any one of claims 33-39 and/or the pharmaceutical composition of claim 40.

57. A method of activating a CD3-expressing T cell in vivo or in vitro, comprising contacting the CD3-expressing T cell with the antibody or antigen-binding fragment thereof of any one of claims 1-32 and/or the chimeric antigen receptor of any one of claims 33-39 and/or the pharmaceutical composition of claim 40.

58. A method of modulating CD3 activity in a CD3-expressing cell, comprising exposing the CD3-expressing cell to the antibody or antigen-binding fragment thereof of any of claims 1-32 and/or the chimeric antigen receptor of any one of claims 33-39 and/or the pharmaceutical composition of claim 40.

59. A method of promoting in vivo or in vitro processing of a second antigen by a CD3-expressing T cell, comprising contacting the CD3-expressing T cell with the bispecific or multi-specific antibody or antigen-binding fragment thereof of any of claims 27-32, wherein the bispecific or multi-specific antibody or antigen-binding fragment thereof is capable of binding to both the CD3-expressing T cell and a second antigen thereby bringing both in close proximity.

60. A method of detecting presence or amount of CD3 in a sample, comprising contacting the sample with the antibody or antigen-binding fragment thereof of any of claims 1-32 and/or the chimeric antigen receptor of any one of claims 33-39 and/or the pharmaceutical composition of claim 40, and determining the presence or the amount of CD3 in the sample.

61. A method of diagnosing a CD3 related disease or condition in a subject, comprising: a) contacting a sample obtained from the subject with the antibody or antigen-binding fragment thereof of any one of claims 1-32 and/or the chimeric antigen receptor of any one of claims 33-39 and/or the pharmaceutical composition of claim 40; b) determining presence or amount of CD3 in the sample; and c) correlating the presence or the amount of CD3 to existence or status of the CD3 related disease or condition in the subject.

62. Use of the antibody or antigen-binding fragment thereof of any of claims 1-32 and/or the chimeric antigen receptor of any one of claims 33-39 and/or the pharmaceutical composition of claim 40 in the manufacture of a medicament for treating a CD3 related disease, disorder or condition in a subject.

63. Use of the antibody or antigen-binding fragment thereof of any of claims 1-32 and/or the chimeric antigen receptor of any one of claims 33-39 and/or the pharmaceutical composition of claim 40 in the manufacture of a diagnostic reagent for diagnosing a CD3 related disease, disorder or condition.

64. A kit comprising the antibody or antigen-binding fragment thereof of any of claims 1-32 and/or the chimeric antigen receptor of any one of claims 33-39 and/or the pharmaceutical composition of claim 40, useful in detecting CD3, optionally recombinant CD3, CD3 expressed on cell surface, or CD3-expressing cells.


SEQUENCE LISTING (245 sequences)

SEQ ID NO: 1 - protein, 5 aa, synthetic construct
SYVIY

SEQ ID NO: 2 - protein, 17 aa, synthetic construct
YINPYKDVTKYSEKFND

SEQ ID NO: 3 - protein, 11 aa, synthetic construct
AIYHDYDGFGY

SEQ ID NO: 4 - protein, 10 aa, synthetic construct
SASSSVTYIN

SEQ ID NO: 5 - protein, 7 aa, synthetic construct
DTSKLAS

SEQ ID NO: 6 - protein, 9 aa, synthetic construct
QQWSRNPLT

SEQ ID NO: 7 - protein, 120 aa, synthetic construct
EVQLQQSGPELVKPGASVKMSCKASGYKFTSYVIYWVKQKPGQGLEWIGYINPYKDVTKYSEKFNDKATLTSDKSSSTAYMEISSLTSEDSAVYYCARAIYHDYDGFGYWGQGTLVIVSA

SEQ ID NO: 8 - protein, 106 aa, synthetic construct
QIVLTQSPAIMSASPGEKVTMTCSASSSVTYINWYQQMSGTSPKRWIYDTSKLASGVPARFSGSGSGTSYSLTISSLEAEDAATYYCQQWSRNPLTFGAGTKLELK

SEQ ID NO: 9 - protein, 5 aa, synthetic construct
SYVIH

SEQ ID NO: 10 - protein, 17 aa, synthetic construct
YINPYNDVIKYNEKFKG

SEQ ID NO: 11 - protein, 11 aa, synthetic construct
DSYYSYDGFAY

SEQ ID NO: 12 - protein, 10 aa, synthetic construct
SASSSVSYMH

SEQ ID NO: 13 - protein, 7 aa, synthetic construct
DTSKLAS

SEQ ID NO: 14 - protein, 9 aa, synthetic construct
QQWSSNPPT

SEQ ID NO: 15 - protein, 120 aa, synthetic construct
EVQLQLSGPELVKPGASVKMSCKASGYTFSSYVIHWVKQKPGQGLEWIGYINPYNDVIKYNEKFKGKATLTSDKSSSTAYMELSSLTSEDSAVYYCARDSYYSYDGFAYWGQGTLVTVSA

SEQ ID NO: 16 - protein, 106 aa, synthetic construct
QIVLTQSPAIMSASPGEKVTMTCSASSSVSYMHWYQQKSGTSPKRWIYDTSKLASGVPARFSGSGSGTSYSLTISSMEAEDAATYYCQQWSSNPPTFGGGTKVEIK

SEQ ID NO: 17 - protein, 5 aa, synthetic construct
DYGMH

SEQ ID NO: 18 - protein, 17 aa, synthetic construct
YISSASGKIYYADTVKG

SEQ ID NO: 19 - protein, 12 aa, synthetic construct
VLRWSVNYVMDY

SEQ ID NO: 20 - protein, 17 aa, synthetic construct
ESSQSVLYSSNQKNYLA

SEQ ID NO: 21 - protein, 7 aa, synthetic construct
WASTRES

SEQ ID NO: 22 - protein, 8 aa, synthetic construct
HQYLSSWT

SEQ ID NO: 23 - protein, 121 aa, synthetic construct
EVQLVESGGGIVKPGGSRKLSCAASGFTFSDYGMHWVRQAPEKGLEWIAYISSASGKIYYADTVKGRFTISRDNAKNILFLQMTSLRSEDTAMYYCARVLRWSVNYVMDYWGQGTSVTVSS

SEQ ID NO: 24 - protein, 112 aa, synthetic construct
DIVMTQSPSSLAVSAGEKVTMNCESSQSVLYSSNQKNYLAWYQQKPGQSPKLLIYWASTRESGVPDRFTGTGSGTDFTLTISSVQAEDLAVYYCHQYLSSWTFGGGTKLEIK

SEQ ID NO: 25 - protein, 5 aa, synthetic construct
RYPIH

SEQ ID NO: 26 - protein, 17 aa, synthetic construct
YINPRNDYTKYTQRFKD

SEQ ID NO: 27 - protein, 10 aa, synthetic construct
SINDYDGFAY

SEQ ID NO: 28 - protein, 10 aa, synthetic construct
SASSSVSYMN

SEQ ID NO: 29 - protein, 7 aa, synthetic construct
DTSKLAS

SEQ ID NO: 30 - protein, 9 aa, synthetic construct
QQWSSNPLT

SEQ ID NO: 31 - protein, 119 aa, synthetic construct
EVQLQQSGAELARPGASVRMSCKASGYTFTRYPIHWVKQRPGQGLEWIGYINPRNDYTKYTQRFKDKATLTADKSSSTAYMQLSSLTSEDSAVYYCAKSINDYDGFAYWGQGTLVTVSA

SEQ ID NO: 32 - protein, 106 aa, synthetic construct
DIVLSQSPTILSASPGEKVTMTCSASSSVSYMNWYQQRSGTSPKRWIYDTSKLASGVPARFSGSGSGTSYSLTISSMEAEDAATYYCQQWSSNPLTFGAGTKLELK

SEQ ID NO: 33 - protein, 5 aa, synthetic construct
DFNIH

SEQ ID NO: 34 - protein, 17 aa, synthetic construct
YINPNDGGSTYNQKFKG

SEQ ID NO: 35 - protein, 5 aa, synthetic construct
PFLDS

SEQ ID NO: 36 - protein, 11 aa, synthetic construct
KASQDINSYLR

SEQ ID NO: 37 - protein, 7 aa, synthetic construct
GASSLVD

SEQ ID NO: 38 - protein, 9 aa, synthetic construct
LQYDEVPLT

SEQ ID NO: 39 - protein, 114 aa, synthetic construct
EVQLQQSGPELVKPGASVKMSCKASGYTFTDFNIHWVKQSLGKSLEWIGYINPNDGGSTYNQKFKGKATLTINKSSSSAYMELRSLTSEDSAVYYCAIPFLDSWGQGTSVTVSS

SEQ ID NO: 40 - protein, 107 aa, synthetic construct
TLMMTQSPSSMYASLGERVTITCKASQDINSYLRWYQQKPGKSPKTLIYGASSLVDGVPSRFSGRGSGQDYSLTISSLEYEDMGIYYCLQYDEVPLTFGAGTKLELK

SEQ ID NO: 41 - protein, 5 aa, synthetic construct
DYYMN

SEQ ID NO: 42 - protein, 17 aa, synthetic construct
DINPIHGGISSNQKFSD

SEQ ID NO: 43 - protein, 12 aa, synthetic construct
RIYYGYDWYFDV

SEQ ID NO: 44 - protein, 12 aa, synthetic construct
SASSSVTSRYFF

SEQ ID NO: 45 - protein, 7 aa, synthetic construct
STSNLAS

SEQ ID NO: 46 - protein, 9 aa, synthetic construct
QQYSTSPST

SEQ ID NO: 47 - protein, 121 aa, synthetic construct
EVQLQQSGPEVVKPGASVKISCKASGYTFTDYYMNWVKQSHGKNLEWIGDINPIHGGISSNQKFSDKATLTVDKSSNTAYLELRSLTSEDSAIYYCARRIYYGYDWYFDVWGTGTTVTVSS

SEQ ID NO: 48 - protein, 108 aa, synthetic construct
DIVLSQSPAIMSASPGERVTMTCSASSSVTSRYFFWYQQKPGSSPKLWIYSTSNLASGVPVRFSGSGSGTSYSLTINNMAAEDAATYYCQQYSTSPSTFGAGTKLELK

SEQ ID NO: 49 - protein, 5 aa, synthetic construct
GYFMN

SEQ ID NO: 50 - protein, 17 aa, synthetic construct
RFNPYNGDTFYNQNFKG

SEQ ID NO: 51 - skipped

SEQ ID NO: 52 - protein, 16 aa, synthetic construct
KSSQSLLDSDGKTYLN

SEQ ID NO: 53 - protein, 7 aa, synthetic construct
LVSKLDS

SEQ ID NO: 54 - protein, 9 aa, synthetic construct
WQGTHFPFT

SEQ ID NO: 55 - protein, 112 aa, synthetic construct
EVQLQQSGPELVKPGASVKISCKASGYSFTGYFMNWVKQSHGKSLEWIGRFNPYNGDTFYNQNFKGKATLTVDKSSSTAHMELRSLTSEDSAVYYCARGDYWGQGTTLTVSS

SEQ ID NO: 56 - protein, 112 aa, synthetic construct
DIQMTQSPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPFTFGSGTKLEIK

SEQ ID NO: 57 - protein, 5 aa, synthetic construct
SYPMS

SEQ ID NO: 58 - protein, 17 aa, synthetic construct
TISSGGGNTYYPDSVKG

SEQ ID NO: 59 - protein, 8 aa, synthetic construct
GLYWYFDV

SEQ ID NO: 60 - protein, 16 aa, synthetic construct
RSSQSIAHSNGDTYLE

SEQ ID NO: 61 - protein, 7 aa, synthetic construct
KVSNRIS

SEQ ID NO: 62 - protein, 9 aa, synthetic construct
FQGSHVPYT

SEQ ID NO: 63 - protein, 117 aa, synthetic construct
EVKLVESGGGLVKPGGSLKLSCAASGFTFSSYPMSWVRQTPAKRLEWVATISSGGGNTYYPDSVKGRFTISRDNARNTLYLQMSSLRSEDTAMYYCARGLYWYFDVWGAGTTVTVSS

SEQ ID NO: 64 - protein, 112 aa, synthetic construct
DIVLTQSPLSLPVSLGDQASISCRSSQSIAHSNGDTYLEWYLQNPGQSPKLLIYKVSNRISGVPDRFSGSGSGTDFTLKISRVEAEDLGVYYCFQGSHVPYTFGGGTKLEIK

SEQ ID NO: 65 - protein, 5 aa, synthetic construct
SYGVS

SEQ ID NO: 66 - protein, 16 aa, synthetic construct
VIWGDGNTNYNSDLKT

SEQ ID NO: 67 - protein, 8 aa, synthetic construct
VSYWYFDV

SEQ ID NO: 68 - protein, 11 aa, synthetic construct
RASENIYSYLA

SEQ ID NO: 69 - protein, 7 aa, synthetic construct
NAKTLPE

SEQ ID NO: 70 - protein, 9 aa, synthetic construct
QHHYGTPPT

SEQ ID NO: 71 - protein, 116 aa, synthetic construct
EVQLVESRPGLVAPSQSLSITCTVSGFSLTSYGVSWVRQPPGKGLEWLGVIWGDGNTNYNSDLKTSLSISKDNSKSQVFLKLDSLQTDDTATYYCAKVSYWYFDVWGTGTTVTVSS

SEQ ID NO: 72 - protein, 107 aa, synthetic construct
DIQLTQPPASLSASVGETVTITCRASENIYSYLAWYQQKQGKSPQLLVYNAKTLPEGVPSRFSGSGSGTQFSLKINSLQPEDFGTYYCQHHYGTPPTFGTGTKLELK

SEQ ID NO: 73 - protein, 5 aa, synthetic construct
TYGMH

SEQ ID NO: 74 - protein, 17 aa, synthetic construct
YISNGGSTIYYADSVKG

SEQ ID NO: 75 - protein, 8 aa, synthetic construct
PTWGGFAY

SEQ ID NO: 76 - protein, 16 aa, synthetic construct
RSSQSIAHSNGDTYLE

SEQ ID NO: 77 - protein, 7 aa, synthetic construct
KVSNRIS

SEQ ID NO: 78 - protein, 9 aa, synthetic construct
FQGSHVPYT

SEQ ID NO: 79 - protein, 117 aa, synthetic construct
EVHLVESGGGLVQPGGSRKLSCAGSGFTFSTYGMHWVRQAPEKGLEWVAYISNGGSTIYYADSVKGRFTISRDNPNDTLFLHMTSLGSEDTAMYYCARPTWGGFAYWGQGTLVTVSA

SEQ ID NO: 80 - protein, 112 aa, synthetic construct; identical to SEQ ID NO: 64

SEQ ID NO: 81 - protein, 5 aa, synthetic construct
SYTMS

SEQ ID NO: 82 - protein, 17 aa, synthetic construct
TISSGGGNTFYADSVKG

SEQ ID NO: 83 - protein, 19 aa, synthetic construct
SLYYYDGTYVRKDYYAMDY

SEQ ID NO: 84 - protein, 11 aa, synthetic construct
RASENIFSNLA

SEQ ID NO: 85 - protein, 7 aa, synthetic construct
AATNLAE

SEQ ID NO: 86 - protein, 9 aa, synthetic construct
QQFWGTPWT

SEQ ID NO: 87 - protein, 128 aa, synthetic construct
EVKLVESGGGLVKPGGSLKLSCAASGFTFSSYTMSWIRQTPAKRLEWVATISSGGGNTFYADSVKGRFTISRDNARNTLNLQMSSLRSEDTAMYYCARSLYYYDGTYVRKDYYAMDYWGQGSSVTVSS

SEQ ID NO: 88 - protein, 107 aa, synthetic construct
DIQMTQSPASLSVSVGETVTISCRASENIFSNLAWYQQKQGKSPQLLVYAATNLAEGVPSRFSGSRSGTQFSLKINSLQSEDFGSYYCQQFWGTPWTFGGGTKLEVK

SEQ ID NO: 89 - protein, 5 aa, synthetic construct
DYYMN

SEQ ID NO: 90 - protein, 17 aa, synthetic construct
EINPNNGGTTYRQTFKG

SEQ ID NO: 91 - skipped

SEQ ID NO: 92 - protein, 16 aa, synthetic construct
RSSQSIVHSNGNTYLE

SEQ ID NO: 93 - protein, 7 aa, synthetic construct
KVSNRFS

SEQ ID NO: 94 - protein, 9 aa, synthetic construct
FQGSHVPRT

SEQ ID NO: 95 - protein, 112 aa, synthetic construct
EVQLQQSGPELVKPGASVKISCKASGNTFTDYYMNWVKQSHGKSLEWIGEINPNNGGTTYRQTFKGKATLTVDKSSTTAYMELRSLTSEDSAVYYCVLSGYWGQGTGLTVSS

SEQ ID NO: 96 - protein, 112 aa, synthetic construct
DIVMTQSPLSLPVSLGDQVAISCRSSQSIVHSNGNTYLEWYLQKPGQSPKLLIYKVSNRFSGVPDRFSGSGSGTDFTLKISRVEAEDLGVYYCFQGSHVPRTFGGGTKLELK

SEQ ID NO: 97 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 226 aa, synthetic construct
KTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 226 aa, synthetic construct
KTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 100 - protein, 119 aa, synthetic construct [sequence omitted]

SEQ ID NO: 101 - protein, 109 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRAD

SEQ ID NO: 102 - protein, 5 aa, synthetic construct
SYWMH

SEQ ID NO: 103 - protein, 17 aa, synthetic construct
NINPSNGDTNYNEKFKS

SEQ ID NO: 104 - protein, 10 aa, synthetic construct
DRSSQYYFDY

SEQ ID NO: 105 - protein, 17 aa, synthetic construct
KSSQSLLNSRTRKNYLA

SEQ ID NO: 106 - protein, 7 aa, synthetic construct
WASTRES

SEQ ID NO: 107 - protein, 8 aa, synthetic construct
KQSYNLRT

SEQ ID NO: 108 - protein, 119 aa, synthetic construct
QVQLQQPGTELVKPGASVKLSCKASGYTFTSYWMHWVKQRPGQGLEWIGNINPSNGDTNYNEKFKSKATLTVDKSSSTAYMQLSSLTSEDSAVYYCARDRSSQYYFDYWGQGTTLTVSS

SEQ ID NO: 109 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLRTFGGGTKLEIK

SEQ ID NO: 110 - protein, 5 aa, synthetic construct
SYWMH

SEQ ID NO: 111 - protein, 17 aa, synthetic construct
NINPSNGDTNYNEKFKS

SEQ ID NO: 112 - protein, 10 aa, synthetic construct
DRSSQYYFDY

SEQ ID NO: 113 - protein, 17 aa, synthetic construct
KSSQSLLNSRTRKNYLA

SEQ ID NO: 114 - protein, 7 aa, synthetic construct
WASTRES

SEQ ID NO: 115 - protein, 8 aa, synthetic construct
KQSFILRT

SEQ ID NO: 116 - protein, 119 aa, synthetic construct
QVQLQQPGTELVKPGASVKLSCKASGYTFTSYWMHWVKQRPEQGLEWIGNINPSNGDTNYNEKFKSKATLTVDKASNTAYMQLSSLTSEDSAVYYCGRDRSSQYYFDYWGQGTTLTVSS

SEQ ID NO: 117 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYFCKQSFILRTFGGGTKLEIK

SEQ ID NO: 118 - protein, 5 aa, synthetic construct
SYWMH

SEQ ID NO: 119 - protein, 17 aa, synthetic construct
NIYPGSSSINYNEKFKS

SEQ ID NO: 120 - protein, 10 aa, synthetic construct
DRSSQYYFDY

SEQ ID NO: 121 - protein, 17 aa, synthetic construct
KSSQSLLNSRTRKNYLA

SEQ ID NO: 122 - protein, 7 aa, synthetic construct
WASTRES

SEQ ID NO: 123 - protein, 8 aa, synthetic construct
TQSYNLRT

SEQ ID NO: 124 - protein, 119 aa, synthetic construct
QVQLQQPGTELVKPGASVKLSCKASGYTFTSYWMHWVKQRPGEGLEWIGNIYPGSSSINYNEKFKSKATLTVDKSSSTAYMQLSSLTSEDSAVYYCARDRSSQYYFDYWGQGTTLTVSS

SEQ ID NO: 125 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCTQSYNLRTFGGGTKLEIK

SEQ ID NO: 126 - protein, 5 aa, synthetic construct
SYWMH

SEQ ID NO: 127 - protein, 17 aa, synthetic construct
NINPSNGDTNFNEKFKS

SEQ ID NO: 128 - protein, 10 aa, synthetic construct
DRSSQYYFDY

SEQ ID NO: 129 - protein, 17 aa, synthetic construct
KSSQSLLNSRTRKNYLA

SEQ ID NO: 130 - protein, 7 aa, synthetic construct
WASTRES

SEQ ID NO: 131 - protein, 8 aa, synthetic construct
KQSYTLRT

SEQ ID NO: 132 - protein, 119 aa, synthetic construct
QVQLQQPGTELVKPGASVKLSCKASGYTFTSYWMHWVKQRPGQGLEWIGNINPSNGDTNFNEKFKSKATLTVDKSSSTAYMQLSSLTSEDSAVYYCARDRSSQYYFDYWGQGTTLTVSS

SEQ ID NO: 133 - protein, 112 aa, synthetic construct
DIVMSQSPSSLAVSAGEKVTMSCNSSQSLLNSRTRKNYLAWYQQKPGQSPKLLIYWASTRESGVPDRFTGSGSGTDFTLTISSVQAEDLAVYYCKQSYTLRTFGGGTKLEIK

SEQ ID NO: 134 - protein, 5 aa, synthetic construct
SYWMH

SEQ ID NO: 135 - protein, 17 aa, synthetic construct
NINPSNGESNYNEKFKS

SEQ ID NO: 136 - protein, 10 aa, synthetic construct
DRSSQYYFDY

SEQ ID NO: 137 - protein, 17 aa, synthetic construct
KSSQSLLNSRTRKNYLA

SEQ ID NO: 138 - protein, 7 aa, synthetic construct
WASTRES

SEQ ID NO: 139 - protein, 8 aa, synthetic construct
KQSYTLRT

SEQ ID NO: 140 - protein, 119 aa, synthetic construct
QVQLQQPGTELVKPGASVKLSCKASGYTFTSYWMHWVKQRPGQGLEWIGNINPSNGESNYNEKFKSKATLTVDKSSSTAYMQLSSLTSEDYAVYYCARDRSSQYYFDYWGQGTTLTVSS

SEQ ID NO: 141 - protein, 112 aa, synthetic construct
DIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYTLRTFGGGTKLEIK

SEQ ID NO: 142 - protein, 5 aa, synthetic construct
SYWMH

SEQ ID NO: 143 - protein, 17 aa, synthetic construct
NLNPSNGEINYNEKFKH

SEQ ID NO: 144 - protein, 10 aa, synthetic construct
DRSSQYYFDY

SEQ ID NO: 145 - protein, 17 aa, synthetic construct
KSSQSLLNSRTRKNYLA

SEQ ID NO: 146 - protein, 7 aa, synthetic construct
WASTRES

SEQ ID NO: 147 - protein, 8 aa, synthetic construct
KQSYILRT

SEQ ID NO: 148 - protein, 119 aa, synthetic construct
QVQLQQPGTELVKPGASVKLSCKASGYTFDSYWMHWVKQRPGQGLEWIGNLNPSNGEINYNEKFKHKATLTVDKSSSTAYMQLSSLTSEDSAVYYCSRDRSSQYYFDYWGQGTTLTVSS

SEQ ID NO: 149 - protein, 112 aa, synthetic construct
DIVMSQSPSSLAVSAGEKVTLSCKSSQSLLNSRTRKNYLAWYQQKSGQSPKLLMYWASTRESGVPDRFTGSGSGTDFTLTITNVQAEDLAVYYCKQSYILRTFGGGTKLEIK

SEQ ID NO: 150 - protein, 5 aa, synthetic construct
SYWMH

SEQ ID NO: 151 - protein, 17 aa, synthetic construct
NINPSNGATNYNEKFKS

SEQ ID NO: 152 - protein, 10 aa, synthetic construct
DRSSQYYFDY

SEQ ID NO: 153 - protein, 17 aa, synthetic construct
KSSQSLLNSRSRKNYLA

SEQ ID NO: 154 - protein, 7 aa, synthetic construct
WASTRES

SEQ ID NO: 155 - protein, 8 aa, synthetic construct
TQSYTLRT

SEQ ID NO: 156 - protein, 119 aa, synthetic construct
QVQLQQPGTELVKPGTSVKLSCKASGYTFTSYWMHWVKQRPGQGLEWIGNINPSNGATNYNEKFKSKATLTVDKSSSTAYMQLSSLTSEDSAVYYCARDRSSQYYFDYWGQGTTLTVSS

SEQ ID NO: 157 - protein, 112 aa, synthetic construct
DIVMSQSPSSLAVSAGEKVTMSCKSSQSLLNSRSRKNYLAWYQQKPGQSPKLLIYWASTRESGVPDRFTGSGSGTDFTLTISSVQAEDLAVYYCTQSYTLRTFGGGTKLEIK

SEQ ID NO: 158 - protein, 125 aa, synthetic construct
EVQLVESGGGLVQPGGSLRLSCAASGFTFNTYAMNWVRQAPGKGLEWVARIRSKYNNYATYYAASVKGRFTISRDDSKNSLYLQMNSLKTEDTAVYYCARHGNFGNSYVSWFAYWGQGTLVTVSS

SEQ ID NO: 159 - protein, 109 aa, synthetic construct
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 160 - protein, 125 aa, synthetic construct
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 161 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 162 - protein, 11 aa, synthetic construct
  SITE 1: note = X = D or E or R
  SITE 2: note = X = S or R
  SITE 3: note = X = Y or F
  SITE 4: note = X = Y or S
  SITE 7: note = X = D or E or R
  SITE 8: note = X = G or A
XXXXSYXXFAY

SEQ ID NO: 163 - protein, 120 aa, synthetic construct
EVQLVQSGAEVKKPGSSVKVSCKASGGTFSSYVIHWVRQAPGQGLEWMGYINPYNDVIKYNEKFKGRVTITADKSTSTAYMELSSLRSEDTAVYYCARDSYYSYDGFAYWGQGTLVTVSS

SEQ ID NO: 164 - protein, 120 aa, synthetic construct
EVQLVQSGAEVKKPGSSVKVSCKASGYTFSSYVIHWVRQAPGQGLEWMGYINPYNDVIKYNEKFKGRVTITADKSTSTAYMELSSLRSEDTAVYYCARDSYYSYDGFAYWGQGTLVTVSS

SEQ ID NO: 165 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYDGFAYWGQGTLVTVSS

SEQ ID NO: 166 - protein, 120 aa, synthetic construct
EVQLVQSGAEVKKPGSSVKVSCKASGYTFSSYVIHWVRQAPGQGLEWIGYINPYNDVIKYNEKFKGRATLTSDKSTSTAYMELSSLRSEDTAVYYCARDSYYSYDGFAYWGQGTLVTVSS

SEQ ID NO: 167 - protein, 9 aa, synthetic construct
  SITE 4: note = X = S or R
  SITE 6: note = X = N or S or R or W
QQWXSXPPT

SEQ ID NO: 168 - protein, 106 aa, synthetic construct
EIVLTQSPDFQSVTPKEKVTITCSASSSVSYMHWYQQKPDQSPKLLIKDTSKLASGVPSRFSGSGSGTDFTLTINSLEAEDAATYYCQQWSSNPPTFGGGTKVEIK

SEQ ID NO: 169 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWSSNPPTFGGGTKVEIK

SEQ ID NO: 170 - protein, 106 aa, synthetic construct
EIVLTQSPDFQSVTPKEKVTITCSASSSVSYMHWYQQKPDQSPKRWIYDTSKLASGVPSRFSGSGSGTDYTLTINSLEAEDAATYYCQQWSSNPPTFGGGTKVEIK

SEQ ID NO: 171 - protein, 17 aa, synthetic construct
YINPYSDVIKYNEKFKG

SEQ ID NO: 172 - protein, 17 aa, synthetic construct
YINPGNDVIKYNEKFKG

SEQ ID NO: 173 - protein, 17 aa, synthetic construct
YINPYNGVIKYNEKFKG

SEQ ID NO: 174 - protein, 11 aa, synthetic construct
ESYYSYDGFAY

SEQ ID NO: 175 - protein, 11 aa, synthetic construct
DSFYSYDGFAY

SEQ ID NO: 176 - protein, 11 aa, synthetic construct
DSYYSYEGFAY

SEQ ID NO: 177 - protein, 11 aa, synthetic construct
DSYYSYDAFAY

SEQ ID NO: 178 - protein, 11 aa, synthetic construct
RSYYSYDGFAY

SEQ ID NO: 179 - protein, 11 aa, synthetic construct
DRYYSYDGFAY

SEQ ID NO: 180 - protein, 11 aa, synthetic construct
DSYSSYDGFAY

SEQ ID NO: 181 - protein, 11 aa, synthetic construct
DSYYSYRGFAY

SEQ ID NO: 182 - protein, 9 aa, synthetic construct
QQWSSSPPT

SEQ ID NO: 183 - protein, 9 aa, synthetic construct
QQWRSNPPT

SEQ ID NO: 184 - protein, 9 aa, synthetic construct
QQWSSRPPT

SEQ ID NO: 185 - protein, 9 aa, synthetic construct
QQWSSWPPT

SEQ ID NO: 186 - protein, 120 aa, synthetic construct
EVQLVQSGAEVKKPGSSVKVSCKASGYTFSSYVIHWVRQAPGQGLEWMGYINPYSDVIKYNEKFKGRVTLTSDKSTSTAYMELSSLRSEDTAVYYCARDSYYSYDGFAYWGQGTLVTVSS

SEQ ID NO: 187 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARESYYSYDGFAYWGQGTLVTVSS

SEQ ID NO: 188 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSFYSYDGFAYWGQGTLVTVSS

SEQ ID NO: 189 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYEGFAYWGQGTLVTVSS

SEQ ID NO: 190 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYDAFAYWGQGTLVTVSS

SEQ ID NO: 191 - protein, 120 aa, synthetic construct
EVQLVQSGAEVKKPGSSVKVSCKASGYTFSSYVIHWVRQAPGQGLEWMGYINPGNDVIKYNEKFKGRVTLTSDKSTSTAYMELSSLRSEDTAVYYCARDSYYSYDGFAYWGQGTLVTVSS

SEQ ID NO: 192 - protein, 120 aa, synthetic construct
EVQLVQSGAEVKKPGSSVKVSCKASGYTFSSYVIHWVRQAPGQGLEWMGYINPYNGVIKYNEKFKGRVTLTSDKSTSTAYMELSSLRSEDTAVYYCARDSYYSYDGFAYWGQGTLVTVSS

SEQ ID NO: 193 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARRSYYSYDGFAYWGQGTLVTVSS

SEQ ID NO: 194 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDRYYSYDGFAYWGQGTLVTVSS

SEQ ID NO: 195 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYSSYDGFAYWGQGTLVTVSS

SEQ ID NO: 196 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYRGFAYWGQGTLVTVSS

SEQ ID NO: 197 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWSSSPPTFGGGTKVEIK

SEQ ID NO: 198 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWRSNPPTFGGGTKVEIK

SEQ ID NO: 199 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWSSRPPTFGGGTKVEIK

SEQ ID NO: 200 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWSSWPPTFGGGTKVEIK

SEQ ID NO: 201 - protein, 17 aa, synthetic construct
  SITE 5: note = X = Y or G
  SITE 6: note = X = N or S
  SITE 7: note = X = D or G
YINPXXXVIKYNEKFKG

SEQ ID NO: 202 - protein, 119 aa, synthetic construct
EVQLVQSGAEVKKPGSSVKVSCKASGYTFTSYWMHWVRQAPGQGLEWMGNINPSNGATNYNEKFKSRVTITADKSTSTAYMELSSLRSEDTAVYYCARDRSSQYYFDYWGQGTTVTVSS

SEQ ID NO: 203 - protein, 119 aa, synthetic construct
EVQLVQSGAEVKKPGSSVKVSCKASGYTFTSYWMHWVRQAPGQGLEWMGNINPSNGATNYNEKFKSRVTLTVDKSTSTAYMELSSLRSEDTAVYYCARDRSSQYYFDYWGQGTTVTVSS

SEQ ID NO: 204 - protein, 119 aa, synthetic construct
EVQLVQSGAEVKKPGSSVKVSCKASGYTFTSYWMHWVRQAPGQGLEWMGNINPSQGATNYNEKFKSRVTLTVDKSTSTAYMELSSLRSEDTAVYYCARDRSSQYYFDYWGQGTTVTVSS

SEQ ID NO: 205 - protein, 119 aa, synthetic construct
EVQLVQSGAEVKKPGSSVKVSCKASGYTFTSYWMHWVRQAPGQGLEWMGNINPSSGATNYNEKFKSRVTLTVDKSTSTAYMELSSLRSEDTAVYYCARDRSSQYYFDYWGQGTTVTVSS

SEQ ID NO: 206 - protein, 119 aa, synthetic construct
EVQLVQSGAEVKKPGSSVKVSCKASGYTFTSYWMHWVRQAPGQGLEWIGNINPSNGATNYNEKFKSRATLTVDKSTSTAYMELSSLRSEDTAVYYCARDRSSQYYFDYWGQGTTVTVSS

SEQ ID NO: 207 - protein, 112 aa, synthetic construct
DIVMTQSPDSLAVSLGERATINCKSSQSLLNSRSRKNYLAWYQQKPGQPPKLLIYWASTRESGVPDRFSGSGSGTDFTLTISSLQAEDVAVYYCTQSYTLRTFGGGTKVEIK

SEQ ID NO: 208 - protein, 112 aa, synthetic construct
DIVMTQSPDSLAVSLGERATINCKSSQSLLNSRSRKNYLAWYQQKPGQSPKLLIYWASTRESGVPDRFSGSGSGTDFTLTISSLQAEDVAVYYCTQSYTLRTFGGGTKVEIK

SEQ ID NO: 209 - protein, 112 aa, synthetic construct
DIQMTQSPSSLSASVGDRVTITCKSSQSLLNSRSRKNYLAWYQQKPGKAPKLLIYWASTRESGVPSRFSGSGSGTDFTLTISSLQPEDFATYYCTQSYTLRTFGGGTKVEIK

SEQ ID NO: 210 - protein, 17 aa, synthetic construct
NINPSQGATNYNEKFKS

SEQ ID NO: 211 - protein, 17 aa, synthetic construct
NINPSSGATNYNEKFKS

SEQ ID NO: 212 - protein, 17 aa, synthetic construct
  SITE 6: note = X= Q, S or N
NINPSXGATNYNEKFKS

SEQ ID NO: 213 - protein, 119 aa, synthetic construct
EVQLVQSGAEVKKPGSSVKVSCKASGGTFSSYWMHWVRQAPGQGLEWMGNINPSNGATNYNEKFKSRVTITADKSTSTAYMELSSLRSEDTAVYYCARDRSSQYYFDYWGQGTTVTVSS

SEQ ID NO: 214 - protein, 9 aa, synthetic construct
QQWRSRPPT

SEQ ID NO: 215 - protein, 9 aa, synthetic construct
QQWRSSPPT

SEQ ID NO: 216 - protein, 9 aa, synthetic construct
QQWRSWPPT

SEQ ID NO: 217 - protein, 5 aa, synthetic construct
DYVIH

SEQ ID NO: 218 - protein, 5 aa, synthetic construct
SYYIH

SEQ ID NO: 219 - skipped

SEQ ID NO: 220 - protein, 17 aa, synthetic construct
YINPGSGVIKYNEKFKG

SEQ ID NO: 221 - protein, 11 aa, synthetic construct
ESFSSYDGFAY

SEQ ID NO: 222 - protein, 17 aa, synthetic construct
YINPGNGVIKYNEKFKG

SEQ ID NO: 223 - protein, 11 aa, synthetic construct
ESYSSYDGFAY

SEQ ID NO: 224 - protein, 11 aa, synthetic construct
ESFYSYDGFAY

SEQ ID NO: 225 - protein, 11 aa, synthetic construct
DSYYSYEAFAY

SEQ ID NO: 226 - protein, 11 aa, synthetic construct
DSYYSYRAFAY

SEQ ID NO: 227 - protein, 11 aa, synthetic construct
DDYYSYDGFAY

SEQ ID NO: 228 - protein, 11 aa, synthetic construct
DGYYSYDGFAY

SEQ ID NO: 229 - protein, 11 aa, synthetic construct
GSYYSYDGFAY

SEQ ID NO: 230 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWRSRPPTFGGGTKVEIK

SEQ ID NO: 231 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWRSSPPTFGGGTKVEIK

SEQ ID NO: 232 - protein, 106 aa, synthetic construct
EIVLTQSPDFQSVTPKEKVTITCSASSSVSYMHWYQQKPDQSPKRLIYDTSKLASGVPSRFSGSGSGTDFTLTINSLEAEDAATYYCQQWRSWPPTFGGGTKVEIK

SEQ ID NO: 233 - protein, 120 aa, synthetic construct
EVQLVQSGAEVKKPGSSVKVSCKASGYTFSSYVIHWVRQAPGQGLEWMGYINPGSGVIKYNEKFKGRVTLTSDKSTSTAYMELSSLRSEDTAVYYCARDSYYSYDGFAYWGQGTLVTVSS

SEQ ID NO: 234 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARESFSSYDGFAYWGQGTLVTVSS

SEQ ID NO: 235 - protein, 120 aa, synthetic construct
EVQLVQSGAEVKKPGSSVKVSCKASGYTFSSYVIHWVRQAPGQGLEWMGYINPGNGVIKYNEKFKGRVTLTSDKSTSTAYMELSSLRSEDTAVYYCARDSYSSYDGFAYWGQGTLVTVSS

SEQ ID NO: 236 - protein, 120 aa, synthetic construct
EVQLVQSGAEVKKPGSSVKVSCKASGYTFSSYVIHWVRQAPGQGLEWMGYINPGNDVIKYNEKFKGRVTLTSDKSTSTAYMELSSLRSEDTAVYYCARESYSSYDGFAYWGQGTLVTVSS

SEQ ID NO: 237 - protein, 120 aa, synthetic construct
EVQLVQSGAEVKKPGSSVKVSCKASGYTFSSYVIHWVRQAPGQGLEWMGYINPYSDVIKYNEKFKGRVTLTSDKSTSTAYMELSSLRSEDTAVYYCARESFYSYDGFAYWGQGTLVTVSS

SEQ ID NO: 238 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYEAFAYWGQGTLVTVSS

SEQ ID NO: 239 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDSYYSYRAFAYWGQGTLVTVSS

SEQ ID NO: 240 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDDYYSYDGFAYWGQGTLVTVSS

SEQ ID NO: 241 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARDGYYSYDGFAYWGQGTLVTVSS

SEQ ID NO: 242 - protein, 120 aa, synthetic construct; identical to SEQ ID NO: 187

SEQ ID NO: 243 - protein, 120 aa, synthetic construct
EVQLVQSGAEVKKPGSSVKVSCKASGYTFSSYVIHWVRQAPGQGLEWMGYINPYNDVIKYNEKFKGRVTLTSDKSTSTAYMELSSLRSEDTAVYYCARGSYYSYDGFAYWGQGTLVTVSS

SEQ ID NO: 244 - protein, 120 aa, synthetic construct
EVQLVQSGAEVKKPGSSVKVSCKASGYTFSDYVIHWVRQAPGQGLEWMGYINPYNDVIKYNEKFKGRVTLTSDKSTSTAYMELSSLRSEDTAVYYCARDSYYSYDGFAYWGQGTLVTVSS

SEQ ID NO: 245 - protein, 120 aa, synthetic construct
EVQLVQSGAEVKKPGSSVKVSCKASGYTFSSYYIHWVRQAPGQGLEWMGYINPYNDVIKYNEKFKGRVTLTSDKSTSTAYMELSSLRSEDTAVYYCARDSYYSYDGFAYWGQGTLVTVSS